SEQUENCE LISTING (47 sequences)

SEQ ID NO: 1 - protein, 1273 aa, Artificial Sequence
  S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - RNA, 3819 nt, Artificial Sequence
  Coding Sequence
auguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauaaaguggaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacaca

SEQ ID NO: 3 - protein, 218 aa, Artificial Sequence
  S Protein RBD Fusion
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 4 - RNA, 654 nt, Artificial Sequence
  Coding Sequence
auguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaa

SEQ ID NO: 5 - protein, 266 aa, Artificial Sequence
  S Protein RBD Fusion
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 6 - RNA, 798 nt, Artificial Sequence
  Coding Sequence
auguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggc

SEQ ID NO: 7 - protein, 1273 aa, Artificial Sequence
  S Protein Variant
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - RNA, 3819 nt, Artificial Sequence
  Coding Sequence
auguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacaca

SEQ ID NO: 9 - RNA, 3819 nt, Artificial Sequence
  Coding Sequence
auguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacaca

SEQ ID NO: 10 - protein, 41 aa, Artificial Sequence
  Foldon Sequence
GSGYIPEAPRDGQAYVRKDGEWVLLSTFLGRSLEVLFQGPG

SEQ ID NO: 11 - RNA, 123 nt, Artificial Sequence
  Coding Sequence
ggaucugguuauauuccugaagcuccaagagaugggcaagcuuacguucguaaagauggcgaauggguauuacuuucuaccuuuuuaggccggucccuggaggugcuguuccagggccccggc

SEQ ID NO: 12 - RNA, 47 nt, Artificial Sequence
  5′-UTR
aacuaguauucuucugguccccacagacucagagagaacccgccacc

SEQ ID NO: 13 - RNA, 278 nt, Artificial Sequence
  3′-UTR
cugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacc

SEQ ID NO: 14 - RNA, 110 nt, Artificial Sequence
  A30L70
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 15 - RNA, 4282 nt, Artificial Sequence
  RBL063.1
gggcgaacuaguauucuucugguccccacagacucagagagaacccgccaccauguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 16 - RNA, 4282 nt, Artificial Sequence
  RBL063.2
gggcgaacuaguauucuucugguccccacagacucagagagaacccgccaccauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 17 - RNA, 1261 nt, Artificial Sequence
  RBL063.3
gggcgaacuaguauucuucugguccccacagacucagagagaacccgccaccauguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggcugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 18 - protein, 250 aa, Artificial Sequence
  S Protein RBD Fusion
VRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 19 - RNA, 4283 nt, Artificial Sequence
  RBP020.1
agaauaaacuaguauucuucugguccccacagacucagagagaacccgccaccauguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 20 - RNA, 4283 nt, Artificial Sequence
  RBP020.2
agaauaaacuaguauucuucugguccccacagacucagagagaacccgccaccauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 21 - RNA, 1262 nt, Artificial Sequence
  RBP020.3
agaauaaacuaguauucuucugguccccacagacucagagagaacccgccaccauguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggcugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 22 - protein, 1879 aa, Artificial Sequence
  Viral Protein
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQ

SEQ ID NO: 23 - protein, 613 aa, Artificial Sequence
  Viral Protein
RFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 24 - RNA, 11917 nt, Artificial Sequence
  RBS004.1
gaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgaaaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagagcuagucacugggcuagggcucacaggcgagcuggucgauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaggcuuuugcuugucaugcagguacucucagagcgcucauagccauuauaagaccuaaaaaggcagugcucugcggagaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacugguagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuaagcauguugaccaagaaagcaugucugcaucugaaucccggcggaaccugugucagcauagguuaugguuacgcugacagggccagcgaaagcaucauuggugcuauagcgcggcaguucaaguuuucccgaguaugcaaaccgaaauccucacuugaggagacggaaguucuguuuguauucauuggguacgaucgcaaggcccguacgcacaauccuuacaagcuaucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaaguuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgauucuucagugacagaaccugaugcagagcuggugagggugcaucccaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaggagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaagaagaagaagauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcuccagaaccagccuaguuuccaccccgccaggcgugaauagggugaucacuagagaggagcucgaagcgcuuaccccgucacgcacuccuagcaggucggucuccagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuaggcaaaccucuggcagcagacgaugaacaugaugaugacaggagaagggcauugcaugaggagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagacuaguauguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgcggccgcaugaauacagcagcaauuggcaagcugcuuacauagaacucgcggcgauuggcaugccgccuuaaaauuuuuauuuuauuuuuucuuuucuuuuccgaaucggauuuuguuuuuaauauuucaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 25 - RNA, 11917 nt, Artificial Sequence
  RBS004.2
gaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgaaaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagagcuagucacugggcuagggcucacaggcgagcuggucgauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaggcuuuugcuugucaugcagguacucucagagcgcucauagccauuauaagaccuaaaaaggcagugcucugcggagaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacugguagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuaagcauguugaccaagaaagcaugucugcaucugaaucccggcggaaccugugucagcauagguuaugguuacgcugacagggccagcgaaagcaucauuggugcuauagcgcggcaguucaaguuuucccgaguaugcaaaccgaaauccucacuugaggagacggaaguucuguuuguauucauuggguacgaucgcaaggcccguacgcacaauccuuacaagcuaucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaaguuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgauucuucagugacagaaccugaugcagagcuggugagggugcaucccaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaggagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaagaagaagaagauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcuccagaaccagccuaguuuccaccccgccaggcgugaauagggugaucacuagagaggagcucgaagcgcuuaccccgucacgcacuccuagcaggucggucuccagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuaggcaaaccucuggcagcagacgaugaacaugaugaugacaggagaagggcauugcaugaggagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagacuaguauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgcggccgcaugaauacagcagcaauuggcaagcugcuuacauagaacucgcggcgauuggcaugccgccuuaaaauuuuuauuuuauuuuuucuuuucuuuuccgaaucggauuuuguuuuuaauauuucaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 26 - RNA, 8896 nt, Artificial Sequence
  RBS004.3
gaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgaaaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagagcuagucacugggcuagggcucacaggcgagcuggucgauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaggcuuuugcuugucaugcagguacucucagagcgcucauagccauuauaagaccuaaaaaggcagugcucugcggagaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacugguagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuaagcauguugaccaagaaagcaugucugcaucugaaucccggcggaaccugugucagcauagguuaugguuacgcugacagggccagcgaaagcaucauuggugcuauagcgcggcaguucaaguuuucccgaguaugcaaaccgaaauccucacuugaggagacggaaguucuguuuguauucauuggguacgaucgcaaggcccguacgcacaauccuuacaagcuaucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaaguuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgauucuucagugacagaaccugaugcagagcuggugagggugcaucccaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaggagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaagaagaagaagauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcuccagaaccagccuaguuuccaccccgccaggcgugaauagggugaucacuagagaggagcucgaagcgcuuaccccgucacgcacuccuagcaggucggucuccagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuaggcaaaccucuggcagcagacgaugaacaugaugaugacaggagaagggcauugcaugaggagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagacuaguauguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggcugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgcggccgcaugaauacagcagcaauuggcaagcugcuuacauagaacucgcggcgauuggcaugccgccuuaaaauuuuuauuuuauuuuuucuuuucuuuuccgaaucggauuuuguuuuuaauauuucaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 27 - RNA, 9079 nt, Artificial Sequence
  RBS004.4
gaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgaaaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagagcuagucacugggcuagggcucacaggcgagcuggucgauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaggcuuuugcuugucaugcagguacucucagagcgcucauagccauuauaagaccuaaaaaggcagugcucugcggagaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacugguagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuaagcauguugaccaagaaagcaugucugcaucugaaucccggcggaaccugugucagcauagguuaugguuacgcugacagggccagcgaaagcaucauuggugcuauagcgcggcaguucaaguuuucccgaguaugcaaaccgaaauccucacuugaggagacggaaguucuguuuguauucauuggguacgaucgcaaggcccguacgcacaauccuuacaagcuaucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaaguuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgauucuucagugacagaaccugaugcagagcuggugagggugcaucccaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaggagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaagaagaagaagauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcuccagaaccagccuaguuuccaccccgccaggcgugaauagggugaucacuagagaggagcucgaagcgcuuaccccgucacgcacuccuagcaggucggucuccagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuaggcaaaccucuggcagcagacgaugaacaugaugaugacaggagaagggcauugcaugaggagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagacuaguauguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggaagcggcagcggaucugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgcggccgcaugaauacagcagcaauuggcaagcugcuuacauagaacucgcggcgauuggcaugccgccuuaaaauuuuuauuuuauuuuuucuuuucuuuuccgaaucggauuuuguuuuuaauauuucaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 28 - protein, 327 aa, Artificial Sequence
  Vaccine Antigen
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCCKFDEDDSEPVLKGVKLHYT

SEQ ID NO: 29 - protein, 311 aa, Artificial Sequence
  BNT162b3c
MFVFLVLLPLVSSQCVNLTVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCC

SEQ ID NO: 30 - RNA, 1397 nt, Artificial Sequence
  BNT162b3c
agaauaaacuaguauucuucugguccccacagacucagagagaacccgccaccauguuuguguuucuugugcugcugccucuugugucuucucagugugugaauuugacagugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggaagcggcagcggaucugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 31 - protein, 314 aa, Artificial Sequence
  BNT162b3d
MDWIWRILFLVGAATGAHSQMQVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCC

SEQ ID NO: 32 - RNA, 1406 nt, Artificial Sequence
  BNT162b3d
agaauaaacuaguauucuucugguccccacagacucagagagaacccgccaccauggauuggauuuggagaauccuguuccucgugggagccgcuacaggagcccacucccagaugcaggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggaagcggcagcggaucugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 33 - protein, 9 aa, Artificial Sequence
  Linker
GSPGSGSGS

SEQ ID NO: 34 - protein, 6 aa, Artificial Sequence
  Linker
GSGSGS

SEQ ID NO: 35 - protein, 26 aa, Artificial Sequence
  Sec
MRVMAPRTLILLLSGALALTETWAGS

SEQ ID NO: 36 - protein, 55 aa, Artificial Sequence
  MITD
IVGIVAGLAVLAVVVIGAVVATVMCRRKSSGGKGGSYSQAASSDSAQGSDVSLTA

SEQ ID NO: 37 - protein, 65 aa, Artificial Sequence
  P2P16
KKQYIKANSKFIGITELKKLGGGKRGGGKKMTNSVDDALINSTKIYSYFPSVISKVNQGAQGKKL

SEQ ID NO: 38 - protein, 10 aa, Artificial Sequence
  GS Linker 1
GGSGGGGSGG

SEQ ID NO: 39 - protein, 14 aa, Artificial Sequence
  GS Linker 2
GSSGGGGSPGGGSS

SEQ ID NO: 40 - protein, 15 aa, Artificial Sequence
  Helper epitope P2
QYIKANSKFIGITEL

SEQ ID NO: 41 - protein, 32 aa, Artificial Sequence
  Helper epitope P16
MTNSVDDALINSTKIYSYFPSVISKVNQGAQG

SEQ ID NO: 42 - protein, 1270 aa, Artificial Sequence
  Vaccine sequence
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 43 - RNA, 3816 nt, Artificial Sequence
  Coding sequence
auguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgugaucuccggcaccaauggcaccaagagauucgacaaccccgugcugcccuucaacgacgggguguacuuugccagcaucgagaaguccaacaucaucagaggcuggaucuucggcaccacacuggacagcaagacccagagccugcugaucgugaacaacgccaccaacguggucaucaaagugugcgaguuccaguucugcaacgaccccuuccuggaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaucgugagagagcccgaggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucgacgagguguucaaugccaccagauucgccucuguguacgccuggaaccggaagcggaucagcaauugcguggccgacuacuccgugcuguacaaccuggcccccuucuucaccuucaagugcuacggcguguccccuaccaagcugaacgaccugugcuucacaaacguguacgccgacagcuucgugauccggggagaugaagugcggcagauugccccuggacagacaggcaacaucgccgacuacaacuacaagcugcccgacgacuucaccggcugugugauugccuggaacagcaacaagcuggacuccaaagucagcggcaacuacaauuaccuguaccggcuguuccggaaguccaaucugaagcccuucgagcgggacaucuccaccgagaucuaucaggccggcaacaagccuuguaacggcguggccggcuucaacugcuacuucccacugcgguccuacagcuuuaggcccacauacggcgugggccaccagcccuacagagugguggugcugagcuucgaacugcugcaugccccugccacagugugcggcccuaagaaaagcaccaaucucgugaagaacaaaugcgugaacuucaacuucaacggccugaagggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccagggcgugaacuguaccgaagugcccguggccauucacgccgaucagcugacaccuacauggcggguguacuccaccggcagcaauguguuucagaccagagccggcugucugaucggagccgaguacgugaacaauagcuacgagugcgacauccccaucggcgcuggaaucugcgccagcuaccagacacagacaaagagccaccggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaaaagagcccugacagggaucgccguggaacaggacaagaacacccaagagguguucgcccaagugaagcagaucuacaagaccccuccuaucaaguacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaagggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccacaaugcccaggcacugaacacccuggucaagcagcuguccuccaaguucggcgccaucagcucugugcugaacgauaucuucagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugauga

SEQ ID NO: 44 - RNA, 4274 nt, Artificial Sequence
  Vaccine RNA
agaauaaacuaguauucuucugguccccacagacucagagagaacccgccaccauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgugaucuccggcaccaauggcaccaagagauucgacaaccccgugcugcccuucaacgacgggguguacuuugccagcaucgagaaguccaacaucaucagaggcuggaucuucggcaccacacuggacagcaagacccagagccugcugaucgugaacaacgccaccaacguggucaucaaagugugcgaguuccaguucugcaacgaccccuuccuggaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaucgugagagagcccgaggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucgacgagguguucaaugccaccagauucgccucuguguacgccuggaaccggaagcggaucagcaauugcguggccgacuacuccgugcuguacaaccuggcccccuucuucaccuucaagugcuacggcguguccccuaccaagcugaacgaccugugcuucacaaacguguacgccgacagcuucgugauccggggagaugaagugcggcagauugccccuggacagacaggcaacaucgccgacuacaacuacaagcugcccgacgacuucaccggcugugugauugccuggaacagcaacaagcuggacuccaaagucagcggcaacuacaauuaccuguaccggcuguuccggaaguccaaucugaagcccuucgagcgggacaucuccaccgagaucuaucaggccggcaacaagccuuguaacggcguggccggcuucaacugcuacuucccacugcgguccuacagcuuuaggcccacauacggcgugggccaccagcccuacagagugguggugcugagcuucgaacugcugcaugccccugccacagugugcggcccuaagaaaagcaccaaucucgugaagaacaaaugcgugaacuucaacuucaacggccugaagggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccagggcgugaacuguaccgaagugcccguggccauucacgccgaucagcugacaccuacauggcggguguacuccaccggcagcaauguguuucagaccagagccggcugucugaucggagccgaguacgugaacaauagcuacgagugcgacauccccaucggcgcuggaaucugcgccagcuaccagacacagacaaagagccaccggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaaaagagcccugacagggaucgccguggaacaggacaagaacacccaagagguguucgcccaagugaagcagaucuacaagaccccuccuaucaaguacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaagggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccacaaugcccaggcacugaacacccuggucaagcagcuguccuccaaguucggcgccaucagcucugugcugaacgauaucuucagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 45 - protein, 1270 aa, Artificial Sequence
  Vaccine sequence
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 46 - RNA, 3816 nt, Artificial Sequence
  Coding sequence
auguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgugaucuccggcaccaauggcaccaagagauucgacaaccccgugcugcccuucaacgacgggguguacuuugccagcaucgagaaguccaacaucaucagaggcuggaucuucggcaccacacuggacagcaagacccagagccugcugaucgugaacaacgccaccaacguggucaucaaagugugcgaguuccaguucugcaacgaccccuuccuggaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaucgugagagagcccgaggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucgacgagguguucaaugccaccagauucgccucuguguacgccuggaaccggaagcggaucagcaauugcguggccgacuacuccgugcuguacaaccuggcccccuucuucaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcaacaagccuuguaacggcguggccggcuucaacugcuacuucccacugcgguccuacagcuuuaggcccacauacggcgugggccaccagcccuacagagugguggugcugagcuucgaacugcugcaugccccugccacagugugcggcccuaagaaaagcaccaaucucgugaagaacaaaugcgugaacuucaacuucaacggccugaagggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccagggcgugaacuguaccgaagugcccguggccauucacgccgaucagcugacaccuacauggcggguguacuccaccggcagcaauguguuucagaccagagccggcugucugaucggagccgaguacgugaacaauagcuacgagugcgacauccccaucggcgcuggaaucugcgccagcuaccagacacagacaaagagccaccggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaaaagagcccugacagggaucgccguggaacaggacaagaacacccaagagguguucgcccaagugaagcagaucuacaagaccccuccuaucaaguacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaagggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccacaaugcccaggcacugaacacccuggucaagcagcuguccuccaaguucggcgccaucagcucugugcugaacgauaucuucagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugauga

SEQ ID NO: 47 - RNA, 4274 nt, Artificial Sequence
  Vaccine RNA
agaauaaacuaguauucuucugguccccacagacucagagagaacccgccaccauguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgugaucuccggcaccaauggcaccaagagauucgacaaccccgugcugcccuucaacgacgggguguacuuugccagcaucgagaaguccaacaucaucagaggcuggaucuucggcaccacacuggacagcaagacccagagccugcugaucgugaacaacgccaccaacguggucaucaaagugugcgaguuccaguucugcaacgaccccuuccuggaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaucgugagagagcccgaggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucgacgagguguucaaugccaccagauucgccucuguguacgccuggaaccggaagcggaucagcaauugcguggccgacuacuccgugcuguacaaccuggcccccuucuucaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcaacaagccuuguaacggcguggccggcuucaacugcuacuucccacugcgguccuacagcuuuaggcccacauacggcgugggccaccagcccuacagagugguggugcugagcuucgaacugcugcaugccccugccacagugugcggcccuaagaaaagcaccaaucucgugaagaacaaaugcgugaacuucaacuucaacggccugaagggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccagggcgugaacuguaccgaagugcccguggccauucacgccgaucagcugacaccuacauggcggguguacuccaccggcagcaauguguuucagaccagagccggcugucugaucggagccgaguacgugaacaauagcuacgagugcgacauccccaucggcgcuggaaucugcgccagcuaccagacacagacaaagagccaccggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaaaagagcccugacagggaucgccguggaacaggacaagaacacccaagagguguucgcccaagugaagcagaucuacaagaccccuccuaucaaguacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaagggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccacaaugcccaggcacugaacacccuggucaagcagcuguccuccaaguucggcgccaucagcucugugcugaacgauaucuucagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0226132 A1
(43) Pub. Date: Jul. 11, 2024

(54) RNA COMPOSITIONS COMPRISING A BUFFER SUBSTANCE AND METHODS FOR PREPARING, STORING AND USING THE SAME

(71) Applicants: BioNTech SE, Mainz (DE); BioNTech Delivery Technologies GmbH, Halle (DE)

(72) Inventors: Steffen Panzner, Halle (DE); Ulrich Kurt Blaschke, Mainz (DE); Jorrit-Jan Krijger, Mainz (DE); Charalampos Koutsoulas, Mainz (DE)

(21) Appl. No.: 18/554,947

(22) PCT Filed: Apr. 11, 2022
(86) PCT No.: PCT/EP22/59555

§ 371 Date: Oct. 11, 2023

(30) Foreign Application Priority Data
Apr. 12, 2021 (WO) PCT/EP21/59460
Dec. 30, 2021 (WO) PCT/EP21/87882

(51) Int. Cl. A61K 31/7105; A61K 9/51; A61K 47/10; A61K 47/18; A61K 47/22; A61K 47/26

(52) U.S. Cl. CPC A61K 31/7105; A61K 9/5123; A61K 47/10; A61K 47/18; A61K 47/183; A61K 47/22; A61K 47/26

(57) ABSTRACT

The present disclosure relates generally to the field of RNA compositions comprising a buffer substance, methods for preparing and storing such compositions, and the use of such compositions in therapy.

### TECHNICAL FIELD

The present disclosure relates generally to the field of RNA compositions comprising a buffer substance, methods for preparing and storing such compositions, and the use of such compositions in therapy.

### BACKGROUND

The use of a recombinant nucleic acid (such as DNA or RNA) for delivery of foreign genetic information into target cells is well known. The advantages of using RNA include transient expression and a non-transforming character. RNA does not need to enter the nucleus in order to be expressed and moreover cannot integrate into the host genome, thereby eliminating diverse risks such as oncogenesis.

A recombinant nucleic acid may be administered in naked form to a subject in need thereof; however, usually a recombinant nucleic acid is administered using a composition. For example, RNA may be delivered to a subject using different delivery vehicles, based mostly on cationic polymers or lipids which together with the RNA form nanoparticles. The nanoparticles are intended to protect the RNA from degradation, enable delivery of the RNA to the target site and facilitate cellular uptake and processing by the target cells. The efficiency of RNA delivery depends, in part, on the molecular composition of the nanoparticle and can be influenced by numerous parameters, including particle size, formulation, and charge or grafting with molecular moieties, such as polyethylene glycol (PEG) or other ligands. The fate of such nanoparticle formulations is controlled by diverse key-factors (e.g., size and size distribution of the nanoparticles; etc.). These factors are, e.g., referred to in the FDA “Liposome Drug Products Guidance” from 2018 as specific attributes which should be analyzed and specified. The limitations to the clinical application of current nanoparticle formulations may lie in the lack of homogeneous, pure and well-characterized nanoparticle formulations.

Nanoparticles comprising ionizable lipids may display advantages in terms of targeting and efficacy in comparison to other RNA nanoparticle products. However, it is challenging to obtain sufficient shelf life as required for regular pharmaceutical use. It is said that for stabilization, nanoparticles comprising ionizable lipids need to be frozen at much lower temperatures, such as −80° C., which poses substantial challenges on the cold chain, or they can only be stored above the freezing temperature, e.g. 5° C., where only limited stability can be obtained.

It is known that RNA in solution or in nanoparticles undergoes slow fragmentation. Furthermore, in the presence of phosphate buffered saline (PBS), RNA has the tendency to adopt a very stable folded form which is hardly accessible for translation. Both mechanisms, i.e., fragmentation and formation of this stable RNA fold (also called “light migrating species (LMS)”), are temperature dependent and result in loss of intact and accessible RNA thereby limiting the stability of the liquid product; however, they are essentially absent in the frozen state.

Thus, there remains a need in the art for (i) compositions which comprise ionizable lipids and RNA and which are stable and can be stored in a temperature range compliant to regular technologies in pharmaceutical practice, in particular at a temperature of about −25° C. or even in liquid form at temperatures between +2 and +20° C.; (ii) compositions which are ready to use; (iii) compositions which, preferably, can repeatably be frozen and thawed; and (iv) methods for preparing and storing such compositions. The present disclosure addresses these and other needs.

The inventors surprisingly found that the compositions and methods described herein fulfill the above-mentioned requirements. In particular, it is demonstrated that by using a specific buffer substance, in particular triethanolamine (TEA) and its protonated form, it is possible to prepare compositions which are stable and which can be stored in liquid form.

### SUMMARY

In a first aspect, the present disclosure provides a composition comprising (i) RNA; (ii) a cationically ionizable lipid; and (iii) an aqueous phase, wherein the aqueous phase comprises a buffer system comprising a buffer substance having the formula N(R¹)(R²)(R³), its N-oxide, or a protonated form thereof, wherein: each of R¹, R², and R³ is independently selected from H, C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is H, CH(C₁₋₅ alkylene-R⁴)₂, or C(C₁₋₅ alkylene-R⁴)₃; or two of R¹, R², and R³ join together with the nitrogen atom to form a 5- or 6-membered N-heterocyclic ring which is optionally substituted with one or two R⁵; each R⁴ is independently selected from —OH, —O-(C₁₋₆ alkylene-OH), and —N(R⁶)_{z}-(C₁₋₆ alkylene-OH)_{2-z}, wherein each z is independently selected from 0 and 1; and each R⁶ is independently selected from H and C₁₋₃ alkyl; and each R⁵ is independently selected from C₁₋₆ alkyl, C₁₋₆ alkylene-R⁴, CH(C₁₋₅ alkylene-R⁴)₂, and C(C₁₋₅ alkylene-R⁴)₃.

As demonstrated in the present application, using a buffer system based on the particular buffer substances specified above, in particular TEA and its protonated form, instead of PBS in an RNA composition inhibits the formation of a very stable folded form (also called “light migrating species (LMS)” herein) of RNA. Furthermore, the present application demonstrates that, surprisingly, by using this buffer system, it is possible to obtain an RNA composition having improved RNA integrity after storage in liquid form for about 3 months. Thus, the claimed composition provides improved stability, can be stored in a temperature range compliant to regular technologies in pharmaceutical practice, and provides a ready-to-use composition.

In a second aspect, the present disclosure provides a composition comprising (i) RNA; and (ii) an aqueous phase, wherein the aqueous phase comprises a buffer system comprising a buffer substance having the formula N(R¹)(R²)(R³), its N-oxide, or a protonated form thereof, wherein each of R¹, R², and R³ is independently selected from H, C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is H, CH(C₁₋₅ alkylene-R⁴)₂, or C(C₁₋₃ alkylene-R⁴)₃; or two of R¹, R², and R³ join together with the nitrogen atom to form a 5- or 6-membered N-heterocyclic ring which is optionally substituted with one or two R⁵; each R⁴ is independently selected from —OH, —O-(C₁₋₆ alkylene-OH), and —N(R⁶)_{z}-(C₁₋₆ alkylene-OH)₂-, wherein each z is independently selected from 0 and 1; and each R⁶ is independently selected from H and C₁₋₃ alkyl; and each R⁵ is independently selected from C₁₋₆ alkyl, C₁₋₆ alkylene-R⁴, CH(C₁₋₅ alkylene-R⁴)₂, and C(C₁₋₅ alkylene-R⁴)₃.

In some embodiments of the first and second aspects, each of R¹, R², and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is CH(C₁₋₅ alkylene-R⁴)₂ or C(C₁₋₅ alkylene-R⁴)₃, preferably each of R¹, R², and R³ is independently selected from C₁₋₄ alkyl, C₁₋₄ alkylene-R⁴, CH(C₁₋₃ alkylene-R⁴)₂, and C(C₁₋₃ alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₃ alkyl, C₁₋₃ alkylene-R⁴, CH(C₁₋₃ alkylene-R⁴)₂, and C(C₁₋₃ alkylene-R⁴)₃, wherein at most one of R¹, R², and R³ is CH(C₁₋₄ alkylene-R⁴)₂ or C(C₁₋₃ alkylene-R⁴)₃, more preferably each of R¹, R², and R³ is independently selected from C₁₋₂ alkyl, C₁₋₂ alkylene-R⁴, CH(C₁₋₂ alkylene-R⁴)₂, and C(C₁₋₂ alkylene-R⁴)₃, wherein at most one of R¹, R², and R³ is CH(C₁₋₂ alkylene-R⁴)₂ or C(C₁₋₂ alkylene-R⁴)₃. For example, each of R¹, R², and R³ may be independently selected from C₁₋₆ alkyl, C₁₋₆ alkylene-R⁴, and C(C₁₋₅ alkylene-R⁴)₃, wherein at most one of R¹, R², and R³ is C(C₁₋₅ alkylene-R⁴)₃, preferably each of R¹, R², and R³ is independently selected from C₁₋₄ alkyl, C₁₋₄ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₃ alkyl, C₁₋₃ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₂ alkyl, C₁₋₂ alkylene-R⁴, and C(C₁₋₂ alkylene-R⁴)₃, wherein at most one of R¹, R², and R³ is C(C₁₋₂ alkylene-R⁴)₃. In some embodiments, each of R¹, R², and R³ is independently selected from C₁₋₆ alkyl and C₁₋₆ alkylene-R⁴, preferably each of R¹, R², and R³ is independently selected from C₁₋₄ alkyl and C₁₋₄ alkylene-R⁴, more preferably each of R¹, R², and R³ is independently selected from C₁₋₃ alkyl and C₁₋₃ alkylene-R⁴, more preferably each of R¹, R², and R³ is independently selected from C₁₋₂ alkyl and C₁₋₂ alkylene-R⁴.

In some embodiments of the first and second aspects, each R⁴ is independently selected from —OH, —O-(C₁₋₄ alkylene-OH), and —N(R⁶)_{z}-(C₁₋₄ alkylene-OH)_{2-z}, wherein each z is independently selected from 0 and 1; and each R⁶ is independently selected from H and C₁₋₃ alkyl, preferably each R⁴ is independently selected from —OH, —O-(C₁₋₃ alkylene-OH), and —N(R⁶)_{z}-(C₁₋₃ alkylene-OH)_{2-z}, wherein each z is independently selected from 0 and 1; and each R⁶ is independently selected from H and C₁₋₃ alkyl, more preferably each R⁴ is independently selected from —OH, —O-(C₁₋₂ alkylene-OH), and —N(R⁶)_{z}-(C₁₋₂alkylene-OH)₂, wherein each z is independently selected from 0 and 1; and each R⁶ is independently selected from H and C₁₋₂ alkyl. For example, each R⁴ may independently be selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂. In some embodiments, each R⁴ is independently selected from —OH, 2-hydroxyethoxy, and bis(2-hydroxyethyl)amino.

In some embodiments of the first and second aspects, wherein any one (or each) of R¹, R², and R³ is C₁₋₆ alkylene-R⁴ and R⁴ is OH, it is preferred that the alkylene group has 2 to 6 carbon atoms, such as 2 to 4, e.g., 2, 3, or 4 carbon atoms. Thus, in these embodiments, any one (or each) of R¹, R², and R³ preferably is C₂₋₆ alkylene-OH, more preferably C₂₋₄ alkylene-OH, more preferably C₂₋₃ alkylene-OH, such as C₂ alkylene-OH.

In some embodiments of the first and second aspects, each of R¹, R², and R³ is independently selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

In some embodiments of the first and second aspects, all of R¹, R², and R³ are the same. For example, all of R¹, R², and R³ may be methyl, ethyl, or 2-hydroxyethyl.

In some embodiments of the first and second aspects, R¹ and R² are the same and R³ differs from R¹ and R². For example, each of R¹ and R² may be 2-hydroxyethyl or methyl; and/or R³ is selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

In some embodiments of the first and second aspects, R¹ and R² join together with the nitrogen atom to form a 5- or 6-membered N-heterocyclic ring which is optionally substituted with one or two R⁵. In some embodiments, R³ is selected from C₁₋₆ alkyl, C₁₋₆ alkylene-R⁴, and C(C₁₋₅ alkylene-R⁴)₃, preferably R³ is selected from C₁₋₄ alkyl, C₁₋₄ alkylene-R⁴, and C(C₁₋₃ alkylene-R⁴)₃, more preferably R³ is selected from C₁₋₃ alkyl, C₁₋₃ alkylene-R⁴, and C(C₁₋₃ alkylene-R⁴)₃, more preferably R³ is selected from C₁₋₂alkyl, C₁₋₂ alkylene-R⁴, and C(C₁₋₂ alkylene-R⁴)₃, more preferably R³ is selected from selected from methyl, ethyl, 2-hydroxyethyl, 2-(2-hydroxyethoxy)ethyl, and 2-[bis(2-hydroxyethyl)amino]ethyl. In some embodiments, the N-heterocyclic ring is a monocyclic ring containing at least one nitrogen ring atom and optionally one further ring heteroatom selected from O and S. For example, the N-heterocyclic ring may be a monocyclic ring containing (i) one nitrogen ring atom; (ii) two nitrogen ring atoms; (iii) one nitrogen ring atom and one oxygen ring atom; (iv) one nitrogen ring atom and one sulfur ring atom; or (v) three nitrogen ring atoms. In some embodiments, the N-heterocyclic ring is a monocyclic 5- or 6-membered N-heterocyclic ring, such as is a monocyclic 6-membered N-heterocyclic ring. Preferred examples of the N-heterocyclic ring include pyrrolidinyl, imidazolidinyl, pyrazolidinyl, oxazolidinyl, thiazolidinyl, piperidinyl, piperazinyl, 1,2-diazinanyl, 1,3-diazinanyl, 1,3,5-triazinanyl, morpholinyl, and thiomorpholinyl. Preferably, the N-heterocyclic ring is selected from piperidinyl, piperazinyl, 1,2-diazinanyl, 1,3-diazinanyl, morpholinyl, and thiomorpholinyl. In some embodiments, the N-heterocyclic ring contains only one nitrogen ring atom; in these embodiments, it is preferred that this nitrogen ring atom is substituted with R³, R³ being other than H. In some embodiments, the N-heterocyclic ring contains more than one nitrogen ring atom; in these embodiments, it is preferred that one nitrogen ring atom is substituted with R³, R³ being other than H, and at least one of the other nitrogen ring atoms, preferably each of the other nitrogen ring atoms, is substituted with R⁵. In some embodiments, each R⁵ is independently selected from C₁₋₆ alkyl, C₁₋₆ alkylene-R⁴, and C(C₁₋₅ alkylene-R⁴)₃, preferably R⁵ is selected from C₁₋₄ alkyl, C₁₋₄ alkylene-R⁴, and C(C₁₋₃ alkylene-R⁴)₃, more preferably R⁵ is selected from C₁₋₃ alkyl, C₁₋₃ alkylene-R⁴, and C(C₁₋₃ alkylene-R⁴)₃, more preferably R⁵ is selected from C₁₋₂ alkyl, C₁₋₂ alkylene-R⁴, and C(C₁₋₂ alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl. In some embodiments, the N-heterocyclic ring is piperidinyl and the ring N atom is substituted with R³, R³ being other than H. In some embodiments, the N-heterocyclic ring is piperazinyl, one the two ring N atoms is substituted with R³, R³ being other than H, and the other ring N atom is optionally substituted with R⁵, preferably the other ring N atom is substituted with R⁵. In some embodiments, the N-heterocyclic ring is piperazinyl and both ring N atoms are substituted, wherein one of the two ring N atoms is substituted with R³, R³ being other than H, and the other ring N atom is substituted with R⁵, wherein preferably R⁵ is selected from C₁₋₆ alkyl, C₁₋₆ alkylene-R⁴, and C(C₁₋₅ alkylene-R⁴)₃, more preferably R⁵ is selected from C₁₋₄ alkyl, C₁₋₄ alkylene-R⁴, and C(C₁₋₃ alkylene-R⁴)₃, more preferably R⁵ is selected from C₁₋₃ alkyl, C₁₋₃ alkylene-R⁴, and C(C₁₋₃ alkylene-R⁴)₃, more preferably R⁵ is selected from C₁₋₂ alkyl, C₁₋₂ alkylene-R⁴, and C(C₁₋₂ alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl. In some of the above embodiments, where R¹ and R² join together with the nitrogen atom to form a 5- or 6-membered N-heterocyclic ring, each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂. In some of the above embodiments, where R¹ and R² join together with the nitrogen atom to form a 5- or 6-membered N-heterocyclic ring, each R⁴ is independently selected from —OH, 2-hydroxyethoxy, and bis(2-hydroxyethyl)amino. In some of the above embodiments, where R¹ and R² join together with the nitrogen atom to form a 5- or 6-membered N-heterocyclic ring, and any one (or each) of R³ and R⁵ is C₁₋₆ alkylene-R⁴ and R⁴ is OH, it is preferred that the alkylene group has 2 to 6 carbon atoms, such as 2 to 4, e.g., 2, 3, or 4 carbon atoms. Thus, in these embodiments, any one (or each) of R³ and R⁵ preferably is C₂₋₆ alkylene-OH, more preferably C₂₋₄ alkylene-OH, more preferably C₂₋₃ alkylene-OH, such as C₂ alkylene-OH. In some of the above embodiments where R¹ and R² join together with the nitrogen atom to form a 5- or 6-membered N-heterocyclic ring, R³ and R⁵ are the same. In some of the above embodiments where R¹ and R² join together with the nitrogen atom to form a 5- or 6-membered N-heterocyclic ring, both of R³ and R⁵ are methyl, ethyl, 2-hydroxyethyl, or 2-(2-hydroxyethoxy)ethyl, preferably, both of R³ and R⁵ are 2-hydroxyethyl. In some of the above embodiments where R¹ and R² join together with the nitrogen atom to form a 5- or 6-membered N-heterocyclic ring, R³ and R⁵ differ from each other.

In some embodiments of the first and second aspects, R¹ is H. In some embodiments, each of R² and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R² and R³ is CH(C₁₋₅ alkylene-R⁴)₂ or C(C₁₋₅ alkylene-R⁴)₃, preferably each of R² and R³ is independently selected from C₁₋₄ alkyl, C₁₋₄ alkylene-R⁴, CH(C₁₋₃ alkylene-R⁴)₂, and C(C₁₋₃ alkylene-R⁴)₃, wherein at most one of R² and R³ is CH(C₁₋₃ alkylene-R⁴)₂ or C(C₁₋₃ alkylene-R⁴)₃, more preferably each of R² and R³ is independently selected from C₁₋₃ alkyl, C₁₋₃ alkylene-R⁴, CH(C₁₋₃alkylene-R⁴)₂, and C(C₁₋₃ alkylene-R⁴)₃, wherein at most one of R² and R³ is CH(C₁₋₃ alkylene-R⁴)₂ or C(C₁₋₃ alkylene-R⁴)₃, more preferably each of R² and R³ is independently selected from C₁₋₂ alkyl, C₁₋₂ alkylene-R⁴, CH(C₁₋₂ alkylene-R⁴)₂, and C(C₁₋₂ alkylene-R⁴)₃, wherein at most one of R² and R³ is CH(C₁₋₂ alkylene-R⁴)₂ or C(C₁₋₂ alkylene-R⁴)₃. For example, each of R² and R³ may be independently selected from C₁₋₆ alkyl, C₁₋₆ alkylene-R⁴, and C(C₁₋₅ alkylene-R⁴)₃, wherein at most one of R² and R³ is C(C₁₋₅ alkylene-R⁴)₃, preferably each of R² and R³ is independently selected from C₁₋₄ alkyl, C₁₋₄ alkylene-R⁴, and C(C₁₋₃ alkylene-R⁴)₃, wherein at most one of R² and R³ is C(C₁₋₃ alkylene-R⁴)₃, more preferably each of R² and R³ is independently selected from C₁₋₃ alkyl, C₁₋₃ alkylene-R⁴, and C(C₁₋₃alkylene-R⁴)₃, wherein at most one of R² and R³ is C(C₁₋₃ alkylene-R⁴)₃, more preferably each of R² and R³ is independently selected from C₁₋₂ alkyl, C₁₋₂ alkylene-R⁴, and C(C₁₋₂ alkylene-R⁴)₃, wherein at most one of R² and R³ is C(C₁₋₂ alkylene-R⁴)₃. In some embodiments, each of R² and R³ is independently selected from C₁₋₆ alkyl and C₁₋₆ alkylene-R⁴, preferably each of R² and R³ is independently selected from C₁₋₄ alkyl and C₁₋₄ alkylene-R⁴, more preferably each of R² and R³ is independently selected from C₁₋₃ alkyl and C₁₋₃ alkylene-R⁴, more preferably each of R² and R³ is independently selected from C₁₋₂ alkyl and C₁₋₂ alkylene-R⁴.

In some embodiments, where R¹ is H, each R⁴ is independently selected from —OH, —O-(C₁₋₄ alkylene-OH), and —N(C₁₋₄ alkylene-OH)₂, preferably each R⁴ is independently selected from —OH, —O-(C₁₋₃alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂. For example, each R⁴ may be independently selected from —OH, 2-hydroxyethoxy, and bis(2-hydroxyethyl)amino.

In some embodiments of the first and second aspects, where R¹ is H, any one (or each) of R² and R³ is C₁₋₆ alkylene-R⁴ and R⁴ is OH, it is preferred that the alkylene group has 2 to 6 carbon atoms, such as 2 to 4, e.g., 2, 3, or 4 carbon atoms. Thus, in these embodiments, any one (or each) of R² and R³ preferably is C₂₋₆ alkylene-OH, more preferably C₂₋₄ alkylene-OH, more preferably C₂₋₃ alkylene-OH, such as C₂ alkylene-OH.

In some embodiments, where R¹ is H, each of R² and R³ is independently selected from 2-hydroxyethyl, 2-(2-hydroxyethoxy)ethyl, and 2-[bis(2-hydroxyethyl)amino]ethyl, preferably, both of R² and R³ are 2-hydroxyethyl or 2-(2-hydroxyethoxy)ethyl.

In some embodiments of the first and second aspects, the buffer substance comprises or is a tertiary amine as defined herein (i.e., N(R¹)(R²)(R³), wherein none of R¹, R², and R³ is H) or a protonated form thereof. Thus, in some embodiments, each of R¹, R², and R³ is independently selected from C₁_₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is CH(C₁₋₅ alkylene-R⁴)₂, or C(C₁₋₅ alkylene-R⁴)₃, as specified above. In some embodiments, the tertiary amine is a monoamine. In some embodiments, the tertiary amine is selected from the group consisting of bis(2-hydroxyethyl)amino-tris(hydroxymethyl)methane (Bis-Tris-methane or BTM), triethanolamine (TEA), ethyldiethanolamine, 2-(diethylamino)ethan-1-ol, triethylamine, and 2-[2-(diethylamino)ethoxy]ethan-1-ol. In some embodiments, the tertiary amine comprises or is triethanolamine (TEA).

In some embodiments of the first and second aspects, the buffer substance comprises or is a cyclic amine as defined herein (i.e., N(R¹)(R²)(R³), wherein two of R¹, R², and R³ join together with the nitrogen atom to form a 5- or 6-membered N-heterocyclic ring which is optionally substituted with one or two R⁵) or a protonated form thereof. Thus, in some embodiments, R¹ and R² join together with the nitrogen atom to form a 5- or 6-membered N-heterocyclic ring which is optionally substituted with one or two R⁵, as specified above. In some embodiments, the cyclic amine is selected from the group consisting of N,N′-bis(2-hydroxyethyl)piperazine and morpholine substituted with one or more C₁₋₆ alkylene-R⁴ (such as 2-hydroxyethyl) moieties.

In some embodiments of the first and second aspects, the buffer substance comprises or is a secondary amine as defined herein (i.e., N(R¹)(R²)(R³), wherein one of R¹, R², and R³ is H) or a protonated form thereof. Thus, in some embodiments, R¹ is H and each of R² and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R² and R³ is CH(C₁₋₅ alkylene-R⁴)₂, or C(C₁₋₅ alkylene-R⁴)₃, as specified above.

In some embodiments of the first and second aspects, the buffer substance comprises or is an N-oxide. In some embodiments, the N-oxide is trimethylamine N-oxide.

In some embodiments of the first and second aspects, the buffer substance comprises at least one C₁₋₆ alkylene-R⁴ moiety. In those cases of these embodiments, where R⁴ is OH, it is preferred that the alkylene group has 2 to 6 carbon atoms, such as 2 to 4, e.g., 2, 3, or 4 carbon atoms. Thus, in these 10 embodiments, wherein the buffer substance comprises at least one C₁₋₆ alkylene-R⁴ moiety and R⁴ is OH, the at least one C₁₋₆ alkylene-R⁴ moiety preferably is C₂₋₆ alkylene-OH, more preferably C₂₋₄ alkylene-OH, more preferably C₂₋₃ alkylene-OH, such as C₂ alkylene-OH or 2-hydroxyethyl.

In some embodiments of the first and second aspects, the buffer substance is selected from bis(2-hydroxyethyl)amino-tris(hydroxymethyl)methane (Bis-Tris-methane or BTM) and its protonated form, triethanolamine (TEA) and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form. In some embodiments, the buffer substance is selected from bis(2-hydroxyethyl)amino-tris(hydroxymethyl)methane (Bis-Tris-methane or BTM) and its protonated form, triethanolamine (TEA) and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, and N,N′-bis(2-hydroxyethyl)piperazine and its protonated form. In some embodiments, the buffer substance comprises or is triethanolamine (TEA) or its protonated form.

In some embodiments of the first and second aspects, the concentration of the buffer substance in the composition is between about 10 mM and about 200 mM, such as between about 20 mM and about 180 mM, between about 30 mM and about 170 mM, between about 40 mM and about 160 mM, between about 50 mM and about 50 mM, between about 60 mM and about 140 mM, between about 70 mM and about 130 mM, between about 80 mM and about 120 mM, between about 90 mM and about 110 mM. In some embodiments, the concentration of the buffer substance in the composition is between about 15 mM and about 100 mM, preferably between about 20 mM and about 80 mM, more preferably between about 40 mM and about 60 mM, such as about 50 mM.

In some embodiments of the first and second aspects, the buffer system further comprises an anion. In some embodiments, this anion can act as further buffer substance. In some embodiments, the anion is selected from anions of inorganic and/or organic acids (in particular, when the desired pH of the composition is at least 2.5 pH units lower than the pKa of the buffer substance the formula N(R¹)(R²)(R³), its N-oxide, or a protonated form thereof). In some embodiments, the anion is selected from the group consisting of chloride, acetate, glycolate, lactate, and the anion of a di- or tricarboxylic acid, such as the anion of citric acid, succinic acid, malonic acid, glutaric acid, or adipic acid. In some embodiments, where the buffer system comprises an anion, the concentration of the anion in the composition is at least equal to the concentration of the buffer substance in the composition. For example, the concentration of the anion in the composition may be higher than the concentration of the buffer substance in the composition. Thus, in those embodiments of the first and second aspects, where the concentration of the buffer substance in the composition is x being within in the range between about 10 mM and about 200 mM, the concentration of the anion in the composition is at least equal to x, e.g., higher than x.

In some embodiments of the first and second aspects, the pH of the composition is between about 4.0 and about 8.0. For example, the pH of the composition may be between about 4.5 and about 8.0, such as between about 5.0 and about 8.0, between about 5.5 and about 8.0, between about 6.0 and about 8.0, between about 6.5 and about 8.0, between about 6.8 and about 7.9, between about 7.0 and about 7.8, or about 7.5.

In some embodiments of the first and second aspects, water is the main component in the composition and/or the total amount of solvent(s) other than water contained in the composition is less than about 1.0% (v/v), such as less than about 0.5% (v/v). For example, the amount of water contained in the composition may be at least 50% (w/w), such as at least at least 55% (w/w), at least 60% (w/w), at least 65% (w/w), at least 70% (w/w), at least 75% (w/w), at least 80% (w/w), at least 85% (w/w), at least 90% (w/w), or at least 95% (w/w). In particular, if the composition comprises a cryoprotectant, the amount of water contained in the composition may be at least 50% (w/w), such as at least at least 55% (w/w), at least 60% (w/w), at least 65% (w/w), at least 70% (w/w), at least 75% (w/w), at least 80% (w/w), at least 85% (w/w), or at least 90% (w/w). If the composition is substantially free of a cryoprotectant, the amount of water contained in the composition may be at least 95% (w/w). Additionally or alternatively, the total amount of solvent(s) other than water contained in the composition may be less than about 1.0% (v/v), such as less than about 0.9% (v/v), less than about 0.8% (v/v), less than about 0.7% (v/v), less than about 0.6% (v/v), less than about 0.5% (v/v), less than about 0.4% (v/v), less than about 0.3% (v/v), less than about 0.2% (v/v), less than about 0.1% (v/v), less than about 0.05% (v/v), or less than about 0.01% (v/v). In this respect, a cryoprotectant which is liquid under normal conditions will not be considered as a solvent other than water but as cryoprotectant. In other words, the above optional limitation that the total amount of solvent(s) other than water contained in the composition may be less than about 1.0% (v/v), such as less than about 0.5% (v/v), does not apply to cryoprotectants which are liquids under normal conditions.

In some embodiments of the first and second aspects, the osmolality of the composition is at most about 1000×10⁻³ osmol/kg, such as between about 100×10⁻³ osmol/kg and about 750×10⁻³ osmol/kg. In some embodiments, the osmolality of the composition is at most about 500×10⁻³ osmol/kg, such as at most about 490×10⁻³ osmol/kg, at most about 480×10⁻³ osmol/kg, at most about 470×10⁻³ osmol/kg, at most about 460×10⁻³ osmol/kg, at most about 450×10⁻³ osmol/kg, at most about 440×10⁻³ osmol/kg, at most about 430×10⁻³ osmol/kg, at most about 420×10⁻³ osmol/kg, at most about 410×10⁻³ osmol/kg, at most about 400×10⁻³ osmol/kg, at most about 390×10⁻³ osmol/kg, at most about 380×10⁻³ osmol/kg, at most about 370×10⁻³ osmol/kg, at most about 360×10⁻³ osmol/kg, at most about 350×10⁻³ osmol/kg, at most about 340×10⁻³ osmol/kg, at most about 330×10⁻³ osmol/kg, at most about 320×10⁻³ osmol/kg, at most about 310×10⁻³ osmol/kg, or at most about 300×10⁻³ osmol/kg. If the composition does not comprise a cryoprotectant, the osmolality of the composition may be below 300×10⁻³ osmol/kg, such as at most about 250×10⁻³ osmol/kg, at most about 200×10⁻³ osmol/kg, at most about 150×10⁻³ osmol/kg, at most about 100×10⁻³ osmol/kg, at most about 50×10⁻³ osmol/kg, at most about 40×10⁻³ osmol/kg, or at most about 30×10⁻³ osmol/kg. If the composition comprises a cryoprotectant, it is preferred that the main part of the osmolality of the composition is provided by the cryoprotectant. For example, the cryoprotectant may provide at least 50%, such as at least 55%, at least 60%, at least 65%, at least 70%, at least 75%, at least 80%, at least 85%, or at least 90%, of the osmolality of the composition.

In some embodiments of the first and second aspects, the concentration of the RNA in the composition is about 5 mg/l to about 500 mg/l, such as about 10 mg/l to about 400 mg/l, about 10 mg/l to about 300 mg/l, about 10 mg/l to about 200 mg/l, about 10 mg/l to about 150 mg/l, or about 10 mg/l to about 100 mg/l, preferably about 10 mg/l to about 140 mg/l, more preferably about 20 mg/l to about 130 mg/l, more preferably about 30 mg/l to about 120 mg/l. In some embodiments, the concentration of the RNA in the composition is about 5 mg/l to about 150 mg/l, such as about 10 mg/l to about 140 mg/l, about 20 mg/l to about 130 mg/l, about 25 mg/l to about 125 mg/l, about 30 mg/l to about 120 mg/l, about 35 mg/l to about 115 mg/l, about 40 mg/l to about 110 mg/l, about 45 mg/l to about 105 mg/l, or about 50 mg/l to about 100 mg/l.

In some embodiments of the first and second aspects, the composition comprises a cryoprotectant, preferably in a concentration of at least about 1% w/v, wherein the cryoprotectant preferably comprises one or more compounds selected from the group consisting of carbohydrates and alcohols (such as sugar alcohols or lower alcohols), more preferably the cryoprotectant is selected from the group consisting of sucrose, glucose, glycerol, 1,2-propanediol, 1,3-propanediol, sorbitol, and a combination thereof (such as from the group consisting of sucrose, glucose, glycerol, 1,2-propanediol, 1,3-propanediol, and a combination thereof or from the group consisting of sucrose, glucose, glycerol, sorbitol, and a combination thereof), more preferably the cryoprotectant comprises sucrose and/or glycerol. In some embodiments, the concentration of the cryoprotectant in the composition is at least 1% w/v, such as at least 2% w/v, at least 3% w/v, at least 4% w/v, at least 5% w/v, at least 6% w/v, at least 7% w/v, at least 8% w/v, or at least 9% w/v. In some embodiments, the concentration of the cryoprotectant in the composition is up to 25% w/v, such as up to 20% w/v, up to 19% w/v, up to 18% w/v, up to 17% w/v, up to 16% w/v, up to 15% w/v, up to 14% w/v, up to 13% w/v, up to 12% w/v, or up to 11% w/v. In some embodiments, the concentration of the cryoprotectant in the composition is 10% w/v to 20% w/v, such as 2% w/v to 19% w/v, 3% w/v to 18% w/v, 4% w/v to 17% w/v, 5% w/v to 16% w/v, 5% w/v to 15% w/v, 6% w/v to 14% w/v, 7% w/v to 13% w/v, 8% w/v to 12% w/v, 9% w/v to 11% w/v, or about 10% w/v. In some embodiments, the composition comprises a cryoprotectant (such as sucrose, glucose, glycerol, 1,2-propanediol, or 1,3-propanediol, in particular, sucrose and/or glycerol) in a concentration of from 5% w/v to 15% w/v, such as from 6% w/v to 14% w/v, from 7% w/v to 13% w/v, from 8% w/v to 12% w/v, or from 9% w/v to 11% w/v, or in a concentration of about 10% w/v.

In some embodiments of the first and second aspects, wherein the composition comprises a cryoprotectant, the cryoprotectant is present in a concentration resulting in an osmolality of the composition in the range of from about 50×10⁻³ osmol/kg to about 1000×10⁻³ osmol/kg (such as from about 50×10⁻³ osmol/kg to about 500×10⁻³ osmol/kg, from about 50×10⁻³ osmol/kg to about 480×10⁻³ osmol/kg, from about 60×10⁻³ osmol/kg to about 460×10⁻³ osmol/kg, from about 70×10⁻³ osmol/kg to about 440×10⁻³ osmol/kg, from about 80×10⁻³ osmol/kg to about 420×10⁻³ osmol/kg, from about 90×10⁻³ osmol/kg to about 400×10⁻³ osmol/kg, from about 100×10⁻³ osmol/kg to about 380×10⁻³ osmol/kg, from about 120×10⁻³ osmol/kg to about 360×10⁻³ osmol/kg, from about 140×10⁻³ osmol/kg to about 340×10⁻³ osmol/kg, from about 160×10⁻³ osmol/kg to about 310×10⁻³ osmol/kg, from about 180×10⁻³ osmol/kg to about 300×10⁻³ osmol/kg, or from about 200×10⁻³ osmol/kg to about 300×10⁻³ osmol/kg), based on the total weight of the composition.

In some embodiments of the first and second aspects, the composition is substantially free of a cryoprotectant.

In some embodiments of the first and second aspects, the buffer substance comprises a tertiary amine as defined herein (i.e., N(R¹)(R²)(R³), wherein none of R¹, R², and R³ is H) or its protonated form, the pH of the composition is between about 4.0 and about 8.0, and the concentration of the RNA in the composition is about 5 mg/l to about 500 mg/l. In this embodiment, it is preferred that the pH of the composition is between about 4.5 and about 8.0 and the concentration of the RNA in the composition is about 20 mg/l to about 130 mg/l, such as about 30 mg/l to about 120 mg/l. In particularly preferred embodiment, the buffer substance comprises a tertiary amine as defined herein or its protonated form; the pH of the composition is between about 5.0 and about 8.0; the concentration of the RNA in the composition is about 30 mg/l to about 120 mg/l; and the composition comprises a cryoprotectant. In an alternative particularly preferred embodiment, the buffer substance comprises a tertiary amine as defined herein or its protonated form; the pH of the composition is between about 5.0 and about 8.0; the concentration of the RNA in the composition is about 30 mg/l to about 120 mg/l; and the composition is substantially free of a cryoprotectant. In some embodiments, the tertiary amine is a monoamine. In some embodiments, the tertiary amine is selected from the group consisting of bis(2-hydroxyethyl)amino-tris(hydroxymethyl)methane (Bis-Tris-methane or BTM), triethanolamine (TEA), ethyldiethanolamine, 2-(diethylamino)ethan-1-ol, triethylamine, and 2-[2-(diethylamino)ethoxy]ethan-1-ol. In some embodiments, the tertiary amine comprises or is triethanolamine (TEA).

In some embodiments of the first and second aspects, the buffer substance comprises a cyclic amine as defined herein (i.e., N(R¹)(R²)(R³), wherein two of R¹, R², and R³ join together with the nitrogen atom to form a 5- or 6-membered N-heterocyclic ring which is optionally substituted with one or two R⁵) or its protonated form, the pH of the composition is between about 4.0 and about 8.0, and the concentration of the RNA in the composition is about 5 mg/l to about 500 mg/l. In this embodiment, it is preferred that the pH of the composition is between about 4.5 and about 8.0 and the concentration of the RNA in the composition is about 20 mg/l to about 130 mg/l, such as about 30 mg/l to about 120 mg/l. In particularly preferred embodiment, the buffer substance comprises a cyclic amine as defined herein or its protonated form; the pH of the composition is between about 5.0 and about 8.0; the concentration of the RNA in the composition is about 30 mg/l to about 120 mg/l; and the composition comprises a cryoprotectant. In an alternative particularly preferred embodiment, the buffer substance comprises a cyclic amine as defined herein or its protonated form; the pH of the composition is between about 5.0 and about 8.0; the concentration of the RNA in the composition is about 30 mg/l to about 120 mg/l; and the composition is substantially free of a cryoprotectant. In some embodiments, the cyclic amine is selected from the group consisting of N,N′-bis(2-hydroxyethyl)piperazine and morpholine substituted with one or more C₁₋₆ alkylene-R⁴ (such as 2-hydroxyethyl) moieties.

In some embodiments of the first aspect, the cationically ionizable lipid comprises a head group which includes at least one nitrogen atom which is capable of being protonated under physiological conditions. In some embodiments of the first aspect, the cationically ionizable lipid has the structure of Formula (X)

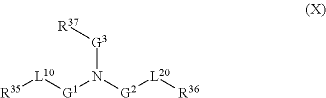

or a pharmaceutically acceptable salt, tautomer, prodrug or stereoisomer thereof, wherein L¹⁰, L²⁰, G¹, G², G³, R³⁵, R³⁶, and R³⁷ are as defined herein. In some embodiments, the cationically ionizable lipid is selected from the following: the structures X-1 to X-36 (shown herein); the structures A to G (shown herein); or N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14). In some embodiments, the cationically ionizable lipid is the lipid having the structure X-3.

In some embodiments of the first aspect, the cationically ionizable lipid has the structure of Formula (XI):

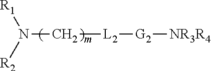

wherein R₁, R₂, R₃, R₄, R₅, R₆, G₁, G₂, and m are as defined herein. In some embodiments, the cationically ionizable lipid is selected from the structures (XIV-1), (XIV-2), and (XIV-3) (shown herein).

In some embodiments of the first aspect, the cationically ionizable lipid comprises from about 20 mol % to about 80 mol %, preferably from about 25 mol % to about 65 mol %, more preferably from about 30 mol % to about 50 mol %, such as from about 40 mol % to about 50 mol %, of the total lipid present in the composition.

In some embodiments of the first aspect, the composition further comprises one or more additional lipids. In some embodiments, the one or more additional lipids are selected from the group consisting of polymer conjugated lipids, neutral lipids, steroids, and combinations thereof. In some embodiments, the composition comprises the cationically ionizable lipid, a polymer conjugated lipid (e.g., a pegylated lipid; or a polysarcosine-lipid conjugate or a conjugate of polysarcosine and a lipid-like material), a neutral lipid (e.g., a phospholipid, such as DSPC), and a steroid (e.g., cholesterol).

In some embodiments of the first aspect, wherein the composition further comprises a polymer conjugated lipid as one of the one or more additional lipids, the polymer conjugated lipid comprises a pegylated lipid. In some embodiments, the pegylated lipid is selected from the group consisting of DSPE-PEG, DOPE-PEG, DPPE-PEG, and DMPE-PEG. In some embodiments, the pegylated lipid has the following structure:

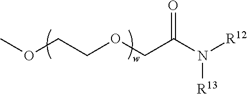

or a pharmaceutically acceptable salt, tautomer or stereoisomer thereof, wherein R¹², R¹³, and w are as defined herein.

In some embodiments of the first aspect, wherein the composition further comprises a polymer conjugated lipid as one of the one or more additional lipids, the polymer conjugated lipid comprises a polysarcosine-lipid conjugate or a conjugate of polysarcosine and a lipid-like material. In some embodiments, the polysarcosine-lipid conjugate or conjugate of polysarcosine and a lipid-like material is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

In some embodiments of the first aspect, wherein the composition further comprises a polymer conjugated lipid as one of the one or more additional lipids, the polymer conjugated lipid comprises from about 0.5 mol % to about 5 mol %, preferably from about 1 mol % to about 5 mol %, more preferably from about 1 mol % to about 4.5 mol % of the total lipid present in the composition.

In some embodiments of the first aspect, wherein the composition further comprises a neutral lipid as one of the one or more additional lipids, the neutral lipid is a phospholipid. In some embodiments, the phospholipid is selected from the group consisting of phosphatidylcholines, phosphatidylethanolamines, phosphatidylglycerols, phosphatidic acids, phosphatidylserines and sphingomyelins. In some embodiments, the phospholipid is selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidyleholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE). In some embodiments, the neutral lipid is DSPC.

In some embodiments of the first aspect, wherein the composition further comprises a neutral lipid as one of the one or more additional lipids, the neutral lipid comprises from about 5 mol % to about 40 mol %, preferably from about 5 mol % to about 20 mol %, more preferably from about 5 mol % to about 15 mol % of the total lipid present in the composition.

In some embodiments of the first aspect, wherein the composition further comprises a steroid as one of the one or more additional lipids, the steroid is a sterol such as cholesterol.

In some embodiments of the first aspect, wherein the composition further comprises a steroid as one of the one or more additional lipids, the steroid comprises from about 10 mol % to about 65 mol %, preferably from about 20 mol % to about 60 mol %, more preferably from about 30 mol % to about 50 mol % of the total lipid present in the composition.

In some embodiments of the first aspect, the composition comprises a cationically ionizable lipid, a polymer conjugated lipid, a neutral lipid (e.g., a phospholipid), and a steroid, wherein the cationically ionizable lipid comprises from about 30 mol % to about 50 mol %, such as from about 40 mol % to about 50 mol %, of the total lipid present in the composition; the polymer conjugated lipid comprises from about 1 mol % to about 4.5 mol % of the total lipid present in the composition; the neutral lipid (e.g., phospholipid) comprises from about 5 mol % to about 15 mol % of the total lipid present in the composition; and the steroid comprises from about 30 mol % to about 50 mol % of the total lipid present in the composition.

In some embodiments of the first and second aspects, at least a portion of the RNA and, if present, of one or more lipids, is present in particles, such as lipid nanoparticles (LNPs), liposomes, and/or lipoplexes (LPXs). In some embodiments of the first and second aspects, the RNA is encapsulated within or associated with the particles. In some embodiments, the particles comprise at least about 75% of the RNA comprised in the composition. In some embodiments, the particles comprise at least about 76%, such as at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95% of the RNA comprised in the composition. In some embodiments, the particles have a size of from about 30 nm to about 500 nm.

In some embodiments of the first and second aspects, the RNA is mRNA or inhibitory RNA.

In some embodiments of the first and second aspects, the RNA (such as mRNA) (i) comprises a modified nucleoside in place of uridine; (ii) has a coding sequence which is codon-optimized; and/or (iii) has a coding sequence whose G/C content is increased compared to the wild-type coding sequence. In some embodiments, the modified nucleoside is selected from pseudouridine (ψ), N1-methyl-pseudouridine (m1ψ), and 5-methyl-uridine (m5U).

In some embodiments of the first and second aspects, the RNA (such as mRNA) comprises at least one or more of the following: a 5′ cap; a 5′ UTR; a 3′ UTR; and a poly-A sequence. In some embodiments, the RNA (such as mRNA) comprises all of the following: a 5′ cap; a 5′ UTR; a 3′ UTR; and a poly-A sequence. In some embodiments, the poly-A sequence comprises at least 100 A nucleotides, wherein the poly-A sequence preferably is an interrupted sequence of A nucleotides. In some embodiments, the 5′ cap is a cap1 or cap2 structure.

In some embodiments of the first and second aspects, the RNA (such as mRNA) encodes one or more polypeptides. In some embodiments, the one or more polypeptides are pharmaceutically active polypeptides and/or comprise an epitope for inducing an immune response against an antigen in a subject.

In some embodiments of the first and second aspects, the pharmaceutically active polypeptide and/or the antigen or epitope is derived from or is a protein of a pathogen, an immunogenic variant of the protein, or an immunogenic fragment of the protein or the immunogenic variant thereof. In some embodiments, the pathogen is a pathogen causing an infectious disease.

In some embodiments of the first and second aspects, the pharmaceutically active polypeptide and/or the antigen or epitope is derived from or is a SARS-CoV-2 spike (S) protein, an immunogenic variant thereof, or an immunogenic fragment of the SARS-CoV-2 S protein or the immunogenic variant thereof.

In some embodiments, the RNA (such as mRNA) comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof. In some embodiments, the SARS-CoV2 S protein variant has proline residue substitutions at positions 986 and 987 of SEQ ID NO: 1. In some embodiments, the SARS-CoV2 S protein variant has at least 80% identity to the amino acid sequence of amino acids 17 to 1273 of SEQ ID NO: 7 or the amino acid sequence of amino acids 17 to 1273 of SEQ ID NO: 1. In some embodiments, the fragment comprises the receptor binding domain (RBD) of the SARS-CoV-2 S protein. In some embodiments, the fragment of (i) the SARS-CoV-2 S protein or (ii) the immunogenic variant of the SARS-CoV-2 S protein has at least 80% identity to the amino acid sequence of amino acids 327 to 528 of SEQ ID NO: 1.

In some embodiments of the first aspect, the RNA is inhibitory RNA (such as siRNA) and selectively hybridizes to and/or is specific for a target mRNA. In some embodiments, the target mRNA comprises an ORF encoding a pharmaceutically active peptide or polypeptide, in particular a pharmaceutically active peptide or polypeptide whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

In some embodiments of the first and second aspects, the composition is in liquid form, preferably at a temperature of about 2° C. to about 10° C.

In some embodiments of the first and second aspects, the RNA integrity of the composition after storage for at least one week (such as for at least 2 weeks, at least three weeks, at least four weeks, at least one month, at least two months, at least three months, at least 4 months, or at least 6 months), preferably at a temperature of 0° C. or higher, such as about 2° C. to about 8° C., is sufficient to produce the desired effect, e.g., to induce an immune response. In some embodiments, the RNA integrity of the composition after storage for at least one week (such as for at least 2 weeks, at least three weeks, at least four weeks, at least one month, at least two months, at least three months, at least 4 months, or at least 6 months), preferably at a temperature of 0° C. or higher, such as about 2° C. to about 8° C., is at least 50%, such as at least 60%, at least 70%, at least 80%, at least 85%, at least 90%, at least 95%, at least 97% or at least 98%, compared to the RNA integrity before storage. In some embodiments, the RNA integrity of the composition after storage for at least one week (such as for at least 2 weeks, at least three weeks, at least four weeks, at least one month, at least two months, at least three months, at least 4 months, or at least 6 months), preferably at a temperature of 0° C. or higher, such as about 2° C. to about 8° C., is at least 90%, compared to the RNA integrity before storage. In some embodiments, the RNA integrity of the composition after storage for at least four weeks (e.g., for at least three months), preferably at a temperature of 0° C. or higher, such as about 2° C. to about 8° C., is at least 50%, such as at least 60%, at least 70%, at least 80%, at least 85%, at least 90%, at least 95%, at least 97% or at least 98%, compared to the RNA integrity before storage. In some embodiments, the RNA integrity of the composition after storage for at least four weeks (e.g., for at least three months), preferably at a temperature of 0° C. or higher, such as about 2° C. to about 8° C., is at least 90%, compared to the RNA integrity before storage.

In some embodiments of the first and second aspects, the initial RNA integrity of the composition (i.e., after its preparation but before storage) is at least 50% and the RNA integrity of the composition after storage for at least one week (such as for at least 2 weeks, at least three weeks, at least four weeks, at least one month, at least two months, or at least 3 months), preferably at a temperature of 0° C. or higher, such as about 2° C. to about 8° C., is at least 90%, preferably at least 95%, more preferably at least 97%, more preferably at least 98%, of the initial RNA integrity. In some embodiments of the first and second aspects, the initial RNA integrity of the composition (i.e., after its preparation but before storage) is at least 50% and the RNA integrity of the composition after storage for at least one week (such as for at least four weeks or at least 3 months), preferably at a temperature of 0° C. or higher, such as about 2° C. to about 8° C., is at least 90% of the initial RNA integrity.

Additionally or alternatively, in some embodiments of the first and second aspects, the size (Z_{average}) (and/or size distribution and/or polydispersity index (PDI)) of the RNA particles of the liquid composition after storage for at least one week (such as for at least 2 weeks, at least three weeks, at least four weeks, at least one month, at least two months, at least three months, at least 4 months, or at least 6 months), preferably at a temperature of 0° C. or higher, such as about 2° C. to about 8° C., is sufficient to produce the desired effect, e.g., to induce an immune response. In some embodiments, the size (Z_{average}) (and/or size distribution and/or polydispersity index (PDI) of the RNA particles of the liquid composition after storage for at least one week (such as for at least four weeks or at least three months), preferably at a temperature of 0° C. or higher, such as about 2° C. to about 8° C., is essentially equal to the size (Z_{average}) (and/or size distribution and/or PDI) of the RNA particles of the initial composition, i.e., before storage. In some embodiments, the size (Z_{average}) of the RNA particles after storage for at least one week (such as for at least four weeks or at least three months), preferably at a temperature of 0° C. or higher, such as about 2° C. to about 8° C., is between about 50 nm and about 500 nm, preferably between about 40 nm and about 200 nm, more preferably between about 40 nm and about 120 nm. In some embodiments, the PDI of the RNA particles after storage for at least one week (such as for at least four weeks or at least three months), preferably at a temperature of 0° C. or higher, such as about 2° C. to about 8° C., is less than 0.3, preferably less than 0.2, more preferably less than 0.1. In some embodiments, the size (Z_{average}) of the RNA particles after storage for at least one week (such as for at least four weeks or at least three months), preferably at a temperature of 0° C. or higher, such as about 2° C. to about 8° C., is between about 50 nm and about 500 inn, preferably between about 40 nm and about 200 nm, more preferably between about 40 nm and about 120 nm, and the size (Z_{average}) (and/or size distribution and/or PDI) of the RNA particles after storage of the liquid composition for at least one week (such as for at least four weeks or at least three months), preferably at a temperature of 0° C. or higher, such as about 2° C. to about 8° C., is essentially equal to the size (Z_{average}) (and/or size distribution and/or PDI) of the RNA particles before storage. In one embodiment, the size (Z_{average}) of the RNA particles after storage of the liquid composition for at least one week (such as for at least four weeks or at least three months), preferably at a temperature of 0° C. or higher, such as about 2° C. to about 8° C., is between about 50 nm and about 500 nm, preferably between about 40 nm and about 200 nm, more preferably between about 40 nm and about 120 nm, and the PDI of the RNA particles after storage of the liquid composition for at least one week (such as for at least four weeks or at least three months), preferably at a temperature of 0° C. or higher, such as about 2° C. to about 8° C., e.g., at 0° C. or higher, is less than 0.3 (preferably less than 0.2, more preferably less than 0.1).

In some embodiments of the first and second aspects, the composition is in frozen form (e.g., at −20° C.). In some embodiments of the first and second aspects, where the composition is in frozen form, it is preferred that the composition (a) comprises a cryoprotectant; (b) has a pH between 4.0 and 8.0, preferably between 5.0 and 7.0, more preferably between 5.5 and 6.5 and most preferably about 5.5; or (c) comprises a cryoprotectant and has a pH between 4.0 and 8.0, preferably between 5.0 and 7.0, more preferably between 5.5 and 6.5 and most preferably about 5.5. In some embodiments, the cryoprotectant is (i) selected from the cryoprotectants disclosed herein; and/or (ii) is present in a concentration as disclosed herein. For example, the cryoprotectant may be selected from the group consisting of sucrose, glucose, glycerol, 1,2-propanediol, and 1,3-propanediol, such as from the group consisting of sucrose, glycerol and glucose; and/or may be present in a concentration of between about 100 mM and about 600 mM, preferably between about 200 mM and about 600 mM and more preferably between about 300 mM and about 500 mM. In some embodiments, the cryoprotectant is glycerol, which is optionally present in a concentration of between about 100 mM and about 600 mM, preferably between about 200 mM and about 600 mM and more preferably between about 300 mM and about 500 mM.

In some embodiments, the RNA integrity after thawing the frozen composition is at least 50%, such as at least 60%, at least 70%, at least 80%, at least 85%, at least 90%, at least 95%, at least 97%, at least 98%, or substantially 100%, compared to the RNA integrity before the composition has been frozen. In some embodiments, the RNA integrity after thawing the frozen composition is at least 90%, at least 95%, at least 97%, at least 98%, or substantially 100%, compared to the RNA integrity before the composition has been frozen. In some embodiments, the size (Z_{average}) and/or size distribution and/or polydispersity index (PDI) of RNA particles (in particular LNPs) after thawing the frozen composition is essentially equal to the size (Z_{average}) and/or size distribution and/or PDI of the RNA particles before the composition has been frozen.

In some embodiments of the first and second aspects, the initial RNA integrity of the composition (i.e., after its preparation but before freezing) is at least 50% and the RNA integrity of the composition after thawing the frozen composition is at least 90%, preferably at least 95%, more preferably at least 97%, more preferably at least 98%, more preferably substantially 100%, of the initial RNA integrity.

Additionally or alternatively, in some embodiments of the first and second aspects, the size (Z_{average}) (and/or size distribution and/or polydispersity index (PDI)) of the RNA particles after thawing the frozen composition is essentially equal to the size (Z_{average}) (and/or size distribution and/or PDI) of the RNA particles before the composition has been frozen. In some embodiments, the size (Z_{average}) of the RNA particles after thawing the frozen composition is between about 50 nm and about 500 nm, preferably between about 40 nm and about 200 nm, more preferably between about 40 nm and about 120 nm. In some embodiments, the PDI of the RNA particles after thawing the frozen composition is less than 0.3, preferably less than 0.2, more preferably less than 0.1. In some embodiments, the size (Z_{average}) of the RNA particles after thawing the frozen composition is between about 50 nm and about 500 nm, preferably between about 40 nm and about 200 nm, more preferably between about 40 nm and about 120 nm, and the size (Z_{averge}) (and/or size distribution and/or PDI) of the RNA particles after thawing the frozen composition is essentially equal to the size (Z_{average}) (and/or size distribution and/or PDI) of the RNA particles before freezing. In some embodiments, the size (Z_{average}) of the RNA particles after thawing the frozen composition is between about 50 nm and about 500 nm, preferably between about 40 nm and about 200 in, more preferably between about 40 nm and about 120 nm, and the PDI of the RNA particles after thawing the frozen composition is less than 0.3 (preferably less than 0.2, more preferably less than 0.1).

In some embodiments of the first and second aspects, the size of the RNA particles and the RNA integrity of the composition after one freeze/thaw cycle, preferably after two freeze/thaw cycles, more preferably after three freeze/thaw cycles, more preferably after four freeze/thaw cycles, more preferably after five freeze/thaw cycles or more, are substantially the same as (i.e., are essentially equal to) the size of the RNA particles and the RNA integrity of the initial composition (i.e., before the composition has been frozen for the first time).

In a third aspect, the present disclosure provides a method of preparing a composition comprising LNPs dispersed in a final aqueous phase, wherein the LNPs comprise a cationically ionizable lipid and RNA; the final aqueous phase comprises a buffer system comprising a final buffer substance, the final buffer substance having the formula N(R¹)(R²)(R³), its N-oxide, or a protonated form thereof, wherein R¹, R², and R³ are as defined in the first aspect; wherein the method comprises:
(I) preparing a formulation comprising LNPs dispersed in the final aqueous phase, wherein the LNPs comprise the cationically ionizable lipid and RNA; and
(II) optionally freezing the formulation to about −10° C. or below, thereby obtaining the composition, wherein step (I) comprises:
(a) preparing an RNA solution containing water and a first buffer system;
(b) preparing an ethanolic solution comprising the cationically ionizable lipid and, if present, one or more additional lipids;
(c) mixing the RNA solution prepared under (a) with the ethanolic solution prepared under (b), thereby preparing a (first) intermediate formulation comprising the LNPs dispersed in a (first) intermediate aqueous phase comprising the first buffer system; and
(d) filtrating the first intermediate formulation prepared under (c) using a final aqueous buffer solution comprising the final buffer system, thereby preparing the formulation comprising the LNPs dispersed in the final aqueous phase.

As demonstrated in the present application, using a particular buffer system based on the above specified buffer substances, in particular TEA and its protonated form, instead of PBS in a composition comprising LNPs inhibits the formation of a very stable folded form (also called “light migrating species (LMS)” herein) of RNA. Furthermore, the present application demonstrates that, surprisingly, by using this buffer system, it is possible to obtain an LNP RNA composition having improved RNA integrity after storage in liquid form for about 3 months. Thus, the composition prepared by the claimed method provides improved stability, can be stored in a temperature range compliant to regular technologies in pharmaceutical practice, and provides a ready-to-use preparation.

In some embodiments of the third aspect, the final buffer substance is a tertiary amine (i.e., none of R¹, R², and R³ is H) or a protonated form thereof. Thus, in some embodiments, each of R¹, R², and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is CH(C₁₋₅ alkylene-R⁴)₂, or C(C₁₋₅ alkylene-R⁴)₃, as specified above. In some embodiments, the tertiary amine is a monoamine. In some embodiments, the tertiary amine is selected from the group consisting of bis(2-hydroxyethyl)amino-tris(hydroxymethyl)methane (Bis-Tris-methane or BTM), triethanolamine (TEA), ethyldiethanolamine, 2-(diethylamino)ethan-1-ol, triethylamine, and 2-[2-(diethylamino)ethoxy]ethan-1-ol. In some embodiments, the tertiary amine comprises or is triethanolamine (TEA).

In some embodiments of the third aspect, the final buffer substance is a cyclic amine (i.e., N(R¹)(R²)(R³), wherein two of R¹, R², and R³ join together with the nitrogen atom to form a 5- or 6-membered N-heterocyclic ring which is optionally substituted with one or two R⁵) or a protonated form thereof. Thus, in some embodiments, R¹ and R² join together with the nitrogen atom to form a 5- or 6-membered N-heterocyclic ring which is optionally substituted with one or two R⁵, as specified above. In some embodiments, the cyclic amine is selected from the group consisting of N,N′-bis(2-hydroxyethyl)piperazine and morpholine substituted with one or more C₁₋₆ alkylene-R⁴ (such as 2-hydroxyethyl) moieties.

In some embodiments of the third aspect, the final buffer substance is a secondary amine (i.e., one of R¹, R², and R³ is H) or a protonated form thereof. Thus, in some embodiments, R¹ is H and each of R² and R³ is independently selected from C_{J-6} alkyl, C₁₋₆ alkylene-R⁴, CH(C₁₋₅ alkylene-R⁴)₂, and C(C₁₋₅ alkylene-R⁴)₃, wherein at most one of R² and R³ is CH(C₁₋₅ alkylene-R⁴)₂, or C(C₁₋₅ alkylene-R⁴)₃, as specified above.

In some embodiments of the third aspect, the final buffer substance comprises at least one C₁₋₆ alkylene-R⁴ moiety. In those cases of these embodiments, where R⁴ is OH, it is preferred that the alkylene group has 2 to 6 carbon atoms, such as 2 to 4, e.g., 2, 3, or 4 carbon atoms. Thus, in these embodiments, wherein the final buffer substance comprises at least one C₁₋₆ alkylene-R⁴ moiety and R⁴ is OH, the at least one C₁₋₆ alkylene-R⁴ moiety preferably is C₂₋₆ alkylene-OH, more preferably C₂₋₄ alkylene-OH, more preferably C₂₋₃ alkylene-OH, such as C₂ alkylene-OH or 2-hydroxyethyl.

In some embodiments of third aspect, the final buffer substance is selected from bis(2-hydroxyethyl)amino-tris(hydroxymethyl)methane (Bis-Tris-methane or BTM) and its protonated form, triethanolamine (TEA) and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form. In some embodiments, the final buffer substance is selected from bis(2-hydroxyethyl)amino-tris(hydroxymethyl)methane (Bis-Tris-methane or BTM) and its protonated form, triethanolamine (TEA) and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, and N,N′-bis(2-hydroxyethyl)piperazine and its protonated form. In some embodiments, the final buffer substance comprises or is triethanolamine (TEA) or its protonated form.

In some embodiments of third aspect, in particular if it is desired to prepare a composition in frozen form, the method of the third aspect comprises (II) freezing the formulation to about −10° C. or below.

Thus, in these embodiments, conducting the method of the third aspect results in a composition in frozen form.

In some embodiments of the third aspect, in particular those where the composition is in frozen form, it is preferred that the composition (a) comprises a cryoprotectant; (b) has a pH between 4.0 and 8.0, preferably between 5.0 and 7.0, more preferably between 5.5 and 6.5 and most preferably about 5.5; or (c) comprises a cryoprotectant and has a pH between 4.0 and 8.0, preferably between 5.0 and 7.0, more preferably between 5.5 and 6.5 and most preferably about 5.5. In some embodiments, the cryoprotectant is (i) selected from the cryoprotectants disclosed herein; and/or (ii) is present in a concentration as disclosed herein. For example, the cryoprotectant may be selected from the group consisting of sucrose, glucose, glycerol, 1,2-propanediol, and 1,3-propanediol, such as from the group consisting of sucrose, glycerol and glucose; and/or may be present in a concentration of between about 100 mM and about 600 mM, preferably between about 200 mM and about 600 mM and more preferably between about 300 mM and about 500 mM. In some embodiments, the cryoprotectant is glycerol, which is optionally present in a concentration of between about 100 mM and about 600 mM, preferably between about 200 mM and about 600 mM and more preferably between about 300 mM and about 500 mM.

In some alternative embodiments, in particular if it is desired to prepare a composition in liquid form, the method of the third aspect does not comprise step (II). Thus, in these embodiments, conducting the method of the third aspect results in a composition in liquid form.

In some embodiments of the third aspect, step (I) further comprises one or more steps selected from diluting and filtrating, such as tangential flow filtrating and diafiltrating, after step (c). For example, a diluting step may comprise adding a dilution solution to an intermediate formulation. Such dilution solution may comprise one or more additional compounds and optionally the final buffer system, wherein the one or more additional compounds may comprise a cryoprotectant. The one or more filtrating steps (including steps (d), (f′), (g′), and (h′)) may be used to remove unwanted compounds (e.g., ethanol and/or one or more di- and/or polybasic organic acids) from the intermediate formulation and/or for increasing the RNA concentration of the intermediate formulation and/or for changing the pH and/or the buffer system of the intermediate formulation. To this end, an aqueous buffer solution can be used, which does not contain the unwanted compounds (such that the unwanted compounds are washed out from the intermediate formulation and into the aqueous buffer solution) and/or which is hypertonic compared to the aqueous buffer solution (such that water flows from the intermediate formulation to the aqueous buffer solution) and/or which has a pH and/or buffer system other than the pH and/or buffer system of the intermediate formulation.

In some embodiments of the third aspect, step (1) comprises:
(a′) providing an aqueous RNA solution;
(b′) providing a first aqueous buffer solution comprising a first buffer system;
(c′) mixing the aqueous RNA solution provided under (a′) with the first aqueous buffer solution provided under (b′) thereby preparing an RNA solution containing water and the first buffer system;
(d′) preparing an ethanolic solution comprising the cationically ionizable lipid and, if present, one or more additional lipids;
(e′) mixing the RNA solution prepared under (c′) with the ethanolic solution prepared under (d′), thereby preparing a first intermediate formulation comprising LNPs dispersed in a first aqueous phase comprising the first buffer system;
(f′) optionally filtrating the first intermediate formulation prepared under (e′) using a further aqueous buffer solution comprising a further buffer system, thereby preparing a further intermediate formulation comprising the LNPs dispersed in a further aqueous phase comprising the further buffer system, wherein the further aqueous buffer solution may be identical to or different from the first aqueous buffer solution;
(g′) optionally repeating step (f′) once or two or more times, wherein the further intermediate formulation comprising the LNPs dispersed in the further aqueous phase comprising the further buffer system obtained after step (f′) of one cycle is used as the first intermediate formulation of the next cycle, wherein in each cycle the further aqueous buffer solution may be identical to or different from the first aqueous buffer solution;
(h′) filtrating the first intermediate formulation obtained in step (e′), if step (f′) is absent, or the further intermediate formulation obtained in step (f′), if step (f′) is present and step (g′) is not present, or the further intermediate formulation obtained after step (g′), if steps (f′) and (g′) are present, using a final aqueous buffer solution comprising the final buffer system; and
(i′) optionally diluting the formulation obtained in step (h′) with a dilution solution; thereby preparing the formulation comprising the LNPs dispersed in the final aqueous phase.

In some embodiments of the third aspect, the concentration of the final buffer substance, in particular the total concentration of the final buffer substance and its protonated form, in the composition is between about 10 mM and about 200 mM, such as between about 20 mM and about 180 mM, between about 30 mM and about 170 mM, between about 40 mM and about 160 mM, between about 50 mM and about 50 mM, between about 60 mM and about 140 mM, between about 70 mM and about 130 mM, between about 80 mM and about 120 mM, between about 90 mM and about 110 mM. In some embodiments, the concentration of the final buffer substance, in particular the total concentration of the final buffer substance and its protonated form, in the composition is between about 15 mM and about 100 mM, preferably between about 20 mM and about 80 mM, more preferably between about 40 mM and about 60 mM, such as about 50 mM.

In some embodiments of the third aspect, the final buffer system further comprises an anion, which is preferably selected from the group consisting of chloride, acetate, glycolate, lactate, and the anion of a di- or tricarboxylic acid, such as the anion of citric acid, succinic acid, malonic acid, glutaric acid, or adipic acid. In some embodiments, where the final buffer system comprises an anion, the concentration of the anion in the composition is at least equal to the concentration of the final buffer substance in the composition. For example, the concentration of the anion in the composition may be higher than the concentration of the final buffer substance in the composition. Thus, in those embodiments of the third aspect, where the concentration of the final buffer substance in the composition is x being within the range between about 10 mM and about 200 mM, the concentration of the anion in the composition is at least equal to x, e.g., higher than x.

In some embodiments of the third aspect, wherein the formulation obtained in step (I) and/or the composition comprise(s) a cryoprotectant, said cryoprotectant comprises one or more compounds selected from the group consisting of carbohydrates and alcohols (such as sugar alcohols or lower alcohols). For example, the cryoprotectant may be selected from the group consisting of sucrose, glucose, glycerol, 1,2-propanediol, 1,3-propanediol, sorbitol, and a combination thereof (such as from the group consisting of sucrose, glucose, glycerol, 1,2-propanediol, 1,3-propanediol, and a combination thereof or from the group consisting of sucrose, glucose, glycerol, sorbitol, and a combination thereof). In some embodiments, the formulation obtained in step (I) and/or the composition comprise(s) sucrose and/or glycerol as cryoprotectant.

In some embodiments of the third aspect, wherein the formulation obtained in step (I) and/or the composition comprise(s) a cryoprotectant, the concentration of the cryoprotectant in the formulation and/or composition is at least 1% w/v, such as at least 2% w/v, at least 3% w/v, at least 4% w/v, at least 5% w/v, at least 6% w/v, at least 7% w/v, at least 8% w/v or at least 9% w/v. In some embodiments, the concentration of the cryoprotectant in the formulation and/or composition is up to 25% w/v, such as up to 20% w/v, up to 19% w/v, up to 18% w/v, up to 17% w/v, up to 16% w/v, up to 15% w/v, up to 14% w/v, up to 13% w/v, up to 12% w/v, or up to 11% w/v. In some embodiments, the concentration of the cryoprotectant in the formulation and/or composition is 1% w/v to 20% w/v, such as 2% w/v to 19% w/v, 3% w/v to 18% w/v, 4% w/v to 17% w/v, 5% w/v to 16% w/v, 5% w/v to 15% w/v, 6% w/v to 14% w/v, 7% w/v to 13% w/v, 8% w/v to 12% w/v, 9% w/v to 11% w/v, or about 10% w/v. In some embodiments, the formulation and/or composition comprise(s) a cryoprotectant (such as sucrose, glucose, glycerol, 1,2-propanediol, 1,3-propanediol, or a combination thereof, in particular, sucrose and/or glycerol) in a concentration of from 5% w/v to 15% w/v, such as from 6% w/v to 14% w/v, from 7% w/v to 13% w/v, from 8% w/v to 12% w/v, or from 9% w/v to 11% w/v, or in a concentration of about 10% w/v. For example, the method of the third aspect may comprise a diluting step using a dilution solution, wherein the dilution solution comprises a sufficient amount of a cryoprotectant in order to achieve the above concentrations of cryoprotectant in the formulation obtained in step (1) and/or the composition.

In some embodiments of the third aspect, wherein the formulation obtained in step (1) and/or the composition comprise(s) a cryoprotectant, the cryoprotectant is present in a concentration resulting in an osmolality of the composition in the range of from about 50×10⁻³ osmol/kg to about 1000×10⁻³ osmol/kg (such as from about 50×10⁻³ osmol/kg to about 500×10⁻³ osmol/kg, from about 50×10⁻³ osmol/kg to about 480×10⁻³ osmol/kg, from about 60×10⁻³ osmol/kg to about 460×10⁻³ osmol/kg, from about 70×10⁻³ osmol/kg to about 440×10⁻³ osmol/kg, from about 80×10⁻³ osmol/kg to about 420×10⁻³ osmol/kg, from about 90×10⁻³ osmol/kg to about 400×10⁻³ osmol/kg, from about 100×10⁻³ osmol/kg to about 380×10⁻³ osmol/kg, from about 120×10⁻³ osmol/kg to about 360×10⁻³ osmol/kg, from about 140×10⁻³ osmol/kg to about 340×10⁻³ osmol/kg, from about 160×10⁻³ osmol/kg to about 310×10⁻³ osmol/kg, from about 180×10⁻³ osmol/kg to about 300×10⁻³ osmol/kg, or from about 200×10⁻³ osmol/kg to about 300×10⁻³ osmol/kg), based on the total weight of the formulation/composition. For example, the method of the third aspect may comprise a diluting step using a dilution solution, wherein the dilution solution comprises a sufficient amount of a cryoprotectant in order to achieve the above osmolality values in the formulation obtained in step (I) and/or the composition.

In some embodiments of the third aspect, the formulation obtained in step (I) and/or the composition is/are substantially free of a cryoprotectant.

In some embodiments of the third aspect, the pH of the final buffer system (and the pH of the composition) is between about 4.0 and about 8.0. For example, the pH of the final buffer system (and the pH of the composition) may be between about 4.5 and about 8.0, such as between about 5.0 and about 8.0, between about 5.5 and about 8.0, between about 6.0 and about 8.0, between about 6.5 and about 8.0, between about 6.8 and about 7.9, between about 7.0 and about 7.8 or about 7.5. In some embodiments of the third aspect, the first buffer system (and the pH of the RNA solution obtained in step (a)) has a pH of below 6.0, preferably at most about 5.5, such as at most about 5.0, at most about 4.9, at most about 4.8, at most about 4.7, at most about 4.6, or at most about 4.5. For example, the pH of first buffer system (and the pH of the RNA solution obtained in step (a)) may be between about 3.5 and about 5.9, such as between about 4.0 and about 5.5, or between about 4.5 and about 5.0. To this end, the RNA solution obtained in step (a) may further comprises one or more di- and/or polybasic organic acids (e.g., citrate anions and/or anions of EDTA). In some embodiments, it is preferred that step (d) is conducted under conditions which remove one or more unwanted substances (e.g., ethanol and/or the one or more di- and/or polybasic organic acids) resulting in the formulation comprising the LNPs dispersed in a final aqueous phase with the final aqueous phase being substantially free of such one or more unwanted substances. For example, such conditions can include subjecting the intermediate formulation comprising the LNPs dispersed in the intermediate aqueous phase obtained in step (c) to at least one step of filtrating, such as tangential flow filtrating or diafiltrating, using a final buffer solution comprising the final buffer system (i.e., the final buffer substance), wherein the final buffer solution does not contain the one or more unwanted substances. Alternatively, such conditions can include (i) subjecting the intermediate formulation comprising the LNPs dispersed in the intermediate aqueous phase obtained in step (c) (i.e., a first intermediate formulation) to at least one step of filtrating, such as tangential flow filtrating or diafiltrating, using a further aqueous buffer solution comprising a further buffer system, thereby preparing a further intermediate formulation comprising the LNPs dispersed in a further aqueous phase comprising the further buffer system, wherein the further buffer system of the further aqueous buffer solution may be identical to or different from the buffer system used in step (a); (ii) optionally repeating step (i) once or two or more times, wherein the further intermediate formulation comprising the LNPs dispersed in the further aqueous phase obtained after step (i) of one cycle is used as the first intermediate formulation of the next cycle, wherein in each cycle the further buffer system of the further aqueous buffer solution may be identical to or different from the first buffer system used in step (a); and (iii) subjecting the intermediate formulation obtained in step (i) (if step (ii) is not present), or the intermediate formulation obtained in step (ii) (if step (ii) is present) to at least one step of filtrating, such as tangential flow filtrating or diafiltrating, using the final aqueous buffer solution, wherein at least one of the intermediate and final aqueous buffer solutions (preferably all intermediate and final aqueous buffer solutions) does not contain the one or more unwanted substances.

Similarly, in some embodiments of the third aspect, where step (I) comprises steps (a′) to (e′) and (h′) (and optionally one or more of steps (f′), (g′) and (i′)), the first aqueous buffer solution (and the pH of the RNA solution obtained under step (c′)) has a pH of below 6.0, preferably at most about 5.5, such as at most about 5.0, at most about 4.9, at most about 4.8, at most about 4.7, at most about 4.6, or at most about 4.5. For example, the pH of the first aqueous buffer solution (and the pH of the RNA solution obtained under step (c′)) may be between about 3.5 and about 5.9, such as between about 4.0 and about 5.5, or between about 4.5 and about 5.0. To this end, the first aqueous buffer solution provided under (b′) (and the first aqueous phase) may further comprises one or more di- and/or polybasic organic acids (e.g., citrate anions and/or anions of EDTA). In these embodiments, it is preferred that least one of steps (f′) to (h′) is conducted under conditions which remove one or more unwanted substances (e.g., ethanol and/or the one or more di- and/or polybasic organic acids) from the first intermediate formulation and/or from the further intermediate formulation resulting in a further inter formulation comprising the LNPs dispersed in a further aqueous phase or in the final aqueous phase with the further and/or final aqueous phase being substantially free of the one or more unwanted substances. For example, such conditions can include using a further aqueous buffer solution and/or a final buffer solution, wherein at least one of the further aqueous buffer solution(s) and the final buffer solution (preferably all of the further aqueous buffer solution(s) and the final buffer solution) does not contain the one or more unwanted substances. In some embodiments, the filtrating steps can be tangential flow filtrating or diafiltrating, preferably tangential flow filtrating.

In some embodiments of the third aspect, the first buffer system used in step (a) comprises the final buffer substance used in step (d), preferably the buffer system and pH of the first buffer system used in step (a) are identical to the buffer system and pH of the final aqueous buffer solution used in step (d). For example, only one aqueous buffer solution is used in this embodiment of the third aspect.

Similarly, in some embodiments of the third, where step (I) comprises steps (a′) to (e′) and (h′) (and optionally one or more of steps (f′), (g′) and (i′)), each of the first buffer system and every further buffer system used in steps (b′), (f′) and (g′) comprises the final buffer substance used in step (h′), preferably the buffer system and pH of each of the first aqueous buffer solution and of every further aqueous buffer solution used in steps (b′), (f′) and (g′) are identical to the buffer system and pH of the final aqueous buffer solution. For example, the aqueous buffer solutions used in steps (b′), (f′), if present, (g′), if present, and (h′) of this embodiment of the third aspect are identical.

In some embodiments of the third aspect, the formulation and/or composition comprise(s) water as the main component and/or the total amount of solvent(s) other than water contained in the composition is less than about 1.0% (v/v), such as less than about 0.5% (v/v). For example, the amount of water contained in the formulation and/or composition may be at least 50% (w/w), such as at least at least 55% (w/w), at least 60% (w/w), at least 65% (w/w), at least 70% (w/w), at least 75% (w/w), at least 80% (w/w), at least 85% (w/w), at least 90% (w/w), or at least 95% (w/w). In particular, if the formulation and/or composition comprise(s) a cryoprotectant, the amount of water contained in the formulation and/or composition comprise(s) may be at least 50% (w/w), such as at least at least 55% (w/w), at least 60% (w/w), at least 65% (w/w), at least 70% (w/w), at least 75% (w/w), at least 80% (w/w), at least 85% (w/w), or at least 90% (w/w). If the formulation and/or composition is/are substantially free of a cryoprotectant, the amount of water contained in the formulation and/or composition may be at least 95% (w/w). Additionally or alternatively, the total amount of solvent(s) other than water contained in the composition may be less than about 1.0% (v/v), such as less than about 0.9% (v/v), less than about 0.8% (v/v), less than about 0.7% (v/v), less than about 0.6% (v/v), less than about 0.5% (v/v), less than about 0.4% (v/v), less than about 0.3% (v/v), less than about 0.2% (v/v), less than about 0.1% (v/v), less than about 0.05% (v/v), or less than about 0.01% (v/v). In this respect, a cryoprotectant which is liquid under normal conditions will not be considered as a solvent other than water but as cryoprotectant. In other words, the above optional limitation that the total amount of solvent(s) other than water contained in the composition may be less than about 1.0% (v/v), such as less than about 0.5% (v/v), does not apply to cryoprotectants which are liquids under normal conditions.

In some embodiments of the third aspect, the osmolality of the composition is at most about 1000×10⁻³ osmol/kg, such as between about 100×10⁻³ osmol/kg and about 750×10⁻³ osmol/kg. In some embodiments, the osmolality of the composition is at most about 500×10⁻³ osmol/kg, such as at most about 490×10⁻³ osmol/kg, at most about 480×10⁻³ osmol/kg, at most about 470×10⁻³ osmol/kg, at most about 460×10⁻³ osmol/kg, at most about 450×10⁻³ osmol/kg, at most about 440×10⁻³ osmol/kg, at most about 430×10⁻³ osmol/kg, at most about 420×10⁻³ osmol/kg, at most about 410×10⁻³ osmol/kg, at most about 400×10⁻³ osmol/kg, at most about 390×10⁻³ osmol/kg, at most about 380×10⁻³ osmol/kg, at most about 370×10⁻³ osmol/kg, at most about 360×10⁻³ osmol/kg, at most about 350×10⁻³ osmol/kg, at most about 340×10⁻³ osmol/kg, at most about 330×10⁻³ osmol/kg, at most about 320×10⁻³ osmol/kg, at most about 310×10⁻³ osmol/kg, or at most about 300×10⁻³ osmol/kg. If the composition does not comprise a cryoprotectant, the osmolality of the composition may be below 300×10⁻³ osmol/kg, such as at most about 250×10⁻³ osmol/kg, at most about 200×10⁻³ osmol/kg, at most about 150×10⁻³ osmol/kg, at most about 100×10⁻³ osmol/kg, at most about 50×10⁻³ osmol/kg, at most about 40×10⁻³ osmol/kg, or at most about 30×10⁻³ osmol/kg. If the composition comprises a cryoprotectant, it is preferred that the main part of the osmolality of the composition is provided by the cryoprotectant. For example, the cryoprotectant may provide at least 50%, such as at least 55%, at least 60%, at least 65%, at least 70%, at least 75%, at least 80%, at least 85%, or at least 90%, of the osmolality of the composition.

In some embodiments of the third aspect, the concentration of the RNA in the composition is about 5 mg/l to about 500 mg/l, such as about 10 mg/l to about 400 mg/l, about 10 mg/l to about 300 mg/l, about 10 mg/l to about 200 mg/l, about 10 mg/l to about 150 mg/l, or about 10 mg/l to about 100 mg/l, preferably about 10 mg/l to about 140 mg/l, more preferably about 20 mg/l to about 130 mg/l, more preferably about 30 mg/l to about 120 mg/l. In some embodiments, the concentration of the RNA in the composition is about 5 mg/l to about 150 mg/l. For example, the concentration of the RNA in the composition may be about 10 mg/l to about 140 mg/l, such as about 20 mg/l to about 130 mg/l, about 25 mg/l to about 125 mg/l, about 30 mg/l to about 120 mg/l, about 35 mg/l to about 115 mg/l, about 40 mg/l to about 110 mg/l, about 45 mg/l to about 105 mg/l, or about 50 mg/l to about 100 mg/l.

In some embodiments of the third aspect, the final buffer substance comprises a tertiary amine as defined herein (i.e., none of R¹, R², and R³ is H) or its protonated form, the pH of the composition is between about 4.0 and about 8.0, and the concentration of the RNA in the composition is about 5 mg/l to about 500 mg/l. In this embodiment, it is preferred that the pH of the composition is between about 4.5 and about 8.0 and the concentration of the RNA in the composition is about 20 mg/l to about 130 mg/l, such as about 30 mg/l to about 120 mg/l. In particularly preferred embodiment, the final buffer substance comprises a tertiary amine as defined herein or its protonated form; the pH of the composition is between about 5.0 and about 8.0; the concentration of the RNA in the composition is about 30 mg/l to about 120 mg/l; and the composition comprises a cryoprotectant. In an alternative particularly preferred embodiment, the final buffer substance comprises a tertiary amine as defined herein or its protonated form; the pH of the composition is between about 5.0 and about 8.0; the concentration of the RNA in the composition is about 30 mg/l to about 120 mg/l; and the composition is substantially free of a cryoprotectant. In some embodiments, the tertiary amine is a monoamine. In some embodiments, the tertiary amine is selected from the group consisting of bis(2-hydroxyethyl)amino-tris(hydroxymethyl)methane (Bis-Tris-methane or BTM), triethanolamine (TEA), ethyldiethanolamine, 2-(diethylamino)ethan-1-ol, triethylamine, and 2-[2-(diethylamino)ethoxy]ethan-1-ol. In some embodiments, the tertiary amine comprises or is triethanolamine (TEA).

In some embodiments of the third aspect, the final buffer substance comprises a cyclic amine as defined herein (i.e., N(R¹)(R²)(R³), wherein two of R¹, R², and R³ join together with the nitrogen atom to form a 5- or 6-membered N-heterocyclic ring which is optionally substituted with one or two R⁵) or its protonated form, the pH of the composition is between about 4.0 and about 8.0, and the concentration of the RNA in the composition is about 5 mg/l to about 500 mg/l. In this embodiment, it is preferred that the pH of the composition is between about 4.5 and about 8.0 and the concentration of the RNA in the composition is about 20 mg/l to about 130 mg/l, such as about 30 mg/l to about 120 mg/l. In particularly preferred embodiment, the final buffer substance comprises a cyclic amine as defined herein or its protonated form; the pH of the composition is between about 5.0 and about 8.0; the concentration of the RNA in the composition is about 30 mg/l to about 120 mg/l; and the composition comprises a cryoprotectant. In an alternative particularly preferred embodiment, the final buffer substance comprises a cyclic amine as defined herein or its protonated form; the pH of the composition is between about 5.0 and about 8.0; the concentration of the RNA in the composition is about 30 mg/l to about 120 mg/l; and the composition is substantially free of a cryoprotectant. In some embodiments, the cyclic amine is selected from the group consisting of N,N′-bis(2-hydroxyethyl)piperazine and morpholine substituted with one or more C₁₋₆ alkylene-R⁴ (such as 2-hydroxyethyl) moieties.

In some embodiments of the third aspect, the cationically ionizable lipid comprises a head group which includes at least one nitrogen atom which is capable of being protonated under physiological conditions.

In some embodiments of the third aspect, the cationically ionizable lipid has the structure of Formula (X):

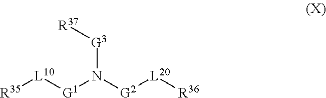

or a pharmaceutically acceptable salt, tautomer, prodrug or stereoisomer thereof, wherein L¹⁰, L²⁰, G¹, G², G³, R³⁵, R³⁶, and R³⁷ are as defined herein. In some embodiments, the cationically ionizable lipid is selected from the following: structures X-1 to X-36 (shown herein); and/or structures A to F (shown herein); and/or N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14). In some embodiments, the cationically ionizable lipid is the lipid having the structure X-3.

In some embodiments of the third aspect, the cationically ionizable lipid has the structure of Formula (XI):

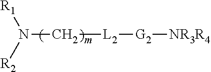

wherein R₁, R₂, R₃, R₄, R₅, R₆, G₁, G₂, and m are as defined herein. In some embodiments, the cationically ionizable lipid is selected from the structures (XIV-1), (XIV-2), and (XIV-3) (shown herein).

In some embodiments of the third aspect, the cationically ionizable lipid comprises from about 20 mol % to about 80 mol %, preferably from about 25 mol % to about 65 mol %, more preferably from about 30 mol % to about 50 mol %, such as from about 40 mol % to about 50 mol %, of the total lipid present in the composition.

In one embodiment of the third aspect, the ethanolic solution prepared in step (b) or (d′) further comprises one or more additional lipids and the LNPs further comprise the one or more additional lipids. Preferably, the one or more additional lipids are selected from the group consisting of polymer conjugated lipids, neutral lipids, steroids, and combinations thereof. In some embodiments of the third aspect, the one or more additional lipids comprise a polymer conjugated lipid (e.g., a pegylated lipid; or a polysarcosine-lipid conjugate or a conjugate of polysarcosine and a lipid-like material), a neutral lipid (e.g., a phospholipid, such as DSPC), and a steroid (e.g., cholesterol), such that the LNPs comprise the cationically ionizable lipid as described herein, a polymer conjugated lipid (e.g., a pegylated lipid; or a polysarcosine-lipid conjugate or a conjugate of polysarcosine and a lipid-like material), a neutral lipid (e.g., a phospholipid, such as DSPC), and a steroid (e.g., cholesterol).

In some embodiments of the third aspect, wherein the one or more additional lipids comprise a polymer conjugated lipid, the polymer conjugated lipid is a pegylated lipid. For example, the pegylated lipid is selected from the group consisting of DSPE-PEG, DOPE-PEG, DPPE-PEG, and DMPE-PEG. In some embodiments, the pegylated lipid may have the following structure:

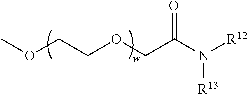

or a pharmaceutically acceptable salt, tautomer or stereoisomer thereof, wherein R¹², R¹³, and w are as defined herein.

In some embodiments of the third aspect, wherein the one or more additional lipids comprise a polymer conjugated lipid, the polymer conjugated lipid is a polysarcosine-lipid conjugate or a conjugate of polysarcosine and a lipid-like material. For example, the polysarcosine-lipid conjugate or conjugate of polysarcosine and a lipid-like material may be a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

In some embodiments of the third aspect, wherein the one or more additional lipids comprise a polymer conjugated lipid, the polymer conjugated lipid comprises from about 0.5 mol % to about 5 mol %, preferably from about 1 mol % to about 5 mol %, more preferably from about 1 mol % to about 4.5 mol % of the total lipid present in the composition.

In some embodiments of the third aspect, wherein the one or more additional lipids comprise a neutral lipid, the neutral lipid is a phospholipid. Such phospholipid is preferably selected from the group consisting of phosphatidylcholines, phosphatidylethanolamines, phosphatidylglycerols, phosphatidic acids, phosphatidylserines and sphingomyelins. Particular examples of phospholipids include distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE). In some embodiments, the neutral lipid is DSPC.

In some embodiments of the third aspect, wherein the one or more additional lipids comprise a neutral lipid (such as a phospholipid), the neutral lipid comprises from about 5 mol % to about 40 mol %, preferably from about 5 mol % to about 20 mol %, more preferably from about 5 mol % to about 15 mol % of the total lipid present in the composition.

In some embodiments of the third aspect, wherein the one or more additional lipids comprise a steroid, the steroid is a sterol such as cholesterol.

In some embodiments of the third aspect, wherein the one or more additional lipids comprise a steroid, the steroid comprises from about 10 mol % to about 65 mol %, preferably from about 20 mol % to about 60 mol %, more preferably from about 30 mol % to about 50 mol % of the total lipid present in the composition.

In some embodiments of the third aspect, the ethanolic solution comprises the cationically ionizable lipid, the polymer conjugated lipid, the neutral lipid (e.g., a phospholipid), and the steroid in a molar ratio of 20% to 60% of the cationically ionizable lipid, 0.5% to 15% of the polymer conjugated lipid, 5% to 25% of the neutral lipid, and 25% to 55% of the steroid, based on the total molar amount of lipids in the ethanolic solution. For example, the molar ratio may be 40% to 55% of the cationically ionizable lipid, 1.0% to 10% of the polymer conjugated lipid, 5% to 15% of the neutral lipid, and 30% to 50% of the steroid, such as 45% to 55% of the cationically ionizable lipid, 1.0% to 5% of the polymer conjugated lipid, 8% to 12% of the neutral lipid, and 35% to 45% of the steroid, based on the total molar amount of lipids in the ethanolic solution.

In some embodiments of the third aspect, the LNPs comprise at least about 75% of the RNA comprised in the composition. For example, the LNPs may comprise at least about 76%, such as at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95% of the RNA comprised in the composition.

In some embodiments of the third aspect, the RNA is mRNA.

In some embodiments of the third aspect, the RNA (such as mRNA) is encapsulated within or associated with the LNPs.

In some embodiments of the third aspect, the RNA (such as mRNA) (i) comprises a modified nucleoside in place of uridine; (ii) has a coding sequence which is codon-optimized; and/or (iii) has a coding sequence whose G/C content is increased compared to the wild-type coding sequence. In some embodiments, the modified nucleoside is selected from pseudouridine (W), N1-methyl-pseudouridine (m1 ψ), and 5-methyl-uridine (m5U).

In some embodiments of the third aspect, the RNA (such as mRNA) comprises one or more of the following (a) a 5′ cap, such as a cap1 or cap2 structure; (b) a 5′ UTR; (c) a 3′ UTR; and (d) a poly-A sequence. In some embodiments, the poly-A sequence comprises at least 100 A nucleotides, wherein the poly-A sequence preferably is an interrupted sequence of A nucleotides.

In some embodiments of the third aspect, the RNA (such as mRNA) encodes one or more polypeptides. In some embodiments, the one or more polypeptides are pharmaceutically active polypeptides and/or comprise an epitope for inducing an immune response against an antigen in a subject.

In some embodiments of the third aspect, the pharmaceutically active polypeptide and/or the antigen or epitope is derived from or is a protein of a pathogen, an immunogenic variant of the protein, or an immunogenic fragment of the protein or the immunogenic variant thereof. In some embodiments, the pathogen is a pathogen causing an infectious disease.

In some embodiments of the third aspect, the pharmaceutically active polypeptide and/or the antigen or epitope is derived from or is a SARS-CoV-2 spike (S) protein, an immunogenic variant thereof, or an immunogenic fragment of the SARS-CoV-2 S protein or the immunogenic variant thereof. In some embodiments, the RNA (such as mRNA) comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof. In some embodiments of the third aspect, the SARS-CoV2 S protein variant has proline residue substitutions at positions 986 and 987 of SEQ ID NO: 1. In some embodiments, the SARS-CoV2 S protein variant has at least 80% identity to the amino acid sequence of amino acids 17 to 1273 of SEQ ID NO:7 or the amino acid sequence of amino acids 17 to 1273 of SEQ ID NO: 1. In some embodiments, the fragment comprises the receptor binding domain (RBD) of the SARS-CoV-2 S protein. In some embodiments, the fragment of (i) the SARS-CoV-2 S protein or (ii) the immunogenic variant of the SARS-CoV-2 S protein has at least 80% identity to the amino acid sequence of amino acids 327 to 528 of SEQ ID NO: 1.

In a fourth aspect, the present disclosure provides a method of preparing an aqueous RNA composition, wherein the method comprises (I) preparing a formulation comprising RNA and an aqueous phase, wherein the aqueous phase comprises a buffer substance, the buffer substance having the formula N(R¹)(R²)(R³), its N-oxide, or a protonated form thereof, wherein R¹, R², and R³ are as defined in the first aspect; and (II) optionally freezing the formulation to about −10° C. or below, thereby obtaining the composition.

In some embodiments of the fourth aspect, the method comprises step (II) (i.e., freezing the formulation to about −10° C. or below).

In some embodiments of the fourth aspect, in particular those where the method comprises step (II), it is preferred that the composition (a) comprises a cryoprotectant; (b) has a pH between 4.0 and 8.0, preferably between 5.0 and 7.0, more preferably between 5.5 and 6.5 and most preferably about 5.5; or (c) comprises a cryoprotectant and has a pH between 4.0 and 8.0, preferably between 5.0 and 7.0, more preferably between 5.5 and 6.5 and most preferably about 5.5. In some embodiments, the cryoprotectant is (i) selected from the cryoprotectants disclosed herein; and/or (ii) is present in a concentration as disclosed herein. For example, the cryoprotectant may be selected from the group consisting of sucrose, glucose, glycerol, 1,2-propanediol, 1,3-propanediol, and a combination thereof, such as from the group consisting of sucrose, glycerol and glucose; and/or may be present in a concentration of between about 100 mM and about 600 mM, preferably between about 200 mM and about 600 mM and more preferably between about 300 mM and about 500 mM. In some embodiments, the cryoprotectant is glycerol, which is optionally present in a concentration of between about 100 mM and about 600 mM, preferably between about 200 mM and about 600 mM and more preferably between about 300 mM and about 500 mM.

In some embodiments of the fourth aspect, the buffer substance is selected from BTM and its protonated form, TEA and its protonated form, ethyldiethanolamine and its protonated form, 2-(diethylamino)ethan-1-ol and its protonated form, triethylamine and its protonated forn, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form. In some embodiments, the buffer substance comprises or is triethanolamine (TEA) or its protonated form.

It is understood that any embodiment described herein in the context of the first, second or third aspect may also apply to any embodiment of the fourth aspect.

In a fifth aspect, the present disclosure provides a method of storing a composition, comprising preparing a composition according to the method of the third aspect and storing the composition at a temperature ranging from about −90° C. to about −10° C., such as from about −90° C. to about −40° C. or from about −40° C. to about −25° C. or from about −25° C. to about −10° C., or a temperature of about −20° C. In some embodiments of the fifth aspect, storing the frozen composition is for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. In some embodiments of the fifth aspect, storing the frozen composition is for at least 4 weeks, preferably at least 1 month, more preferably at least 2 months, more preferably at least 3 months, more preferably at least 6 months at −20° C. In some embodiments of the fifth aspect, the composition can be stored at −70° C.

In some embodiments of the fifth aspect, it is preferred that the composition (a) comprises a cryoprotectant; (b) has a pH between 4.0 and 8.0, preferably between 5.0 and 7.0, more preferably between 5.5 and 6.5 and most preferably about 5.5; or (c) comprises a cryoprotectant and has a pH between 4.0 and 8.0, preferably between 5.0 and 7.0, more preferably between 5.5 and 6.5 and most preferably about 5.5. In some embodiments, the cryoprotectant is (i) selected from the cryoprotectants disclosed herein; and/or (ii) is present in a concentration as disclosed herein. For example, the cryoprotectant may be selected from the group consisting of sucrose, glucose, glycerol, 1,2-propanediol, 1,3-propanediol, and a combination thereof, such as from the group consisting of sucrose, glycerol and glucose; and/or may be present in a concentration of between about 100 mM and about 600 mM, preferably between about 200 mM and about 600 mM and more preferably between about 300 mM and about 500 mM. In some embodiments, the cryoprotectant is glycerol, which is optionally present in a concentration of between about 100 mM and about 600 mM, preferably between about 200 mM and about 600 mM and more preferably between about 300 mM and about 500 mM.

In some embodiments of the fifth aspect, the buffer substance is selected from BTM and its protonated form, TEA and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediainine and its protonated form, and trimethylamine N-oxide and its protonated form. In some embodiments, the buffer substance comprises or is triethanolamine (TEA) or its protonated form.

In some embodiments of the fifth aspect, the method of storing a composition comprises preparing a composition according to the method of the third aspect comprising step (II) (i.e., freezing the formulation to about −10° C. or below); storing the frozen composition at a temperature ranging from about −90° C. to about −10° C. for a certain period of time (e.g., at least one week, such as at least 2 weeks, at least three weeks, at least four weeks, at least one month, at least two months, at least three months, at least 4 months, at least 6 months, at least 12 months, at least 24 months, or at least 36 months); and storing the frozen composition a temperature ranging from about 0° C. to about 20° C. for a certain period of time (e.g., at least four weeks, such as at least one month, at least two months, at least three months, at least 4 months, or at least 6 months).

It is understood that any embodiment described herein in the context of the first, second or third aspect may also apply to any embodiment of the fifth aspect.

In a sixth aspect, the present disclosure provides a method of storing a composition, comprising preparing a liquid composition according to the method of the third aspect and storing the liquid composition at a temperature ranging from about 0° C. to about 20° C., such as from about 1° C. to about 15° C., from about 2° C. to about 10° C., or from about 2° C. to about 8° C., or at a temperature of about 5° C.

In some embodiments of the sixth aspect, storing the liquid composition is for at least 1 week, such as at least 2 weeks, at least 3 weeks, at least 4 weeks, at least 1 month, at least 2 months, at least 3 months, at least 6 months, at least 12 months, or at least 24 months, preferably at least 4 weeks. In some embodiments of the sixth aspect, storing the liquid composition is for at least 4 weeks, preferably at least 1 month, more preferably at least 2 months, more preferably at least 3 months, more preferably at least 6 months at 5° C.

In some embodiments of the sixth aspect, the method of storing a composition comprises preparing a composition according to the method of the third aspect comprising step (II) (i.e., freezing the formulation to about −10° C. or below); and storing the frozen composition at a temperature ranging from about 0° C. to about 20° C. for a certain period of time (e.g., at least four weeks).

In some embodiments of the sixth aspect, the buffer substance is selected from BTM and its protonated form, TEA and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form. In some embodiments, the buffer substance comprises or is triethanolamine (TEA) or its protonated form.

It is understood that any embodiment described herein in the context of the first, second or third aspect may also apply to any embodiment of the sixth aspect.

In a seventh aspect, the present disclosure provides a composition preparable by the method of the, third, fourth, fifth, or sixth aspect. In some embodiments of the seventh aspect, the composition can be in frozen form which, preferably, can be stored at a temperature of about −90° C. or higher, such as about −90° C. to about −10° C. For example, the frozen composition of the seventh aspect can be stored at a temperature ranging from about −90° C. to about −40° C. or from about −40° C. to about −25° C. or from about −25° C. to about −10° C., or a temperature to about −20°. In some embodiments of the seventh aspect, the composition can be stored for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. For example, the frozen composition can be stored for at least 4 weeks, preferably at least 1 month, more preferably at least 2 months, more preferably at least 3 months, more preferably at least 6 months at −20° C.

In some embodiments of the seventh aspect, where the composition is in frozen form, it is preferred that the composition (a) comprises a cryoprotectant; (b) has a pH between 4.0 and 8.0, preferably between 5.0 and 7.0, more preferably between 5.5 and 6.5 and most preferably about 5.5; or (c) comprises a cryoprotectant and has a pH between 4.0 and 8.0, preferably between 5.0 and 7.0, more preferably between 5.5 and 6.5 and most preferably about 5.5. In some embodiments, the cryoprotectant is (i) selected from the cryoprotectants disclosed herein; and/or (ii) is present in a concentration as disclosed herein. For example, the cryoprotectant may be selected from the group consisting of sucrose, glucose, glycerol, 1,2-propanediol, 1,3-propanediol, and a combination thereof, such as from the group consisting of sucrose, glycerol and glucose; and/or may be present in a concentration of between about 100 mM and about 600 mM, preferably between about 200 mM and about 600 mM and more preferably between about 300 mM and about 500 mM. In some embodiments, the cryoprotectant is glycerol, which is optionally present in a concentration of between about 100 mM and about 600 mM, preferably between about 200 mM and about 600 mM and more preferably between about 300 mM and about 500 mM.

In some embodiments of the seventh aspect, the buffer substance is selected from BTM and its protonated form, TEA and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form. In some embodiments, the buffer substance comprises or is triethanolamine (TEA) or its protonated form.

In some embodiments of the seventh aspect, where the composition is in frozen form, the RNA integrity after thawing the frozen composition is at least 50%, such as at least 60%, at least 70%, at least 80%, at least 85%, at least 90%, at least 95%, at least 97%, at least 98% or substantially 100%, e.g., after thawing the frozen composition which has been stored at −20° C., compared to the RNA integrity of the composition before the composition has been frozen.

In some embodiments, the initial RNA integrity of the composition (i.e., after its preparation but before freezing) is at least 50% and the RNA integrity of the composition after thawing the frozen composition is at least 90%, preferably at least 95%, more preferably at least 97%, more preferably at least 98%, more preferably substantially 100%, of the initial RNA integrity.

Additionally or alternatively, in some embodiments of the seventh aspect, where the composition is in frozen form, the size (Z_{average}) (and/or size distribution and/or polydispersity index (PDI)) of the RNA particles after thawing the frozen composition is essentially equal to the size (Z_{average}) (and/or size distribution and/or PDI) of the RNA particles before the composition has been frozen. In some embodiments, the size (Z_{average}) of the RNA particles after thawing the frozen composition is between about 50 nm and about 500 nm, preferably between about 40 nm and about 200 nm, more preferably between about 40 nm and about 120 nm. In some embodiments, the PDI of the RNA particles after thawing the frozen composition is less than 0.3, preferably less than 0.2, more preferably less than 0.1. In some embodiments, the size (Z_{average}) of the RNA particles after thawing the frozen composition is between about 50 nm and about 500 nm, preferably between about 40 nm and about 200 nm, more preferably between about 40 nm and about 120 nm, and the size (Z_{average}) (and/or size distribution and/or PDI) of the RNA particles after thawing the frozen composition is essentially equal to the size (Z_{average}) (and/or size distribution and/or PDI) of the RNA particles before freezing. In some embodiments, the size (Z_{average}) of the RNA particles after thawing the frozen composition is between about 50 nm and about 500 nm, preferably between about 40 nm and about 200 nm, more preferably between about 40 nm and about 120 nm, and the PDI of the RNA particles after thawing the frozen composition is less than 0.3 (preferably less than 0.2, more preferably less than 0.1).

In some embodiments, the size of the RNA particles and the RNA integrity of the composition after one freeze/thaw cycle, preferably after two freeze/thaw cycles, more preferably after three freeze/thaw cycles, more preferably after four freeze/thaw cycles, more preferably after five freeze/thaw cycles or more, are substantially the same as (i.e., are essentially equal to) the size of the RNA particles and the RNA integrity of the initial composition (i.e., before the composition has been frozen for the first time).

In an alternative embodiment of the seventh aspect, the composition is in liquid form.

In some embodiments of the seventh aspect, where the composition is in liquid form, the RNA integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), is sufficient to produce the desired effect, e.g., to induce an immune response. For example, the RNA integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), may be at least 50%, such as at least 60%, at least 70%, at least 80%, at least 85%, at least 90%, at least 95%, at least 97% or at least 98%, compared to the RNA integrity before storage. In some embodiments, the RNA integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), may be at least 90%, at least 95%, at least 97% or at least 98%, compared to the RNA integrity before storage. In some embodiments, the RNA integrity of the composition after storage for at least four weeks, preferably at a temperature of 0° C. or higher, such as about 2° C. to about 8° C., is at least 50%, such as at least 60%, at least 70%, at least 80%, at least 85%, at least 90%, at least 95%, at least 97% or at least 98%, compared to the RNA integrity before storage. In some embodiments, the RNA integrity of the composition after storage for at least four weeks, preferably at a temperature of 0° C. or higher, such as about 2° C. to about 8° C., is at least 90%, at least 95%, at least 97% or at least 98%, compared to the RNA integrity before storage.

In some embodiments, the initial RNA integrity of the liquid composition (i.e., after its preparation but before storage) is at least 50% and the RNA integrity of the liquid composition after storage for at least one week (such as for at least 2 weeks, at least three weeks, at least four weeks, at least one month, at least two months, or at least 3 months), preferably at a temperature of 0° C. or higher, such as about 2° C. to about 8° C., is at least 90%, preferably at least 95%, more preferably at least 97%, more preferably at least 98%, of the initial RNA integrity. In some embodiments, the initial RNA integrity of the liquid composition (i.e., after its preparation but before storage) is at least 50% and the RNA integrity of the liquid composition after storage for at least one week (such as for at least 2 weeks, at least three weeks, at least four weeks, at least one month, at least two months, or at least 3 months), preferably at a temperature of 0° C. or higher, such as about 2° C. to about 8° C., is at least 90% of the initial RNA integrity.

Additionally or alternatively, in some embodiments of the seventh aspect, where the composition is in liquid form, the size (Z_{average}) (and/or size distribution and/or polydispersity index (PDI)) of the RNA particles of the liquid composition, when stored, e.g., at 0° C. or higher for at least one week (such as for at least 2 weeks, at least three weeks, at least four weeks, at least one month, at least two months, at least three months, at least 4 months, or at least 6 months), is sufficient to produce the desired effect, e.g., to induce an immune response. For example, the size (Z_{average}) (and/or size distribution and/or polydispersity index (PDI)) of the RNA particles of the liquid composition, when stored, e.g., at 0° C. or higher for at least one week (such as for at least 2 weeks, at least three weeks, at least four weeks, at least one month, at least two months, at least three months, at least 4 months, or at least 6 months), is essentially equal to the size (Z_{average}) (and/or size distribution and/or PDI) of the RNA particles of the initial composition, i.e., before storage. In some embodiments, the size (Z_{average}) of the RNA particles after storage of the liquid composition, e.g., at 0° C. or higher for at least one week (such as for at least four weeks or at least three months) is between about 50 nm and about 500 nm, preferably between about 40 nm and about 200 nm, more preferably between about 40 nm and about 120 nm. In some embodiments, the PDI of the RNA particles after storage of the liquid composition, e.g., at 0° C. or higher for at least one week (such as for at least four weeks or at least three months) is less than 0.3, preferably less than 0.2, more preferably less than 0.1. In some embodiments, the size (Z_{average}) of the RNA particles after storage of the liquid composition, e.g., at 0° C. or higher for at least one week (such as for at least four weeks or at least three months) is between about 50 nm and about 500 nm, preferably between about 40 nm and about 200 nm, more preferably between about 40 nm and about 120 nm, and the size (Z_{average}) (and/or size distribution and/or PDI) of the RNA particles after storage of the liquid composition, e.g., at 0° C. or higher for at least one week (such as for at least four weeks or at least three months) is essentially equal to the size (Z_{average}) (and/or size distribution and/or PDI) of the RNA particles before storage. In some embodiments, the size (Z_{average}) of the RNA particles after storage of the liquid composition, e.g., at 0° C. or higher for at least one week (such as for at least four weeks or at least three months) is between about 50 nm and about 500 nm, preferably between about 40 nm and about 200 nm, more preferably between about 40 nm and about 120 nm, and the PDI of the RNA particles after storage of the liquid composition, e.g., at 0° C. or higher for at least one week (such as for at least four weeks or at least three months) is less than 0.3 (preferably less than 0.2, more preferably less than 0.1).

It is understood that any embodiment described herein in the context of the first, second, third, fourth, fifth, or sixth aspect may also apply to any embodiment of the seventh aspect.

In an eighth aspect, the present disclosure provides a method for preparing a ready-to-use pharmaceutical composition, the method comprising the steps of providing a frozen composition prepared by the method of the third, fourth, or fifth aspect and thawing the frozen composition thereby obtaining the ready-to-use pharmaceutical composition.

It is understood that any embodiment described herein in the context of the first, second, third, fourth, fifth, sixth, or seventh aspect may also apply to any embodiment of the eighth aspect.

In a ninth aspect, the present disclosure provides a method for preparing a ready-to-use pharmaceutical composition, the method comprising the steps of providing a liquid composition prepared by the method of the third, fourth, or sixth aspect thereby obtaining the ready-to-use pharmaceutical composition.

It is understood that any embodiment described herein in the context of the first, second, third, fourth, fifth, sixth, seventh, or eighth aspect may also apply to any embodiment of the ninth aspect.

In a tenth aspect, the present disclosure provides a ready-to-use pharmaceutical composition preparable by the method of the eighth or ninth aspect.

It is understood that any embodiment described herein in the context of the first, second, third, fourth, fifth, sixth, seventh, eighth, or ninth aspect may also apply to any embodiment of the tenth aspect.

In an eleventh aspect, the present disclosure provides a composition of any one of the first, seventh, and tenth aspect for use in therapy.

It is understood that any embodiment described herein in the context of the first, second, third, fourth, fifth, sixth, seventh, eighth, ninth, or tenth aspect may also apply to any embodiment of the eleventh aspect.

In a twelfth aspect, the present disclosure provides a composition of any one of the first, seventh, and tenth aspect for use in inducing an immune response.

It is understood that any embodiment described herein in the context of the first, second, third, fourth, fifth, sixth, seventh, eighth, ninth, tenth, or eleventh aspect may also apply to any embodiment of the twelfth aspect.

In a thirteenth aspect, the present disclosure provides a method of transfecting cells, comprising adding a composition of any one of the first, second, seventh or tenth aspect to cells; and incubating the mixture of the composition and cells for a sufficient amount of time. In some embodiments, in particular those, where the RNA (such as mRNA) encodes a pharmaceutically active protein, the mixture of the composition and cells is incubated for a time sufficient to allow the expression of the pharmaceutically active protein. In some embodiments, in particular those, where the RNA is inhibitory RNA (such as siRNA) directed against a target mRNA, the mixture of the composition and cells is incubated for a time sufficient to allow the inhibition of the transcription and/or translation of the target mRNA. In some embodiments, the sufficient amount of time is at least one hour (such at least about 2 hours, at least about 3 hours, at least about 4 hours, at least about 5 hours, at least about 6 hours, at least about 9 hours, at least about 12 hours) and/or up to about 48 hours (such as up to about 36 or up to about 24 hours). In some embodiments of the thirteenth aspect, the method is conducted in vivo (i.e., the cells form part of an organ, a tissue and/or an organism of a subject). In some embodiments of the thirteenth aspect, the method is conducted in vitro (i.e., the cells do not form part of an organ, a tissue and/or an organism of a subject, e.g., the cells are an ex vivo cell culture).

It is understood that any embodiment described herein in the context of the first, second, third, fourth, fifth, sixth, seventh, eighth, ninth, tenth, eleventh or twelfth aspect may also apply to any embodiment of the thirteenth aspect.

In a fourteenth aspect, the present disclosure provides a use of a composition of any one of the first, second, seventh or tenth aspect for transfecting cells. In some embodiments of the fourteenth aspect, the use is an in vivo use (i.e., the cells form part of an organ, a tissue and/or an organism of a subject). In some embodiments of the fourteenth aspect, the use is an in vitro use (i.e., the cells do not form part of an organ, a tissue and/or an organism of a subject, e.g., the cells are an ex vivo cell culture).

It is understood that any embodiment described herein in the context of the first, second, third, fourth, fifth, sixth, seventh, eighth, ninth, tenth, eleventh, twelfth or thirteenth aspect may also apply to any embodiment of the fourteenth aspect.

In a further aspect, the present disclosure provides a kit comprising a composition of any one of the first, second, seventh, tenth, eleventh, or twelfth aspect or a pharmaceutical composition as described herein.

In some embodiments, the kit is for use in therapy, such as for inducing an immune response. In some embodiments, the kit is for use in inducing an immune response against a pathogen, such as for treating or preventing an infectious disease.

Further itemised embodiments are as follows:

1. A composition comprising (i) RNA; (ii) a cationically ionizable lipid; and (iii) an aqueous phase, wherein the aqueous phase comprises a buffer system comprising a buffer substance having the formula N(R¹)(R²)(R³), its N-oxide, or a protonated form thereof, wherein:
each of R¹, R², and R³ is independently selected from H, C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is H, CH(C₁₋₅ alkylene-R⁴)₂, or C(C₁₋₅ alkylene-R⁴)₃; or two of R¹, R², and R³ join together with the nitrogen atom to form a 5- or 6-membered N-heterocyclic ring which is optionally substituted with one or two R⁵;
each R⁴ is independently selected from —OH, —O-(C₁₋₆ alkylene-OH), and —N(R⁶)_{z}-(C₁₋₆ alkylene-OH)_{2-z}, wherein each z is independently selected from 0 and 1; and each R⁶ is independently selected from H and C₁₋₃ alkyl; and
each R⁵ is independently selected from C₁₋₆ alkyl, C₁₋₆ alkylene-R⁴, CH(C₁₋₅ alkylene-R⁴)₂, and C(C₁₋₅ alkylene-R⁴)₃.

2. The composition of item 1, wherein each of R¹, R², and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is CH(C₁₋₅ alkylene-R⁴)₂ or C(C₁₋₅ alkylene-R⁴)₃, preferably each of R¹, R², and R³ is independently selected from C₁_₄ alkyl, C₁₋₄ alkylene-R⁴, CH(C₁₋₃ alkylene-R⁴)₂, and C(C₁₋₃ alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₃ alkyl, C₁₋₃ alkylene-R⁴, CH(C₁₋₃ alkylene-R⁴)₂, and C(C₁a alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₂ alkyl, C₁₋₂ alkylene-R⁴, CH(C₁₋₂ alkylene-R⁴)₂, and C(C₁₋₂ alkylene-R⁴)₃, wherein at most one of R¹, R², and R³ is CH(C₁₋₂ alkylene-R⁴)₂ or C(C₁₋₂ alkylene-R⁴)₃.

3. The composition of item 1 or 2, wherein each of R¹, R², and R³ is independently selected from C₁₋₆ alkyl, C₁₋₆ alkylene-R⁴, and C(C₁₋₅ alkylene-R⁴)₃, wherein at most one of R¹, R², and R³ is C(C₁₋₅ alkylene-R⁴)₃, preferably each of R¹, R², and R³ is independently selected from C₁₋₄ alkyl, C₁₋₄ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₄ alkyl, C₁₋₃ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₂ alkyl, C₁₋₂ alkylene-R⁴, and C(C₁₋₂ alkylene-R⁴)₃, wherein at most one of R¹, R², and R³ is C(C₁₋₂ alkylene-R⁴)₃.

4. The composition of any one of items 1 to 3, wherein each of R¹, R², and R³ is independently selected from C₁₋₆ alkyl and C₁₋₆ alkylene-R⁴, preferably each of R¹, R², and R³ is independently selected from C₁₋₄ alkyl and C₁₋₄ alkylene-R⁴, more preferably each of R¹, R², and R³ is independently selected from C₁₋₃ alkyl and C₁₋₃ alkylene-R⁴, more preferably each of R¹, R², and R³ is independently selected from C₁₋₂ alkyl and C₁₋₂ alkylene-R⁴.

5. The composition of any one of items 1 to 4, wherein each R⁴ is independently selected from —OH, —O-(C₁₋₄ alkylene-OH), and —N(R⁶)_{z}-(C₁₋₄ alkylene-OH)₂—Z, wherein each z is independently selected from 0 and 1; and each R⁶ is independently selected from H and C₁₋₃ alkyl, preferably each R⁴ is independently selected from —OH, —O-(C₁₋₃ alkylene-OH), and —N(R⁶)_{z}-(C₁₋₃ alkylene-OH)_{2-z}, wherein each z is independently selected from 0 and 1; and each R⁶ is independently selected from H and C₁₋₃alkyl, more preferably each R⁴ is independently selected from —OH, —O-(C₁₋₂ alkylene-OH), and —N(R⁶)—(C₁₋₂ alkylene-OH)_{2-z}, wherein each z is independently selected from 0 and 1; and each R⁶ is independently selected from H and C₁₋₂ alkyl.

6. The composition of any one of items 1 to 5, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

7. The composition of any one of items 1 to 6, wherein each R⁴ is independently selected from —OH, 2-hydroxyethoxy, and bis(2-hydroxyethyl)amino.

8. The composition of any one of items 1 to 7, wherein each of R¹, R², and R³ is independently selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

9. The composition of any one of items 1 to 8, wherein all of R¹, R², and R³ are the same.

10. The composition of item 9, wherein all of R¹, R², and R³ are methyl, ethyl, or 2-hydroxyethyl.

11. The composition of any one of items 1 to 8, wherein R¹ and R² are the same and R³ differs from R¹ and R².

12. The composition of item 11, wherein each of R¹ and R² is 2-hydroxyethyl, ethyl, or methyl.

13. The composition of item 11 or 12, wherein R³ is selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

14. The composition of item 1, wherein R¹ and R² join together with the nitrogen atom to form a 5- or 6-membered N-heterocyclic ring which is optionally substituted with one or two R⁵.

15. The composition of item 14, wherein R³ is selected from C₁₋₆ alkyl, C₁₋₆ alkylene-R⁴, and C(C₁₋₅ alkylene-R⁴)₃, preferably R³ is selected from C₁₋₄ alkyl, C₁₋₄ alkylene-R⁴, and C(C₁₋₃ alkylene-R⁴)₃, more preferably R³ is selected from C₁₋₃ alkyl, C₁₋₃ alkylene-R⁴, and C(C₁₋₃ alkylene-R⁴)₃, more preferably R³ is selected from C₁₋₂ alkyl, C₁₋₂ alkylene-R⁴, and C(C₁₋₂ alkylene-R⁴)₃, more preferably R³ is selected from selected from methyl, ethyl, 2-hydroxyethyl, 2-(2-hydroxyethoxy)ethyl, and 2-[bis(2-hydroxyethyl)amino]ethyl.

16. The composition of item 14 or 15, wherein the N-heterocyclic ring is a monocyclic ring containing at least one nitrogen ring atom and optionally one further ring heteroatom selected from O and S.

17. The composition of any one of items 14 to 16, wherein the N-heterocyclic ring is a monocyclic ring containing (i) one nitrogen ring atom; (ii) two nitrogen ring atoms; (iii) one nitrogen ring atom and one oxygen ring atom; (iv) one nitrogen ring atom and one sulfur ring atom; or (v) three nitrogen ring atoms.

18. The composition of any one of item 14 to 17, wherein the N-heterocyclic ring is a monocyclic 5- or 6-membered N-heterocyclic ring, such as is a monocyclic 6-membered N-heterocyclic ring.

19. The composition of any one of items 14 to 18, wherein the N-heterocyclic ring is selected from pyrrolidinyl, imidazolidinyl, pyrazolidinyl, oxazolidinyl, thiazolidinyl, piperidinyl, piperazinyl, 1,2-diazinanyl, 1,3-diazinanyl, 1,3,5-triazinanyl, morpholinyl, and thiomorpholinyl, preferably selected from piperidinyl, piperazinyl, 1,2-diazinanyl, 1,3-diazinanyl, morpholinyl, and thiomorpholinyl.

20. The composition of any one of items 14 to 19, wherein, if the N-heterocyclic ring contains only one nitrogen ring atom, this nitrogen ring atom is substituted with R³, R³ being other than H, or, if the N-heterocyclic ring contains more than one nitrogen ring atom, one nitrogen ring atom is substituted with R³, R³ being other than H, and at least one of the other nitrogen ring atoms, preferably each of the other nitrogen ring atoms, is substituted with R⁵.

21. The composition of any one of items 14 to 20, wherein each R⁵ is independently selected from C₁₋₆ alkyl, C₁₋₆ alkylene-R⁴, and C(C₁₋₅ alkylene-R⁴)₃, preferably R⁵ is selected from C₁₋₄ alkyl, C₁₋₄ alkylene-R⁴, and C(C₁₋₃ alkylene-R⁴)₃, more preferably R⁵ is selected from C₁₋₃ alkyl, C₁₋₃ alkylene-R⁴, and C(C₁₋₃ alkylene-R⁴)₃, more preferably R⁵ is selected from C₁₋₂ alkyl, C₁₋₂ alkylene-R⁴, and C(C₁₋₂ alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl.

22. The composition of any one of items 14 to 21, wherein the N-heterocyclic ring is piperidinyl and the ring N atom is substituted with R³, R³ being other than H.

23. The composition of any one of items 14 to 21, wherein the N-heterocyclic ring is piperazinyl, one the two ring N atoms is substituted with R³, R³ being other than H, and the other ring N atom is optionally substituted with R⁵, preferably the other ring N atom is substituted with R⁵.

24. The composition of item 23, wherein both ring N atoms are substituted and R⁵ is selected from C₁₋₆ alkyl, C₁₋₆ alkylene-R⁴, and C(C₁₋₅ alkylene-R⁴)₃, preferably R⁵ is selected from C₁₋₄ alkyl, C₁₋₄ alkylene-R⁴, and C(C₁₋₃ alkylene-R⁴)₃, more preferably R⁵ is selected from C₁₋₃ alkyl, C₁₋₃ alkylene-R⁴, and C(C₁₋₃ alkylene-R⁴)₃, more preferably R⁵ is selected from C₁₋₂ alkyl, C₁₋₂ alkylene-R⁴, and C(C₁₋₂ alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl.

25. The composition of any one of items 14 to 24, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

26. The composition of any one of items 14 to 25, wherein each R⁴ is independently selected from —OH, 2-hydroxyethoxy, and bis(2-hydroxyethyl)amino.

27. The composition of any one of items 14 to 26, wherein R¹ and R⁵ are the same.

28. The composition of item 27, wherein both of R³ and R⁵ are methyl, ethyl, 2-hydroxyethyl, or 2-(2-hydroxyethoxy)ethyl, preferably, both of R³ and R⁵ are 2-hydroxyethyl.

29. The composition of any one of items 14 to 26, wherein R³ and R⁵ differ from each other.

30. The composition of item 1, wherein R¹ is H.

31. The composition of item 30, wherein each of R² and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R² and R³ is CH(C₁₋₅ alkylene-R⁴)₂ or C(C₁₋₅ alkylene-R⁴)₃, preferably each of R² and R³ is independently selected from C₁₋₄ alkyl, C₁₋₄ alkylene-R⁴, CH(C₁₋₃ alkylene-R⁴)₂, and C(C₁₋₃ alkylene-R⁴)₃, wherein at most one of R² and R³ is CH(C₁₋₃ alkylene-R⁴)₂ or C(C₁₋₃ alkylene-R⁴)₃, more preferably each of R² and R³ is independently selected from C₁₋₃ alkyl, C₁₋₃ alkylene-R⁴, CH(C₁₋₃ alkylene-R⁴)₂, and C(C₁₋₃ alkylene-R⁴)₃, wherein at most one of R² and R³ is CH(C₁₋₃ alkylene-R⁴)₂ or C(C₁₋₃ alkylene-R⁴)₃, more preferably each of R² and R³ is independently selected from C₁₋₂ alkyl, C₁₋₂ alkylene-R⁴, CH(C₁₋₂ alkylene-R⁴)₂, and C(C₁₋₂ alkylene-R⁴)₃, wherein at most one of R² and R³ is CH(C₁₋₂ alkylene-R⁴)₂ or C(C₁₋₂ alkylene-R⁴)₃.

32. The composition of item 30 or 31, wherein each of R² and R³ is independently selected from C₁₋₆ alkyl, C₁₋₆ alkylene-R⁴, and C(C₁₋₅ alkylene-R⁴)₃, wherein at most one of R² and R³ is C(C₁₋₅ alkylene-R⁴)₃, preferably each of R¹ and R³ is independently selected from C₁₋₄ alkyl, C₁₋₄ alkylene-R⁴, and C(C₁₋₃ alkylene-R⁴)₃, wherein at most one of R² and R³ is C(C₁₋₃ alkylene-R⁴)₃, more preferably each of R² and R³ is independently selected from C₁₋₃ alkyl, C₁₋₅ alkylene-R⁴, and C(C₁₋₃ alkylene-R⁴)₃, wherein at most one of R² and R³ is C(C₁₋₃ alkylene-R⁴)₃, more preferably each of R² and R³ is independently selected from C₁₋₂ alkyl, C₁₋₂ alkylene-R⁴, and C(C₁₋₂ alkylene-R⁴)₃, wherein at most one of R² and R³ is C(C₁₋₂ alkylene-R⁴)₃.

33. The composition of any one of items 30 to 32, wherein each of R² and R³ is independently selected from C₁₋₆ alkyl and C₁₋₆ alkylene-R⁴, preferably each of R² and R³ is independently selected from C₁₋₄ alkyl and C₁₋₄ alkylene-R⁴, more preferably each of R² and R³ is independently selected from C₁₋₃ alkyl and C₁₋₃ alkylene-R⁴, more preferably each of R² and R³ is independently selected from C₁₋₂ alkyl and C₁₋₂ alkylene-R⁴.

34. The composition of any one of items 30 to 33, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

35. The composition of any one of items 30 to 34, wherein each R⁴ is independently selected from —OH, 2-hydroxyethoxy, and bis(2-hydroxyethyl)amino.

36. The composition of any one of items 30 to 35, wherein each of R² and R³ is independently selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl, preferably, both of R² and R¹ are 2-hydroxyethyl or 2-(2-hydroxyethoxy)ethyl.

36a. The composition of any one of items 1 to 36, wherein the buffer substance is an N-oxide.

37. The composition of any one of items 1 to 36a, wherein the buffer substance is selected from bis(2-hydroxyethyl)amino-tris(hydroxymethyl)methane (Bis-Tris-methane or BTM) and its protonated form, triethanolamine (TEA) and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form.

38. The composition of any one of items 1 to 37, wherein the buffer substance comprises at least one C₁₋₆ alkylene-R⁴ (such as 2-hydroxyethyl) moiety.

39. A composition comprising (i) RNA; and (ii) an aqueous phase, wherein the aqueous phase comprises a buffer system comprising a buffer substance having the formula N(R¹)(R²)(R³), its N-oxide, or a protonated form thereof, wherein:
each of R¹, R², and R³ is independently selected from H, C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is H, CH(C₁₋₅ alkylene-R⁴)₂, or C(C₁₋₅ alkylene-R⁴)₃; or two of R¹, R², and R³ join together with the nitrogen atom to form a 5- or 6-membered N-heterocyclic ring which is optionally substituted with one or two R⁵;
each R⁴ is independently selected from —OH, —O-(C₁₋₆ alkylene-OH), and —N(R⁶)_{z}-(C₁₋₆ alkylene-OH)_{2-z}, wherein each z is independently selected from 0 and 1; and each R⁶ is independently selected from H and C₁₋₃ alkyl; and
each R⁵ is independently selected from C₁₋₆ alkyl, C₁₋₆ alkylene-R⁴, CH(C₁₋₅ alkylene-R⁴)₂, and C(C₁₋₅ alkylene-R⁴)₃.

40. The composition of item 39, wherein each of R¹, R², and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is CH(C₁₋₅ alkylene-R⁴)₂ or C(Cis alkylene-R⁴)₃, preferably each of R¹, R², and R³ is independently selected from C₁₋₄ alkyl, C₁₋₄ alkylene-R⁴, CH(C₁₋₃ alkylene-R⁴)₂, and C(C₁₋₃ alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₃ alkyl, C₁₋₃ alkylene-R⁴, CH(C₁₋₅ alkylene-R⁴)₂, and C(C₁₋₃ alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₂ alkyl, C₁₋₂ alkylene-R⁴, CH(C₁₋₂ alkylene-R⁴)₂, and C(C₁₋₂ alkylene-R⁴)₃, wherein at most one of R¹, R², and R³ is CH(C₁₋₂ alkylene-R⁴)₂ or C(C₁₋₂ alkylene-R⁴)₃.

41. The composition of item 39 or 40, wherein each of R¹, R², and R³ is independently selected from C₁₋₆ alkyl, C₁₋₆ alkylene-R⁴, and C(C₁₋₅ alkylene-R⁴)₃, wherein at most one of R¹, R², and R³ is C(C₁₋₅alkylene-R⁴)₃, preferably each of R¹, R², and R³ is independently selected from C₁₋₄ alkyl, C₁₋₄ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₃ alkyl, C₁₋₃ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₂ alkyl, C₁₋₂ alkylene-R⁴, and C(C₁₋₂ alkylene-R⁴)₃, wherein at most one of R¹, R², and R³ is C(C₁₋₂ alkylene-R⁴)₃.

42. The composition of any one of items 39 to 41, wherein each of R¹, R², and R³ is independently selected from C₁₋₆ alkyl and C₁₋₆ alkylene-R⁴, preferably each of R¹, R², and R³ is independently selected from C₁₋₄ alkyl and C₁₋₄ alkylene-R⁴, more preferably each of R¹, R², and R³ is independently selected from C₁₋₃ alkyl and C₁₋₃ alkylene-R⁴, more preferably each of R¹, R², and R³ is independently selected from C₁₋₂ alkyl and C₁₋₂ alkylene-R⁴.

43. The composition of any one of items 39 to 42, wherein each R⁴ is independently selected from —OH, —O-(C₁₋₄ alkylene-OH), and —N(R⁶)_{z}-(C₁₋₄ alkylene-OH)_{2-z}, wherein each z is independently selected from 0 and 1; and each R⁶ is independently selected from H and C₁₋₃ alkyl, preferably each R⁴ is independently selected from —OH, —O-(C₁₋₃ alkylene-OH), and —N(R⁶)_{z}-(C₁₋₃ alkylene-OH)_{2-z}, wherein each z is independently selected from 0 and 1; and each R⁶ is independently selected from H and C₁₋₃ alkyl, more preferably each R⁴ is independently selected from —OH, —O-(C₁₋₂ alkylene-OH), and —N(R⁶)₂-(C₁₋₂ alkylene-OH)_{2-z}, wherein each z is independently selected from 0 and 1; and each R⁶ is independently selected from H and C₁₋₂ alkyl. 44. The composition of any one of items 39 to 43, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₅ alkylene-OH)₂, more preferably each R⁴ is independently selected from —OH, —O-(C₁₋₂ alkylene-OH), and —N(C₁₋₂ alkylene-OH)₂.

45. The composition of any one of items 39 to 44, wherein each R⁴ is independently selected from —OH, 2-hydroxyethoxy, and bis(2-hydroxyethyl)amino.

46. The composition of any one of items 39 to 45, wherein each of R¹, R², and R³ is independently selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

47. The composition of any one of items 39 to 46, wherein all of R¹, R², and R³ are the same.

48. The composition of item 47, wherein all of R¹, R², and R³ are methyl, ethyl, or 2-hydroxyethyl.

49. The composition of any one of items 39 to 46, wherein R¹ and R² are the same and R³ differs from R¹ and R².

50. The composition of item 49, wherein each of R¹ and R² is 2-hydroxyethyl, ethyl, or methyl.

51. The composition of item 49 or 50, wherein R³ is selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

52. The composition of item 39, wherein R¹ and R² join together with the nitrogen atom to form a 5- or 6-membered N-heterocyclic ring which is optionally substituted with one or two R.

53. The composition of item 52, wherein R³ is selected from C₁₋₆ alkyl, C₁₋₆ alkylene-R⁴, and C(C₁₋₅ alkylene-R⁴)₃, preferably R³ is selected from C₁₋₄ alkyl, C₁₋₄ alkylene-R⁴, and C(C₁₋₃ alkylene-R⁴)₃, more preferably R³ is selected from C₁₋₃ alkyl, C₁₋₃ alkylene-R⁴, and C(C₁₋₃ alkylene-R⁴)₃, more preferably R³ is selected from C₁₋₂ alkyl, C₁₋₂ alkylene-R⁴, and C(C₁₋₂ alkylene-R⁴)₃, more preferably R³ is selected from selected from methyl, ethyl, 2-hydroxyethyl, 2-(2-hydroxyethoxy)ethyl, and 2-[bis(2-hydroxyethyl)amino]ethyl.

53a. The composition of item 52 or 53, wherein the N-heterocyclic ring is a monocyclic ring containing at least one nitrogen ring atom and optionally one further ring heteroatom selected from O and S.

53b. The composition of any one of items 52 to 53a, wherein the N-heterocyclic ring is a monocyclic ring containing (i) one nitrogen ring atom; (ii) two nitrogen ring atoms; (iii) one nitrogen ring atom and one oxygen ring atom; (iv) one nitrogen ring atom and one sulfur ring atom; or (v) three nitrogen ring atoms.

53c. The composition of any one of item 52 to 53b, wherein the N-heterocyclic ring is a monocyclic 5- or 6-membered N-heterocyclic ring, such as is a monocyclic 6-membered N-heterocyclic ring.

53d. The composition of any one of items 52 to 53c, wherein the N-heterocyclic ring is selected from pyrrolidinyl, imidazolidinyl, pyrazolidinyl, oxazolidinyl, thiazolidinyl, piperidinyl, piperazinyl, 1,2-diazinanyl, 1,3-diazinanyl, 1,3,5-triazinanyl, morpholinyl, and thiomorpholinyl, preferably selected from piperidinyl, piperazinyl, 1,2-diazinanyl, 1,3-diazinanyl, morpholinyl, and thiomorpholinyl.

54. The composition of any one of items 52 to 53d, wherein, if the N-heterocyclic ring contains only one nitrogen ring atom, this nitrogen ring atom is substituted with R³, R³ being other than H, or, if the N-heterocyclic ring contains more than one nitrogen ring atom, one nitrogen ring atom is substituted with R³, R³ being other than H, and at least one of the other nitrogen ring atoms, preferably each of the other nitrogen ring atoms, is substituted with R⁵.

55. The composition of any one of items 52 to 54, wherein each R⁵ is independently selected from C₁₋₆ alkyl, C₁₋₆alkylene-R⁴, and C(C₁₋₅ alkylene-R⁴)₃, preferably R⁵ is selected from C₁₋₄ alkyl, C₁₋₄alkylene-R⁴, and C(C₁₋₃ alkylene-R⁴)₃, more preferably R⁵ is selected from C₁₋₃ alkyl, C₁₋₃ alkylene-R⁴, and C(C₁₋₃ alkylene-R⁴)₃, more preferably R⁵ is selected from C₁₋₂ alkyl, C₁₋₂ alkylene-R⁴, and C(C₁₋₂alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl.

56. The composition of any one of items 52 to 55, wherein the N-heterocyclic ring is piperidinyl and the ring N atom is substituted with R³, wherein R³ is other than H.

57. The composition of any one of items 52 to 55, wherein the N-heterocyclic ring is piperazinyl, one the two ring N atoms is substituted with R³, R³ being other than H, and the other ring N atom is optionally substituted with R⁵, preferably the other ring N atom is substituted with R⁵.

58. The composition of item 57, wherein both ring N atoms are substituted and R⁵ is selected from C₁₋₆ alkyl, C₁₋₆ alkylene-R⁴, and C(C₁₋₅ alkylene-R⁴)₃, preferably R⁵ is selected from C₁₋₄ alkyl, C_{J}-4 alkylene-R⁴, and C(C₁₋₃ alkylene-R⁴)₃, more preferably R⁵ is selected from C₁₋₃ alkyl, C₁₋₃ alkylene-R⁴, and C(C₁₋₃ alkylene-R⁴)₃, more preferably R⁵ is selected from C₁₋₂ alkyl, C₁₋₂ alkylene-R⁴, and C(C₁₋₂alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl.

59. The composition of any one of items 52 to 58, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

60. The composition of any one of items 52 to 59, wherein each R⁴ is independently selected from —OH, 2-hydroxyethoxy, and bis(2-hydroxyethyl)amino.

61. The composition of any one of items 52 to 60, wherein R³ and R⁵ are the same.

62. The composition of item 61, wherein both of R³ and R⁵ are methyl, ethyl, 2-hydroxyethyl, or 2-(2-hydroxyethoxy)ethyl, preferably, both of R³ and R⁵ are 2-hydroxyethyl.

63. The composition of any one of items 52 to 60, wherein R³ and R⁵ differ from each other.

63a. The composition of item 39, wherein R¹ is H.

63b. The composition of item 63a, wherein each of R² and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R² and R³ is CH(C₁₋₅ alkylene-R⁴)₂ or C(C₁₋₅ alkylene-R⁴)₃, preferably each of R² and R³ is independently selected from C₁₋₄ alkyl, C₁₋₄ alkylene-R⁴, CH(C₁₋₃ alkylene-R⁴)₂, and C(C₁₋₃ alkylene-R⁴)₃, wherein at most one of R² and R³ is CH(C₁₋₃ alkylene-R⁴)₂ or C(C₁₋₃ alkylene-R⁴)₃, more preferably each of R² and R³ is independently selected from C₁₋₃ alkyl, C₁₋₃ alkylene-R⁴, CH(C₁₋₃ alkylene-R⁴)₂, and C(C₁₋₃ alkylene-R⁴)₃, wherein at most one of R² and R³ is CH(C₁₋₃ alkylene-R⁴)₂ or C(C₁₋₃ alkylene-R⁴)₃, more preferably each of R² and R³ is independently selected from C₁₋₂ alkyl, Cp2 alkylene-R⁴, CH(C₁₋₂ alkylene-R⁴)₂, and C(C₁₋₂ alkylene-R⁴)₃, wherein at most one of R² and R³ is CH(C₁₋₂ alkylene-R⁴)₂ or C(C₁₋₂ alkylene-R⁴)₃.

63c. The composition of item 63a or 63b, wherein each of R² and R³ is independently selected from C₁₋₆ alkyl, C₁₋₆ alkylene-R⁴, and C(C₁₋₅ alkylene-R⁴)₃, wherein at most one of R² and R³ is C(C₁₋₅ alkylene-R⁴)₃, preferably each of R² and R³ is independently selected from C₁₋₄ alkyl, C₁₋₄ alkylene-R⁴, and C(C₁₋₃ alkylene-R⁴)₃, wherein at most one of R² and R³ is C(C₁₋₃ alkylene-R⁴)₃, more preferably each of R² and R³ is independently selected from C₁₋₃ alkyl, C₃ alkylene-R⁴, and C(C₁₋₃ alkylene-R⁴)₃, wherein at most one of R² and R³ is C(C₁₋₃ alkylene-R⁴)₃, more preferably each of R² and R³ is independently selected from C₁₋₂ alkyl, C₁₋₂ alkylene-R⁴, and C(C₁₋₂ alkylene-R⁴)₃, wherein at most one of R² and R³ is C(C₁₋₂ alkylene-R⁴)₃.

63d. The composition of any one of items 63a to 63c, wherein each of R² and R³ is independently selected from C₁₋₆ alkyl and C₁₋₆ alkylene-R⁴, preferably each of R² and R³ is independently selected from C₁₋₄ alkyl and C₁₋₄ alkylene-R⁴, more preferably each of R² and R³ is independently selected from C₁₋₃ alkyl and C₁₋₃ alkylene-R⁴, more preferably each of R² and R³ is independently selected from C₁₋₂alkyl and C₁₋₂ alkylene-R⁴.

63e. The composition of any one of items 63a to 63d, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OI)₂, more preferably each R⁴ is independently selected from —OH, —O-(C₁₋₂ alkylene-OH), and —N(C₁₋₂ alkylene-OH)₂.

63f. The composition of any one of items 63a to 63e, wherein each R⁴ is independently selected from —OH, 2-hydroxyethoxy, and bis(2-hydroxyethyl)amino.

63g. The composition of any one of items 63a to 63f, wherein each of R² and R³ is independently selected from 2-hydroxyethyl, 2-(2-hydroxyethoxy)ethyl, and 2-[bis(2-hydroxyethyl)amino]ethyl, preferably, both of R² and R³ are 2-hydroxyethyl or 2-(2-hydroxyethoxy)ethyl.

63h. The composition of any one of items 39 to 63g, wherein the buffer substance is an N-oxide.

64. The composition of any one of items 39 to 63h, wherein the buffer substance is selected from bis(2-hydroxyethyl)amino-tris(hydroxymethyl)methane (Bis-Tris-methane or BTM) and its protonated form, triethanolamine (TEA) and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form.

65. The composition of any one of items 39 to 64, wherein the buffer substance comprises at least one C₁₋₆ alkylene-R⁴ (such as 2-hydroxyethyl) moiety.

66. The composition of any one of items 1 to 65, wherein the buffer system further comprises an anion selected from the group consisting of chloride, acetate, glycolate, lactate, and the anion of a di- or tricarboxylic acid, such as the anion of citric acid, succinic acid, malonic acid, glutaric acid, or adipic acid.

67. The composition of any one of items 1 to 66, wherein the concentration of the buffer substance in the composition is between about 10 mM and about 200 mM, preferably between about 15 mM and about 100 mM, more preferably between about 20 mM and about 80 mM, more preferably between about 40 mM and about 60 mM, such as about 50 mM.

68. The composition of any one of items 1 to 67, wherein the pH of the composition is between about 4.0 and about 8.0, preferably between about 4.5 and about 8.0, such as between about 5.0 and about 8.0, between about 5.5 and about 8.0, between about 6.0 and about 8.0, between about 6.5 and about 8.0, between about 6.8 and about 7.9, or between about 7.0 and about 7.8.

69. The composition of any one of items 1 to 68, wherein water is the main component in the composition and/or the total amount of solvent(s) other than water contained in the composition is less than about 0.5% (v/v).

70. The composition of any one of items 1 to 69, wherein the osmolality of the composition is at most about 1000×10⁻³ osmol/kg, preferably between about 100×10⁻³ osmol/kg and about 750×10⁻³ osmol/kg, such as between about 100×10⁻³ osmol/kg and about 500×10⁻³ osmol/kg, more preferably about 300×10⁻³ osmol/kg.

71. The composition of any one of items 1 to 70, wherein the concentration of the RNA in the composition is about 5 mg/l to about 500 mg/l, such as about 10 mg/l to about 400 mg/l, about 10 mg/l to about 300 mg/l, about 10 mg/l to about 200 mg/l, about 10 mg/l to about 150 mg/l, or about 10 mg/l to about 100 mg/l, preferably about 10 mg/l to about 140 mg/l, more preferably about 20 mg/l to about 130 mg/l, more preferably about 30 mg/l to about 120 mg/l.

72. The composition of any one of items 1 to 71, wherein the composition comprises a cryoprotectant, preferably in a concentration of at least about 1% w/v, wherein the cryoprotectant preferably comprises one or more compounds selected from the group consisting of carbohydrates and alcohols (such as sugar alcohols or lower alcohols), more preferably the cryoprotectant is selected from the group consisting of sucrose, glucose, glycerol, 1,2-propanediol, 1,3-propanediol, sorbitol, and a combination thereof (such as from the group consisting of sucrose, glucose, glycerol, 1,2-propanediol, 1,3-propanediol, and a combination thereof or from the group consisting of sucrose, glucose, glycerol, sorbitol, and a combination thereof), more preferably the cryoprotectant comprises sucrose and/or glycerol.

72a. The composition of any one of items 1 to 71, wherein the composition is substantially free of a cryoprotectant.

73. The composition of any one of items 1 to 38 and 66 to 72, wherein the cationically ionizable lipid comprises a head group which includes at least one nitrogen atom which is capable of being protonated under physiological conditions.

74. The composition of any one of items 1 to 38 and 66 to 73, wherein the cationically ionizable lipid has the structure of Formula (X)

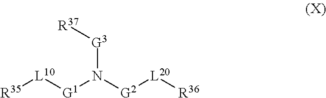

or a pharmaceutically acceptable salt, tautomer, prodrug or stereoisomer thereof, wherein: one of L¹⁶ and L²¹ is —O(C═O)—, —(C═O)O—, —C(═O)—, —O—, —S(O)ₓ-, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is —O(C═O)—, —(C═O)O—, —C(═O)—, —O—, —S(O)X—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₁₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆₋₂₄ alkyl or C₆₋₂₄ alkenyl;
R³⁷ is H, OR⁵⁰, CN, —C(═O)OR⁴⁰, —OC(═O)R⁴⁰ or —NR⁵⁰C(═O)R⁴⁰;
R⁴⁰ is C₁₋₁₂ alkyl;
R⁵⁰ is H or C₁₋₆ alkyl; and
x is 0, 1 or 2.

74a. The composition of any one of items 1 to 38 and 66 to 74, wherein:
(α) the cationically ionizable lipid is selected from the following structures X-1 to X-36:

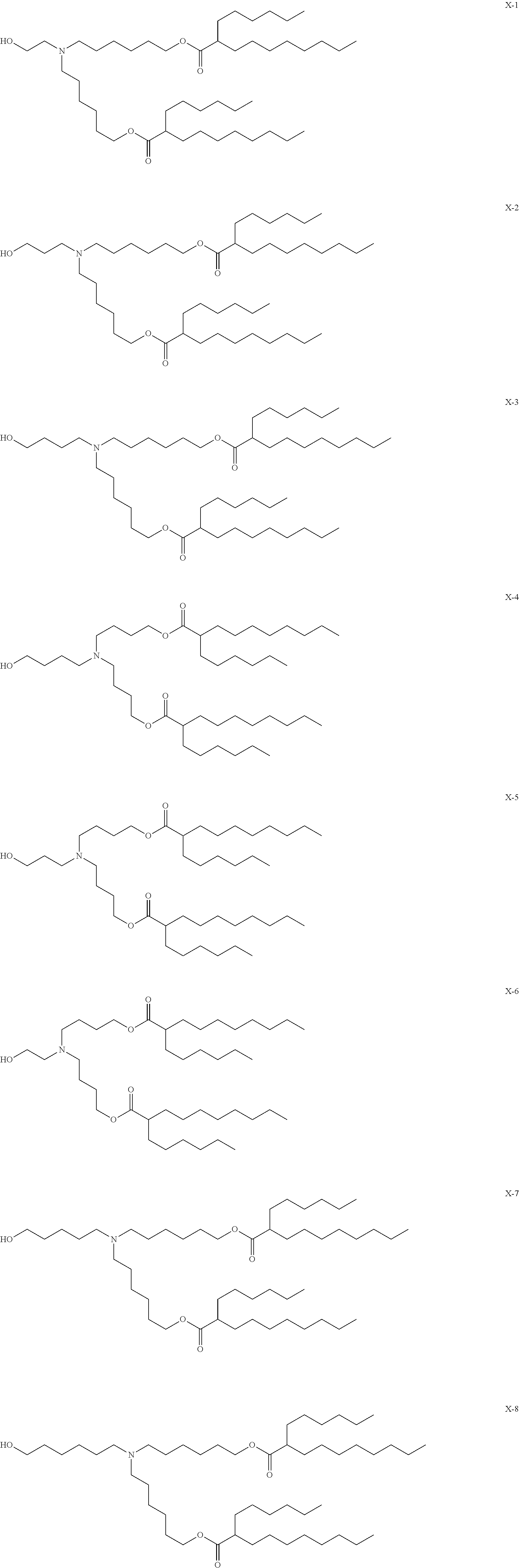

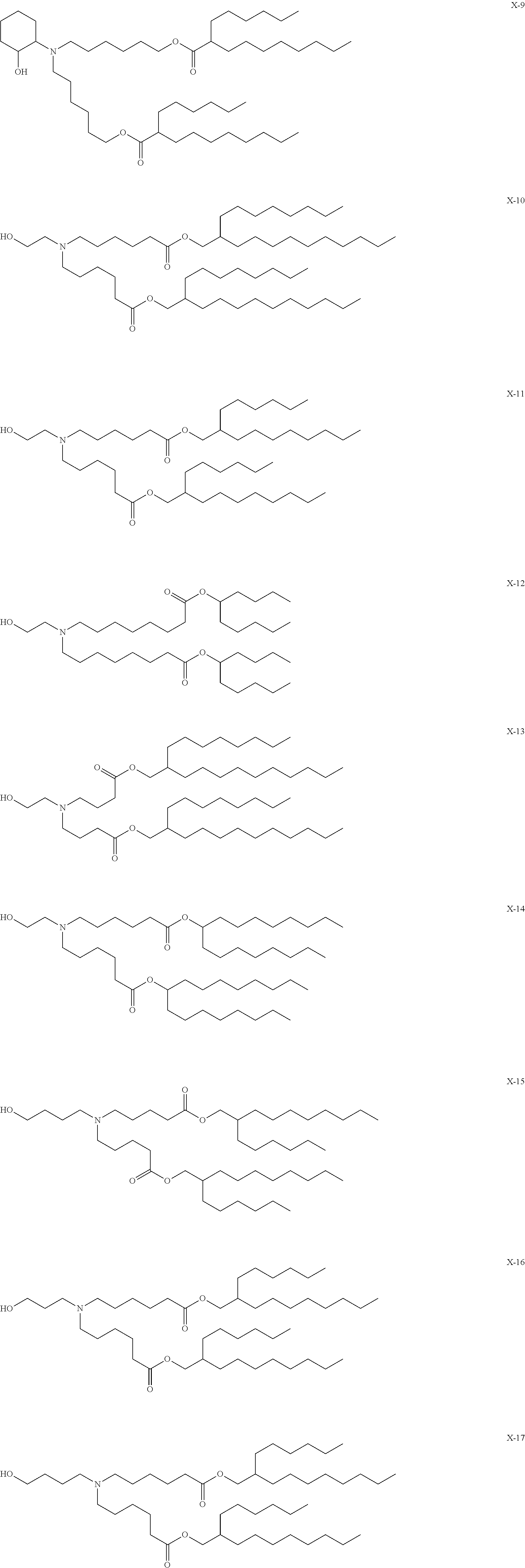

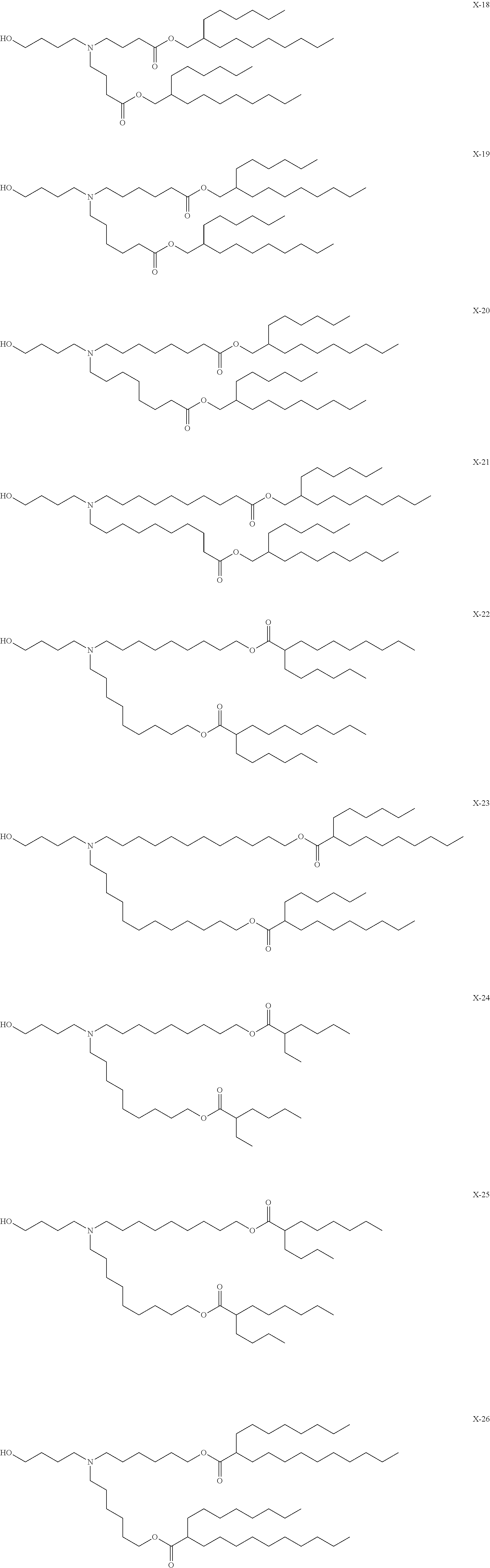

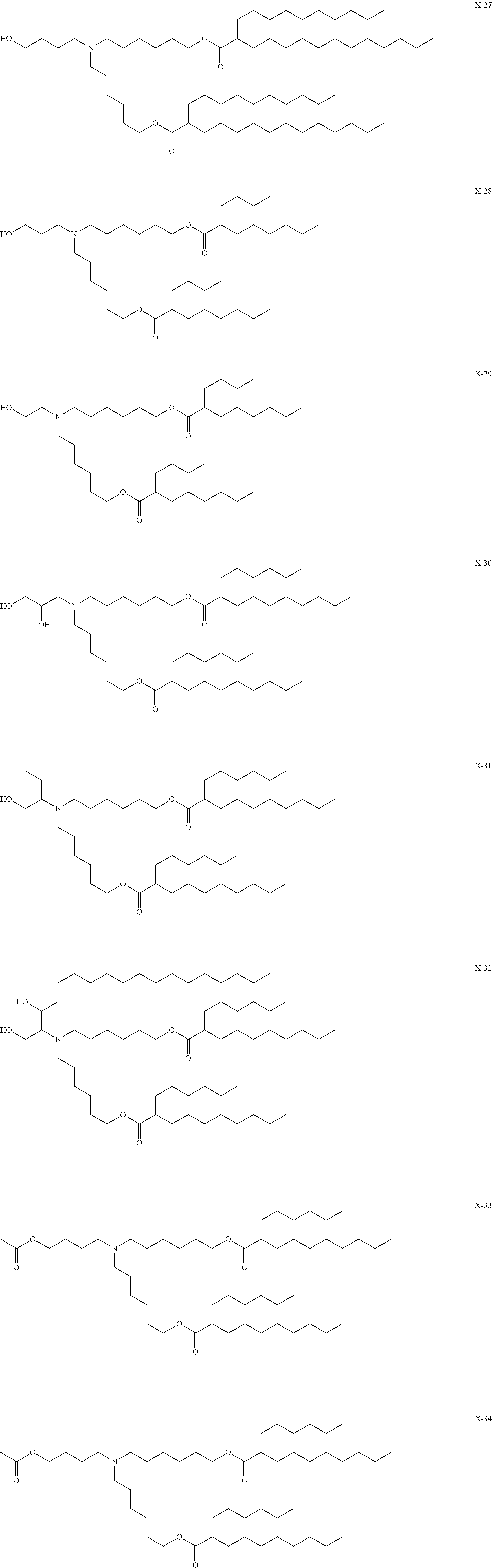

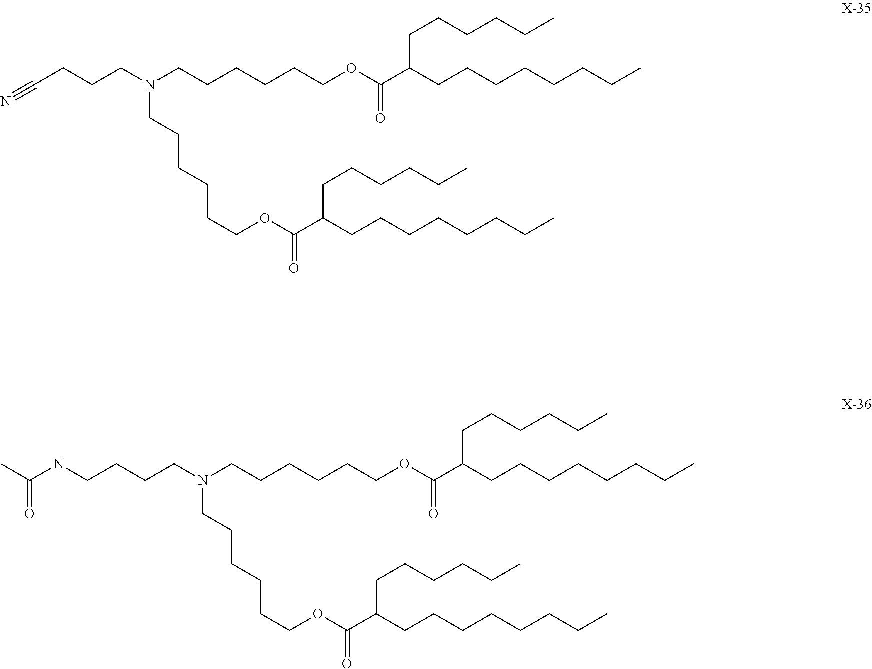

(β) the cationically ionizable lipid is selected from the following structures A to G:

| No. | Structure |
|---|---|
| A | 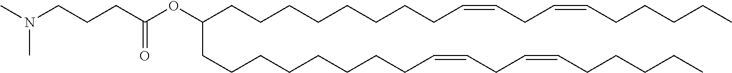 [SMILES: CCCCC/C=C\C/C=C\CCCCCCCCC(CCCCCCCC/C=C\C/C=C\CCCCC)OC(=O)CCCN(C)C] |
| B | 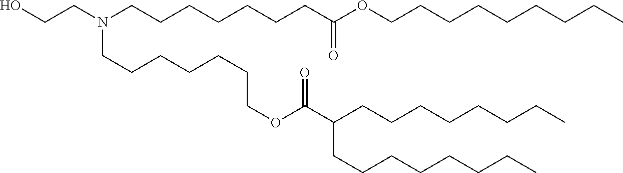 [SMILES: CCCCCCCCCOC(=O)CCCCCCCN(CCO)CCCCCCCOC(=O)C(CCCCCCCC)CCCCCCCC] |
| C | 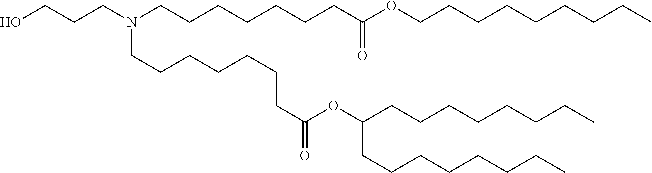 [SMILES: CCCCCCCCCOC(=O)CCCCCCCN(CCCO)CCCCCCCC(=O)OC(CCCCCCCC)CCCCCCCC] |
| D | 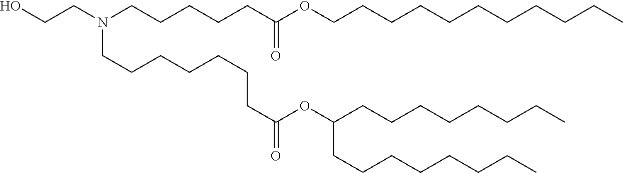 [SMILES: CCCCCCCCCCCOC(=O)CCCCCN(CCO)CCCCCCCC(=O)OC(CCCCCCCC)CCCCCCCC] |
| E | 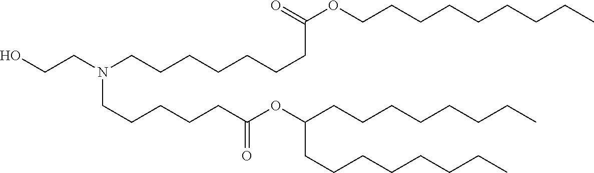 [SMILES: CCCCCCCCCOC(=O)CCCCCCCN(CCO)CCCCCC(=O)OC(CCCCCCCC)CCCCCCCC] |
| F | 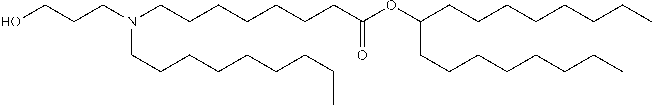 [SMILES: CCCCCCCCCN(CCCO)CCCCCCCC(=O)OC(CCCCCCCC)CCCCCCCC] |
| G | 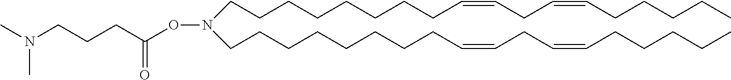 [SMILES: CCCCC/C=C\C/C=C\CCCCCCCCN(CCCCCCCC/C=C\C/C=C\CCCCC)OC(=O)CCCN(C)C] |

or
(γ) the cationically ionizable lipid is the lipid having the structure X-3.

75. The composition of any one of items 1 to 38 and 66 to 73, wherein the cationic or cationically ionizable lipid has the structure of Formula (XI):

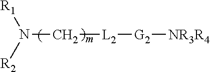

wherein
each of R₁ and R₂ is independently R₅ or -G₁-L₁-R₆, wherein at least one of R₁ and R₂ is -G₁-L₁-R₆;
each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—, —C(═O)—, —O—, —S(O)ₓ, —S—S—, —C(═O)S—, —SC(═O)—, —NRaC(═O)—, —C(═O)NRa—,
—NRaC(═O)NRa—, —OC(═O)NRa— and —NRaC(═O)O—;
Ra is H or C₁₋₁₂ alkyl;
m is 0, 1, 2, 3, or 4; and
x is 0, 1 or 2.

75a. The composition of any one of items 1 to 38, 66 to 73, and 75, wherein the cationically ionizable lipid is selected from the following structures (XIV-1), (XIV-2), and (XIV-3):

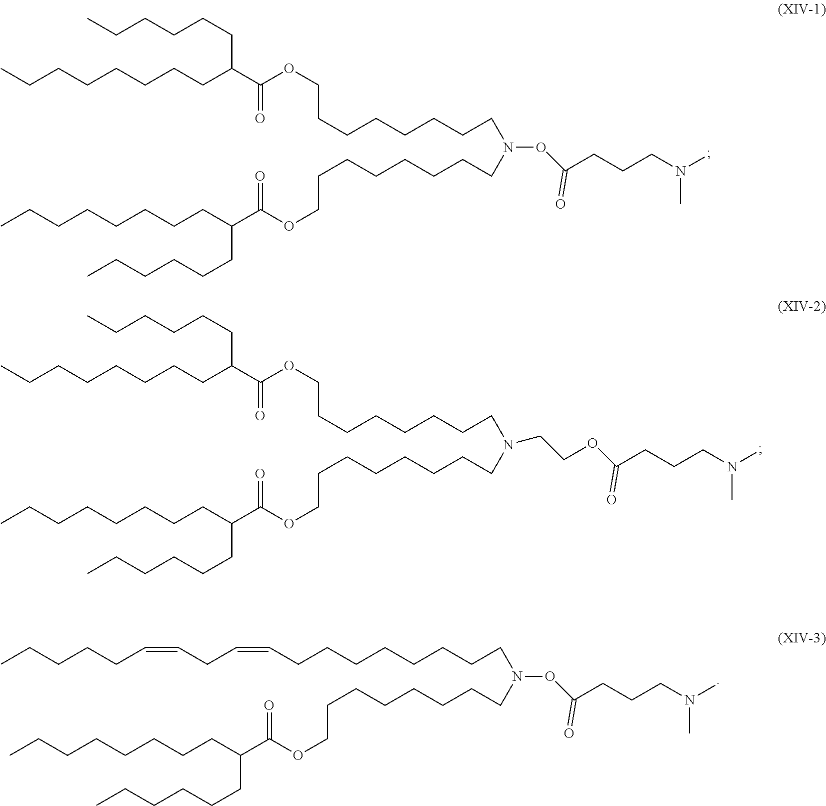

76. The composition of any one of items 1 to 38 and 66 to 75a, wherein the cationically ionizable lipid comprises from about 20 mol % to about 80 mol %, preferably from about 25 mol % to about 65 mol %, more preferably from about 30 mol % to about 50 mol %, such as from about 40 mol % to about 50 mol %, of the total lipid present in the composition.

77. The composition of any one of items 1 to 76, which further comprises one or more additional lipids, preferably selected from the group consisting of polymer conjugated lipids, neutral lipids, steroids, and combinations thereof, more preferably the composition comprises the cationically ionizable lipid, a polymer conjugated lipid, a neutral lipid (e.g., a phospholipid), and a steroid.

78. The composition of item 77, wherein the polymer conjugated lipid comprises a pegylated lipid, wherein the pegylated lipid preferably (i) is selected from the group consisting of DSPE-PEG, DOPE-PEG, DPPE-PEG, and DMPE-PEG; or (ii) has the following structure:

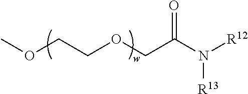

or a pharmaceutically acceptable salt, tautomer or stereoisomer thereof, wherein:

R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

79. The composition of item 77, wherein the polymer conjugated lipid comprises a polysarcosine-lipid conjugate or a conjugate ofpolysarcosine and a lipid-like material, wherein the polysarcosine-lipid conjugate or conjugate of polysarcosine and a lipid-like material preferably is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

80. The composition of any one of items 77 to 79, wherein the polymer conjugated lipid comprises from about 0.5 mol % to about 5 mol %, preferably from about 1 mol % to about 5 mol %, more preferably from about 1 mol % to about 4.5 mol % of the total lipid present in the composition.

81. The composition of any one of items 77 to 80, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

82. The composition of any one of items 77 to 81, wherein the neutral lipid comprises from about 5 mol % to about 40 mol %, preferably from about 5 mol % to about 20 mol %, more preferably from about 5 mol % to about 15 mol % of the total lipid present in the composition.

83. The composition of any one of items 77 to 82, wherein the steroid comprises a sterol such as cholesterol.

84. The composition of any one of items 77 to 83, wherein the steroid comprises from about 10 mol % to about 65 mol %, preferably from about 20 mol % to about 60 mol %, more preferably from about 30 mol % to about 50 mol % of the total lipid present in the composition.

85. The composition of any one of items 77 to 84, which comprises a cationically ionizable lipid, a polymer conjugated lipid, a neutral lipid (e.g., a phospholipid), and a steroid, wherein the cationically ionizable lipid comprises from about 30 mol % to about 50 mol %, such as from about 40 mol % to about 50 mol %, of the total lipid present in the composition; the polymer conjugated lipid comprises from about 1 mol % to about 4.5 mol % of the total lipid present in the composition; the neutral lipid (e.g., phospholipid) comprises from about 5 mol % to about 15 mol % of the total lipid present in the composition; and the steroid comprises from about 30 mol % to about 50 mol % of the total lipid present in the composition.

86. The composition of any one of items 1 to 85, wherein at least a portion of the RNA and, if present, of one or more lipids, is present in particles, such as lipid nanoparticles (LNPs), liposomes, and/or lipoplexes (LPXs).

86a. The composition of item 86, the particles comprise at least about 75%, preferably at least about 80% of the RNA comprised in the composition.

86b. The composition of item 86 or 86a, wherein the RNA is encapsulated within or associated with the particles.

87. The composition of any one of items 86 to 86b, wherein the particles have a size of from about 30 nm to about 500 nm.

88. The composition of any one of items 1 to 87, wherein the RNA is mRNA or inhibitory RNA.

89. The composition of any one of items 1 to 88, wherein the RNA (i) comprises a modified nucleoside in place of uridine, wherein the modified nucleoside is preferably selected from pseudouridine (ψ), N1-methyl-pseudouridine (mlyi), and 5-methyl-uridine (m5U); (ii) has a coding sequence which is codon-optimized; and/or (iii) has a coding sequence whose G/C content is increased compared to the wild-type coding sequence.

90. The composition of any one of items 1 to 89, wherein the RNA comprises at least one of the following, preferably all of the following: a 5′ cap; a 5′ UTR; a 3′ UTR; and a poly-A sequence.

91. The composition of item 90, wherein the poly-A sequence comprises at least 100 A nucleotides, wherein the poly-A sequence preferably is an interrupted sequence of A nucleotides.

92. The composition of item 90 or 91, wherein the 5′ cap is a cap1 or cap2 structure.

93. The composition of any one of items 1 to 92, wherein the RNA encodes one or more polypeptides, wherein preferably the one or more polypeptides are pharmaceutically active polypeptides and/or comprise an epitope for inducing an immune response against an antigen in a subject.

94. The composition of item 93, wherein the pharmaceutically active polypeptide and/or the antigen or epitope is derived from or is a protein of a pathogen, an immunogenic variant of the protein, or an immunogenic fragment of the protein or the immunogenic variant thereof.

95. The composition of item 93 or 94, wherein the pharmaceutically active polypeptide and/or the antigen or epitope is derived from or is a SARS-CoV-2 spike (S) protein, an immunogenic variant thereof, or an immunogenic fragment of the SARS-CoV-2 S protein or the immunogenic variant thereof.

95a. The composition of item 94 or 95, wherein the RNA comprises an open reading frame (ORF) encoding an amino acid sequence comprising a SARS-CoV-2 S protein, an immunogenic variant thereof, or an immunogenic fragment of the SARS-CoV-2 S protein or the immunogenic variant thereof.

95b. The composition of item 95 or 95a, wherein the SARS-CoV2 S protein variant has proline residue substitutions at positions 986 and 987 of SEQ ID NO: 1.

95c. The composition of any one of items 95 to 95b, wherein the SARS-CoV2 S protein variant has at least 80% identity to the amino acid sequence of amino acids 17 to 1273 of SEQ ID NO: 7 or the amino acid sequence of amino acids 17 to 1273 of SEQ ID NO: 1.

95d. The composition of any one of items 95 to 95c, wherein the fragment comprises the receptor binding domain (RBD) of the SARS-CoV-2 S protein.

95e. The composition of item 95d, wherein the fragment of (i) the SARS-CoV-2 S protein or (ii) the immunogenic variant of the SARS-CoV-2 S protein has at least 80% identity to the amino acid sequence of amino acids 327 to 528 of SEQ ID NO: 1.

96. The composition of any one of items 1 to 95e, wherein the composition is in liquid form, preferably at a temperature of about 2° C. to about 10° C.

97. The composition of any one of items 1 to 96, wherein the RNA integrity of the composition after storage for at least one week, preferably at a temperature of about 2° C. to about 8° C., is at least 50% compared to the RNA integrity before storage.

98. The composition of any one of items 87 to 97, wherein the size (Z_{average}) and/or size distribution and/or polydispersity index (PDI) of RNA particles (in particular LNPs) after storage of the composition 10 is essentially equal to the size (Z_{average}) and/or size distribution and/or PDI of the RNA particles before storage.

99. The composition of any one of items 1 to 95e, wherein the composition is in frozen form.

99a. The composition of item 99, wherein the pH of the composition is between 4.0 and 8.0, preferably between 5.0 and 7.0, more preferably between 5.5 and 6.5 and most preferably about 5.5.

99b. The composition of item 99 or 99a, further comprising a cryoprotectant.

99c. The composition of item 99b, wherein the cryoprotectant is selected from the group consisting of sucrose, glucose, glycerol, 1,2-propanediol, 1,3-propanediol, and a combination thereof, such as from the group consisting of sucrose, glycerol and glucose, for example the cryoprotectant is glycerol.

99d. The composition of item 99b or 99c, wherein the cryoprotectant is present in a concentration of between about 100 mM and about 600 mM, preferably between about 200 mM and about 600 mM and more preferably between about 300 mM and about 500 mM.

100. The composition of any one of items 99 to 99d, wherein the RNA integrity after thawing the frozen composition is at least 50% compared to the RNA integrity before the composition has been frozen.

101. The composition of item 99 or 100, wherein the size (Z_{average}) and/or size distribution and/or polydispersity index (PDI) of RNA particles (in particular LNPs) after thawing the frozen composition is essentially equal to the size (Z_{average}) and/or size distribution and/or PDI of the RNA particles before the composition has been frozen.

102. A method of preparing a composition comprising LNPs dispersed in a final aqueous phase, wherein the LNPs comprise a cationically ionizable lipid and RNA; the final aqueous phase comprises a final buffer system comprising a final buffer substance, the final buffer substance having the formula N(R¹)(R²)(R³), its N-oxide, or a protonated form thereof, wherein R¹, R², and R³ are as defined in any one of items 1 to 38;
wherein the method comprises:
(I) preparing a formulation comprising LNPs dispersed in the final aqueous phase, wherein the LNPs comprise the cationically ionizable lipid and RNA; and
(II) optionally freezing the formulation to about −10° C. or below,
thereby obtaining the composition,
wherein step (I) comprises:
(a) preparing an RNA solution containing water and a first buffer system;
(b) preparing an ethanolic solution comprising the cationically ionizable lipid and, if present, one or more additional lipids;
(c) mixing the RNA solution prepared under (a) with the ethanolic solution prepared under (b), thereby preparing a first intermediate formulation comprising the LNPs dispersed in a first aqueous phase comprising the first buffer system; and
(d) filtrating the first intermediate formulation prepared under (c) using a final aqueous buffer solution comprising the final buffer system, thereby preparing the formulation comprising the LNPs dispersed in the final aqueous phase.

103. The method of item 102, wherein step (1) further comprises one or more steps selected from diluting and filtrating.

104. The method of item 102 or 103, wherein step (1) comprises:
(a′) providing an aqueous RNA solution;
(b′) providing a first aqueous buffer solution comprising a first buffer system;
(c′) mixing the aqueous RNA solution provided under (a′) with the first aqueous buffer solution provided under (b′) thereby preparing an RNA solution containing water and the first buffer system;
(d′) preparing an ethanolic solution comprising the cationically ionizable lipid and, if present, one or more additional lipids;
(e′) mixing the RNA solution prepared under (c′) with the ethanolic solution prepared under (d′), thereby preparing a first intermediate formulation comprising LNPs dispersed in a first aqueous phase comprising the first buffer system;
(f′) optionally filtrating the first intermediate formulation prepared under (e′) using a further aqueous buffer solution comprising a further buffer system, thereby preparing a further intermediate formulation comprising the LNPs dispersed in a further aqueous phase comprising the further buffer system, wherein the further aqueous buffer solution may be identical to or different from the first aqueous buffer solution;
(g′) optionally repeating step (f′) once or two or more times, wherein the further intermediate formulation comprising the LNPs dispersed in the further aqueous phase comprising the further buffer system obtained after step (f′) of one cycle is used as the first intermediate formulation of the next cycle, wherein in each cycle the further aqueous buffer solution may be identical to or different from the first aqueous buffer solution;
(h′) filtrating the first intermediate formulation obtained in step (e′), if step (f′) is absent, or the further intermediate formulation obtained in step (f′), if step (f′) is present and step (g′) is not present, or the further intermediate formulation obtained after step (g′), if steps (f′) and (g′) are present, using a final aqueous buffer solution comprising the final buffer system; and
(i′) optionally diluting the formulation obtained in step (h′) with a dilution solution; thereby preparing the formulation comprising the LNPs dispersed in the final aqueous phase.

105. The method of any one of items 102 to 104, wherein filtrating is tangential flow filtrating or diafiltrating, preferably tangential flow filtrating.

106. The method of any one of items 102 to 105, which comprises (II) freezing the formulation to about −10° C. or below.

106a. The method of item 106, wherein the pH of the composition is between 4.0 and 8.0, preferably between 5.0 and 7.0, more preferably between 5.5 and 6.5 and most preferably about 5.5.

107. The method of item 106 or 106a, further comprising a cryoprotectant (preferably the formulation obtained in step (I) and/or the composition comprise(s) a cryoprotectant).

107a. The method of item 107, wherein the cryoprotectant is selected from the group consisting of sucrose, glucose, glycerol, 1,2-propanediol, 1,3-propanediol, and a combination thereof, such as from the group consisting of sucrose, glycerol and glucose, for example the cryoprotectant is glycerol.

107b. The method of item 107 or 107a, wherein the cryoprotectant is present in a concentration of between about 100 mM and about 600 mM, preferably between about 200 mM and about 600 mM and more preferably between about 300 mM and about 500 mM.

108. The method of any one of items 102 to 107b, wherein the final buffer substance is selected from BTM and its protonated form, TEA and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form.

108a. The method of any one of items 102 to 108, wherein the final buffer system further comprises an anion, preferably selected from the group consisting of chloride, acetate, glycolate, lactate, the anion of morpholinoethanesulfonic acid (MES), the anion of 3-(N-morpholino)propanesulfonic acid (MOPS), the anion of 2-[4-(2-hydroxyethyl)piperazin-1-yl]ethanesulfonic acid (HEPES), and the anion of a di- or tricarboxylic acid, such as the anion of citric acid, succinic acid, malonic acid, glutaric acid, or adipic acid.

109. The method of any one of items 102 to 108a, wherein the concentration of the final buffer substance in the composition is between about 10 rmM to about 200 mM, preferably between about 15 mM to about 100 mM, more preferably between about 20 mM to about 80 mM, more preferably between about 40 mM to about 60 mM, such as about 50 mM.

110. The method of any one of items 102 to 109, wherein (i) the RNA solution obtained in step (a) has a pH of below 6.0, preferably at most about 5.0, more preferably at most about 4.5; or (ii) the first aqueous buffer solution has a pH of below 6.0, preferably at most about 5.0, more preferably at most about 4.5.

111. The method of any one of items 102 to 110, wherein (i) the first buffer system used in step (a) comprises the final buffer substance used in step (d), preferably the buffer system and pH of the first buffer system used in step (a) are identical to the buffer system and pH of the final aqueous buffer solution used in step (d); or (ii) each of the first buffer system and every further buffer system used in steps (b′), (f′) and (g′) comprises the final buffer substance used in step (h′), preferably the buffer system and pH of each of the first aqueous buffer solution and of every further aqueous buffer solution used in steps (b′), (f′) and (g′) are identical to the buffer system and pH of the final aqueous buffer solution.

112. The method of any one of items 102 to 111, wherein the pH of the composition is between about 4.0 and about 8.0, preferably between about 4.5 and about 8.0, such as between about 5.0 and about 8.0, between about 5.5 and about 8.0, between about 6.0 and about 8.0, between about 6.5 and about 8.0, between about 6.8 and about 7.9, or between about 7.0 and about 7.

113. The method of any one of items 102 to 112, wherein water is the main component in the formulation and/or composition and/or the total amount of solvent(s) other than water contained in the composition is less than about 0.5% (v/v).

114. The method of any one of items 102 to 113, wherein the osmolality of the composition is at most about 1000×10⁻³ osmol/kg, preferably between about 100×10⁻³ osmol/kg and about 750×10⁻³ osmol/kg, such as between about 100×10⁻³ osmol/kg and about 500×10⁻³ osmol/kg, more preferably about 300×10⁻³ osmol/kg.

115. The method of any one of items 102 to 114, wherein the concentration of the RNA in the composition is about 5 mg/l to about 500 mg/l, such as about 10 mg/l to about 400 mg/l, about 10 mg/l to about 300 mg/l, about 10 mg/l to about 200 mg/l, about 10 mg/l to about 150 mg/l, or about 10 mg/l to about 100 mg/l, preferably about 10 mg/l to about 140 mg/l, more preferably about 20 mg/l to about 130 mg/l, more preferably about 30 mg/l to about 120 mg/l.

115a. The method of any one of items 102 to 115, wherein (i) step (1) further comprises diluting the formulation prepared under (d) with a dilution solution, or step (i′) is present, wherein the dilution solution comprises a cryoprotectant; and/or (ii) the formulation obtained in step (I) and the composition comprise a cryoprotectant, preferably in a concentration of at least about 1% w/v, wherein the cryoprotectant preferably comprises one or more selected from the group consisting of carbohydrates and alcohols (such as sugar alcohols or lower alcohols), more preferably the cryoprotectant is selected from the group consisting of sucrose, glucose, glycerol, 1,2-propanediol, 1,3-propanediol, sorbitol, and a combination thereof (such as from the group consisting of sucrose, glucose, glycerol, 1,2-propanediol, 1,3-propanediol, and a combination thereof or from the group consisting of sucrose, glucose, glycerol, sorbitol, and a combination thereof), more preferably the cryoprotectant comprises sucrose and/or glycerol.

115b. The method of any one of items 102 to 106a and 108 to 115, wherein the formulation obtained in step (I) and the composition is substantially free of a cryoprotectant.

116. The method of any one of items 102 to 115b, wherein the cationically ionizable lipid comprises a head group which includes at least one nitrogen atom which is capable of being protonated under physiological conditions.

116a. The method of anyone of items 102 to 116, wherein the cationically ionizable lipid is as defined in any one of items 73 to 75a.

117. The method of any one of items 102 to 116a, wherein the ethanolic solution prepared in step (b) or (d′) further comprises one or more additional lipids and the LNPs further comprise the one or more additional lipids, wherein the one or more additional lipids are preferably selected from the group consisting of polymer conjugated lipids, neutral lipids, steroids, and combinations thereof, more preferably the one or more additional lipids comprise a polymer conjugated lipid, a neutral lipid (e.g., a phospholipid), and a steroid.

117a. The method of item 117, wherein the polymer conjugated lipid is as defined in any one of items 78 to 80.

117b. The method of item 117 or 117a, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

117c. The method of any one of items 117 to 117b, wherein the steroid comprises a sterol such as cholesterol.

118. The method of any one of items 102 to 117c, wherein the cationically ionizable lipid, the polymer conjugated lipid, the neutral lipid, and the steroid are present in the ethanolic solution in a molar ratio of 20% to 60% of the cationically ionizable lipid, 0.5% to 15% of the polymer conjugated lipid, 5% to 25% of the neutral lipid (e.g., phospholipid), and 25% to 55% of the steroid, preferably in a molar ratio of 45% to 55% of the cationically ionizable lipid, 1.0% to 5% of the polymer conjugated lipid, 8% to 12% of the neutral lipid, and 35% to 45% of the steroid.

118a. The method of any one of items 102 to 118, wherein the LNPs comprise at least about 75%, preferably at least about 80% of the RNA comprised in the composition.

119. The method of any one of items 102 to 118a, wherein the RNA is as defined in any one of items 88 and 89 to 95e.

120. The method of any one of items 102 to 105 and 107 to 119, which does not comprise step (II).

121. A method of preparing an aqueous RNA composition, wherein the method comprises:
(I) preparing a formulation comprising RNA and an aqueous phase, wherein the aqueous phase comprises a buffer substance, the buffer substance having the formula N(R¹)(R²)(R³), its N-oxide, or a protonated form thereof, wherein R¹, R², and R³ are as defined in any one of items 1 to 38; and
(II) optionally freezing the formulation to about −10° C. or below, thereby obtaining the composition.

122. The method of item 121, which comprises (II) freezing the formulation to about −10° C. or below.

122a. The method of item 122, wherein the pH of the composition is between 4.0 and 8.0, preferably between 5.0 and 7.0, more preferably between 5.5 and 6.5 and most preferably about 5.5.

123. The method of item 122 or 122a, wherein the composition comprises a cryoprotectant.

123a. The method of item 123, wherein the cryoprotectant is selected from the group consisting of sucrose, glycerol, glucose, 1,2-propanediol, 1,3-propanediol, and a combination thereof, such as from the group consisting of sucrose, glycerol and, glucose, for example the cryoprotectant is glycerol.

123b. The method of item 123 or 123a, wherein the cryoprotectant is present in a concentration of between about 100 mM and about 600 mM, preferably between about 200 mM and about 600 mM and more preferably between about 300 mM and about 500 mM.

124. The method of item 121, which does not comprise step (II).

125. The method of any one of items 121 to 124, wherein the buffer substance is selected from BTM and its protonated form, TEA and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form.

126. A method of storing a composition, comprising preparing a composition according to the method of any one of items 102 to 119, 121, 122, and 125 and storing the composition at a temperature ranging from about −90° C. to about −10° C., such as from about −90° C. to about −40° C. or from about −25° C. to about −10° C.

126a. The method of item 126, wherein the pH of the composition is between 4.0 and 8.0, preferably between 5.0 and 7.0, more preferably between 5.5 and 6.5 and most preferably about 5.5.

127. The method of item 126 or 126a, wherein the composition comprises a cryoprotectant.

127a. The method of item 127, wherein the cryoprotectant is selected from the group consisting of sucrose, glucose, glycerol, 1,2-propanediol, 1,3-propanediol, and a combination thereof, such as from the group consisting of sucrose, glycerol and glucose, for example the cryoprotectant is glycerol.

127b. The method of item 127 or 127a, wherein the cryoprotectant is present in a concentration of between about 100 mM and about 600 mM, preferably between about 200 mM and about 600 mM and more preferably between about 300 mM and about 500 mM.

128. The method of any one of items 126 to 127b, wherein storing the composition is for at least 1 month, such as at least 2 months, at least 3 months, at least 6 months, at least 12 months, at least 24 months, or at least 36 months.

129. A method of storing a composition, comprising preparing a composition according to the method of any one of items 102 to 128 and storing the composition at a temperature ranging from about 0° C. to about 20° C., such as from about 1° C. to about 15° C., from about 2° C. to about 10° C., or from about 2° C. to about 8° C., or at a temperature of about 5° C.

130. The method of item 129, wherein storing the composition is for at least 1 week, such as at least 2 weeks, at least 3 weeks, at least 4 weeks, at least 1 month, at least 2 months, at least 3 months, at least 6 months, at least 12 months, or at least 24 months.

131. A composition preparable by the method of any one of items 102 to 130.

132. The composition of item 131, which is in frozen form.

132a. The composition of item 132, wherein the pH of the composition is between 4.0 and 8.0, preferably between 5.0 and 7.0, more preferably between 5.5 and 6.5 and most preferably about 5.5.

133. The composition of item 132 or 132a, further comprising a cryoprotectant.

133a. The composition of item 133, wherein the cryoprotectant is selected from the group consisting of sucrose, glucose, glycerol, 1,2-propanediol, 1,3-propanediol, and a combination thereof, such as from the group consisting of sucrose, glycerol and glucose, for example the cryoprotectant is glycerol.

133b. The composition of item 133 or 133a, wherein the cryoprotectant is present in a concentration of between about 100 mM and about 600 mM, preferably between about 200 mM and about 600 mM and more preferably between about 300 mM and about 500 mM.

134. The composition of any one of items 132 to 133b, wherein the RNA integrity after thawing the frozen composition is at least 50% compared to the RNA integrity of the composition before the composition has been frozen.

135. The composition of any one of items 132 to 134, wherein the size (Z_{average}) and/or size distribution and/or polydispersity index (PDI) of RNA particles after thawing the frozen composition is essentially equal to the size (Z_{average}) and/or size distribution and/or PDI of the RNA particles before the composition has been frozen.

136. The composition of item 131, which is in liquid form.

137. The composition of item 136, wherein the RNA integrity after storage of the composition for at least 1 week is at least 50% compared to the RNA integrity before storage.

138. The composition of item 136 or 137, wherein the size (Z_{average}) and/or size distribution and/or polydispersity index (PDI) of RNA particles after storage of the composition for at least one week is essentially equal to the size (Z_{average}) and/or size distribution and/or PDI of the RNA particles before storage.

139. A method for preparing a ready-to-use pharmaceutical composition, the method comprising the steps of providing a frozen composition prepared by the method of any one of items 102 to 119, 121 to 123b, and 125 to 128, and thawing the frozen composition thereby obtaining the ready-to-use pharmaceutical composition.

140. A method for preparing a ready-to-use pharmaceutical composition, the method comprising the step of providing a liquid composition prepared by the method of any one of items 102 to 105, 107 to 121, 124, 125, 129, and 130, thereby obtaining the ready-to-use pharmaceutical composition.

141. A ready-to-use pharmaceutical composition preparable by the method of item 139 or 140.

142. A composition of any one of items 1 to 101, 131 to 138, and 141 for use in therapy.

143. A composition of any one of items 1 to 101, 131 to 138, and 141 for use in inducing an immune response in a subject.

143a. A method of transfecting cells, comprising adding a composition of any one of items 1 to 101, 131 to 138, and 141 to cells; and incubating the mixture of the composition and cells for a sufficient amount of time.

143b. Use of a composition of any one of items 1 to 101, 131 to 138, and 141 for transfecting cells.

143c. A kit comprising a composition of any one of items 1 to 101, 131 to 138, and 141 to 143 or a pharmaceutical composition as described herein.

143d. The kit of item 143c, which is for use in therapy, such as for inducing an immune response.

143e. The kit of item 143c or 143d, which is for use in inducing an immune response against a pathogen, such as for treating or preventing an infectious disease.

Further aspects of the present disclosure are disclosed herein.

### BRIEF DESCRIPTION OF THE FIGURES

FIG. 1: Degradation of RNA in relation of the type of buffer substance. RNA LNPs were incubated for 12 weeks at room temperature in various buffer systems (100 mM, pH 7.4).

FIG. 2: Generation of LMS in relation to the presence of anionic moieties. RNA LNPs were incubated for 12 weeks at room temperature in various buffer systems (100 mM, pH 7.4).

FIG. 3: RNA integrity versus buffer chemistry.

FIG. 4: Impact of the presence of different cryoprotectants in LNP RNA compositions subjected to at least one freeze/thaw cycle on the size of the LNPs.

### DESCRIPTION OF THE SEQUENCES

The following table provides a listing of certain sequences referenced herein.

TABLE 1
Description of the sequences

| | | |
|---|---|---|
| SEQ | | |
| ID | | |
| NO: | Description | Sequence |

Antigenic S protein sequences

| | | |
|---|---|---|
| 1 | S protein | MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQS |
| | (aa) | LLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGI |
| | | NITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASV |
| | | YAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQ |
| | | TLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYT |
| | | MSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPS |
| | | KRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNS |
| | | AIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGK |
| | | GYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT |
| 2 | S protein | auguuuguguuucuugugcugcugccucuugugucuucucagugugugaauuugacaacaagaacacagcugccaccagcuuauacaaauucuuuuaccagaggaguguauuauccug |
| | (CDS) | auaaaguguuuagaucuucugugcugcacagcacacaggaccuguuucugccauuuuuuagcaaugugacaugguuucaugcaauucaugugucuggaacaaauggaacaaaaagauuugaua |
| | | auccugugcugccuuuuaaugauggaguguauuuugcuucaacagaaaagucaaauauuauuagaggauggauuuuuggaacaacacuggauucuaaaacacagucucugcugauugugaaua |
| | | augcaacaaauguggugauuaaagugugugaauuucaguuuuguaaugauccuuuucugggaguguauuaucacaaaaauaauaaaucuuggauggaaucugaauuuagaguguauuccucug |
| | | caaauaauuguacauuugaauaugugucucagccuuuucugauggaucuggaaggaaaacagggcaauuuuaaaaaucugagagaauuuguguuuaaaaauauugauggauauuuuaaaauuu |
| | | auucuaaacacacaccaauuaauuuagugagagaucugccucagggauuuucugcucuggaaccucugguggaucugccaauuggcauuaauauuacaagauuucagacacugcuggcucugc |
| | | acagaucuuaucugacaccuggagauucuucuucuggauggacagccggagcugcagcuuauuaugugggcuaucugcagccaagaacauuucugcugaaauauaaugaaaauggaacaauua |
| | | cagaugcuguggauugugcucuggauccucugucugaaaaaaauguacauuaaaaucuuuuacaguggaaaaaggcauuuaucagacaucuaauuuuagagugcagccaacagaaucuauugu |
| | | gagauuuccaaauauuacaaaucuguguccauuuggagaaguguuuaaugcaacaagauuugcaucuguguaugcauggaauagaaaaagaauuucuaauuguguggcugauuauucugugcu |
| | | guauaauagugcuucuuuuuccacauuuaaauguuauggagugucuccaacaaaauuaaaugauuuauguuuuacaaauguguaugcugauucuuuugugaucagaggugaugaagugagaca |
| | | gauugcccccggacagacaggaaaaauugcugauuacaauuacaaacugccugaugauuuuacaggaugugugauugcuuggaauucuaauaauuuagauucuaaagugggaggaaauuacaa |
| | | uuaucuguacagacuguuuagaaaaucaaaucugaaaccuuuugaaagagauauuucaacagaaauuuaucaggcuggaucaacaccuuguaauggaguggaaggauuuaauuguuauuuucc |
| | | auuacagagcuauggauuucagccaaccaauggugugggauaucagccauauagagugguggugcugucuuuugaacugcugcaugcaccugcaacaguguguggaccuaaaaaaucuacaaa |
| | | uuuagugaaaaauaaaugugugaauuuuaauuuuaauggauuaacaggaacaggagugcugacagaaucuaauaaaaaauuucugccuuuucagcaguuuggcagagauauugcagauaccac |
| | | agaugcagugagagauccucagacauuagaaauucuggauauuacaccuuguuuuuugggggugugucugugauuacguuucagacaagagcaggaugucugauuggagcagaacaugugaau |
| | | aauucuuaugaaugugauauuccaauuggagcaggcauuugugcaucuuaucagacacagacaaauuccccaaggagagcaagaucuguggcaucucagucuauuauugcauacaccaugucu |
| | | cugggagcagaaaauucuguggcauauucuaauaauucuauugcuauuccaacaaauuuuaccauuucugugacaacagaaauuuuaccugugucuaugacaaaaacaucuguggauuguacc |
| | | auguacauuuguggagauucuacagaauguucuaaucugcugcugcaguauggaucuuuuuguacacagcugaauagagcuuuaacaggaauugcuguggaacaggauaaaaauacacaggaa |
| | | guguuugcucaggugaaacagauuuacaaaacaccaccaauuaaagauuuuggaggauuuaauuuuagccagauucugccugauccuucuaaaccuucuaaaagaucuuuuauugaagaucug |
| | | cuguuuaauaaagugacacuggcagaugcaggauuuauuaaacaguauggagauugccugggugauauugcugcaagagaucugauuugugcucagaaauuuaauggacugacagugcugccu |
| | | ccucugcugacagaugaaaugauugcucaguacacaucugcuuuacuggcuggaacaauuacaagcggauggacauuuggagcuggagcugcucugcagauuccuuuugcaaugcagauggcu |
| | | uacagauuuaauggaauuggagugacacagaauguguuauaugaaaaucagaaacugauugcaaaucaguuuaauucugcaauuggcaaaauucaggauucucugucuucuacagcuucugcu |
| | | cugggaaaacugcaggauguggugaaucagaaugcacaggcacugaauacucuggugaaacagcugucuagcaauuuuggggcaauuucuucugugcugaaugauauucugucuagacuggau |
| | | aaaguggaagcugaagugcagauugauagacugaucacaggaagacugcagucucugcagacuuaugugacacagcagcugauuagagcugcugaaauuagagcuucugcuaaucuggcugcu |
| | | acaaaaaugucugaaugugugcugggacagucaaaaagaguggauuuuuguggaaaaggauaucaucugaugucuuuuccacagucugcuccacauggagugguguuuuuacaugugacauau |
| | | gugccagcacaggaaaagaauuuuaccacagcaccagcaauuugucaugauggaaaagcacauuuuccaagagaaggaguguuugugucuaauggaacacauugguuugugacacagagaaau |
| | | uuuuaugaaccucagauuauuacaacagauaauacauuugugucaggaaauugugauguggugauuggaauugugaauaauacaguguaugauccacugcagccagaacuggauuuuuuaaag |
| | | aagaacuggauaaauauuuuaaaaaucacacaucuccugauguggauuuaggagauauuucuggaaucaaugcaucuguggugaauauucagaaagaaauugauagacugaaugaaguggcca |
| | | aaaaucugaaugaaucucugauugaucugcaggaacuuggaaaauaugaacaguacauuaaauggccuugguacauuuggcuuggauuuauugcaggauuaauugcaauugugauggugacaa |
| | | uuauguuauguuguaugacaucauguuguucuuguuuaaaaggauguuguucuuguggaagcuguuguaaauuugaugaagaugauucugaaccuguguuaaaaggagugaaauugcauuaca |
| | | ca |
| 3 | S protein | MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLP |
| | RBD | DDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK |
| | (amino | |
| | acid)(V05) | |
| 4 | S protein | auguuuguguuucuugugcugcugccucuugugucuucucaguguguggugagauuuccaaauauuacaaaucuguguccauuuggagaaguguuuaaugcaacaagauuugcaucuguguau |
| | RBD | gcauggaauagaaaaagaauuucuaauuguguggcugauuauucugugcuguauaauagugcuucuuuuuccacauuuaaauguuauggagugucuccaacaaaauuaaaugauuuauguuuu |
| | (CDS) (V05) | acaaauguguaugcugauucuuuugugaucagaggugaugaagugagacagauugcccccggacagacaggaaaaauugcugauuacaauuacaaacugccugaugauuuuacaggaugugug |
| | | auugcuuggaauucuaauaauuuagauucuaaagugggaggaaauuacaauuaucuguacagacuguuuagaaaaucaaaucugaaaccuuuugaaagagauauuucaacagaaauuuaucag |
| | | gcuggaucaacaccuuguaauggaguggaaggauuuaauuguuauuuuccauuacagagcuauggauuucagccaaccaauggugugggauaucagccauauagagugguggugcugucuuuu |
| | | gaacugcugcaugcaccugcaacaguguguggaccuaaa |
| 5 | S protein | MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLP |
| | RBD/ | DDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIP |
| | Fibritin | EAPRDGQAYVRKDGEWVLLSTFLGRSLEVLFQGPG |
| | (amino | |
| | acid) (V05) | |
| 6 | S protein | auguuuguguuucuugugcugcugccucuugugucuucucaguguguggugagauuuccaaauauuacaaaucuguguccauuuggagaaguguuuaaugcaacaagauuugcaucuguguau |
| | RBD/ | gcauggaauagaaaaagaauuucuaauuguguggcugauuauucugugcuguauaauagugcuucuuuuuccacauuuaaauguuauggagugucuccaacaaaauuaaaugauuuauguuuu |
| | Fibritin | acaaauguguaugcugauucuuuugugaucagaggugaugaagugagacagauugcccccggacagacaggaaaaauugcugauuacaauuacaaacugccugaugauuuuacaggaugugug |
| | (CDS) | auugcuuggaauucuaauaauuuagauucuaaagugggaggaaauuacaauuaucuguacagacuguuuagaaaaucaaaucugaaaccuuuugaaagagauauuucaacagaaauuuaucag |
| | (V05) | gcuggaucaacaccuuguaauggaguggaaggauuuaauuguuauuuuccauuacagagcuauggauuucagccaaccaauggugugggauaucagccauauagagugguggugcugucuuuu |
| | | gaacugcugcaugcaccugcaacaguguguggaccuaaaggcucccccggcuccggcuccggaucugguuauauuccugaagcuccaagagaugggcaagcuuacguucguaaagauggcgaa |
| | | uggguauuacuuucuaccuuuuuaggccggucccuggaggugcuguuccagggccccggc |
| 7 | S protein | MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQS |
| | PP (amino | LLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGI |
| | acid) | NITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASV |
| | (V08/V09) | YAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFE |
| | | RDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQ |
| | | TLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYT |
| | | MSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPS |
| | | KRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNS |
| | | AIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGK |
| | | GYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT |
| 8 | S protein | auguuuguguuucuugugcugcugccucuugugucuucucagugugugaauuugacaacaagaacacagcugccaccagcuuauacaaauuuuuuaccagaggaguguauuauccugauaaag |
| | PP | uguuuagaucuucugugcugcacagcacacaggaccuguuucugccauuuuuuagcaaugugacaugguuucaugcaauucaugugucuggaacaaauggaacaaaaagauuugauaauccug |
| | (CDS) (V08) | ugcugccuuuuaaugauggaguguauuuugcuucaacagaaaagucaaauauuauuagaggauggauuuuuggaacaacacuggauucuaaaacacagucucugcugauugugaauaaugcaa |
| | | caaauguggugauuaaagugugugaauuucaguuuuguaaugauccuuuucugggaguguauuaucacaaaaauaauaaaucuuggauggaaucugaauuuagaguguauuccucugcaaaua |
| | | auuguacauuugaauaugugucucagccuuuucugauggaucuggaaggaaaacagggcaauuuuaaaaaucugagagaauuuguguuuaaaaauauugauggauauuuuaaaauuuauucua |
| | | aacacacaccaauuaauuuagugagagaucugccucagggauuuucugcucuggaaccucugguggaucugccaauuggcauuaauauuacaagauuucagacacugcuggcucugcacagau |
| | | cuuaucugacaccuggagauucuucuucuggauggacagccggagcugcagcuuauuaugugggcuaucugcagccaagaacauuucugcugaaauauaaugaaaauggaacaauuacagaug |
| | | cuguggauugugcucuggauccucugucugaaacaaaauguacauuaaaaucuuuuacaguggaaaaaggcauuuaucagacaucuaauuuuagagugcagccaacagaaucuauugugagau |
| | | uuccaaauauuacaaaucuguguccauuuggagaaguguuuaaugcaacaagauuugcaucuguguaugcauggaauagaaaaagaauuucuaauuguguggcugauuauucugugcuguaua |
| | | auagugcuuuuuuuccacauuuaaauguuauggagugucuccaacaaaauuaaaugauuuauguuuuacaaauguguaugcugauucuuuugugaucagaggugaugaagugagacagauugc |
| | | ccccggacagacaggaaaaauugcugauuacaauuacaaacugccugaugauuuuacaggaugugugauugcuuggaauucuaauaauuuagauucuaaagugggaggaaauuacaauuaucu |
| | | guacagacuguuuagaaaaucaaaucugaaaccuuuugaaagagauauuucaacagaaauuuaucaggcuggaucaacaccuuguaauggaguggaaggauuuaauuguuauuuuccauuaca |
| | | gagcuauggauuucagccaaccaauggugugggauaucagccauauagagugguggugcugucuuuugaacugcugcaugcaccugcaacaguguguggaccuaaaaaaucuacaaauuuagu |
| | | gaaaaauaaaugugugaauuuuaauuuuaauggauuaacaggaacaggagugcugacagaaucuaauaaaaaauuucugccuuuucagcaguuuggcagagauauugcagauaccacagaugc |
| | | agugagagauccucagacauuagaaauucuggauauuacaccuuguuuuuugggggugugucugugauuacaccuggaacaaauacaucuaaucagguggcugugcuguaucaggaugugaau |
| | | uguacagaagugccaguggcaauucaugcagaucagcugacaccaacauggagaguguauucuacaggaucuaauguguuucagacaagagcaggaugucugauuggagcagaacaugugaau |
| | | aauucuuaugaaugugauauuccaauuggagcaggcauuugugcaucuuaucagacacagacaaauuccccaaggagagcaagaucuguggcaucucagucuauuauugcauacaccaugucu |
| | | cugggagcagaaaauucuguggcauauucuaauaauucuauugcuauuccaacaaauuuuaccauuucugugacaacagaaauuuuaccugugucuaugacaaaaacaucuguggauuguacc |
| | | auguacauuuguggagauucuacagaauguucuaaucugcugcugcaguauggaucuuuuuguacacagcugaauagagcuuuaacaggaauugcuguggaacaggauaaaaauacacaggaa |
| | | guguuugcucaggugaaacagauuuacaaaacaccaccaauuaaagauuuuggaggauuuaauuuuagccagauucugccugauccuucuaaaccuucuaaaagaucuuuuauugaagaucug |
| | | cuguuuaauaaagugacacuggcagaugcaggauuuauuaaacaguauggagauugccugggugauauugcugcaagagaucugauuugugcucagaaauuuaauggacugacagugcugccu |
| | | ccucugcugacagaugaaaugauugcucaguacacaucugcuuuacuggcuggaacaauuacaagcggauggacauuuggagcuggagcugcucugcagauuccuuuugcaaugcagauggcu |
| | | uacagauuuaauggaauuggagugacacagaauguguuauaugaaaaucagaaacugauugcaaaucaguuuaauucugcaauuggcaaaauucaggauucucugucuucuacagcuucugcu |
| | | cugggaaaacugcaggauguggugaaucagaaugcacaggcacugaauacucuggugaaacagcugucuagcaauuuuggggcaauuucuucugugcugaaugauauucugucuagacuggau |
| | | ccuccugaagcugaagugcagauugauagacugaucacaggaagacugcagucucugcagacuuaugugacacagcagcugauuagagcugcugaaauuagagcuucugcuaaucuggcugcu |
| | | acaaaaaugucugaaugugugcugggacagucaaaaagaguggauuuuuguggaaaaggauaucaucugaugucuuuuccacagucugcuccacauggagugguguuuuuacaugugacauau |
| | | gugccagcacaggaaaagaauuuuaccacagcaccagcaauuugucaugauggaaaagcacauuuuccaagagaaggaguguuugugucuaauggaacacauugguuugugacacagagaaau |
| | | uuuuaugaaccucagauuauuacaacagauaauacauuugugucaggaaauugugauguggugauuggaauugugaauaauacaguguaugauccacugcagccagaacuggauuuuuuaaag |
| | | aagaacuggauaaauauuuuaaaaaucacacaucuccugauguggauuuaggagauauuucuggaaucaaugcaucuguggugaauauucagaaagaaauugauagacugaaugaaguggcca |
| | | aaaaucugaaugaaucucugauugaucugcaggaacuuggaaaauaugaacaguacauuaaauggccuugguacauuuggcuuggauuuauugcaggauuaauugcaauugugauggugacaa |
| | | uuauguuauguuguaugacaucauguuguucuuguuuaaaaggauguuguucuuguggaagcuguuguaaauuugaugaagaugauucugaaccuguguuaaaaggagugaaauugcauuaca |
| | | ca |
| 9 | S protein | auguucguguuccuggugcugcugccucugguguccagccagugugugaaccugaccaccagaacacagcugccuccagccuacaccaacagcuuuaccagaggcguguacuaccccgacaag |
| | PP | guguucagauccagcgugcugcacucuacccaggaccuguuccugccuuucuucagcaacgugaccugguuccacgccauccacguguccggcaccaauggcaccaagagauucgacaacccc |
| | (CDS) (V09) | gugcugcccuucaacgacgggguguacuuugccagcaccgagaaguccaacaucaucagaggcuggaucuucggcaccacacuggacagcaagacccagagccugcugaucgugaacaacgcc |
| | | accaacguggucaucaaagugugcgaguuccaguucugcaacgaccccuuccugggcgucuacuaccacaagaacaacaagagcuggauggaaagcgaguuccggguguacagcagcgccaac |
| | | aacugcaccuucgaguacgugucccagccuuuccugauggaccuggaaggcaagcagggcaacuucaagaaccugcgcgaguucguguuuaagaacaucgacggcuacuucaagaucuacagc |
| | | aagcacaccccuaucaaccucguggggaucugccucagggcuucucugcucuggaaccccugguggaucugcccaucggcaucaacaucacccgguuucagacacugcuggcccugcacagaa |
| | | gcuaccugacaccuggcgauagcagcagcggauggacagcuggugccgccgcuuacuaugugggcuaccugcagccuagaaccuuccugcugaaguacaacgagaacggcaccaucaccgacg |
| | | ccguggauugugcucuggauccucugagcgagacaaagugcacccugaaguccuucaccguggaaaagggcaucuaccagaccagcaacuuccgggugcagcccaccgaauccaucgugcggu |
| | | uccccaauaucaccaaucugugccccuucggcgagguguucaaugccaccagauucgccucuguguacgccuggaaccggaagcggaucagcaauugcguggccgacuacuccgugcuguaca |
| | | acuccgccagcuucagcaccuucaagugcuacggcguguccccuaccaagcugaacgaccugugcuucacaaacguguacgccgacagcuucgugauccggggagaugaagugcggcagauug |
| | | ccccuggacagacaggcaagaucgccgacuacaacuacaagcugcccgacgacuucaccggcugugugauugccuggaacagcaacaaccuggacuccaaagucggcggcaacuacaauuacc |
| | | uguaccggcuguuccggaaguccaaucugaagcccuucgaggggacaucuccaccgagaucuaucaggccggcagcaccccuuguaacggcguggaaggcuucaacugcuacuucccacugca |
| | | guccuacggcuuucagcccacaaauggcgugggcuaucagcccuacagagugguggugcugagcuucgaacugcugcaugccccugccacagugugcggcccuaagaaaagcaccaaucucgu |
| | | gaagaacaaaugcgugaacuucaacuucaacggccugaccggcaccggcgugcugacagagagcaacaagaaguuccugccauuccagcaguuuggccgggauaucgccgauaccacagacgc |
| | | cguuagagauccccagacacuggaaauccuggacaucaccccuugcagcuucggcggagugucugugaucaccccuggcaccaacaccagcaaucagguggcagugcuguaccaggacgugaa |
| | | cuguaccgaagugcccguggccauucacgccgaucagcugacaccuacauggcggguguacuccaccggcagcaauguguuucagaccagagccggcugucugaucggagccgagcacgugaa |
| | | caauagcuacgagugcgacauccccaucggcgcuggaaucugcgccagcuaccagacacagacaaacagcccucggagagccagaagcguggccagccagagcaucauugccuacacaauguc |
| | | ucugggcgccgagaacagcguggccuacuccaacaacucuaucgcuauccccaccaacuucaccaucagcgugaccacagagauccugccuguguccaugaccaagaccagcguggacugcac |
| | | cauguacaucugcggcgauuccaccgagugcuccaaccugcugcugcaguacggcagcuucugcacccagcugaauagagcccugacagggaucgccguggaacaggacaagaacacccaaga |
| | | gguguucgcccaagugaagcagaucuacaagaccccuccuaucaaggacuucggcggcuucaauuucagccagauucugcccgauccuagcaagcccagcaagcggagcuucaucgaggaccu |
| | | gcuguucaacaaagugacacuggccgacgccggcuucaucaagcaguauggcgauugucugggcgacauugccgccagggaucugauuugcgcccagaaguuuaacggacugacagugcugcc |
| | | uccucugcugaccgaugagaugaucgcccaguacacaucugcccugcuggccggcacaaucacaagcggcuggacauuuggagcaggcgccgcucugcagauccccuuugcuaugcagauggc |
| | | cuaccgguucaacggcaucggagugacccagaaugugcuguacgagaaccagaagcugaucgccaaccaguucaacagcgccaucggcaagauccaggacagccugagcagcacagcaagcgc |
| | | ccugggaaagcugcaggacguggucaaccagaaugcccaggcacugaacacccuggucaagcagcuguccuccaacuucggcgccaucagcucugugcugaacgauauccugagcagacugga |
| | | cccuccugaggccgaggugcagaucgacagacugaucacaggcagacugcagagccuccagacauacgugacccagcagcugaucagagccgccgagauuagagccucugccaaucuggccgc |
| | | caccaagaugucugagugugugcugggccagagcaagagaguggacuuuugcggcaagggcuaccaccugaugagcuucccucagucugccccucacggcgugguguuucugcacgugacaua |
| | | ugugcccgcucaagagaagaauuucaccaccgcuccagccaucugccacgacggcaaagcccacuuuccuagagaaggcguguucgugudcaacggcacccauugguucgugacacagcggaa |
| | | cuucuacgagccccagaucaucaccaccgacaacaccuucgugucuggcaacugcgacgucgugaucggcauugugaacaauaccguguacgacccucugcagcccgagcuggacagcuucaa |
| | | agaggaacuggacaaguacuuuaagaaccacacaagccccgacguggaccugggcgauaucagcggaaucaaugccagcgucgugaacauccagaaagagaucgaccggcugaacgagguggc |
| | | caagaaucugaacgagagccugaucgaccugcaagaacuggggaaguacgagcaguacaucaaguggcccugguacaucuggcugggcuuuaucgccggacugauugccaucgugauggucac |
| | | aaucaugcuguguugcaugaccagcugcuguagcugccugaagggcuguuguagcuguggcagcugcugcaaguucgacgaggacgauucugagcccgugcugaagggcgugaaacugcacua |
| | | caca |

Foldon

| | | |
|---|---|---|
| 10 | Foldon | GSGYIPEAPRDGQAYVRKDGEWVLLSTFLGRSLEVLFQGPG |
| | (aa) | |
| 11 | Foldon | ggaucugguuauauuccugaagcuccaagagaugggcaagcuuacguucguaaagauggcgaauggguauuacuuucuaccuuuuuaggccggucccuggaggugcuguuccagggccccggc |
| | (CDS) | |

5′-UTR (hAg-Kozak)

| | | |
|---|---|---|
| 12 | 5′-UTR | AACUAGUAUUCUUCUGGUCCCCACAGACUCAGAGAGAACCCGCCACC |

3′-UTR (FI element)

| | | |
|---|---|---|
| 13 | 3′-UTR | CUGGUACUGCAUGCACGCAAUGCUAGCUGCCCCUUUCCCGUCCUGGGUACCCCGAGUCUCCCCCGACCUCGGGUCCCAGGUAUGCUCCCACCUCCACCUGCCCCACUCA |
| | | CCACCUCUGCUAGUUCCAGACACCUCCCAAGCACGCAGCAAUGCAGCUCAAAACGCUUAGCCUAGCCACACCCCCACGGGAAACAGCAGUGAUUAACCUUUAGCAAUAAA |
| | | CGAAAGUUUAACUAAGCUAUACUAACCCCAGGGUUGGUCAAUUUCGUGCCAGCCACACC |

A30L70

| | | |
|---|---|---|
| 14 | A30L70 | AAAAAAAAAAAAAAAAAAAAAAAAAAAAAAGCAUAUGACUAAAAAAAAAAAAAAAAAAAAAAAAAAAAAAAAAAAAAAAAAAAAAAAAAAAAAAAAAAAAAAAAAAAAAA |

SARS-CoV-2 Vaccine RNA constructs

| | | | |
|---|---|---|---|
| 20 | Construct 2 | agaauaaacu aguauucuuc ugguccccac agacucagag agaacccgcc accauguucg | 60 |
| | | uguuccuggu gcugcugccu cuggugucca gccagugugu gaaccugacc accagaacac | 120 |
| | | agcugccucc agccuacacc aacagcuuua ccagaggcgu guacuacccc gacaaggugu | 180 |
| | | ucagauccag cgugcugcac ucuacccagg accuguuccu gccuuucuuc agcaacguga | 240 |
| | | ccugguucca cgccauccac guguccggca ccaauggcac caagagauuc gacaaccccg | 300 |
| | | ugcugcccuu caacgacggg guguacuuug ccagcaccga gaaguccaac aucaucagag | 360 |
| | | gcuggaucuu cggcaccaca cuggacagca agacccagag ccugcugauc gugaacaacg | 420 |
| | | ccaccaacgu ggucaucaaa gugugcgagu uccaguucug caacgacccc uuccugggcg | 480 |
| | | ucuacuacca caagaacaac aagagcugga uggaaagcga guuccgggug uacagcagcg | 540 |
| | | ccaacaacug caccuucgag uacguguccc agccuuuccu gauggaccug gaaggcaagc | 600 |
| | | agggcaacuu caagaaccug cgcgaguucg uguuuaagaa caucgacggc uacuucaaga | 660 |
| | | ucuacagcaa gcacaccccu aucaaccucg ugcgggaucu gccucagggc uucucugcuc | 720 |
| | | uggaaccccu gguggaucug cccaucggca ucaacaucac ccgguuucag acacugcugg | 780 |
| | | cccugcacag aagcuaccug acaccuggcg auagcagcag cggauggaca gcuggugccg | 840 |
| | | ccgcuuacua ugugggcuac cugcagccua gaaccuuccu gcugaaguac aacgagaacg | 900 |
| | | gcaccaucac cgacgccgug gauugugcuc uggauccucu gagcgagaca aagugcaccc | 960 |
| | | ugaaguccuu caccguggaa aagggcaucu accagaccag caacuuccgg gugcagccca | 1020 |
| | | ccgaauccau cgugcgguuc cccaauauca ccaaucugug ccccuucggc gagguguuca | 1080 |
| | | augccaccag auucgccucu guguacgccu ggaaccggaa gcggaucagc aauugcgugg | 1140 |
| | | ccgacuacuc cgugcuguac aacuccgcca gcuucagcac cuucaagugc uacggcgugu | 1200 |
| | | ccccuaccaa gcugaacgac cugugcuuca caaacgugua cgccgacagc uucgugaucc | 1260 |
| | | ggggagauga agugcggcag auugccccug gacagacagg caagaucgcc gacuacaacu | 1320 |
| | | acaagcugcc cgacgacuuc accggcugug ugauugccug gaacagcaac aaccuggacu | 1380 |
| | | ccaaagucgg cggcaacuac aauuaccugu accggcuguu ccggaagucc aaucugaagc | 1440 |
| | | ccuucgagcg ggacaucucc accgagaucu aucaggccgg cagcaccccu uguaacggcg | 1500 |
| | | uggaaggcuu caacugcuac uucccacugc aguccuacgg cuuucagccc acaaauggcg | 1560 |
| | | ugggcuauca gcccuacaga gugguggugc ugagcuucga acugcugcau gccccugcca | 1620 |
| | | cagugugcgg cccuaagaaa agcaccaauc ucgugaagaa caaaugcgug aacuucaacu | 1680 |
| | | ucaacggccu gaccggcacc ggcgugcuga cagagagcaa caagaaguuc cugccauucc | 1740 |
| | | agcaguuugg ccgggauauc gccgauacca cagacgccgu uagagauccc cagacacugg | 1800 |
| | | aaauccugga caucaccccu ugcagcuucg gcggaguguc ugugaucacc ccuggcacca | 1860 |
| | | acaccagcaa ucagguggca gugcuguacc aggacgugaa cuguaccgaa gugcccgugg | 1920 |
| | | ccauucacgc cgaucagcug acaccuacau ggcgggugua cuccaccggc agcaaugugu | 1980 |
| | | uucagaccag agccggcugu cugaucggag ccgagcacgu gaacaauagc uacgagugcg | 2040 |
| | | acauccccau cggcgcugga aucugcgcca gcuaccagac acagacaaac agcccucgga | 2100 |
| | | gagccagaag cguggccagc cagagcauca uugccuacac aaugucucug ggcgccgaga | 2160 |
| | | acagcguggc cuacuccaac aacucuaucg cuauccccac caacuucacc aucagcguga | 2220 |
| | | ccacagagau ccugccugug uccaugacca agaccagcgu ggacugcacc auguacaucu | 2280 |
| | | gcggcgauuc caccgagugc uccaaccugc ugcugcagua cggcagcuuc ugcacccagc | 2340 |
| | | ugaauagagc ccugacaggg aucgccgugg aacaggacaa gaacacccaa gagguguucg | 2400 |
| | | cccaagugaa gcagaucuac aagaccccuc cuaucaagga cuucggcggc uucaauuuca | 2460 |
| | | gccagauucu gcccgauccu agcaagccca gcaagcggag cuucaucgag gaccugcugu | 2520 |
| | | ucaacaaagu gacacuggcc gacgccggcu ucaucaagca guauggcgau ugucugggcg | 2580 |
| | | acauugccgc cagggaucug auuugcgccc agaaguuuaa cggacugaca gugcugccuc | 2640 |
| | | cucugcugac cgaugagaug aucgcccagu acacaucugc ccugcuggcc ggcacaauca | 2700 |
| | | caagcggcug gacauuugga gcaggcgccg cucugcagau ccccuuugcu augcagaugg | 2760 |
| | | ccuaccgguu caacggcauc ggagugaccc agaaugugcu guacgagaac cagaagcuga | 2820 |
| | | ucgccaacca guucaacagc gccaucggca agauccagga cagccugagc agcacagcaa | 2880 |
| | | gcgcccuggg aaagcugcag gacgugguca accagaaugc ccaggcacug aacacccugg | 2940 |
| | | ucaagcagcu guccuccaac uucggcgcca ucagcucugu gcugaacgau auccugagca | 3000 |
| | | gacuggaccc uccugaggcc gaggugcaga ucgacagacu gaucacaggc agacugcaga | 3060 |
| | | gccuccagac auacgugacc cagcagcuga ucagagccgc cgagauuaga gccucugcca | 3120 |
| | | aucuggccgc caccaagaug ucugagugug ugcugggcca gagcaagaga guggacuuuu | 3180 |
| | | gcggcaaggg cuaccaccug augagcuucc cucagucugc cccucacggc gugguguuuc | 3240 |
| | | ugcacgugac auaugugccc gcucaagaga agaauuucac caccgcucca gccaucugcc | 3300 |
| | | acgacggcaa agcccacuuu ccuagagaag gcguguucgu guccaacggc acccauuggu | 3360 |
| | | ucgugacaca gcggaacuuc uacgagcccc agaucaucac caccgacaac accuucgugu | 3420 |
| | | cuggcaacug cgacgucgug aucggcauug ugaacaauac cguguacgac ccucugcagc | 3480 |
| | | ccgagcugga cagcuucaaa gaggaacugg acaaguacuu uaagaaccac acaagccccg | 3540 |
| | | acguggaccu gggcgauauc agcggaauca augccagcgu cgugaacauc cagaaagaga | 3600 |
| | | ucgaccggcu gaacgaggug gccaagaauc ugaacgagag ccugaucgac cugcaagaac | 3660 |
| | | uggggaagua cgagcaguac aucaaguggc ccugguacau cuggcugggc uuuaucgccg | 3720 |
| | | gacugauugc caucgugaug gucacaauca ugcuguguug caugaccagc ugcuguagcu | 3780 |
| | | gccugaaggg cuguuguagc uguggcagcu gcugcaaguu cgacgaggac gauucugagc | 3840 |
| | | ccgugcugaa gggcgugaaa cugcacuaca caugaugacu cgagcuggua cugcaugcac | 3900 |
| | | gcaaugcuag cugccccuuu cccguccugg guaccccgag ucucccccga ccucgggucc | 3960 |
| | | cagguaugcu cccaccucca ccugccccac ucaccaccuc ugcuaguucc agacaccucc | 4020 |
| | | caagcacgca gcaaugcagc ucaaaacgcu uagccuagcc acacccccac gggaaacagc | 4080 |
| | | agugauuaac cuuuagcaau aaacgaaagu uuaacuaagc uauacuaacc ccaggguugg | 4140 |
| | | ucaauuucgu gccagccaca cccuggagcu agcaaaaaaa aaaaaaaaaa aaaaaaaaaa | 4200 |
| | | aaagcauaug acuaaaaaaa aaaaaaaaaa aaaaaaaaaa aaaaaaaaaa aaaaaaaaaa | 4260 |
| | | aaaaaaaaaa aaaaaaaaaa aaa | 4283 |
| 21 | Construct 1 | agaauaaacu aguauucuuc ugguccccac agacucagag agaacccgcc accauguuug | 60 |
| | | uguuucuugu gcugcugccu cuugugucuu cucagugugu ggugagauuu ccaaauauua | 120 |
| | | caaaucugug uccauuugga gaaguguuua augcaacaag auuugcaucu guguaugcau | 180 |
| | | ggaauagaaa aagaauuucu aauugugugg cugauuauuc ugugcuguau aauagugcuu | 240 |
| | | cuuuuuccac auuuaaaugu uauggagugu cuccaacaaa auuaaaugau uuauguuuua | 300 |
| | | caaaugugua ugcugauucu uuugugauca gaggugauga agugagacag auugcccccg | 360 |
| | | gacagacagg aaaaauugcu gauuacaauu acaaacugcc ugaugauuuu acaggaugug | 420 |
| | | ugauugcuug gaauucuaau aauuuagauu cuaaaguggg aggaaauuac aauuaucugu | 480 |
| | | acagacuguu uagaaaauca aaucugaaac cuuuugaaag agauauuuca acagaaauuu | 540 |
| | | aucaggcugg aucaacaccu uguaauggag uggaaggauu uaauuguuau uuuccauuac | 600 |
| | | agagcuaugg auuucagcca accaauggug ugggauauca gccauauaga gugguggugc | 660 |
| | | ugucuuuuga acugcugcau gcaccugcaa cagugugugg accuaaaggc ucccccggcu | 720 |
| | | ccggcuccgg aucugguuau auuccugaag cuccaagaga ugggcaagcu uacguucgua | 780 |
| | | aagauggcga auggguauua cuuucuaccu uuuuaggccg gucccuggag gugcuguucc | 840 |
| | | agggccccgg cugaugacuc gagcugguac ugcaugcacg caaugcuagc ugccccuuuc | 900 |
| | | ccguccuggg uaccccgagu cucccccgac cucggguccc agguaugcuc ccaccuccac | 960 |
| | | cugccccacu caccaccucu gcuaguucca gacaccuccc aagcacgcag caaugcagcu | 1020 |
| | | caaaacgcuu agccuagcca cacccccacg ggaaacagca gugauuaacc uuuagcaaua | 1080 |
| | | aacgaaaguu uaacuaagcu auacuaaccc caggguuggu caauuucgug ccagccacac | 1140 |
| | | ccuggagcua gcaaaaaaaa aaaaaaaaaa aaaaaaaaaa aagcauauga cuaaaaaaaa | 1200 |
| | | aaaaaaaaaa aaaaaaaaaa aaaaaaaaaa aaaaaaaaaa aaaaaaaaaa aaaaaaaaaa | 1260 |
| | | aa | 1262 |
| 30 | Construct 3c | agaauaaacu aguauucuuc ugguccccac agacucagag agaacccgcc accauguuug | 60 |
| | | uguuucuugu gcugcugccu cuugugucuu cucagugugu gaauuugaca gugagauuuc | 120 |
| | | caaauauuac aaaucugugu ccauuuggag aaguguuuaa ugcaacaaga uuugcaucug | 180 |
| | | uguaugcaug gaauagaaaa agaauuucua auuguguggc ugauuauucu gugcuguaua | 240 |
| | | auagugcuuc uuuuuccaca uuuaaauguu auggaguguc uccaacaaaa uuaaaugauu | 300 |
| | | uauguuuuac aaauguguau gcugauucuu uugugaucag aggugaugaa gugagacaga | 360 |
| | | uugcccccgg acagacagga aaaauugcug auuacaauua caaacugccu gaugauuuua | 420 |
| | | caggaugugu gauugcuugg aauucuaaua auuuagauuc uaaaguggga ggaaauuaca | 480 |
| | | auuaucugua cagacuguuu agaaaaucaa aucugaaacc uuuugaaaga gauauuucaa | 540 |
| | | cagaaauuua ucaggcugga ucaacaccuu guaauggagu ggaaggauuu aauuguuauu | 600 |
| | | uuccauuaca gagcuaugga uuucagccaa ccaauggugu gggauaucag ccauauagag | 660 |
| | | ugguggugcu gucuuuugaa cugcugcaug caccugcaac agugugugga ccuaaaggcu | 720 |
| | | cccccggcuc cggcuccgga ucugguuaua uuccugaagc uccaagagau gggcaagcuu | 780 |
| | | acguucguaa agauggcgaa uggguauuac uuucuaccuu uuuaggaagc ggcagcggau | 840 |
| | | cugaacagua cauuaaaugg ccuugguaca uuuggcuugg auuuauugca ggauuaauug | 900 |
| | | caauugugau ggugacaauu auguuauguu guaugacauc auguuguucu uguuuaaaag | 960 |
| | | gauguuguuc uuguggaagc uguuguugau gacucgagcu gguacugcau gcacgcaaug | 1020 |
| | | cuagcugccc cuuucccguc cuggguaccc cgagucuccc ccgaccucgg gucccaggua | 1080 |
| | | ugcucccacc uccaccugcc ccacucacca ccucugcuag uuccagacac cucccaagca | 1140 |
| | | cgcagcaaug cagcucaaaa cgcuuagccu agccacaccc ccacgggaaa cagcagugau | 1200 |
| | | uaaccuuuag caauaaacga aaguuuaacu aagcuauacu aaccccaggg uuggucaauu | 1260 |
| | | ucgugccagc cacacccugg agcuagcaaa aaaaaaaaaa aaaaaaaaaa aaaaaaagca | 1320 |
| | | uaugacuaaa aaaaaaaaaa aaaaaaaaaa aaaaaaaaaa aaaaaaaaaa aaaaaaaaaa | 1380 |
| | | aaaaaaaaaa aaaaaaa | 1397 |
| 32 | Construct 3d | agaauaaacu aguauucuuc ugguccccac agacucagag agaacccgcc accauggauu | 60 |
| | | ggauuuggag aauccuguuc cucgugggag ccgcuacagg agcccacucc cagaugcagg | 120 |
| | | ugagauuucc aaauauuaca aaucuguguc cauuuggaga aguguuuaau gcaacaagau | 180 |
| | | uugcaucugu guaugcaugg aauagaaaaa gaauuucuaa uuguguggcu gauuauucug | 240 |
| | | ugcuguauaa uagugcuucu uuuuccacau uuaaauguua uggagugucu ccaacaaaau | 300 |
| | | uaaaugauuu auguuuuaca aauguguaug cugauucuuu ugugaucaga ggugaugaag | 360 |
| | | ugagacagau ugcccccgga cagacaggaa aaauugcuga uuacaauuac aaacugccug | 420 |
| | | augauuuuac aggaugugug auugcuugga auucuaauaa uuuagauucu aaagugggag | 480 |
| | | gaaauuacaa uuaucuguac agacuguuua gaaaaucaaa ucugaaaccu uuugaaagag | 540 |
| | | auauuucaac agaaauuuau caggcuggau caacaccuug uaauggagug gaaggauuua | 600 |
| | | auuguuauuu uccauuacag agcuauggau uucagccaac caauggugug ggauaucagc | 660 |
| | | cauauagagu gguggugcug ucuuuugaac ugcugcaugc accugcaaca guguguggac | 720 |
| | | cuaaaggcuc ccccggcucc ggcuccggau cugguuauau uccugaagcu ccaagagaug | 780 |
| | | ggcaagcuua cguucguaaa gauggcgaau ggguauuacu uucuaccuuu uuaggaagcg | 840 |
| | | gcagcggauc ugaacaguac auuaaauggc cuugguacau uuggcuugga uuuauugcag | 900 |
| | | gauuaauugc aauugugaug gugacaauua uguuauguug uaugacauca uguuguucuu | 960 |
| | | guuuaaaagg auguuguucu uguggaagcu guuguugaug acucgagcug guacugcaug | 1020 |
| | | cacgcaaugc uagcugcccc uuucccgucc uggguacccc gagucucccc cgaccucggg | 1080 |
| | | ucccagguau gcucccaccu ccaccugccc cacucaccac cucugcuagu uccagacacc | 1140 |
| | | ucccaagcac gcagcaaugc agcucaaaac gcuuagccua gccacacccc cacgggaaac | 1200 |
| | | agcagugauu aaccuuuagc aauaaacgaa aguuuaacua agcuauacua accccagggu | 1260 |
| | | uggucaauuu cgugccagcc acacccugga gcuagcaaaa aaaaaaaaaa aaaaaaaaaa | 1320 |
| | | aaaaaagcau augacuaaaa aaaaaaaaaa aaaaaaaaaa aaaaaaaaaa aaaaaaaaaa | 1380 |
| | | aaaaaaaaaa aaaaaaaaaa aaaaaa | 1406 |

Sec/MITD

| | | |
|---|---|---|
| 35 | Sec | MRVMAPRTLILLLSGALALTETWAGS |
| 36 | MITD | IVGIVAGLAVLAVVVIGAVVATVMCRRKSSGGKGGSYSQAASSDSAQGSDVSLTA |

P2P16

| | | |
|---|---|---|
| 37 | P2P16 | KKQYIKANSKFIGITELKKLGGGKRGGGKKMTNSVDDALINSTKIYSYFPSVISKVNQGAQGKKL |

GS Linker

| | | |
|---|---|---|
| 38 | GS Linker 1 | GGSGGGGSGG |
| 39 | GS Linker 2 | GSSGGGGSPGGGSS |

Helper epitopdes

| | | |
|---|---|---|
| 40 | P2 | QYIKANSKFIGITEL |
| 41 | P16 | MTNSVDDALINSTKIYSYFPSVISKVNQGAQG |

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional chemistry, biochemistry, cell biology, immunology, and recombinant DNA techniques which are explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; R⁵mpp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

All methods described herein can be performed in any suitable order unless otherwise indicated herein or otherwise clearly contradicted by the context.

The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±10%, such as ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.2%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “enhance” and “increase” as used herein means the ability to cause an overall increase, or enhancement, for example, by at least about 5% or greater, about 10% or greater, about 15% or greater, about 20% or greater, about 25% or greater, about 30% or greater, about 40% or greater, about 50% or greater, about 75% or greater, or about 100% or greater in the level. In some embodiments, these terms relate to an increase or enhancement by at least about 10%, at least about 20%, at least about 30%, at least about 40%, at least about 50%, at least about 80%, or at least about 100%.

“Physiological pH” as used herein refers to a pH of about 7.5 or about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

“Physiological conditions” as used herein refer to the conditions (in particular pH and temperature) in a living subject, in particular a human. Preferably, physiological conditions mean a physiological pH and/or a temperature of about 37° C.

As used in the present disclosure, “% (w/v)” (or “% w/v”) refers to weight by volume percent, which is a unit of concentration measuring the amount of solute in grams (g) expressed as a percent of the total volume of solution in milliliters (ml).

As used in the present disclosure, “% by weight” or “% (w/w)” (or “% w/w”) refers to weight percent, which is a unit of concentration measuring the amount of a substance in grams (g) expressed as a percent of the total weight of the total composition in grams (g).

As used in the present disclosure, “mol %” is defined as the ratio of the number of moles of one component to the total number of moles of all components, multiplied by 100.

As used in the present disclosure, “mol % of the total lipid” is defined as the ratio of the number of moles of one lipid component to the total number of moles of all lipids, multiplied by 100. In this context, in some embodiments, the term “total lipid” includes lipids and lipid-like material.

The term “ionic strength” refers to the mathematical relationship between the number of different kinds of ionic species in a particular solution and their respective charges. Thus, ionic strength I is represented mathematically by the formula:

$$I = \frac{1}{2} \cdot {\sum }_{i} {z}_{i}^{2} \cdot {c}_{i}$$

in which c is the molar concentration of a particular ionic species and z the absolute value of its charge. The sum Σ is taken over all the different kinds of ions (i) in solution.

According to the disclosure, the term “ionic strength” in some embodiments relates to the presence of monovalent ions.

Regarding the presence of divalent inorganic ions, in particular divalent inorganic cations, their concentration or effective concentration (presence of free ions) due to the presence of chelating agents is in one embodiment sufficiently low so as to prevent degradation of the RNA. In one embodiment, the concentration or effective concentration of divalent inorganic ions is below the catalytic level for hydrolysis of the phosphodiester bonds between RNA nucleotides. In one embodiment, the concentration of free divalent inorganic ions is 20 μM or less. In one embodiment, there are no or essentially no free divalent inorganic ions.

“Osmolality” refers to the concentration of a particular solute expressed as the number of osmoles of solute per kilogram of solvent.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “reconstitute” relates to adding a solvent such as water to a dried product to return it to a liquid state such as its original liquid state.

The term “freezing” relates to the solidification of a liquid, usually with the removal of heat.

The term “aqueous phase” as used herein in relation to a composition/formulation comprising particles, in particular LNPs, liposomes, and/or lipoplexes, means the mobile or liquid phase, i.e., the continuous water phase including all components dissolved therein but (formally) excluding the particles. Thus, if particles, such as LNPs, are dispersed in an aqueous phase and the aqueous phase is to be substantially free of compound X, the aqueous phase is free of X is such manner as it is practically and realistically feasible, e.g., the concentration of compound X in the aqueous composition is less than 1% by weight. However, it is possible that, at the same time, the particles dispersed in the aqueous phase may comprise compound X in an amount of more than 1% by weight.

The expression “protonated form” as used herein in relation with a base (e.g., the buffer substance having the formula N(R¹)(R²)(R³) or its N-oxide) means the conjugate acid of the base, wherein the conjugate acid contains a proton which is removable by deprotonation resulting in the base. For example, the protonated form of TEA has the formula [HN(CH₂CH₂OH)₃]⁺. A “buffer substance” as used herein refers to a mixture of the base and its protonated form (e.g., a mixture of TEA and [HN(CH₂CH₂OH)₃]⁺). Consequently, the amount of a buffer substance contained in a composition is the sum of the amounts of both the base and the conjugate acid in the composition.

The term “recombinant” in the context of the present disclosure means “made through genetic engineering”. In some embodiments, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

As used herein, the terms “room temperature” and “ambient temperature” are used interchangeably herein and refer to temperatures from at least about 15° C., preferably from about 15° C. to about 35° C., from about 15° C. to about 30° C., from about 15° C. to about 25° C., or from about 17° C. to about 22° C. Such temperatures will include 15° C., 16° C., 17° C., 18° C., 19° C., 20° C., 21° C. and 22° C.

The term “alkyl” refers to a monoradical of a saturated straight or branched hydrocarbon. Preferably, the alkyl group comprises from 1 to 12 (such as 1 to 10) carbon atoms, i.e., 1, 2, 3, 4, 5, 6, 7, 8, 9, 10, 11, or 12 carbon atoms, abbreviated as C₁₋₂ alkyl, (such as 1, 2, 3, 4, 5, 6, 7, 8, 9, or 10 carbon atoms, abbreviated as C₁₋₁₀ alkyl), more preferably 1 to 8 carbon atoms, such as 1 to 6 or 1 to 4 carbon atoms.

Exemplary alkyl groups include methyl, ethyl, propyl, iso-propyl (also called 2-propyl or 1-methylethyl), butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1^{st} level substituent, as specified herein. Examples of a substituted alkyl include chloromethyl, dichloromethyl, fluoromethyl, and difluoromethyl.

The term “alkylene” refers to a diradical of a saturated straight or branched hydrocarbon. Preferably, the alkylene comprises from 1 to 12 (such as 1 to 10) carbon atoms, i.e., 1, 2, 3, 4, 5, 6, 7, 8, 9, 10, 11, or 12 carbon atoms (such as 1, 2, 3, 4, 5, 6, 7, 8, 9, or 10 carbon atoms), more preferably 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1^{st} level substituent, as specified herein.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenyl group comprises from 2 to 12, abbreviated as C₂₋₁₂ alkenyl, (e.g., 2 to 10) carbon atoms and 1, 2, 3, 4, 5, or 6 (e.g., 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, I-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, and the like. If an alkenyl group is attached to a nitrogen atom, the double bond cannot be alpha to the nitrogen atom. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different).

Preferably, the substituent other than hydrogen is a 1^{st} level substituent as specified herein.

The term “alkynyl” refers to a linear or branched monovalent hydrocarbon moiety having at least one carbon-carbon triple bond in which the total carbon atoms may be six to thirty, typically six to twenty, often six to eighteen. Alkynyl groups can optionally have one or more carbon carbon double bonds.

Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds. Preferably, the alkenylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenylene group comprises from 2 to 12 (such as 2 to 10 carbon) atoms and 1, 2, 3, 4, 5, or 6 (such as 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. If an alkenylene group is attached to a nitrogen atom, the double bond cannot be alpha to the nitrogen atom. A “substituted alkenylene” means that one or more (such as 1 to the maximum number of hydrogen atoms bound to an alkenylene group, e.g., 1, 2, 3, 4, 5, 6, 7, 8, 9, or up to 10, such as between 1 to 5, 1 to 4, or 1 to 3, or 1 or 2) hydrogen atoms of the alkenylene group are replaced with a substituent other than hydrogen (when more than one hydrogen atom is replaced the substituents may be the same or different). Preferably, the substituent other than hydrogen is a 14 level substituent as specified herein.

The term “cycloalkyl” represents cyclic non-aromatic versions of “alkyl” and “alkenyl” with preferably 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The cycloalkyl group may consist of one ring (monocyclic), two rings (bicyclic), or mre than two rings (polycyclic).

The term “cycloalkylene” represents cyclic non-aromatic versions of “alkylene” and is a geminal, vicinal or isolated diradical. In certain embodiments, the cycloalkylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In one embodiment the cycloalkylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered) cycloalkylene. Generally, instead of using the ending “ylene” for cycloalkylene moieties as specified above, one can also use the ending “diyl” (e.g., 1,2-cyclopropylene can also be called cyclopropan-1,2-diyl) Exemplary cycloalkylene groups include cyclohexylene, cycloheptylene, cyclopropylene, cyclobutylene, cyclopentylene, cyclooctylene, bicyclo[3.2.1]octylene, bicyclo[3.2.2]nonylene, and adamantanylene (e.g., tricyclo[3.3.1.1^{3,7}]decan-2,2-diyl). A “substituted cycloalkylene” means that one or more (such as 1 to the maximum number of hydrogen atoms bound to an cycloalkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1^{st} level substituent as specified herein.

The term “cycloalkenylene” represents cyclic non-aromatic versions of “alkenylene” and is a geminal, vicinal or isolated diradical. Generally, the maximal number of carbon-carbon double bonds in the cycloalkenylene group can be equal to the integer which is calculated by dividing the number of carbon atoms in the cycloalkenylene group by 2 and, if the number of carbon atoms in the cycloalkenylene group is uneven, rounding the result of the division down to the next integer. For example, for an cycloalkenylene group having 9 carbon atoms, the maximum number of carbon-carbon double bonds is 4. Preferably, the cycloalkenylene group has 1 to 6 (such as 1 to 4), i.e., 1, 2, 3, 4, 5, or 6, carbon-carbon double bonds. In certain embodiments, the cycloalkenylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In one embodiment the cycloalkenylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered) cycloalkenylene. Exemplary cycloalkenylene groups include cyclohexenylene, cycloheptenylene, cyclopropenylene, cyclobutenylene, cyclopentenylene, and cyclooctenylene. A “substituted cycloalkenylene” means that one or more (such as 1 to the maximum number of hydrogen atoms bound to an cycloalkenylene group, e.g., 1, 2, 3, 4, 5, 6, 7, 8, 9, or up to 10, such as between 1 to 5, 1 to 4, or 1 to 3, or 1 or 2) hydrogen atoms of the cycloalkenylene group are replaced with a substituent other than hydrogen (when more than one hydrogen atom is replaced the substituents may be the same or different). Preferably, the substituent other than hydrogen is a 1^{st} level substituent as specified herein.

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes.

The term “heterocyclyl” or “heterocyclic ring” means a non-aromatic cycloalkyl group as defined above in which from 1, 2, 3, or 4 ring carbon atoms in the cycloalkyl group are replaced by heteroatoms, preferably selected from the group consisting of oxygen, nitrogen, silicon, selenium, phosphorous, and sulfur, more preferably from the group consisting of O, S, and N. A heterocyclyl group has preferably 1 or 2 rings containing from 3 to 10, such as 3, 4, 5, 6, or 7, ring atoms. Preferably, in each ring of the heterocyclyl group the maximum number of O atoms is 1, the maximum number of S atoms is 1, and the maximum total number of O and S atoms is 2. Exemplary heterocyclyl groups include morpholinyl, pyrrolidinyl, imidazolidinyl, pyrazolidinyl, oxazolidinyl, thiazolidinyl, piperidinyl (also called piperidyl), piperazinyl, 1,2-diazinanyl, 1,3-diazinanyl, 1,3,5-triazinanyl, morpholinyl, thiomorpholinyl, di- and tetrahydrofuranyl, di- and tetrahydrothienyl, di- and tetrahydropyranyl, urotropinyl, lactones, lactams, cyclic imides, and cyclic anhydrides. A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). Preferably, the substituent other than hydrogen is a 1^{st} level substituent as specified herein.

The term “N-heterocyclic ring” means a heterocyclic ring as defined above, wherein the heterocyclic ring contains at least one N ring atom and may contain one or more further ring heteroatoms (preferably selected from the group consisting of oxygen, nitrogen, silicon, selenium, phosphorous, and sulfur, more preferably from the group consisting of O, S, and N). Exemplary N-heterocyclic rings include pyrrolidinyl, imidazolidinyl, pyrazolidinyl, oxazolidinyl, thiazolidinyl, piperidinyl, piperazinyl, 1,2-diazinanyl, 1,3-diazinanyl, 1,3,5-triazinanyl, morpholinyl, and thiomorpholinyl, preferably selected from piperidinyl, piperazinyl, 1,2-diazinanyl, 1,3-diazinanyl, morpholinyl, and thiomorpholinyl. A “substituted N-heterocyclic ring” means that one or more (such as 1 to the maximum number of hydrogen atoms bound to an N-heterocyclic ring, e.g., 1, 2, 3, 4, 5, 6, 7, 8, 9, or up to 10, such as between 1 to 5, 1 to 4, or 1 to 3, or 1 or 2) hydrogen atoms of the N-heterocyclic ring are replaced with a substituent other than hydrogen (when more than one hydrogen atom is replaced the substituents may be the same or different). Preferably, the substituent other than hydrogen is a 1^{st} level substituent as specified herein.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present disclosure.

Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present disclosure (even if one ring, such as in 1,2-dihydronaphthyl, is still aromatic). A similar distinction is made within the present application between heteroaryl (i.e., an aryl group as defined above in which one or more carbon atoms in the aryl group are replaced by heteroatoms) and heterocyclyl. For example, indolinyl, i.e., a dihydro variant of indolyl, is classified as heterocyclyl for the purposes of the present disclosure, since only one ring of the bicyclic structure is aromatic and one of the ring atoms is a heteroatom.

Typical 1^{st} level substituents are preferably selected from the group consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. Particularly preferred 1^{st} level substituents are selected from the group consisting of methyl, ethyl, propyl, isopropyl, halogen (such as F, Cl, or Br), and —CF₃, such as halogen (e.g., F, Cl, or Br), and —CF₃.

The expression “at most one of R¹, R², and R³ is H, CH(C₁₋₅ alkylene-R⁴)₂, or C(C₁₋₅ alkylene-R⁴)₃” with respect to the formula N(R¹)(R²)(R³) means that each of the H, CH(C₁₋₅ alkylene-R⁴)₂, and C(C₁₋₅ alkylene-R⁴)₃ can be bound to the N atom of N(R¹)(R²)(R³) only once at the same time. For example, if one of R¹, R², and R³ is H, the other two cannot be H (i.e., the other two must be other than H). Likewise, if one of R¹, R², and R³ is CH(C₁₋₅ alkylene-R⁴)₂, the other two cannot be CH(C₁₋₅ alkylene-R⁴)₂(i.e., the other two must be other than CH(C₁₋₅ alkylene-R⁴)₂). Also, if one of R¹, R², and R³ is C(C₁₋₅ alkylene-R⁴)₃, the other two cannot be C(C₁₋₅ alkylene-R⁴)₃ (i.e., the other two must be other than C(C₁₋₅ alkylene-R⁴)₃). Preferably, the expression means that only one of R¹, R², and R³ can be either H, CH(C₁₋₅ alkylene-R⁴)₂, or C(C₁₋₅ alkylene-R⁴)₃, the other two can neither be H, nor CH(C₁₋₅ alkylene-R⁴)₂, nor C(C₁₋₅alkylene-R⁴)₃(e.g., the other two are independently C₁₋₆ alkyl, or join together with the nitrogen atom to form a 5- or 6-membered N-heterocyclic ring which is optionally substituted with one or two R⁵).

The expression “after thawing the frozen composition”, as used herein in context with a frozen composition, means that the frozen composition has to be thawed before the characteristics (such as RNA integrity and/or size (Z_{average}) and/or size distribution and/or the PDI of the particles (such as LNPs) contained in the composition) can be measured.

A “monovalent” compound relates to a compound having only one functional group of interest. For example, a monovalent anion relates to a compound having only one negatively charged group, preferably under physiological conditions.

A “divalent” or “dibasic” compound relates to a compound having two functional groups of interest. For example, a dibasic organic acid has two acid groups.

A “polyvalent” or “polybasic” compound relates to a compound having three or more functional groups of interest. For example, a polybasic organic acid has three or more acid groups.

The expression “substantially free of X”, as used herein, means that a mixture (such as an aqueous phase of a composition or formulation described herein) is free of X is such manner as it is practically and realistically feasible. For example, if the mixture is substantially free of X, the amount of X in the mixture may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the mixture.

The expression “citrate anion”, as used herein, means any compound which contains a citrate anion and which when solved in an aqueous medium releases the citrate anion. Examples of compounds which contain a citrate anion and which release the citrate anion when solved in an aqueous medium, include citric acid and salts of citric acid.

The expression “anion of EDTA”, as used herein, means any compound which contains an anion of EDTA and which when solved in an aqueous medium releases the anion of EDTA. Examples of compounds which contain an anion of EDTA and which release an anion when solved in an aqueous medium, include ethylenediaminetetraacetic acid (EDTA) and salts of EDTA.

The expression “dibasic organic acid anions”, as used herein, means any organic compound containing two acid groups which are in free form (i.e., protonated), anhydride form or salt form. In this respect, the term “acid group” refers to a carboxylic acid or sulfate group. Preferably, the expression “dibasic organic acids” does not include esters of a carboxylic or sulfate group with one or more organic alcohols. Examples of dibasic organic acids include oxalic acid, malic acid, and tartaric acid.

The expression “polybasic organic acid anions”, as used herein, means any organic compound containing three or more acid groups which are in free form (i.e., protonated), anhydride form or salt form. In this respect, the term “acid group” refers to a carboxylic acid or sulfate group. Preferably, the expression “polybasic organic acids” does not include esters of a carboxylic or sulfate group with one or more organic alcohols. One example of a polybasic organic acid includes citric acid.

The expression “RNA integrity” means the percentage of the full-length (i.e., non-fragmented) RNA to the total amount of RNA (i.e., non-fragmented plus fragmented RNA) contained in a sample. The RNA integrity may be determined by chromatographically separating the RNA (e.g., using capillary electrophoresis), determining the peak area of the main RNA peak (i.e., the peak area of the full-length (i.e., non-fragmented) RNA), determining the peak area of the total RNA, and dividing the peak area of the main RNA peak by the peak area of the total RNA.

The term “cryoprotectant” relates to a substance that is added to a preparation (e.g., formulation or composition) in order to protect the active ingredients of the preparation during the freezing stages.

The term “lyoprotectant” relates to a substance that is added to a formulation in order to protect the active ingredients during the drying stages.

According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “polypeptide” refers to large peptides, in particular peptides having at least about 151 amino acids. “Peptides” and “polypeptides” are both protein molecules, although the terms “protein” and “polypeptide” are used herein usually as synonyms.

A “therapeutic protein” has a positive or advantageous effect on a condition or disease state of a subject when provided to the subject in a therapeutically effective amount. In some embodiments, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, antigens for vaccination and immunostimulants such as cytokines.

According to various embodiments of the present disclosure, a nucleic acid such as RNA (e.g., mRNA) encoding a peptide, polypeptide or protein is taken up by or introduced, i.e. transfected or transduced, into a cell which cell may be present in vitro or in a subject, resulting in expression of said peptide, polypeptide or protein. The cell may express the encoded peptide, polypeptide or protein intracellularly (e.g. in the cytoplasm and/or in the nucleus), may secrete the encoded peptide, polypeptide or protein, and/or may express it on the surface.

According to the present disclosure, terms such as “nucleic acid expressing” and “nucleic acid encoding” or similar terms are used interchangeably herein and with respect to a particular peptide, polypeptide or protein mean that the nucleic acid, if present in the appropriate environment, preferably within a cell, can be expressed to produce said peptide, polypeptide or protein.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may any portion from 0.1% to 99.9% (such as 0.1%, 0.5%, 1%, 5%, 10%, 50%, 90%, or 99%) of said composition.

“Fragment”, with reference to an amino acid sequence (peptide, polypeptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence. A fragment of an amino acid sequence comprises, e.g., a sequence of up to 8, in particular up to 10, up to 12, up to 15, up to 20, up to 30 or up to 55, consecutive amino acids of the amino acid sequence.

According to the present disclosure, a part or fragment of a peptide, polypeptide or protein preferably has at least one functional property of the peptide, polypeptide or protein from which it has been derived. Such functional properties comprise a pharmacological activity, the interaction with other peptides, polypeptides or proteins, an enzymatic activity, the interaction with antibodies, and the selective binding of nucleic acids. E.g., a pharmacological active fragment of a peptide, polypeptide or protein has at least one of the pharmacological activities of the peptide, polypeptide or protein from which the fragment has been derived. A part or fragment of a peptide, polypeptide or protein preferably comprises a sequence of at least 6, in particular at least 8, at least 10, at least 12, at least 15, at least 20, at least 30 or at least 50, consecutive amino acids of the peptide or protein. A part or fragment of a peptide or protein preferably comprises a sequence of up to 8, in particular up to 10, up to 12, up to 15, up to 20, up to 30 or up to 55, consecutive amino acids of the peptide or protein.

“Variant”, as used herein and with reference to an amino acid sequence (peptide, polypeptide, or protein), is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference as compared to the parent amino acid sequence, e.g., from 1 to about 20 amino acid differences, and preferably from 1 to about 10 or from 1 to about 5 amino acid differences compared to the parent.

By “wild type” or “WT” or “native” herein is meant an amino acid sequence that is found in nature, including allelic variations. A wild type amino acid sequence, peptide, polypeptide or protein has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. In some embodiments, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

In some embodiments, the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, -2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In some embodiments, a fragment or variant of an amino acid sequence (peptide, polypeptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. In some embodiments, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In some embodiments, “isolated” means altered or removed (e.g., purified) from the natural state or from an artificial composition, such as a composition from a production process. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell. In some embodiments, the RNA (such as mRNA) used in the present disclosure is in substantially purified form. In some embodiments, a solution (preferably an aqueous solution) of RNA (such as mRNA) in substantially purified form contains a first buffer system.

The term “genetic modification” or simply “modification” includes the transfection of cells with nucleic acid. The termn “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro or in vivo, e.g. the cell can form part of an organ, a tissue and/or an organism of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding antigen is transiently transfected into cells. RNA can be transfected into cells to transiently express its coded protein.

The disclosure includes analogs of a peptide, polypeptide or protein. According to the present disclosure, an analog of a peptide, polypeptide or protein is a modified form of said peptide, polypeptide or protein from which it has been derived and has at least one functional property of said peptide, polypeptide or protein. E.g., a pharmacological active analog of a peptide, polypeptide or protein has at least one of the pharmacological activities of the peptide, polypeptide or protein from which the analog has been derived. Such modifications include any chemical modification and comprise single or multiple substitutions, deletions and/or additions of any molecules associated with the protein, polypeptide or peptide, such as carbohydrates, lipids and/or proteins or peptides. In one embodiment, “analogs” of proteins, polypeptides or peptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said proteins, polypeptides and peptides.

“Activation” or “stimulation”, as used herein, refers to the state of a cell (e.g., an immune effector cell such as T cell) that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In some embodiments, the term is preferably used in the context of an immunological response in which immune effector cells are stimulated by an antigen, proliferate, and the specific immune effector cell recognizing said antigen is amplified. In some embodiments, expansion leads to differentiation of the immune effector cells.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, preferably a peptide or protein, that reacts specifically with antibodies or T-lymphocytes (T-cells). According to the present disclosure, the term “antigen” comprises any molecule which comprises at least one epitope, such as a T cell epitope. Preferably, an antigen in the context of the present disclosure is a molecule which, optionally after processing, induces an immune reaction, which is preferably specific for the antigen (including cells expressing the antigen). In one embodiment, an antigen is a disease-associated antigen, such as a tumor antigen, a viral antigen, or a bacterial antigen, or an epitope derived from such antigen.

According to the present disclosure, any suitable antigen may be used, which is a candidate for an immune response, wherein the immune response may be a humoral or cellular immune response or both. In the context of some embodiments of the present disclosure, the antigen is presented by a cell, preferably by an antigen presenting cell, in the context of MHC molecules, which results in an immune response against the antigen. An antigen may be a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present disclosure, an antigen may correspond to a naturally occurring product, for example, a viral protein, or a part thereof.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. Disease-associated antigens include pathogen-associated antigens, i.e., antigens which are associated with infection by microbes, typically microbial antigens (such as bacterial or viral antigens), or antigens associated with cancer, typically tumors, such as tumor antigens.

In some embodiments, the antigen is a tumor antigen, i.e., a part of a tumor cell, in particular those which primarily occur intracellularly or as surface antigens of tumor cells. In another embodiment, the antigen is a pathogen-associated antigen, i.e., an antigen derived from a pathogen, e.g., from a virus, bacterium, unicellular organism, or parasite, for example a viral antigen such as viral ribonucleoprotein or coat protein. In particular, the antigen should be presented by MHC molecules which results in modulation, in particular activation of cells of the immune system, preferably CD4+ and CD8+ lymphocytes, in particular via the modulation of the activity of a T-cell receptor.

The term “tumor antigen” or “tumor-associated antigen” refers to a constituent of cancer cells which may be derived from the cytoplasm, the cell surface or the cell nucleus. In particular, it refers to those antigens which are produced intracellularly or as surface antigens on tumor cells. For example, tumor antigens include the carcinoembryonal antigen, α1-fetoprotein, isoferritin, and fetal sulphoglycoprotein, α2-H-ferroprotein and γ-fetoprotein, as well as various virus tumor antigens. According to some embodiments of the present disclosure, a tumor antigen comprises any antigen which is characteristic for tumors or cancers as well as for tumor or cancer cells with respect to type and/or expression level.

The term “viral antigen” refers to any viral component having antigenic properties, i.e., being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “epitope” refers to an antigenic determinant in a molecule such as an antigen, i.e., to a part in or fragment of the molecule that is recognized by the immune system, for example, that is recognized by antibodies T cells or B cells, in particular when presented in the context of MHC molecules. An epitope of a protein may comprise a continuous or discontinuous portion of said protein and, e.g., may be between about 5 and about 100, between about 5 and about 50, between about 8 and about 0, between about 10 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, the epitope in the context of the present disclosure is a T cell epitope.

Terms such as “epitope”, “fragment of an antigen”, “immunogenic peptide” and “antigen peptide” are used interchangeably herein and, e.g., may relate to an incomplete representation of an antigen which is, e.g., capable of eliciting an immune response against the antigen or a cell expressing or comprising and presenting the antigen. In some embodiments, the terms relate to an immunogenic portion of an antigen. Preferably, it is a portion of an antigen that is recognized (i.e., specifically bound) by a T cell receptor, in particular if presented in the context of MHC molecules. Certain preferred immunogenic portions bind to an MHC class I or class 11 molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and protein antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to the peptide or protein.

In some embodiments, vaccine antigen, i.e., an antigen whose inoculation into a subject induces an immune response, is recognized by an immune effector cell. In some embodiments, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen is preferably presented or present on the surface of a cell, preferably an antigen presenting cell. In some embodiments, an antigen is presented by a diseased cell (such as tumor cell or an infected cell). In some embodiments, an antigen receptor is a TCR which binds to an epitope of an antigen presented in the context of MHC. In some embodiment, binding of a TCR when expressed by T cells and/or present on T cells to an antigen presented by cells such as antigen presenting cells results in stimulation, priming and/or expansion of said T cells. In some embodiments, binding of a TCR when expressed by T cells and/or present on T cells to an antigen presented on diseased cells results in cytolysis and/or apoptosis of the diseased cells, wherein said T cells preferably release cytotoxic factors, e.g., perforins and granzymes.

In some embodiments, an antigen receptor is an antibody or B cell receptor which binds to an epitope in an antigen. In some embodiments, an antibody or B cell receptor binds to native epitopes of an antigen.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part may be, e.g., at least 4, at least 8, at least 12, or at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by, e.g., antigen-specific antibodies added to the cells. In some embodiments, an antigen expressed on the surface of cells is an integral membrane protein having an extracellular portion which may be recognized by a CAR.

The term “extracellular portion” or “exodomain” in the context of the present disclosure refers to a part of a molecule such as a protein that is facing the extracellular space of a cell and preferably is accessible from the outside of said cell, e.g., by binding molecules such as antibodies located outside the cell. In some embodiments, the tenn refers to one or more extracellular loops or domains or a fragment thereof.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured. In certain embodiments of the present disclosure, the RNA (in particular mRNA) encodes at least one epitope.

The term “target” shall mean an agent such as a cell or tissue which is a target for an immune response such as a cellular immune response. Targets include cells that present an antigen or an antigen epitope, i.e., a peptide fragment derived from an antigen. In one embodiment, the target cell is a cell expressing an antigen and preferably presenting said antigen with class I MHC.

“Antigen processing” refers to the degradation of an antigen into processing products which are fragments of said antigen (e.g., the degradation of a protein into peptides) and the association of one or more of these fragments (e.g., via binding) with MHC molecules for presentation by cells, preferably antigen-presenting cells to specific T-cells. Antigen-presenting cells can be distinguished in professional antigen presenting cells and non-professional antigen presenting cells.

By “antigen-responsive CTL” is meant a CD8⁺ T-cell that is responsive to an antigen or a peptide derived from said antigen, which is presented with class I MHC on the surface of antigen presenting cells.

According to the disclosure, CTL responsiveness may include sustained calcium flux, cell division, production of cytokines such as IFN-γ and TNF-α, up-regulation of activation markers such as CD44 and CD69, and specific cytolytic killing of tumor antigen expressing target cells. CTL responsiveness may also be determined using an artificial reporter that accurately indicates CTL responsiveness.

The terms “immune response” and “immune reaction” are used herein interchangeably in their conventional meaning and refer to an integrated bodily response to an antigen and may refer to a cellular immune response, a humoral immune response, or both. According to the disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, such as CD4⁺ and CD8⁺ T-lymphocytes, e.g., CD8⁺ T-lymphocytes, which may be detected in various proliferation or cytokine production tests in vitro.

The terms “inducing an immune response” and “eliciting an immune response” and similar terms in the context of the present disclosure refer to the induction of an immune response, such as the induction of a cellular immune response, a humoral immune response, or both. The immune response may be protective/preventive/prophylactic and/or therapeutic. The immune response may be directed against any immunogen or antigen or antigen peptide, preferably against a tumor-associated antigen or a pathogen-associated antigen (e.g., an antigen of a virus (such as influenza virus (A, B, or C), CMV or RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. In some embodiments, after inducing an immune response in an individual, said individual is protected from developing a disease such as an infectious disease or a cancerous disease or the disease condition is ameliorated by inducing an immune response.

The terms “cellular immune response”, “cellular response”, “cell-mediated immunity” or similar terms are meant to include a cellular response directed to cells characterized by expression of an antigen and/or presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The term “antibody” as used herein, refers to an immunoglobulin molecule, which is able to specifically bind to an epitope on an antigen. In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3, the CDRs of a VL are termed LCDR1, LCDR2 and LCDR3. The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the imnnunoglobulin to host tissues or factors, including various cells of the immune system (e.g., effector cells) and the first component (Clq) of the classical complement system. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The term “immunoglobulin” relates to proteins of the immunoglobulin superfamily, such as to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies. Immunoglobulins generally comprise several chains, typically two identical heavy chains and two identical light chains which are linked via disulfide bonds. These chains are primarily composed of immunoglobulin domains, such as the V_{L} (variable light chain) domain, C_{L} (constant light chain) domain, V_{H} (variable heavy chain) domain, and the C_{H} (constant heavy chain) domains C_{H}1, C_{H}2, C_{H}3, and C_{H}4. There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The terms “vaccination” and “immunization” describe the process of treating an individual for therapeutic or prophylactic reasons and relate to the procedure of administering one or more immunogen(s) or antigen(s) or derivatives thereof, in particular in the form of RNA (especially mRNA) coding therefor, as described herein to an individual and stimulating an immune response against said one or more immunogen(s) or antigen(s) or cells characterized by presentation of said one or more iminunogen(s) or antigen(s).

By “cell characterized by presentation of an antigen” or “cell presenting an antigen” or “MHC molecules which present an antigen on the surface of an antigen presenting cell” or similar expressions is meant a cell such as a diseased cell, in particular a tumor cell or an infected cell, or an antigen presenting cell presenting the antigen or an antigen peptide, either directly or following processing, in the context of MHC molecules, preferably MHC class I and/or MHC class I molecules, most preferably MHC class I molecules.

In the context of the present disclosure, the term “transcription” relates to a process, wherein the genetic code in a DNA sequence is transcribed into RNA (especially mRNA). Subsequently, the RNA (especially mRNA) may be translated into peptide, polypeptide or protein.

With respect to RNA, the term “expression” or “translation” relates to the process in the ribosomes of a cell by which a strand of mRNA directs the assembly of a sequence of amino acids to make a peptide or protein.

A medical preparation, in particular kit, described herein may comprise instructional material or instructions. As used herein, “instructional material” or “instructions” includes a publication, a recording, a diagram, or any other medium of expression which can be used to communicate the usefulness of the compositions and methods of the present disclosure. The instructional material of the kit of the present disclosure may, for example, be affixed to a container which contains the compositions of the present disclosure or be shipped together with a container which contains the compositions. Alternatively, the instructional material may be shipped separately from the container with the intention that the instructional material and the compositions be used cooperatively by the recipient.

The term “optional” or “optionally” as used herein means that the subsequently described event, circumstance or condition may or may not occur, and that the description includes instances where said event, circumstance, or condition occurs and instances in which it does not occur.

Prodrugs of a particular compound described herein are those compounds that upon administration to an individual undergo chemical conversion under physiological conditions to provide the particular compound. Additionally, prodrugs can be converted to the particular compound by chemical or biochemical methods in an ex vivo environment. For example, prodrugs can be slowly converted to the particular compound when, for example, placed in a transdermal patch reservoir with a suitable enzyme or chemical reagent. Exemplary prodrugs are esters (using an alcohol or a carboxy group contained in the particular compound) or amides (using an amino or a carboxy group contained in the particular compound) which are hydrolyzable in vivo. Specifically, any amino group which is contained in the particular compound and which bears at least one hydrogen atom can be converted into a prodrug form. Typical N-prodrug forms include carbamates, Mannich bases, enamines, and enaminones.

In the present specification, a structural formula of a compound may represent a certain isomer of said compound. It is to be understood, however, that the present disclosure includes all isomers such as geometrical isomers, optical isomers based on an asymmetrical carbon, stereoisomers, tautomers and the like which occur structurally and isomer mixtures and is not limited to the description of the formula. Furthermore, in the present specification, a structural formula of a compound may represent a specific salt and/or solvate of said compound. It is to be understood, however, that the present disclosure includes all salts (e.g., pharmaceutically acceptable salts) and solvates (e.g., hydrates) and is not limited to the description of the specific salt and/or solvate.

“Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (±). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimensional forms of (hetero)cyclic rings, such as chair, half-chair, boat, and twist-boat forms of cyclohexane.

The term “solvate” as used herein refers to an addition complex of a dissolved material in a solvent (such as an organic solvent (e.g., an aliphatic alcohol (such as methanol, ethanol, n-propanol, isopropanol), acetone, acetonitrile, ether, and the like), water or a mixture of two or more of these liquids), wherein the addition complex exists in the form of a crystal or mixed crystal. The amount of solvent contained in the addition complex may be stoichiometric or non-stoichiometric. A “hydrate” is a solvate wherein the solvent is water.

In isotopically labeled compounds one or more atoms are replaced by a corresponding atom having the same number of protons but differing in the number of neutrons. For example, a hydrogen atom may be replaced by a deuterium or tritium atom. Exemplary isotopes which can be used in the present disclosure include deuterium, tritium, ¹¹C, ¹³C, ¹⁴C, ¹⁵N, ¹⁸F, ³²P, ³²S, ³⁵S, ³⁶Cl, and ¹²⁵I.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

In some embodiments, the “polydispersity index” is calculated based on dynamic light scattering measurements by the so-called cumulant analysis as mentioned in the definition of the “average diameter”. Under certain prerequisites, it can be taken as a measure of the size distribution of an ensemble of nanoparticles.

The “radius of gyration” (abbreviated herein as R_{g}) of a particle about an axis of rotation is the radial distance of a point from the axis of rotation at which, if the whole mass of the particle is assumed to be concentrated, its moment of inertia about the given axis would be the same as with its actual distribution of mass. Mathematically, R_{g} is the root mean square distance of the particle's components from either its center of mass or a given axis. For example, for a macromolecule composed of n mass elements, of masses mᵢ (i=1, 2, 3, . . . , n), located at fixed distances sᵢ from the center of mass, R_{g} is the square-root of the mass average of sᵢ² over all mass elements and can be calculated as follows:

$${R}_{g} = {( {\sum }_{i = 1}^{n} {m}_{i} \cdot {s}_{i}^{2} / {\sum }_{i = 1}^{n} {m}_{i} )}^{1 / 2}$$

The radius of gyration can be determined or calculated experimentally, e.g., by using light scattering. In particular, for small scattering vectors q the structure function S is defined as follows:

$$S ( \vec{q} ) \approx N \cdot ( 1 - \frac{{q}^{2} \cdot {R}_{g}^{2}}{3} )$$

wherein N is the number of components (Guinier's law).

The “D10 value”, in particular regarding a quantitative size distribution of particles, is the diameter at which 10% of the particles have a diameter less than this value. The D10 value is a means to describe the proportion of the smallest particles within a population of particles (such as within a particle peak obtained from a field-flow fractionation).

“D50 value”, in particular regarding a quantitative size distribution of particles, is the diameter at which 50% of the particles have a diameter less than this value. The D50 value is a means to describe the mean particle size of a population of particles (such as within a particle peak obtained from a field-flow fractionation).

The “D90 value”, in particular regarding a quantitative size distribution of particles, is the diameter at which 90% of the particles have a diameter less than this value. The “D95”, “D99”, and “D100” values have corresponding meanings. The D90, D95, D99, and D100 values are means to describe the proportion of the larger particles within a population of particles (such as within a particle peak obtained from a field-flow fractionation).

The “hydrodynamic radius” (which is sometimes called “Stokes radius” or “Stokes-Einstein radius”) of a particle is the radius of a hypothetical hard sphere that diffuses at the same rate as said particle. The hydrodynamic radius is related to the mobility of the particle, taking into account not only size but also solvent effects. For example, a smaller charged particle with stronger hydration may have a greater hydrodynamic radius than a larger charged particle with weaker hydration. This is because the smaller particle drags a greater number of water molecules with it as it moves through the solution. Since the actual dimensions of the particle in a solvent are not directly measurable, the hydrodynamic radius may be defined by the Stokes-Einstein equation:

$${R}_{h} = \frac{{k}_{B} \cdot T}{6 \cdot π \cdot η \cdot D}$$

wherein k_{B} is the Boltzmann constant; T is the temperature; ρ is the viscosity of the solvent; and D is the diffusion coefficient. The diffusion coefficient can be determined experimentally, e.g., by using dynamic light scattering (DLS). Thus, one procedure to determine the hydrodynamic radius of a particle or a population of particles (such as the hydrodynamic radius of particles such as LNPs contained in a formulation or composition as disclosed herein or the hydrodynamic radius of a particle peak obtained from subjecting such a formulation or composition to field-flow fractionation) is to measure the DLS signal of said particle or population of particles (such as DLS signal of particles such as LNPs contained in a formulation or composition as disclosed herein or the DLS signal of a particle peak obtained from subjecting such a formulation or composition to field-flow fractionation).

The term “aggregate” as used herein relates to a cluster of particles, wherein the particles are identical or very similar and adhere to each other in a non-covalently manner (e.g., via ionic interactions, H bridge interactions, dipole interactions, and/or van der Waals interactions).

The expression “light scattering” as used herein refers to the physical process where light is forced to deviate from a straight trajectory by one or more paths due to localized non-uniformities in the medium through which the light passes.

The term “UV” means ultraviolet and designates a band of the electromagnetic spectrum with a wavelength from 10 nm to 400 nm, i.e., shorter than that of visible light but longer than X-rays.

The expression “multi-angle light scattering” or “MALS” as used herein relates to a technique for measuring the light scattered by a sample into a plurality of angles. “Multi-angle” means in this respect that scattered light can be detected at different discrete angles as measured, for example, by a single detector moved over a range including the specific angles selected or an array of detectors fixed at specific angular locations. In one preferred embodiment, the light source used in MALS is a laser source (MALLS: multi-angle laser light scattering). Based on the MALS signal of a composition comprising particles and by using an appropriate formalism (e.g., Zimm plot, Berry plot, or Debye plot), it is possible to determine the radius of gyration (R_{g}) and, thus, the size of said particles. Preferably, the Zimm plot is a graphical presentation using the following equation:

$$\frac{{R}_{θ}}{{K}^{*} c} = {M}_{w} P ( θ ) - 2 {A}_{2} c {M}_{w}^{2} {P}^{2} ( θ )$$

wherein c is the mass concentration of the particles in the solvent (g/mL); A₂ is the second virial coefficient (mol-mL/g²); P(θ) is a form factor relating to the dependence of scattered light intensity on angle; R_{θ} is the excess Rayleigh ratio (cm⁻¹); and K* is an optical constant that is equal to 4π²ρₒ (dn/dc)²λ₀⁻⁴N_{A}⁻¹, where ρₒ is the refractive index of the solvent at the incident radiation (vacuum) wavelength, λ₀ is the incident radiation (vacuum) wavelength (nm), N_{A} is Avogadro's number (mol⁻¹), and dn/dc is the differential refractive index increment (mL/g) (cf., e.g., Buchholz et al. (Electrophoresis 22 (2001), 4118-4128); B. H. Zimm (J. Chem. Phys. 13 (1945), 141; P. Debye (J. Appl. Phys. 15 (1944): 338; and W. Burchard (Anal. Chem. 75 (2003), 4279-4291). Preferably, the Berry plot is calculated the following term or the reciprocal thereof:

$$\sqrt{\frac{{R}_{θ}}{{K}^{*} c}}$$

wherein c, R_{θ} and K* are as defined above. Preferably, the Debye plot is calculated the following term or the reciprocal thereof:

$$\frac{{K}^{*} c}{{R}_{θ}}$$

wherein c, R_{θ} and K* are as defined above.

The expression “dynamic light scattering” or “DLS” as used herein refers to a technique to determine the size and size distribution profile of particles, in particular with respect to the hydrodynamic radius of the particles. A monochromatic light source, usually a laser, is shot through a polarizer and into a sample. The scattered light then goes through a second polarizer where it is detected and the resulting image is projected onto a screen. The particles in the solution are being hit with the light and diffract the light in all directions. The diffracted light from the particles can either interfere constructively (light regions) or destructively (dark regions). This process is repeated at short time intervals and the resulting set of speckle patterns are analyzed by an autocorrelator that compares the intensity of light at each spot over time.

The expression “static light scattering” or “SLS” as used herein refers to a technique to determine the size and size distribution profile of particles, in particular with respect to the radius of gyration of the particles, and/or the molar mass of particles. A high-intensity monochromatic light, usually a laser, is launched in a solution containing the particles. One or many detectors are used to measure the scattering intensity at one or many angles. The angular dependence is needed to obtain accurate measurements of both molar mass and size for all macromolecules of radius. Hence simultaneous measurements at several angles relative to the direction of incident light, known as multi-angle light scattering (MALS) or multi-angle laser light scattering (MALLS), is generally regarded as the standard implementation of static light scattering.

### Nucleic Acids

The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” (abbreviated herein as “N”) relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine.

The five standard nucleosides which usually make up naturally occurring nucleic acids are uridine, adenosine, thymidine, cytidine and guanosine. The five nucleosides are commonly abbreviated to their one letter codes U, A, T, C and G, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In RNA, they would be represented as A, C and G, whereas in DNA they would be represented as dA, dC and dG.

A modified purine (A or G) or pyrimidine (C, T, or U) base moiety is preferably modified by one or more alkyl groups, more preferably one or more C₁₋₄ alkyl groups, even more preferably one or more methyl groups. Particular examples of modified purine or pyrimidine base moieties include N⁷-alkyl-guanine, N⁶-alkyl-adenine, 5-alkyl-cytosine, 5-alkyl-uracil, and N(1)-alkyl-uracil, such as N⁷—C₁₋₄ alkyl-guanine, N′-C₁₋₄ alkyl-adenine, 5-C₁₋₄ alkyl-cytosine, 5-C₁₋₄ alkyl-uracil, and N(1)-C₁₋₄ alkyl-uracil, preferably N⁷-methyl-guanine, N⁶-methyl-adenine, 5-methyl-cytosine, 5-methyl-uracil, and N(1)-methyl-uracil.

Herein, the term “DNA” relates to a nucleic acid molecule which includes deoxyribonucleotide residues. In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

DNA may be recombinant DNA and may be obtained by cloning of a nucleic acid, in particular cDNA. The eDNA may be obtained by reverse transcription of RNA.

### RNA

According to the present disclosure, the term “RNA” means a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides can be referred to as analogs of naturally occurring nucleotides, and the corresponding RNAs containing such altered/modified nucleotides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

“RNA” includes mRNA, tRNA, ribosomal RNA (rRNA), small nuclear RNA (snRNA), self-amplifying RNA (saRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In some embodiments, “RNA” refers to mRNA.

In a preferred embodiment, the RNA comprises an open reading frame (ORF) encoding a peptide, polypeptide or protein.

The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)).

mRNA

In some embodiments of all aspects of the disclosure, the RNA is mRNA.

According to the present disclosure, the term “mRNA” means “messenger-RNA” and includes a “transcript” which may be generated by using a DNA template. Generally, mRNA encodes a peptide, polypeptide or protein. Typically, an mRNA comprises a 5′-UTR, a peptide/protein coding region, and a 3′-UTR. In the context of the present disclosure, mRNA is preferably generated by in vitro transcription (IVT) from a DNA template. As set forth above, the in vitro transcription methodology is known to the skilled person, and a variety of in vitro transcription kits is commercially available.

mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide, polypeptide or protein.

In some embodiments, the RNA which preferably encodes a peptide, polypeptide or protein has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

As established in the art, the RNA (such as mRNA) generally contains a 5′ untranslated region (5′-UTR), a peptide/polypeptide/protein coding region and a 3′ untranslated region (3′-UTR). In some embodiments, the RNA (such as mRNA) is produced by in vitro transcription or chemical synthesis. In one embodiment, the RNA (such as mRNA) is produced by in vitro transcription using a DNA template.

The in vitro transcription methodology is known to the skilled person; cf., e.g., Molecular Cloning: A Laboratory Manual, 2^{nd} Edition, J. Sambrook et al. eds., Cold Spring Harbor Laboratory Press, Cold Spring Harbor 1989. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™). For providing modified RNA (such as mRNA), correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription.

In some embodiments, RNA (such as mRNA) is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA. In some embodiments of the present disclosure, the RNA (such as mRNA) is “replicon RNA” (such as “replicon mRNA”) or simply a “replicon”, in particular “self-replicating RNA” (such as “self-replicating mRNA”) or “self-amplifying RNA” (or “self-amplifying mRNA”). In one particularly preferred embodiment, the replicon or self-replicating RNA (such as self-replicating mRNA) is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning nearthe 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments of the present disclosure, the RNA (such as mRNA) described herein (e.g., contained in the compositions of the present disclosure and/or used in the methods of the present disclosure) contains one or more modifications, e.g., in order to increase its stability and/or increase translation efficiency and/or decrease immunogenicity and/or decrease cytotoxicity. For example, in order to increase expression of the RNA (such as mRNA), it may be modified within the coding region, i.e., the sequence encoding the expressed peptide or protein, preferably without altering the sequence of the expressed peptide or protein. Such modifications are described, for example, in WO 2007/036366 and PCT/EP2019/056502, and include the following: a 5′-cap structure; an extension or truncation of the naturally occurring poly(A) tail; an alteration of the 5′- and/or 3′-untranslated regions (UTR) such as introduction of a UTR which is not related to the coding region of said RNA; the replacement of one or more naturally occurring nucleotides with synthetic nucleotides; and codon optimization (e.g., to alter, preferably increase, the GC content of the RNA). The term “modification” in the context of modified mRNA according to the present disclosure preferably relates to any modification of an mRNA which is not naturally present in said RNA (such as mRNA).

In some embodiments, the RNA (such as mRNA) described herein comprises a 5′-cap structure. In one embodiment, the mRNA does not have uncapped 5′-triphosphates. In one embodiment, the RNA (such as mRNA) described herein may comprise a conventional 5′-cap and/or a 5′-cap analog. The term “conventional 5′-cap” refers to a cap structure found on the 5′-end of an mRNA molecule and generally consists of a guanosine 5′-triphosphate (Gppp) which is connected via its triphosphate moiety to the 5′-end of the next nucleotide of the mRNA (i.e., the guanosine is connected via a 5′ to 5′ triphosphate linkage to the rest of the mRNA). The guanosine may be methylated at position N⁷ (resulting in the cap structure m⁷Gppp). The term “5′-cap analog” refers to a 5′-cap which is based on a conventional 5′-cap but which has been modified at either the 2′- or 3′-position of the m⁷guanosine structure in order to avoid an integration of the 5′-cap analog in the reverse orientation (such 5′-cap analogs are also called anti-reverse cap analogs (ARCAs)). Particularly preferred 5′-cap analogs are those having one or more substitutions at the bridging and non-bridging oxygen in the phosphate bridge, such as phosphorothioate modified 5′-cap analogs at the β-phosphate (such as m₂₇′2° G(5′)ppSp(5′)G (referred to as beta-S-ARCA or β-S-ARCA)), as described in PCT/EP2019/056502. Providing an RNA (such as mRNA) with a 5′-cap structure as described herein may be achieved by in vitro transcription of a DNA template in presence of a corresponding 5′-cap compound, wherein said 5′-cap structure is co-transcriptionally incorporated into the generated RNA (such as mRNA) strand, or the RNA (such as mRNA) may be generated, for example, by in vitro transcription, and the 5′-cap structure may be attached to the mRNA post-transcriptionally using capping enzymes, for example, capping enzymes of vaccinia virus.

In some embodiments, the RNA (such as mRNA) comprises a 5′-cap structure selected from the group consisting of m₂^{7,2′O}G(5′)ppSp(5′)G (in particular its D1 diastereomer), m₂^{7,3′O} G(5′)ppp(5′)G, and m₂^{7,3′-O}Gppp(m₁^{2′-O})ApG. In some embodiments, RNA encoding a peptide, polypeptide or protein comprising an antigen or epitope comprises m₂^{7,2′O} G(5′)ppSp(5′)G (in particular its D1 diastereomer) as 5′-cap structure.

In some embodiments, the RNA (such as mRNA) comprises a cap0, cap1, or cap2, preferably cap1 or cap2. According to the present disclosure, the term “cap0” means the structure “m⁷GpppN”, wherein N is any nucleoside bearing an OH moiety at position 2′. According to the present disclosure, the term “cap1” means the structure “m⁷GpppNm”, wherein Nm is any nucleoside bearing an OCH₃ moiety at position 2′. According to the present disclosure, the term “cap2” means the structure “m⁷GpppNmNm”, wherein each Nm is independently any nucleoside bearing an OCH₃ moiety at position 2′.

The 5′-cap analog beta-S-ARCA (β-S-ARCA) has the following structure:

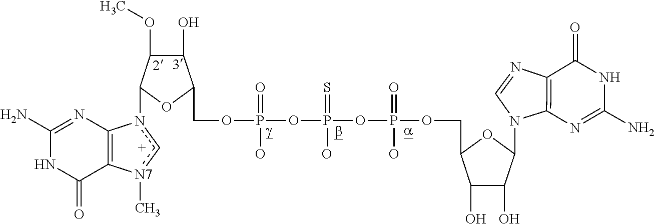

The “D1 diastereomer of beta-S-ARCA” or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time. The HPLC preferably is an analytical HPLC. In one embodiment, a Supelcosil LC-18-T RP column, preferably of the format: 5 sm, 4.6×250 mm is used for separation, whereby a flow rate of 1.3 ml/min can be applied. In one embodiment, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

The 5′-cap analog m₂^{7,3′-O} Gppp(m₁^{2′-O})ApG (also referred to as m₂^{7,3′-O} G(5′)ppp(5′)m^{2′-O}ApG) which is a building block of a cap1 has the following structure:

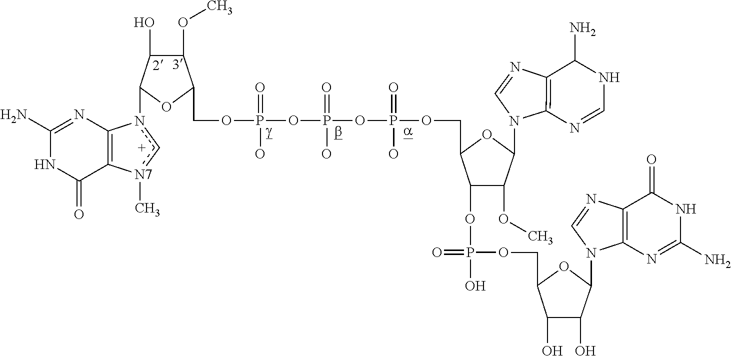

An exemplary cap0 mRNA comprising β-S-ARCA and mRNA has the following structure:

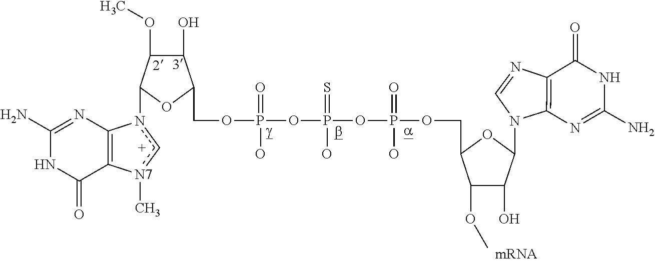

An exemplary cap0 mRNA comprising m₂^{7,3′O}G(5′)ppp(5′)G and mRNA has the following structure:

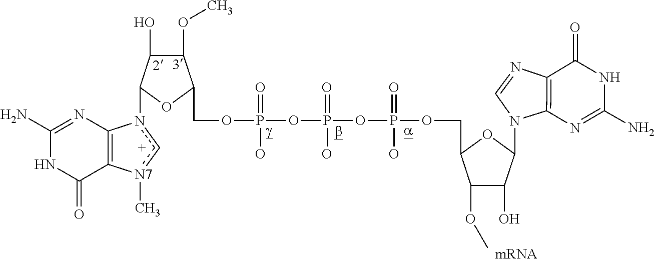

An exemplary cap1 mRNA comprising m₂^{7,3′-O} Gppp(m₁^{2′-O})ApG and mRNA has the following structure:

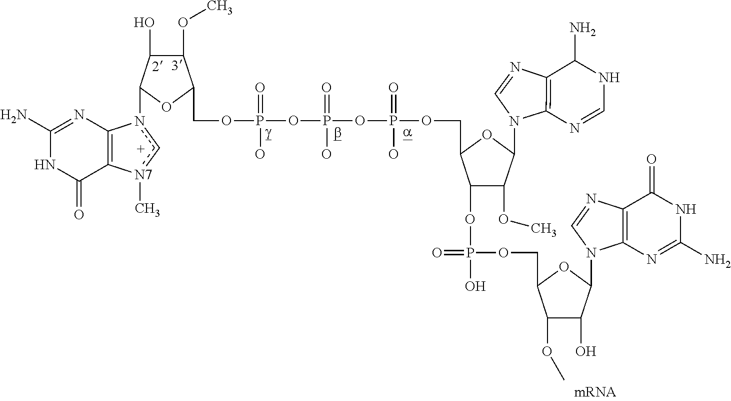

As used herein, the term “poly-A tail” or “poly-A sequence” refers to an uninterrupted or interrupted sequence of adenylate residues which is typically located at the 3′-end of an RNA (such as mRNA) molecule. Poly-A tails or poly-A sequences are known to those of skill in the art and may follow the 3′-UTR in the RNAs (such as mRNAs) described herein. An uninterrupted poly-A tail is characterized by consecutive adenylate residues. In nature, an uninterrupted poly-A tail is typical. RNAs (such as mRNAs) disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of mRNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA (in particular, mRNA) molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A.

In some embodiments, a poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises at least 100 nucleotides. In some embodiments, the poly-A tail comprises about 150 nucleotides. In some embodiments, the poly-A tail comprises about 120 nucleotides. In some embodiments, the poly-A tail comprises or consists of the nucleotide sequence of SEQ ID NO: 14. In some embodiments, the poly-A sequence has a nucleotide sequence having at least 99%, 98%, 97%, 96%, 95%, 90%, 85%, or 80% identity to the nucleotide sequence of SEQ ID NO: 14.

In some embodiments, RNA (such as mRNA) used in present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does preferably not include the poly-A sequence. Thus, the 3′-UTR is upstream of the poly-A sequence (if present), e.g., directly adjacent to the poly-A sequence. Incorporation of a 3′-UTR into the 3′-non translated region of an RNA (preferably mRNA) molecule can result in an enhancement in translation efficiency. A synergistic effect may be achieved by incorporating two or more of such 3′-UTRs (which are preferably arranged in a head-to-tail orientation; cf., e.g., Holtkamp et al., Blood 108, 4009-4017 (2006)). The 3′-UTRs may be autologous or heterologous to the RNA (preferably mRNA) into which they are introduced. In one particular embodiment the 3′-UTR is derived from a globin gene or mRNA, such as a gene or mRNA of alpha2-globin, alpha1-globin, or beta-globin, preferably beta-globin, more preferably human beta-globin. For example, the RNA (preferably mRNA) may be modified by the replacement of the existing 3′-UTR with or the insertion of one or more, preferably two copies of a 3′-UTR derived from a globin gene, such as alpha2-globin, alpha1-globin, beta-globin, preferably beta-globin, more preferably human beta-globin.

In some embodiments, the RNA (such as mRNA) used in present disclosure comprises a 5′-UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In some embodiments, the RNA (such as mRNA) used in present disclosure comprises a 3′-UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

The RNA (such as mRNA) described herein may have modified ribonucleotides in order to increase its stability and/or decrease immunogenicity and/or decrease cytotoxicity. For example, in some embodiments, uridine in the RNA (such as mRNA) described herein is replaced (partially or completely, preferably completely) by a modified nucleoside. In some embodiments, the modified nucleoside is a modified uridine.

In some embodiments, the modified uridine replacing uridine is selected from the group consisting of pseudouridine (ψ), N1-methyl-pseudouridine (m1ψ), 5-methyl-uridine (m5U), and combinations thereof.

In some embodiments, the modified nucleoside replacing (partially or completely, preferably completely) uridine in the RNA (such as mRNA) may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cnmm5s2U), 5-propynyl-uridine, 1-propynyl-pseudouridine, 5-taurinomethyl-uridine (tim5U), 1-taurinomethyl-pseudouridine, 5-taurinomethyl-2-thio-uridine(τm5s2U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 W), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

An RNA (preferably mRNA) which is modified by pseudouridine (replacing partially or completely, preferably completely, uridine) is referred to herein as “Ψ-modified”, whereas the term “m1Ψ-modified” means that the RNA (preferably mRNA) contains N(1)-methylpseudouridine (replacing partially or completely, preferably completely, uridine). Furthermore, the term “m5U-modified” means that the RNA (preferably mRNA) contains 5-methyluridine (replacing partially or completely, preferably completely, uridine). Such Ψ- or m1Ψ- or m5U-modified RNAs usually exhibit decreased immunogenicity compared to their unmodified forms and, thus, are preferred in applications where the induction of an immune response is to be avoided or minimized. In some embodiments, the RNA (preferably mRNA) contains N(1)-methylpseudouridine replacing completely uridine.

The codons of the RNA (preferably mRNA) described in the present disclosure may further be optimized, e.g., to increase the GC content of the RNA and/or to replace codons which are rare in the cell (or subject) in which the peptide or protein of interest is to be expressed by codons which are synonymous frequent codons in said cell (or subject). In some embodiments, the amino acid sequence encoded by the RNA described in the present disclosure is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present disclosure, coding regions are preferably codon-optimized for optimal expression in a subject to be treated using the RNA (preferably mRNA) described herein. Codon-optimization is based on the finding that the translation efficiency is also determined by a different frequency in the occurrence of tRNAs in cells. Thus, the sequence of RNA (preferably mRNA) may be modified such that codons for which frequently occurring tRNAs are available are inserted in place of “rare codons”.

In some embodiments, the guanosine/cytosine (G/C) content of the coding region of the RNA (preferably mRNA) described herein is increased compared to the G/C content of the corresponding coding sequence of the wild type RNA, wherein the amino acid sequence encoded by the RNA (preferably mRNA) is preferably not modified compared to the amino acid sequence encoded by the wild type RNA. This modification of the RNA sequence is based on the fact that the sequence of any RNA region to be translated is important for efficient translation of that RNA (preferably mRNA). Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the RNA (preferably mRNA), there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA (in particular, mRNA) described herein is increased by at least 10%, at least 20%, at least 30%, at least 40%, at least 50%, at least 55%, or even more compared to the G/C content of the coding region of the wild type RNA.

A combination of the above described modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (W) or N(1)-methylpseudouridine (m1Ψ) or 5-methyluridine (m5U) for uridine), and codon optimization, has a synergistic influence on the stability of RNA (preferably mRNA) and increase in translation efficiency. Thus, in some embodiments, the RNA (preferably mRNA) described in the present disclosure, in particular an RNA (preferably mRNA) encoding an antigen or epitope for inducing an immune response disclosed herein, contains a combination of at least two, at least three, at least four or all five of the above-mentioned modifications, i.e., (i) incorporation of a 5′-cap structure; (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (Ψ) or N(1)-methylpseudouridine (m1Ψ) or 5-methyluridine (m5U) for uridine); and (v) codon optimization. In some embodiments, the RNA (preferably mRNA) described in the present disclosure comprises a cap1 or cap2, preferably a cap1 structure. In some embodiments, the poly-A sequence comprises at least 100 nucleotides. In some embodiments, the poly-A sequence comprises or consists of the nucleotide sequence of SEQ ID NO: 14. In some embodiments, the a 5′-UTR comprises the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12. In some embodiments, the 3′-UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

Some aspects of the disclosure involve the targeted delivery of the RNA (preferably mRNA) disclosed herein to certain cells or tissues. In some embodiments, the disclosure involves targeting the lymphatic system, in particular secondary lymphoid organs, more specifically spleen. Targeting the lymphatic system, in particular secondary lymphoid organs, more specifically spleen is in particular preferred if the RNA (preferably mRNA) administered is RNA (preferably mRNA) encoding an antigen or epitope for inducing an immune response. In some embodiments, the target cell is a spleen cell. In some embodiments, the target cell is an antigen presenting cell such as a professional antigen presenting cell in the spleen. In some embodiments, the target cell is a dendritic cell in the spleen. The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naive lymphocytes and initiate an adaptive immune response.

Lipid-based RNA (such as mRNA) delivery systems have an inherent preference to the liver. Liver accumulation is caused by the discontinuous nature of the hepatic vasculature or the lipid metabolism (liposomes and lipid or cholesterol conjugates). In some embodiments, the target organ is liver and the target tissue is liver tissue. The delivery to such target tissue is preferred, in particular, if presence of mRNA or of the encoded peptide or protein in this organ or tissue is desired and/or if it is desired to express large amounts of the encoded peptide or protein and/or if systemic presence of the encoded peptide or protein, in particular in significant amounts, is desired or required.

In some embodiments, after administration of the RNA (in particular, mRNA) compositions described herein, at least a portion of the RNA is delivered to a target cell or target organ. In some embodiments, at least a portion of the RNA is delivered to the cytosol of the target cell. In some embodiments, the RNA is RNA (preferably mRNA) encoding a peptide or protein and the RNA is translated by the target cell to produce the peptide or protein. In some embodiments, the target cell is a cell in the liver. In some embodiments, the target cell is a muscle cell. In some embodiments, the target cell is an endothelial cell. In some embodiments, the target cell is a tumor cell or a cell in the tumor microenvironment. In some embodiments, the target cell is a blood cell. In some embodiments, the target cell is a cell in the lymph nodes. In some embodiments, the target cell is a cell in the lung In some embodiments, the target cell is a cell in the skin. In some embodiments, the target cell is a spleen cell. In some embodiments, the target cell is an antigen presenting cell such as a professional antigen presenting cell in the spleen. In some embodiments, the target cell is a dendritic cell in the spleen. In some embodiments, the target cell is a T cell. In some embodiments, the target cell is a B cell. In some embodiments, the target cell is a NK cell. In some embodiments, the target cell is a monocyte. Thus, RNA LNP compositions described herein may be used for delivering RNA (preferably mRNA) to such target cell. Accordingly, the present disclosure also relates to a method for delivering RNA (preferably mRNA) to a target cell in a subject comprising the administration of the RNA compositions described herein to the subject. In some embodiments, the RNA is delivered to the cytosol of the target cell. In some embodiments, the RNA is RNA (preferably mRNA) encoding a peptide or protein and the RNA is translated by the target cell to produce the peptide or protein.

### Inhibitory RNA

In some embodiments of all aspects of the disclosure, the RNA is an inhibitory RNA.

The term “inhibitory RNA” as used herein means RNA which selectively hybridizes to and/or is specific for a target mRNA, thereby inhibiting (e.g., reducing) transcription and/or translation thereof. Inhibitory RNA includes RNA molecules having sequences in the antisense orientation relative to the target mRNA. Suitable inhibitory oligonucleotides typically vary in length from five to several hundred nucleotides, more typically about 20 to 70 nucleotides in length or shorter, even more typically about 10 to 30 nucleotides in length. Examples of inhibitory RNA include antisense RNA, ribozyme, iRNA, siRNA and miRNA. In some embodiments of all aspects of the disclosure, the inhibitory RNA is siRNA.

The term “antisense RNA” as used herein refers to an RNA which hybridizes under physiological conditions to DNA comprising a particular gene or to mRNA of said gene, thereby inhibiting transcription of said gene and/or translation of said mRNA. An antisense RNA or of a part thereof may form a duplex with naturally occurring mRNA and thus prevent accumulation of or translation of the mRNA. Another possibility is the use of ribozymes for inactivating a nucleic acid. The antisense RNA may hybridize with an N-terminal or 5′ upstream site such as a translation initiation site, transcription initiation site or promoter site. In some embodiments, the antisense RNA may hybridize with a 3′-untranslated region or mRNA splicing site.

The size of the antisense RNA may vary from 15 nucleotides to 15,000, preferably 20 to 12,000, in particular 100 to 10,000, 150 to 8,000, 200 to 7,000, 250 to 6,000, 300 to 5,000 nucleotides, such as 15 to 2,000, 20 to 1,000, 25 to 800, 30 to 600, 35 to 500, 40 to 400, 45 to 300, 50 to 250, 55 to 200, 60 to 150, or 65 to 100 nucleotides. In one embodiment, the antisense RNA has a length of at least 2,700 nucleotides (such as at least 2,800, at least 2,900, at least 3,000, at least 3,100, at least 3,200, at least 3,300, at least 3,400, at least 3,500, at least 3,600, at least 3,700, at least 3,800, at least 3,900, at least 4,000, at least 4,100, at least 4,200, at least 4,300, at least 4,400, at least 4,500, at least 4,600, at least 4,700, at least 4,800, at least 4,900, at least 5,000 nucleotides).

The stability of antisense RNA may be modified as required. For example, antisense RNA may be stabilized by one or more modifications having a stabilizing effect. Such modifications include modified phosphodiester linkages (such as methylphosphonate, phosphorothioate, phosphorodithioate or phosphoramidate linkages instead of naturally occurring phosphodiester linkages) and 2′-substitutions (e.g., 2′-fluoro, 2′-O-alkyl (such as 2′-O-methyl, 2′-O-propyl, or 2′-O-pentyl) and 2′-O-allyl). For example, in some embodiments of the antisense RNA, phosphorothioate linkages are substituted partially for phosphodiester linkages. Alternatively or additionally, in some embodiments of the 35 antisense RNA, the ribose moiety is substituted partially at the 2′-position with O-alkyl (such as 2′-O-methyl).

An antisense RNA can be targeted to any stretch of approximately 19 to 25 contiguous nucleotides in any of the target mRNA sequences (the “target sequence”). Generally, a target sequence on the target mRNA can be selected from a given cDNA sequence corresponding to the target mRNA, preferably beginning 50 to 100 nt downstream (i.e., in the 3′-direction) from the start codon. The target sequence can, however, be located in the 5′- or 3′-untranslated regions, or in the region nearby the start codon.

Antisense RNA can be obtained using a number of techniques known to those of skill in the art. For example, antisense RNA can be chemically synthesized or recombinantly produced using methods known in the art. Preferably, antisense RNA is transcribed from recombinant circular or linear DNA plasmids using any suitable promoter.

Selection of plasmids suitable for expressing antisense RNA, methods for inserting nucleic acid sequences for expressing the antisense RNA into the plasmid, and IVT methods of in vitro transcription of said antisense RNA are within the skill in the art.

By “small interfering RNA” or “siRNA” as used herein is meant an RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that is capable of binding specifically to a portion of a target mRNA. This binding induces a process, in which said portion of the target mRNA is cut or degraded and thereby the gene expression of said target mRNA inhibited. A range of 19 to 25 nucleotides is the most preferred size for siRNAs. Although, in principle, the sense and antisense strands of siRNAs can comprise two complementary, single-stranded RNA molecules, the siRNAs, according to the present disclosure, comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker, but is preferably a polynucleotide linker. Without wishing to be bound by any theory, it is believed that the hairpin area of the siRNA molecule is cleaved intracellularly by the “Dicer” protein (or its equivalent) to form an siRNA of two individual base-paired RNA molecules.

The siRNA can also comprise a 3′-overhang. As used herein, a “3′-overhang” refers to at least one unpaired nucleotide extending from the 3′-end of an RNA strand. Thus, in some embodiments, the siRNA comprises at least one 3′-overhang of from 1 to about 6 nucleotides (which includes ribonucleotides or deoxynucleotides) in length, preferably from 1 to about 5 nucleotides in length, more preferably from 1 to about 4 nucleotides in length, and particularly preferably from about 2 to about 4 nucleotides in length. In the embodiments in which both strands of the siRNA molecule (i.e., after the siRNA molecule is cleaved intracellularly by the “Dicer” protein) comprise a 3′-overhang, the length of the overhangs can be the same or different for each strand. In some preferred embodiments, the 3′-overhang is present on both strands of the siRNA, and is 2 nucleotides in length. For example, each strand of the siRNA can comprise 3′-overhangs of dideoxythymidylic acid (“TT”) or diuridylic acid (“uu”).

In order to enhance the stability of the siRNA, the 3′-overhangs can be also stabilized against degradation. In some embodiments, the overhangs are stabilized by including purine nucleotides, such as adenosine or guanosine nucleotides. Alternatively, substitution of pyrimidine nucleotides by modified analogues, e.g., substitution of uridine nucleotides in the 3′-overhangs with 2′-deoxythymidine, is tolerated and does not affect the efficiency of RNAi degradation. In particular, the absence of a 2′-hydroxyl in the 2′-deoxythymidine significantly enhances the nuclease resistance of the 3′-overhang in tissue culture medium.

As used herein, “target mRNA” refers to an RNA molecule that is a target for downregulation. In some embodiments, the target mRNA comprises an ORF encoding a pharmaceutically active peptide or polypeptide as specified herein. In some embodiments, the pharmaceutically active peptide or polypeptide is one whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

According to the present disclosure, siRNA can be targeted to any stretch of approximately 19 to 25 contiguous nucleotides in any of the target mRNA sequences (the “target sequence”). Techniques for selecting target sequences for siRNA are given, for example, in Tuschl T. et al., “The siRNA User Guide”, revised Oct. 11, 2002, the entire disclosure of which is herein incorporated by reference. “The siRNA User Guide” is available on the world wide web at a website maintained by Dr. Thomas Tuschl, Laboratory of RNA Molecular Biology, Rockefeller University, New York, USA, and can be found by accessing the website of the Rockefeller University and searching with the keyword “siRNA”. Further guidance with respect to the selection of target sequences and/or the design of siRNA can be found on the webpages of Protocol Online (www.protocol-online.com) using the keyword “siRNA”. Thus, in some embodiments, the sense strand of the siRNA used in the present disclosure comprises a nucleotide sequence substantially identical to any contiguous stretch of about 19 to about 25 nucleotides in the target mRNA.

Generally, a target sequence on the target mRNA can be selected from a given cDNA sequence corresponding to the target mRNA, preferably beginning 50 to 100 nt downstream (i.e., in the 3′-direction) from the start codon. The target sequence can, however, be located in the 5′- or 3′-untranslated regions, or in the region nearby the start codon.

siRNA can be obtained using a number of techniques known to those of skill in the art. For example, siRNA can be chemically synthesized or recombinantly produced using methods known in the art, such as the Drosophila in vitro system described in U.S. application no. 2002/0086356 of Tuschl et al., the entire disclosure of which is herein incorporated by reference. siRNA can be expressed from pol III expression vectors without a change in targeting site, as expression of RNAs from pol III promoters is only believed to be efficient when the first transcribed nucleotide is a purine.

Preferably, siRNA is transcribed from recombinant circular or linear DNA plasmids using any suitable promoter. Suitable promoters for transcribing siRNA used in the present disclosure from a plasmid include, for example, the U6 or H1 RNA pol III promoter sequences and the cytomegalovirus promoter. Selection of other suitable promoters is within the skill in the art.

Selection of plasmids suitable for transcribing siRNA, methods for inserting nucleic acid sequences for expressing the siRNA into the plasmid, and IVT methods of in vitro transcription of said siRNA are within the skill in the art.

The term “miRNA” (microRNA) as used herein relates to non-coding RNAs which have a length of 21 to 25 (such as 21 to 23, preferably 22) nucleotides and which induce degradation and/or prevent translation of target mRNAs. miRNAs are typically found in plants, animals and some viruses, wherein they are encoded by eukaryotic nuclear DNA in plants and animals and by viral DNA (in viruses whose genome is based on DNA), respectively. miRNAs are post-transcriptional regulators that bind to complementary sequences on target messenger RNA transcripts (mRNAs), usually resulting in translational repression or target degradation and gene silencing.

miRNA can be obtained using a number of techniques known to those of skill in the art. For example, miRNA can be chemically synthesized or recombinantly produced using methods known in the art (e.g., by using commercially available kits such as the miRNA cDNA Synthesis Kit sold by Applied Biological Materials Inc.). Preferably, miRNA is transcribed from recombinant circular or linear DNA plasmids using any suitable promoter.

### Pharmaceutically Active Peptides or Polypeptides

“Encoding” refers to the inherent property of specific sequences of nucleotides in a polynucleotide, such as a gene, a cDNA, or an RNA (preferably mRNA), to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of RNA (preferably mRNA) corresponding to that gene produces the protein in a cell or other biological system. Both the coding strand, the nucleotide sequence of which is identical to the RNA sequence and is usually provided in sequence listings, and the non-coding strand, used as the template for transcription of a gene or cDNA, can be referred to as encoding the protein or other product of that gene or cDNA.

In some embodiments, RNA (preferably mRNA) described in the present disclosure comprises a nucleic acid sequence (e.g., an ORF) encoding one or more polypeptides, e.g., a peptide or protein, preferably a pharmaceutically active peptide or protein.

In some embodiments, RNA (preferably mRNA) described in the present disclosure comprises a nucleic acid sequence (e.g., an ORF) encoding a peptide or protein, preferably a pharmaceutically active peptide or protein, and is capable of expressing said peptide or protein, in particular if transferred into a cell or subject. Thus, in some embodiments, the RNA (preferably mRNA) described in the present disclosure contains a coding region (ORF) encoding a peptide or protein, preferably encoding a pharmaceutically active peptide or protein. In this respect, an “open reading frame” or “ORF” is a continuous stretch of codons beginning with a start codon and ending with a stop codon. Such RNA (preferably mRNA) encoding a pharmaceutically active peptide or protein is also referred to herein as “pharmaceutically active RNA” (or “pharmaceutically active mRNA”). In some embodiments, RNA (preferably mRNA) described in the present disclosure comprises a nucleic acid sequence encoding more than one peptide or polypeptide, e.g., two, three, four or more peptides or polypeptides.

According to the present disclosure, the term “pharmaceutically active peptide or protein” means a peptide or protein that can be used in the treatment of an individual where the expression of the peptide or protein would be of benefit, e.g., in ameliorating the symptoms of a disease or disorder. Preferably, a pharmaceutically active peptide or protein has curative or palliative properties and may be administered to ameliorate, relieve, alleviate, reverse, delay onset of or lessen the severity of one or more symptoms of a disease or disorder. In some embodiments, a pharmaceutically active peptide or protein has a positive or advantageous effect on the condition or disease state of an individual when administered to the individual in a therapeutically effective amount. A pharmaceutically active peptide or protein may have prophylactic properties and may be used to delay the onset of a disease or disorder or to lessen the severity of such disease or disorder. The term “pharmaceutically active peptide or protein” includes entire proteins or polypeptides, and can also refer to pharmaceutically active fragments thereof. It can also include pharmaceutically active analogs of a peptide or protein.

Specific examples of phamaceutically active peptides and proteins include, but are not limited to, immunostimulants, e.g., cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins. In some embodiments, the pharmaceutically active peptide and polypeptide includes a replacement protein.

An “immunostimulant” is any substance that stimulates the immune system by inducing activation or increasing activity of any of the immune system's components, in particular immune effector cells. The immunostimulant may be pro-inflammatory (e.g., when treating infections or cancer), or anti-inflammatory (e.g., when treating autoimmune diseases).

In some embodiments, the immunostimulant is a cytokine or a variant thereof. Examples of cytokines include interferons, such as interferon-alpha (IFN-α) or interferon-gamma (IFN-γ), interleukins, such as IL2, IL7, IL12, IL15 and 1L23, colony stimulating factors, such as M-CSF and GM-CSF, and tumor necrosis factor. According to another aspect, the immunostimulant includes an adjuvant-type immunostimulatory agent such as APC Toll-like Receptor agonists or costimulatory/cell adhesion membrane proteins. Examples of Toll-like Receptor agonists include costimulatory/adhesion proteins such as CD80, CD86, and ICAM-1.

The term “cytokines” relates to proteins which have a molecular weight of about 5 to 60 kDa (such as about 5 to 20 kDa) and which participate in cell signaling (e.g., paracrine, endocrine, and/or autocrine signaling). In particular, when released, cytokines exert an effect on the behavior of cells around the place of their release. Examples of cytokines include lymphokines, interleukins, chemokines, interferons, and tumor necrosis factors (TNFs). According to the present disclosure, cytokines do not include hormones or growth factors. Cytokines differ from hormones in that (i) they usually act at much more variable concentrations than hormones and (ii) generally are made by a broad range of cells (nearly all nucleated cells can produce cytokines). Interferons are usually characterized by antiviral, antiproliferative and immunomodulatory activities. Interferons are proteins that alter and regulate the transcription of genes within a cell by binding to interferon receptors on the regulated cell's surface, thereby preventing viral replication within the cells. The interferons can be grouped into two types. IFN-gamma is the sole type II interferon; all others are type I interferons. Particular examples of cytokines include erythropoietin (EPO), colony stimulating factor (CSF), granulocyte colony stimulating factor (G-CSF), granulocyte-macrophage colony stimulating factor (GM-CSF), tumor necrosis factor (TNF), bone morphogenetic protein (BMP), interferon alfa (IFNα), interferon beta (IFNβ), interferon gamma (INFγ), interleukin 2 (IL-2), interleukin 4 (IL-4), interleukin 10 (IL-10), interleukin 11 (IL-11), interleukin 12 (IL-12), and interleukin 21 (IL-21).

According to the disclosure, a cytokine may be a naturally occurring cytokine or a functional fragment or variant thereof. A cytokine may be human cytokine and may be derived from any vertebrate, especially any mammal. One particularly preferred cytokine is interferon-α.

Immunostimulants may be provided to a subject by administering to the subject RNA encoding an immunostimulant in a formulation for preferential delivery of RNA to liver or liver tissue. The delivery of RNA to such target organ or tissue is preferred, in particular, if it is desired to express large amounts of the immunostimulant and/or if systemic presence of the immunostimulant, in particular in significant amounts, is desired or required. RNA delivery systems have an inherent preference to the liver. This pertains to lipid-based particles, cationic and neutral nanoparticles, in particular lipid nanoparticles.

Examples of suitable immunostimulants for targeting liver are cytokines involved in T cell proliferation and/or maintenance. Examples of suitable cytokines include IL2 or IL7, fragments and variants thereof, and fusion proteins of these cytokines, fragments and variants, such as extended-PK cytokines.

In another embodiment, RNA encoding an immunostimulant may be administered in a formulation for preferential delivery of RNA to the lymphatic system, in particular secondary lymphoid organs, more specifically spleen. The delivery of an immunostimulant to such target tissue is preferred, in particular, if presence of the immunostimulant in this organ or tissue is desired (e.g., for inducing an immune response, in particular in case immunostimulants such as cytokines are required during T-cell priming or for activation of resident immune cells), while it is not desired that the immunostimulant is present systemically, in particular in significant amounts (e.g., because the immunostimulant has systemic toxicity).

Examples of suitable immunostimulants are cytokines involved in T cell priming. Examples of suitable cytokines include IL12, IL15, IFN-α, or IFN-β, fragments and variants thereof, and fusion proteins of these cytokines, fragments and variants, such as extended-PK cytokines.

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses.

Based on the type of receptor through which they signal, interferons are typically divided among three classes: type I interferon, type II interferon, and type III interferon.

All type I interferons bind to a specific cell surface receptor complex known as the IFN-α/P receptor (IFNAR) that consists of IFNAR1 and IFNAR2 chains.

The type I interferons present in humans are IFNα, IFNβ, IFNε, IFNκ and IFNω. In general, type 1 interferons are produced when the body recognizes a virus that has invaded it. They are produced by fibroblasts and monocytes. Once released, type I interferons bind to specific receptors on target cells, which leads to expression of proteins that will prevent the virus from producing and replicating its RNA and DNA.

The IFNα proteins are produced mainly by plasmacytoid dendritic cells (pDCs). They are mainly involved in innate immunity against viral infection. The genes responsible for their synthesis come in 13 subtypes that are called IFNA1, IFNA2, IFNA4, IFNA5, IFNA6, IFNA7, IFNA8, IFNA10, IFNA13, IFNA14, IFNA16, IFNA17, IFNA21. These genes are found together in a cluster on chromosome 9.

The IFNβ proteins are produced in large quantities by fibroblasts. They have antiviral activity that is involved mainly in innate immune response. Two types of IFNβ have been described, IFNβ1 and IFNβ3.

The natural and recombinant forms of IFNβ1 have antiviral, antibacterial, and anticancer properties.

Type II interferon (IFNγ in humans) is also known as immune interferon and is activated by IL12. Furthermore, type II interferons are released by cytotoxic T cells and T helper cells.

Type III interferons signal through a receptor complex consisting of IL10R2 (also called CRF2-4) and IFNLR1 (also called CRF2-12). Although discovered more recently than type I and type II IFNs, recent information demonstrates the importance of type III IFNs in some types of virus or fungal infections.

In general, type I and II interferons are responsible for regulating and activating the immune response.

According to the disclosure, a type I interferon is preferably IFNα or IFNβ, more preferably IFNα.

According to the disclosure, an interferon may be a naturally occurring interferon or a functional fragment or variant thereof. An interferon may be human interferon and may be derived from any vertebrate, especially any mammal.

Interleukins (ILs) are a group of cytokines (secreted proteins and signal molecules) that can be divided into four major groups based on distinguishing structural features. However, their amino acid sequence similarity is rather weak (typically 15-25% identity). The human genome encodes more than 50 interleukins and related proteins.

According to the disclosure, an interleukin may be a naturally occurring interleukin or a functional fragment or variant thereof. An interleukin may be human interleukin and may be derived from any vertebrate, especially any mammal.

Immunostimulant polypeptides described herein can be prepared as fusion or chimeric polypeptides that include an immunostimulant portion and a heterologous polypeptide (i.e., a polypeptide that is not an immunostimulant). The immunostimulant may be fused to an extended-PK group, which increases circulation half-life. Non-limiting examples of extended-PK groups are described infra. It should be understood that other PK groups that increase the circulation half-life of immunostimulants such as cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16(7):903-15 which is herein incorporated by reference in its entirety. As used herein, an “extended-PK” immunostimulant refers to an immunostimulant moiety in combination with an extended-PK group. In some embodiments, the extended-PK immunostimulant is a fusion protein in which an immunostimulant moiety is linked or fused to an extended-PK group.

In certain embodiments, the serum half-life of an extended-PK immunostimulant is increased relative to the immunostimulant alone (i.e., the immunostimulant not fused to an extended-PK group). In certain embodiments, the serum half-life of the extended-PK immunostimulant is at least 20%, at least 40%, at least 60%, at least 80%, at least 100%, at least 120%, at least 150%, at least 180%, at least 200%, at least 400%, at least 600%, at least 800%, or at least 1000% longer relative to the serum half-life of the immunostimulant alone. In certain embodiments, the serum half-life of the extended-PK immunostimulant is at least 1.5-fold, 2-fold, 2.5-fold, 3-fold, 3.5-fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the immunostimulant alone. In certain embodiments, the serum half-life of the extended-PK immunostimulant is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

As used herein, “half-life” refers to the time taken for the serum or plasma concentration of a compound such as a peptide or polypeptide to reduce by 50%, in vivo, for example due to degradation and/or clearance or sequestration by natural mechanisms. An extended-PK immunostimulant suitable for use herein is stabilized in vivo and its half-life increased by, e.g., fusion to serum albumin (e.g., HSA or MSA), which resist degradation and/or clearance or sequestration. The half-life can be determined in any manner known per se, such as by phannacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular an immunostimulant. The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In some embodiments, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver, that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789. The terms, human serum albumin (HSA) and human albumin (HA) are used interchangeably herein. The terms “albumin” and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

In some embodiments, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used. In other embodiments, the albumin fusion protein has a therapeutic protein fused to both the N-terminus and the C-terminus of albumin. In a preferred embodiment, the therapeutic proteins fused at the N- and C-termini are the same therapeutic proteins. In another preferred embodiment, the therapeutic proteins fused at the N- and C-termini are different therapeutic proteins. In some embodiments, the different therapeutic proteins are both cytokines.

In some embodiments, the therapeutic protein(s) is (are) joined to the albumin through (a) peptide linker(s). A peptide linker between the fused portions may provide greater physical separation between the moieties and thus maximize the accessibility of the therapeutic protein portion, for instance, for binding to its cognate receptor. The peptide linker may consist of amino acids such that it is flexible or more rigid. The linker sequence may be cleavable by a protease or chemically.

As used herein, the term “Fe region” refers to the portion of a native immunoglobulin formed by the respective Fe domains (or Fe moieties) of its two heavy chains. As used herein, the term “Fe domain” refers to a portion or fragment of a single immunoglobulin (Ig) heavy chain wherein the Fe domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fe domain comprises at least one of: a hinge (e.g., upper, middle, and/or lower hinge region) domain, a CH2 domain, a CH3 domain, a CH4 domain, or a variant, portion, or fragment thereof.

In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain.

In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fe domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fe domain may be derived from an immunoglobulin of any species and/or any subtype, including, but not limited to, a human IgG1, IgG2, IgG3, IgG4, IgD, IgA, IgE, or IgM antibody. The Fe domain encompasses native Fe and Fe variant molecules. As set forth herein, it will be understood by one of ordinary skill in the art that any Fe domain may be modified such that it varies in amino acid sequence from the native Fe domain of a naturally occurring immunoglobulin molecule. In certain embodiments, the Fe domain has reduced effector function (e.g., FcγR binding).

The Fe domains of a polypeptide described herein may be derived from different immunoglobulin molecules. For example, an Fe domain of a polypeptide may comprise a CH2 and/or CH3 domain derived from an IgG1 molecule and a hinge region derived from an IgG3 molecule. In another example, an Fe domain can comprise a chimeric hinge region derived, in part, from an IgG1 molecule and, in part, from an IgG3 molecule. In another example, an Fe domain can comprise a chimeric hinge derived, in part, from an IgG1 molecule and, in part, from an IgG4 molecule.

In certain embodiments, an extended-PK group includes an Fe domain or fragments thereof or variants of the Fe domain or fragments thereof (all of which for the purpose of the present disclosure are comprised by the term “Fc domain”). The Fe domain does not contain a variable region that binds to antigen. Fe domains suitable for use in the present disclosure may be obtained from a number of different sources. In certain embodiments, an Fc domain is derived from a human immunoglobulin. In certain embodiments, the Fe domain is from a human IgG1 constant region. It is understood, however, that the Fe domain may be derived from an immunoglobulin of another mammalian species, including for example, a rodent (e.g. a mouse, rat, rabbit, guinea pig) or non-human primate (e.g. chimpanzee, macaque) species.

Moreover, the Fe domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fe domain gene sequences (e.g., mouse and human constant region gene sequences) are available in the form of publicly accessible deposits. Constant region domains comprising an Fe domain sequence can be selected lacking a particular effector function and/or with a particular modification to reduce immunogenicity. Many sequences of antibodies and antibody-encoding genes have been published and suitable Fe domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

In certain embodiments, the extended-PK immunostimulant, suitable for use according to the disclosure, can employ one or more peptide linkers. As used herein, the term “peptide linker” refers to a peptide or polypeptide sequence which connects two or more domains (e.g., the extended-PK moiety and an immunostimulant moiety) in a linear amino acid sequence of a polypeptide chain. For example, peptide linkers may be used to connect an immunostimulant moiety to a HSA domain.

Linkers suitable for fusing the extended-PK group to, e.g., an immunostimulant are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

In some embodiments, a pharmaceutically active peptide or protein comprises a replacement protein. In these embodiments, the present disclosure provides a method for treatment of a subject having a disorder requiring protein replacement (e.g., protein deficiency disorders) comprising administering to the subject RNA as described herein encoding a replacement protein. The term “protein replacement” refers to the introduction of a protein (including functional variants thereof) into a subject having a deficiency in such protein. The term also refers to the introduction of a protein into a subject otherwise requiring or benefiting from providing a protein, e.g., suffering from protein insufficiency. The term “disorder characterized by a protein deficiency” refers to any disorder that presents with a pathology caused by absent or insufficient amounts of a protein. This term encompasses protein folding disorders, i.e., conformational disorders, that result in a biologically inactive protein product. Protein insufficiency can be involved in infectious diseases, immunosuppression, organ failure, glandular problems, radiation illness, nutritional deficiency, poisoning, or other environmental or external insults.

The term “hormones” relates to a class of signaling molecules produced by glands, wherein signaling usually includes the following steps: (i) synthesis of a hormone in a particular tissue; (ii) storage and secretion; (iii) transport of the hormone to its target; (iv) binding of the hormone by a receptor; (v) relay and amplification of the signal; and (vi) breakdown of the hormone. Hormones differ from cytokines in that (1) hormones usually act in less variable concentrations and (2) generally are made by specific kinds of cells. In some embodiments, a “hormone” is a peptide or protein hormone, such as insulin, vasopressin, prolactin, adrenocorticotropic hormone (ACTH), thyroid hormone, growth hormones (such as human grown hormone or bovine somatotropin), oxytocin, atrial-natriuretic peptide (ANP), glucagon, somatostatin, cholecystokinin, gastrin, and leptins.

The term “adhesion molecules” relates to proteins which are located on the surface of a cell and which are involved in binding of the cell with other cells or with the extracellular matrix (ECM). Adhesion molecules are typically transmembrane receptors and can be classified as calcium-independent (e.g., integrins, immunoglobulin superfamily, lymphocyte homing receptors) and calcium-dependent (cadherins and selectins). Particular examples of adhesion molecules are integrins, lymphocyte homing receptors, selectins (e.g., P-selectin), and addressins.

Integrins are also involved in signal transduction. In particular, upon ligand binding, integrins modulate cell signaling pathways, e.g., pathways of transmembrane protein kinases such as receptor tyrosine kinases (RTK). Such regulation can lead to cellular growth, division, survival, or differentiation or to apoptosis. Particular examples of integrins include: α₁β₁, α₂β₁, α₃β₁, α₄β₁, α₅β₁, α₆β₁, α₇β₁, α_{L}β₂, α_{M}β₂, α_{11b}β₃, αᵥβ₁, αᵥβ₃, αᵥβ₅, αᵥβ₆, αᵥβ₈, and α₆β₄.

The term “immunoglobulins” or “immunoglobulin superfamily” refers to molecules which are involved in the recognition, binding, and/or adhesion processes of cells. Molecules belonging to this superfamily share the feature that they contain a region known as immunoglobulin domain or fold. Members of the immunoglobulin superfamily include antibodies (e.g., IgG), T cell receptors (TCRs), major histocompatibility complex (MHC) molecules, co-receptors (e.g., CD4, CD8, CD19), antigen receptor accessory molecules (e.g., CD-3γ, CD3-δ, CD-3ε, CD79a, CD79b), co-stimulatory or inhibitory molecules (e.g., CD28, CD80, CD86), and other.

The term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells. Immunologically active compounds possess potent immunostimulating activity including, but not limited to, antiviral and antitumor activity, and can also down-regulate other aspects of the immune response, for example shifting the immune response away from a TH2 immune response, which is useful for treating a wide range of TH2 mediated diseases. Immunologically active compounds can be useful as vaccine adjuvants. Particular examples of immunologically active compounds include interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, and antigens, in particular tumor-associated antigens, pathogen-associated antigens (such as bacterial, parasitic, or viral antigens), allergens, and autoantigens. A preferred immunologically active compound is a vaccine antigen, i.e., an antigen whose inoculation into a subject induces an immune response.

In some embodiments, RNA (in particular, mRNA) described in the present disclosure comprises a nucleic acid sequence encoding a peptide or polypeptide comprising an epitope for inducing an immune response against an antigen in a subject. The “peptide or polypeptide comprising an epitope for inducing an immune response against an antigen in a subject” is also designated herein as “vaccine antigen”, “peptide and protein antigen” or simply “antigen”.

In some embodiments, the RNA (in particular, mRNA) encoding vaccine antigen is a single-stranded, 5′ capped mRNA that is translated into the respective protein upon entering cells of a subject being administered the RNA, e.g., antigen-presenting cells (APCs). Preferably, the RNA (i) contains structural elements optimized for maximal efficacy of the RNA with respect to stability and translational efficiency (5′ cap, 5′ UTR, 3′ UTR, poly(A) sequence); (ii) is modified for optimized efficacy of the RNA (e.g., increased translation efficacy, decreased immunogenicity, and/or decreased cytotoxicity) (e.g., by replacing (partially or completely, preferably completely) naturally occurring nucleosides (in particular cytidine) with synthetic nucleosides (e.g., modified nucleosides selected from the group consisting of pseudouridine (W), N1-methyl-pseudouridine (m1ψ), and 5-methyl-uridine); and/or codon-optimization), or (iii) both (i) and (ii).

In some embodiments, beta-S-ARCA(D1) is utilized as specific capping structure at the 5′-end of the RNA. In some embodiments, the 5′-UTR comprises the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12. In some embodiments, the 3′-UTR comprises the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13. In some embodiments, the poly(A) sequence is 110 nucleotides in length and consists of a stretch of 30 adenosine residues, followed by a 10 nucleotide linker sequence and another 70 adenosine residues. This poly(A) sequence was designed to enhance RNA stability and translational efficiency in dendritic cells. In some embodiments, the poly(A) sequence comprises the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14. In some embodiments, the RNA comprises a modified nucleoside in place of uridine.

In some embodiments, the modified nucleoside replacing (partially or completely, preferably completely) uridine is selected from the group consisting of pseudouridine (ψ), N1-methyl-pseudouridine (mly), and 5-methyl-uridine. In some embodiments, the RNA encoding the vaccine antigen has a coding sequence (a) which is codon-optimized, (b) the G/C content of which is increased compared to the wild type coding sequence, or (c) both (a) and (b).

In some embodiments, the RNA encoding the vaccine antigen is expressed in cells of the subject to provide the vaccine antigen. In some embodiments, expression of the vaccine antigen is at the cell surface. In some embodiments, the vaccine antigen is presented in the context of MHC. In some embodiments, the RNA encoding the vaccine antigen is transiently expressed in cells of the subject. In some embodiments, the RNA encoding the vaccine antigen is administered systemically. In some embodiments, after systemic administration of the RNA encoding the vaccine antigen, expression of the RNA encoding the vaccine antigen in spleen occurs. In some embodiments, after systemic administration of the RNA encoding the vaccine antigen, expression of the RNA encoding the vaccine antigen in antigen presenting cells, preferably professional antigen presenting cells occurs. In some embodiments, the antigen presenting cells are selected from the group consisting of dendritic cells, macrophages and B cells. In some embodiments, after systemic administration of the RNA encoding the vaccine antigen, no or essentially no expression of the RNA encoding the vaccine antigen in lung and/or liver occurs. In some embodiments, after systemic administration of the RNA encoding the vaccine antigen, expression of the RNA encoding the vaccine antigen in spleen is at least 5-fold the amount of expression in lung.

The vaccine antigen comprises an epitope for inducing an immune response against an antigen in a subject. Accordingly, the vaccine antigen comprises an antigenic sequence for inducing an immune response against an antigen in a subject. Such antigenic sequence may correspond to a target antigen or disease-associated antigen, e.g., a protein of an infectious agent (e.g., viral or bacterial antigen) or tumor antigen, or may correspond to an immunogenic variant thereof, or an immunogenic fragment of the target antigen or disease-associated antigen or the immunogenic variant thereof. Thus, the antigenic sequence may comprise at least an epitope of a target antigen or disease-associated antigen or an immunogenic variant thereof.

The antigenic sequences, e.g., epitopes, suitable for use according to the disclosure typically may be derived from a target antigen, i.e. the antigen against which an immune response is to be elicited. For example, the antigenic sequences contained within the vaccine antigen may be a target antigen or a fragment or variant of a target antigen.

The antigenic sequence or a procession product thereof, e.g., a fragment thereof, may bind to the antigen receptor such as TCR or CAR carried by immune effector cells. In some embodiments, the antigenic sequence is selected from the group consisting of the antigen expressed by a target cell to which the immune effector cells are targeted or a fragment thereof, or a variant of the antigenic sequence or the fragment.

A vaccine antigen which may be provided to a subject according to the present disclosure by administering RNA encoding the vaccine antigen, preferably results in the induction of an immune response, e.g., in the stimulation, priming and/or expansion of immune effector cells, in the subject being provided the vaccine antigen. Said immune response, e.g., stimulated, primed and/or expanded immune effector cells, is preferably directed against a target antigen, in particular a target antigen expressed by diseased cells, tissues and/or organs, i.e., a disease-associated antigen. Thus, a vaccine antigen may comprise the disease-associated antigen, or a fragment or variant thereof. In some embodiments, such fragment or variant is immunologically equivalent to the disease-associated antigen.

In the context of the present disclosure, the term “fragment of an antigen” or “variant of an antigen” means an agent which results in the induction of an immune response, e.g., in the stimulation, priming and/or expansion of immune effector cells, which immune response, e.g., stimulated, primed and/or expanded immune effector cells, targets the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope of the disease-associated antigen to which the antigen receptor of the immune effector cells is targeted or a sequence which is homologous to an epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of inducing an immune response against, e.g., stimulating, priming and/or expanding immune effector cells carrying an antigen receptor binding to, the antigen or cells expressing the antigen. It is preferred that the vaccine antigen (similar to the disease-associated antigen) provides the relevant epitope for binding by the antigen receptor present on the immune effector cells. In some embodiments, the vaccine antigen or a fragment thereof (similar to the disease-associated antigen) is expressed on the surface of a cell such as an antigen-presenting cell (optionally in the context of MHC) so as to provide the relevant epitope for binding by immune effector cells. The vaccine antigen may be a recombinant antigen.

In some embodiments of all aspects of the invention, the RNA encoding the vaccine antigen is expressed in cells of a subject to provide the antigen or a procession product thereof for binding by the antigen receptor expressed by immune effector cells, said binding resulting in stimulation, priming and/or expansion of the immune effector cells. An “antigen” according to the present disclosure covers any substance that will elicit an immune response and/or any substance against which an immune response or an immune mechanism such as a cellular response and/or humoral response is directed. This also includes situations wherein the antigen is processed into antigen peptides and an immune response or an immune mechanism is directed against one or more antigen peptides, in particular if presented in the context of MHC molecules. In particular, an “antigen” relates to any substance, such as a peptide or polypeptide, that reacts specifically with antibodies or T-lymphocytes (T-cells). The term “antigen” may comprise a molecule that comprises at least one epitope, such as a T cell epitope. In some embodiments, an antigen is a molecule which, optionally after processing, induces an immune reaction, which may be specific for the antigen (including cells expressing the antigen). In some embodiments, an antigen is a disease-associated antigen, such as a tumor antigen, a viral antigen, or a bacterial antigen, or an epitope derived from such antigen.

In some embodiments, an antigen is presented or present on the surface of cells of the immune system such as antigen presenting cells like dendritic cells or macrophages. An antigen or a procession product thereof such as a T cell epitope is in some embodiments bound by an antigen receptor. Accordingly, an antigen or a procession product thereof may react specifically with immune effector cells such as T-lymphocytes (T cells).

The term “autoantigen” or “self-antigen” refers to an antigen which originates from within the body of a subject (i.e., the autoantigen can also be called “autologous antigen”) and which produces an abnormally vigorous immune response against this normal part of the body. Such vigorous immune reactions against autoantigens may be the cause of “autoimmune diseases”.

In some embodiments, an antigen is expressed on the surface of a diseased cell (such as tumor cell or an infected cell). In some embodiments, an antigen receptor is a CAR which binds to an extracellular domain or to an epitope in an extracellular domain of an antigen. In some embodiments, a CAR binds to native epitopes of an antigen present on the surface of living cells. In some embodiments, binding of a CAR when expressed by T cells and/or present on T cells to an antigen present on cells such as antigen presenting cells results in stimulation, priming and/or expansion of said T cells. In some embodiments, binding of a CAR when expressed by T cells and/or present on T cells to an antigen present on diseased cells results in cytolysis and/or apoptosis of the diseased cells, wherein said T cells preferably release cytotoxic factors, e.g., perforins and granzymes.

According to some embodiments, an amino acid sequence enhancing antigen processing and/or presentation is fused, either directly or through a linker, to an antigenic peptide or polypeptide (antigenic sequence). Accordingly, in some embodiments, the RNA described herein comprises at least one coding region encoding an antigenic peptide or polypeptide and an amino acid sequence enhancing antigen processing and/or presentation.

In some embodiments, antigen for vaccination which may be administered in the form of RNA coding therefor comprises a naturally occurring antigen or a fragment such as an epitope thereof.

Such amino acid sequences enhancing antigen processing and/or presentation are preferably located at the C-terminus of the antigenic peptide or polypeptide (and optionally at the C-terminus of an amino acid sequence which breaks immunological tolerance), without being limited thereto. Amino acid sequences enhancing antigen processing and/or presentation as defined herein preferably improve antigen processing and presentation. In some embodiments, the amino acid sequence enhancing antigen processing and/or presentation as defined herein includes, without being limited thereto, sequences derived from the human MHC class I complex (HLA-B51, haplotype A2, B27/B51, Cw2/Cw3), in particular a sequence comprising the amino acid sequence of SEQ ID NO: 36 or a functional variant thereof.

In some embodiments, a secretory sequence, e.g., a sequence comprising the amino acid sequence of SEQ ID NO: 35, may be fused to the N-terminus of the antigenic peptide or polypeptide.

In some embodiments, an amino acid sequence enhancing antigen processing and/or presentation comprises the amino acid sequence of SEQ ID NO: 36, an amino acid sequence having at least 99%, 98%, 97%, 96%, 95%, 90%, 85%, or 80% identity to the amino acid sequence of SEQ ID NO: 36, or a functional fragment of the amino acid sequence of SEQ ID NO: 36, or the amino acid sequence having at least 99%, 98%, 97%, 96%, 95%, 90%, 85%, or 80% identity to the amino acid sequence of SEQ ID NO: 36. In some embodiments, an amino acid sequence enhancing antigen processing and/or presentation comprises the amino acid sequence of SEQ ID NO: 36.

Accordingly, in some embodiments, the RNA described herein comprises at least one coding region encoding an antigenic peptide or polypeptide and an amino acid sequence enhancing antigen processing and/or presentation, said amino acid sequence enhancing antigen processing and/or presentation preferably being fused to the antigenic peptide or polypeptide, more preferably to the C-terminus of the antigenic peptide or polypeptide as described herein.

Furthermore, a secretory sequence, e.g., a sequence comprising the amino acid sequence of SEQ ID NO: 35, may be fused to the N-terminus of the antigenic peptide or polypeptide.

Amino acid sequences derived from tetanus toxoid of Clostridium tetani may be employed to overcome self-tolerance mechanisms in order to efficiently mount an immune response to self-antigens by providing T-cell help during priming.

It is known that tetanus toxoid heavy chain includes epitopes that can bind promiscuously to MHC class II alleles and induce CD4⁺ memory T cells in almost all tetanus vaccinated individuals. In addition, the combination of tetanus toxoid (TT) helper epitopes with tumor-associated antigens is known to improve the immune stimulation compared to application of tumor-associated antigen alone by providing CD4⁺-mediated T-cell help during priming. To reduce the risk of stimulating CD8⁺ T cells with the tetanus sequences which might compete with the intended induction of tumor antigen-specific T-cell response, not the whole fragment C of tetanus toxoid is used as it is known to contain CD8⁺ T-cell epitopes. Two peptide sequences containing promiscuously binding helper epitopes were selected alternatively to ensure binding to as many MHC class II alleles as possible. Based on the data of the ex vivo studies the well-known epitopes p2 (QYIKANSKFIGITEL; TT₈₃₀₋₈₄₄; SEQ ID NO: 40) and p16 (MTNSVDDALINSTKIYSYFPSVISKVNQGAQG; TT₅₇₈₋₆₀₉; SEQ ID NO: 41) were selected. The p2 epitope was already used for peptide vaccination in clinical trials to boost anti-melanoma activity. Non-clinical data showed that RNA vaccines encoding both a tumor antigen plus promiscuously binding tetanus toxoid sequences lead to enhanced CD8 T-cell responses directed against the tumor antigen and improved break of tolerance. Immunomonitoring data from patients vaccinated with vaccines including those sequences fused in frame with the tumor antigen-specific sequences reveal that the tetanus sequences chosen are able to induce tetanus-specific T-cell responses in almost all patients.

According to some embodiments, an amino acid sequence which breaks immunological tolerance is fused, either directly or through a linker, e.g., a linker having the amino acid sequence according to SEQ ID NO: 38, to the antigenic peptide or polypeptide.

Such amino acid sequences which break immunological tolerance are preferably located at the C-terminus of the antigenic peptide or polypeptide (and optionally at the N-terminus of the amino acid sequence enhancing antigen processing and/or presentation, wherein the amino acid sequence which breaks immunological tolerance and the amino acid sequence enhancing antigen processing and/or presentation may be fused either directly or through a linker, e.g., a linker having the amino acid sequence according to SEQ ID NO: 39), without being limited thereto. Amino acid sequences which break immunological tolerance as defined herein preferably improve T cell responses. In some embodiments, the amino acid sequence which breaks immunological tolerance as defined herein includes, without being limited thereto, sequences derived from tetanus toxoid-derived helper sequences p2 and p16 (P2P16), in particular a sequence comprising the amino acid sequence of SEQ ID NO: 37 or a functional variant thereof.

In some embodiments, an amino acid sequence which breaks immunological tolerance comprises the amino acid sequence of SEQ ID NO: 37, an amino acid sequence having at least 99%, 98%, 97%, 96%, 95%, 90%, 85%, or 80% identity to the amino acid sequence of SEQ ID NO: 37, or a functional fragment of the amino acid sequence of SEQ ID NO: 37, or the amino acid sequence having at least 99%, 98%, 97%, 96%, 95%, 90%, 85%, or 80% identity to the amino acid sequence of SEQ ID NO: 37. In some embodiments, an amino acid sequence which breaks immunological tolerance comprises the amino acid sequence of SEQ ID NO: 37.

In the following, embodiments of vaccine RNAs are described, wherein certain terms used when describing elements thereof have the following meanings:
cap: 5′-cap structure selected from the group consisting of m₂^{7,2′O}G(5′)ppSp(5′)G (in particular its D1 diastereomer), m₂^{7,3′O}G(5′)ppp(5′)G, and m₂^{7,3′O} Gppp(m₁^{2′-O})ApG.
hAg-Kozak: 5′-UTR sequence of the human alpha-globin mRNA with an optimized ‘Kozak sequence’ to increase translational efficiency.
sec/MITD: Fusion-protein tags derived from the sequence encoding the human MHC class I complex (HLA-B51, haplotype A2, B27/B51, Cw2/Cw3), which have been shown to improve antigen processing and presentation. See corresponds to the 78 bp fragment coding for the secretory signal peptide, which guides translocation of the nascent polypeptide chain into the endoplasmatic reticulum. MITD corresponds to the transmembrane and cytoplasmic domain of the MHC class 1 molecule, also called MHC class I trafficking domain.
Antigen: Sequences encoding the respective vaccine antigen/epitope.
Glycine-serine linker (GS): Sequences coding for short peptide linkers predominantly consisting of the amino acids glycine (G) and serine (S), as commonly used for fusion proteins.
P2P16: Sequence coding for tetanus toxoid-derived helper epitopes to break immunological tolerance.
FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.
A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells.

In some embodiments, vaccine RNA described herein has one of the following structures:

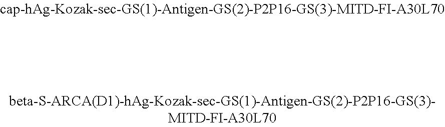

In some embodiments, vaccine antigen described herein has the structure:

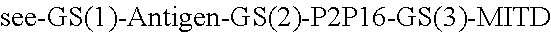

In some embodiments, hAg-Kozak comprises the nucleotide sequence of SEQ ID NO: 12. In some embodiments, sec comprises the amino acid sequence of SEQ ID NO: 35. In some embodiments, P2P16 comprises the amino acid sequence of SEQ ID NO: 37. In some embodiments, MITD comprises the amino acid sequence of SEQ ID NO: 36. In some embodiments, GS(1) comprises the amino acid sequence of SEQ ID NO: 38. In some embodiments, GS(2) comprises the amino acid sequence of SEQ ID NO: 39. In some embodiments, GS(3) comprises the amino acid sequence of SEQ ID NO: 39. In some embodiments, FI comprises the nucleotide sequence of SEQ ID NO: 13. In some embodiments, A30L70 comprises the nucleotide sequence of SEQ ID NO: 14.

In some embodiments, the sequence encoding the vaccine antigen/epitope comprises a modified nucleoside replacing (partially or completely, preferably completely) uridine, wherein the modified nucleoside is selected from the group consisting of pseudouridine (ψ), N1-methyl-pseudouridine (m1 ψ), and 5-methyl-uridine.

In some embodiments, the sequence encoding the vaccine antigen/epitope is codon-optimized.

In some embodiments, the G/C content of the sequence encoding the vaccine antigen/epitope is increased compared to the wild type coding sequence.

The term “professional antigen presenting cells” relates to antigen presenting cells which constitutively express the Major Histocompatibility Complex class 11 (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

The term “dendritic cell” (DC) refers to a subtype of phagocytic cells belonging to the class of antigen presenting cells. In some embodiments, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In some embodiments, the dendritic cells are splenic dendritic cells.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In some embodiments, the macrophages are splenic macrophages.

The term “allergen” refers to a kind of antigen which originates from outside the body of a subject (i.e., the allergen can also be called “heterologous antigen”) and which produces an abnormally vigorous immune response in which the immune system of the subject fights off a perceived threat that would otherwise be harmless to the subject. “Allergies” are the diseases caused by such vigorous immune reactions against allergens. An allergen usually is an antigen which is able to stimulate a type-I hypersensitivity reaction in atopic individuals through immunoglobulin E (IgE) responses. Particular examples of allergens include allergens derived from peanut proteins (e.g., Ara h 2.02), ovalbumin, grass pollen proteins (e.g., Phl p 5), and proteins of dust mites (e.g., Der p 2).

The term “growth factors” refers to molecules which are able to stimulate cellular growth, proliferation, healing, and/or cellular differentiation. Typically, growth factors act as signaling molecules between cells. The term “growth factors” include particular cytokines and hormones which bind to specific receptors on the surface of their target cells. Examples of growth factors include bone morphogenetic proteins (BMPs), fibroblast growth factors (FGFs), vascular endothelial growth factors (VEGFs), such as VEGFA, epidermal growth factor (EGF), insulin-like growth factor, ephrins, macrophage colony-stimulating factor, granulocyte colony-stimulating factor, granulocyte macrophage colony-stimulating factor, neuregulins, neurotrophins (e.g., brain-derived neurotrophic factor (BDNF), nerve growth factor (NGF)), placental growth factor (PGF), platelet-derived growth factor (PDGF), renalase (RNLS) (anti-apoptotic survival factor), T-cell growth factor (TCGF), thrombopoietin (TPO), transforming growth factors (transforming growth factor alpha (TGF-α), transforming growth factor beta (TGF-β)), and tumor necrosis factor-alpha (TNF-α). In one embodiment, a “growth factor” is a peptide or protein growth factor.

The term “protease inhibitors” refers to molecules, in particular peptides or proteins, which inhibit the function of proteases. Protease inhibitors can be classified by the protease which is inhibited (e.g., aspartic protease inhibitors) or by their mechanism of action (e.g., suicide inhibitors, such as serpins). Particular examples of protease inhibitors include serpins, such as alpha 1-antitrypsin, aprotinin, and bestatin.

The term “enzymes” refers to macromolecular biological catalysts which accelerate chemical reactions. Like any catalyst, enzymes are not consumed in the reaction they catalyze and do not alter the equilibrium of said reaction. Unlike many other catalysts, enzymes are much more specific. In one embodiment, an enzyme is essential for homeostasis of a subject, e.g., any malfunction (in particular, decreased activity which may be caused by any of mutation, deletion or decreased production) of the enzyme results in a disease. Examples of enzymes include herpes simplex virus type 1 thymidine kinase (HSV1-TK), hexosaminidase, phenylalanine hydroxylase, pseudocholinesterase, and lactase.

The term “receptors” refers to protein molecules which receive signals (in particular chemical signals called ligands) from outside a cell. The binding of a signal (e.g., ligand) to a receptor causes some kind of response of the cell, e.g., the intracellular activation of a kinase. Receptors include transmembrane receptors (such as ion channel-linked (ionotropic) receptors, G protein-linked (metabotropic) receptors, and enzyme-linked receptors) and intracellular receptors (such as cytoplasmic receptors and nuclear receptors). Particular examples of receptors include steroid hormone receptors, growth factor receptors, and peptide receptors (i.e., receptors whose ligands are peptides), such as P-selectin glycoprotein ligand-1 (PSGL-1). The term “growth factor receptors” refers to receptors which bind to growth factors.

The term “apoptosis regulators” refers to molecules, in particular peptides or proteins, which modulate apoptosis, i.e., which either activate or inhibit apoptosis. Apoptosis regulators can be grouped into two broad classes: those which modulate mitochondrial function and those which regulate caspases. The first class includes proteins (e.g., BCL-2, BCL-xL) which act to preserve mitochondrial integrity by preventing loss of mitochondrial membrane potential and/or release of pro-apoptotic proteins such as cytochrome C into the cytosol. Also to this first class belong proapoptotic proteins (e.g., BAX, BAK, BIM) which promote release of cytochrome C. The second class includes proteins such as the inhibitors of apoptosis proteins (e.g., X1AP) or FLIP which block the activation of caspases.

The term “transcription factors” relates to proteins which regulate the rate of transcription of genetic information from DNA to messenger RNA, in particular by binding to a specific DNA sequence. Transcription factors may regulate cell division, cell growth, and cell death throughout life; cell migration and organization during embryonic development; and/or in response to signals from outside the cell, such as a hormone. Transcription factors contain at least one DNA-binding domain which binds to a specific DNA sequence, usually adjacent to the genes which are regulated by the transcription factors. Particular examples of transcription factors include MECP2, FOXP2, FOXP3, the STAT protein family, and the HOX protein family.

The term “tumor suppressor proteins” relates to molecules, in particular peptides or proteins, which protect a cell from one step on the path to cancer. Tumor-suppressor proteins (usually encoded by corresponding tumor-suppressor genes) exhibit a weakening or repressive effect on the regulation of the cell cycle and/or promote apoptosis. Their functions may be one or more of the following: repression of genes essential for the continuing of the cell cycle; coupling the cell cycle to DNA damage (as long as damaged DNA is present in a cell, no cell division should take place); initiation of apoptosis, if the damaged DNA cannot be repaired; metastasis suppression (e.g., preventing tumor cells from dispersing, blocking loss of contact inhibition, and inhibiting metastasis); and DNA repair. Particular examples of tumor-suppressor proteins include p53, phosphatase and tensin homolog (PTEN), SWI/SNF (SWItch/Sucrose Non-Fermentable), von Hippel-Lindau tumor suppressor (pVHL), adenomatous polyposis coli (APC), CD95, suppression of tumorigenicity 5 (ST5), suppression of tumorigenicity 5 (ST5), suppression of tumorigenicity 14 (ST14), and Yippee-like 3 (YPEL3).

The term “structural proteins” refers to proteins which confer stiffness and rigidity to otherwise-fluid biological components. Structural proteins are mostly fibrous (such as collagen and elastin) but may also be globular (such as actin and tubulin). Usually, globular proteins are soluble as monomers, but polymerize to form long, fibers which, for example, may make up the cytoskeleton. Other structural proteins are motor proteins (such as myosin, kinesin, and dynein) which are capable of generating mechanical forces, and surfactant proteins. Particular examples of structural proteins include collagen, surfactant protein A, surfactant protein B, surfactant protein C, surfactant protein D, elastin, tubulin, actin, and myosin.

The term “reprogramming factors” or “reprogramming transcription factors” relates to molecules, in particular peptides or proteins, which, when expressed in somatic cells optionally together with further agents such as further reprogramming factors, lead to reprogramming or de-differentiation of said somatic cells to cells having stem cell characteristics, in particular pluripotency. Particular examples of reprogramming factors include OCT4, SOX2, c-MYC, KLF4, LIN28, and NANOG.

The term “genomic engineering proteins” relates to proteins which are able to insert, delete or replace DNA in the genome of a subject. Particular examples of genomic engineering proteins include meganucleases, zinc finger nucleases (ZFNs), transcription activator-like effector nucleases (TALENs), and clustered regularly spaced short palindromic repeat-CRISPR-associated protein 9 (CRISPR-Cas9).

The term “blood proteins” relates to peptides or proteins which are present in blood plasma ofa subject, in particular blood plasma of a healthy subject. Blood proteins have diverse functions such as transport (e.g., albumin, transferrin), enzymatic activity (e.g., thrombin or ceruloplasmin), blood clotting (e.g., fibrinogen), defense against pathogens (e.g., complement components and immunoglobulins), protease inhibitors (e.g., alpha 1-antitrypsin), etc. Particular examples of blood proteins include thrombin, serum albumin, Factor VII, Factor VIII, insulin, Factor IX, Factor X, tissue plasminogen activator, protein C, von Willebrand factor, antithrombin III, glucocerebrosidase, erythropoietin, granulocyte colony stimulating factor (G-CSF), modified Factor VIII, and anticoagulants.

Thus, in some embodiments, the pharmaceutically active peptide or protein is (i) a cytokine, preferably selected from the group consisting of erythropoietin (EPO), interleukin 4 (IL-2), and interleukin 10 (IL-11), more preferably EPO; (ii) an adhesion molecule, in particular an integrin; (iii) an immunoglobulin, in particular an antibody; (iv) an immunologically active compound, in particular an antigen, such as a viral or bacterial antigen, e.g., an antigen of SARS-CoV-2; (v) a hormone, in particular vasopressin, insulin or growth hormone; (vi) a growth factor, in particular VEGFA; (vii) a protease inhibitor, in particular alpha 1-antitrypsin; (viii) an enzyme, preferably selected from the group consisting of herpes simplex virus type 1 thymidine kinase (HSV1-TK), hexosaminidase, phenylalanine hydroxylase, pseudocholinesterase, pancreatic enzymes, and lactase; (ix) a receptor, in particular growth factor receptors; (x) an apoptosis regulator, in particular BAX; (xi) a transcription factor, in particular FOXP3; (xii) a tumor suppressor protein, in particular p53; (xiii) a structural protein, in particular surfactant protein B; (xiv) a reprogramming factor, e.g., selected from the group consisting of OCT4, SOX2, c-MYC, KLF4, LIN28 and NANOG; (xv) a genomic engineering protein, in particular clustered regularly spaced short palindromic repeat-CRISPR-associated protein 9 (CRISPR-Cas9); and (xvi) a blood protein, in particular fibrinogen.

In some embodiments, a pharmaceutically active peptide or protein comprises one or more antigens or one or more epitopes, i.e., administration of the peptide or protein to a subject elicits an immune response against the one or more antigens or one or more epitopes in a subject which may be therapeutic or partially or fully protective.

In certain embodiments, the RNA (preferably mRNA) encodes at least one epitope, e.g., at least two epitopes, at least three epitopes, at least four epitopes, at least five epitopes, at least six epitopes, at least seven epitopes, at least eight epitopes, at least nine epitopes, or at least ten epitopes.

In certain embodiments, the target antigen is a tumor antigen and the antigenic sequence (e.g., an epitope) is derived from the tumor antigen. The tumor antigen may be a “standard” antigen, which is generally known to be expressed in various cancers. The tumor antigen may also be a “neo-antigen”, which is specific to an individual's tumor and has not been previously recognized by the immune system.

A neo-antigen or neo-epitope may result from one or more cancer-specific mutations in the genome of cancer cells resulting in amino acid changes. If the tumor antigen is a neo-antigen, the vaccine antigen preferably comprises an epitope or a fragment of said neo-antigen comprising one or more amino acid changes.

Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AMLi, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A12, MAGE-B, MAGE-C, MART-1/Melan-A, MC1R, Myosin/m, MUC1, MUM-1, MUM-2, MUM-3, NA88-A, NFi, NY-ESO-1, NY-BR-1, p190 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by RNA (in particular mRNA) described herein, e.g., as a single polypeptide wherein the epitopes are optionally separated by linkers. In certain embodiments of the present disclosure, the RNA (in particular mRNA) encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include RNA (in particular, mRNA) that encodes at least five epitopes (termed a “pentatope”) and RNA (in particular, mRNA) that encodes at least ten epitopes (termed a “decatope”).

In certain embodiments, the epitope is derived from a pathogen-associated antigen. In some embodiments, the pharmaceutically active polypeptide and/or the antigen or epitope is derived from or is a protein of a pathogen, an immunogenic variant of the protein, or an immunogenic fragment of the protein or the immunogenic variant thereof.

In some embodiments, the pathogen is selected from viruses, bacteria, fungi, parasites, and other microorganisms.

Exemplary viruses include, but are not limited to, are severe acute respiratory syndrome coronavirus (SARS-CoV), such as SARS-CoV2, human immunodeficiency virus (HIV), Epstein-Barr virus (EBV), cytomegalovirus (CMV) (e.g., CMV5), human herpesviruses (HHV) (e.g., HHV6, 7 or 8), herpes simplex viruses (HSV), bovine herpes virus (BHV) (e.g., BHV4), equine herpes virus (EHV) (e.g., EHV2), human T-CeIl leukemia viruses (HTLV)5, Varicella-Zoster virus (VZV), measles virus, papovaviruses (JC and BK), hepatitis viruses (e.g., HBV or HCV), myxoma virus, adenoviruses, rhinoviruses, enteroviruses, parvoviruses, polyoma virus, influenza viruses, papillomaviruses (such as human papillomavirus (HPV)), poxviruses such as vaccinia virus, and molluscum contagiosum virus (MCV), lyssaviruses, rotaviruses, noroviruses, rubella viruses, and mumps viruses. Exemplary diseases caused by viral infection include, but are not limited to, SARS, acquired immune deficiency syndrome (AIDS), measles, chicken pox, cytomegalovirus infections, genital herpes, hepatitis (such as hepatitis B or C), influenza (flu, such as human flu, swine flu, dog flu, horse flu, and avian flu), HPV infection, shingles, rabies, common cold, gastroenteritis, rubella, and mumps.

Exemplary bacteria include, but are not limited to, Campylobacter (such as Campylobacter jejuni), Enterobacter species, Enterococcus faecium, Enterococcus faecalis, Escherichia coli (e.g., E. coli O157:H7), Group A streptococci, Haemophilus influenzae, Helicobacterpylori, listeria, Mycobacterium tuberculosis, Pseudomonas aeruginosa, S. pneumoniae, Salmonella, Shigella, Staphylococcus aureus, Staphylococcus epidermidis, Borrelia and Rickettsia, Chlamydiaceae, Neisseria gonorrhoeae, Bordetella pertussis, Clostridium tetani, Neisseria meningitidis, Streptococcus (such as Streptococcus pneumoniae or Streptococcus pyogenes), and Treponema pallidum. Exemplary diseases caused by bacterial infection include, but are not limited to, anthrax, cholera, diphtheria, foodborne illnesses, leprosy, meningitis, peptic ulcer disease, pneumonia, sepsis, septic shock, tetanus, tuberculosis, typhoid fever, urinary tract infection, Lyme disease, Rocky Mountain spotted fever, chlamydia, gonorrhea, pertussis, tetanus, meningitis, scarlet fever, and syphilis.

Exemplary parasites include, but are not limited to, Plasmodium, Tiypanosoma, Leishmania, Trichomonas, Dientamoeba, Giardia, Entamoeba histolytica, Naegleria, isospora, Toxoplasma, Sarcocystis, Rhinosporidium seeberi, and Balantidium. Exemplary diseases caused by parasite infection include, but are not limited to, malaria, trypanosomiasis, Chagas disease, leishmaniasis, trichomoniasis, dientamoebiasis, giardiasis, amebic dysentery, coccidiosis, toxoplasmosis, sarcocystosis, rhinosporidiosis, and balantidiasis.

In some embodiments, the pathogen is an infectious pathogen, in particular a pathogen causing an infectious disease, such as a viral disease, a bacterial disease, or a parasitic disease. In some embodiments, the pathogen is a virus, bacterium, or parasite. Thus, in these embodiments, the RNA (in particular mRNA) and/or compositions described herein can be used to prevent and/or treat an infectious disease caused by said pathogen.

In certain embodiments, the epitope is derived from a viral antigen.

In some embodiments, the antigen or epitope is derived from a coronavirus protein, an immunogenic variant thereof, or an immunogenic fragment of the coronavirus protein or the immunogenic variant thereof. Thus, in some embodiments, the mRNA used in the present disclosure encodes an amino acid sequence comprising a coronavirus protein, an immunogenic variant thereof, or an immunogenic fragment of the coronavirus protein or the immunogenic variant thereof.

In some embodiments, the antigen or epitope is derived from a coronavirus S protein, an immunogenic variant thereof, or an immunogenic fragment of the coronavirus S protein or the immunogenic variant thereof. Thus, in some embodiments, the RNA (in particular, mRNA) described in the present disclosure encodes an amino acid sequence comprising a coronavirus S protein, an immunogenic variant thereof, or an immunogenic fragment of the coronavirus S protein or the immunogenic variant thereof. In some embodiments, the coronavirus is MERS-CoV. In some embodiments, the coronavirus is SARS-CoV. In some embodiments, the coronavirus is SARS-CoV-2.

Coronaviruses are enveloped, positive-sense, single-stranded RNA ((+) ssRNA) viruses. They have the largest genomes (26-32 kb) among known RNA viruses and are phylogenetically divided into four genera (α, β, γ, and δ), with betacoronaviruses further subdivided into four lineages (A, B, C, and D). Coronaviruses infect a wide range of avian and mammalian species, including humans. Some human coronaviruses generally cause mild respiratory diseases, although severity can be greater in infants, the elderly, and the immunocompromised. Middle East respiratory syndrome coronavirus (MERS-CoV) and severe acute respiratory syndrome coronavirus (SARS-CoV), belonging to betacoronavirus lineages C and B, respectively, are highly pathogenic. Both viruses emerged into the human population from animal reservoirs within the last 15 years and caused outbreaks with high case-fatality rates. The outbreak of severe acute respiratory syndrome coronavirus-2 (SARS-CoV-2) that causes atypical pneumonia (coronavirus disease 2019; COVID-19) has raged in China since mid-December 2019, and has developed to be a public health emergency of international concern. SARS-CoV-2 (MN908947.3) belongs to betacoronavirus lineage B. It has at least 70% sequence similarity to SARS-CoV.

In general, coronaviruses have four structural proteins, namely, envelope (E), membrane (M), nucleocapsid (N), and spike (S). The E and M proteins have important functions in the viral assembly, and the N protein is necessary for viral RNA synthesis. The critical glycoprotein S is responsible for virus binding and entry into target cells. The S protein is synthesized as a single-chain inactive precursor that is cleaved by furin-like host proteases in the producing cell into two noncovalently associated subunits, S1 and S2. The S1 subunit contains the receptor-binding domain (RBD), which recognizes the host-cell receptor. The S2 subunit contains the fusion peptide, two heptad repeats, and a transmembrane domain, all of which are required to mediate fusion of the viral and host-cell membranes by undergoing a large conformational rearrangement. The S1 and S2 subunits trimerize to form a large prefusion spike.

The S precursor protein of SARS-CoV-2 can be proteolytically cleaved into S1 (685 aa) and S2 (588 aa) subunits. The S1 subunit comprises the receptor-binding domain (RBD), which mediates virus entry into sensitive cells through the host angiotensin-converting enzyme 2 (ACE2) receptor.

In some embodiments, the antigen or epitope is derived from a SARS-CoV-2 S protein, an immunogenic variant thereof, or an immunogenic fragment of the SARS-CoV-2 S protein or the immunogenic variant thereof. Thus, in some embodiments, the RNA (preferably mRNA) described in the present disclosure encodes an amino acid sequence comprising a SARS-CoV-2 S protein, an immunogenic variant thereof, or an immunogenic fragment of the SARS-CoV-2 S protein or the immunogenic variant thereof. Thus, in some embodiments, the encoded amino acid sequence comprises an epitope of SARS-CoV-2 S protein or an immunogenic variant thereof for inducing an immune response against coronavirus S protein, in particular SARS-CoV-2 S protein in a subject. In some embodiments, the RNA (preferably mRNA) comprises an ORF encoding a full-length SARS-CoV2 S protein variant with proline residue substitutions at positions 986 and 987 of SEQ ID NO:1. In some embodiments, the SARS-CoV2 S protein variant has at least 80% identity (such as at least 85% identity, at least 90% identity, at least 91% identity, at least 92% identity, at least 93% identity, at least 94% identity, at least 95% identity, at least 96% identity, at least 97% identity, at least 98% identity, or at least 99% identity) to SEQ ID NO:7.

In some embodiments, RNA (in particular, mRNA) is administered to provide (following expression by appropriate target cells) antigen for induction of an immune response, e.g., antibodies and/or immune effector cells, which is targeted to target antigen (coronavirus S protein, in particular SARS-CoV-2 S protein) or a procession product thereof. In some embodiments, the immune response which is to be induced according to the present disclosure is a B cell-mediated immune response, i.e., an antibody-mediated immune response. Additionally or alternatively, in some embodiments, the immune response which is to be induced according to the present disclosure is a T cell-mediated immune response. In some embodiments, the immune response is an anti-coronavirus, in particular anti-SARS-CoV-2 immune response.

In some embodiments, an immunogenic fragment of the SARS-CoV-2 S protein comprises the S1 subunit of the SARS-CoV-2 S protein, or the receptor binding domain (RBD) of the S1 subunit of the SARS-CoV-2 S protein. In some embodiments, the RNA (e.g., mRNA) described in the present disclosure comprises an open reading frame encoding a polypeptide that comprises a receptor-binding portion of a SARS-CoV-2 S protein, which RNA is suitable for intracellular expression of the polypeptide. In some embodiments, such an encoded polypeptide does not comprise the complete S protein. In some embodiments, the encoded polypeptide comprises the receptor binding domain (RBD), for example, as shown in SEQ ID NO: 5. In some embodiments, the encoded polypeptide comprises the peptide according to SEQ ID NO: 29 or 31.

SARS-CoV-2 coronavirus full length spike (S) protein consist of 1273 amino acids and has the following amino acid sequence shown in SEQ ID NO: 1. For purposes of the present disclosure, the amino acid sequence shown in SEQ ID NO: 1 is considered the wildtype SARS-CoV-2 S protein amino acid sequence. Position numberings in SARS-CoV-2 S protein given herein are in relation to the amino acid sequence according to SEQ ID NO: 1 and corresponding positions in SARS-CoV-2 S protein variants.

In specific embodiments, full length spike (S) protein according to SEQ ID NO: 1 is modified in such a way that the prototypical prefusion conformation is stabilized. Stabilization of the prefusion conformation may be obtained by introducing two consecutive proline substitutions at amino acid residues 986 and 987 in the full-length spike protein. Specifically, spike (S) protein stabilized protein variants are obtained in a way that the amino acid residue at position 986 is exchanged to proline and the amino acid residue at position 987 is also exchanged to proline. In some embodiments, a SARS-CoV-2 S protein variant wherein the prototypical prefusion conformation is stabilized comprises the amino acid sequence shown in SEQ ID NO: 7.

Those skilled in the art are aware of various spike variants, and/or resources that document them.

In some embodiments, RNA (in particular, mRNA) described herein (e.g., contained in the compositions of the present disclosure and/or used in the methods of the present disclosure) encodes an amino acid sequence which comprises, consists essentially of or consists of a spike (S) protein of SARS-CoV-2, a variant thereof, or a fragment thereof.

The amino acid sequence of amino acids 327 to 528 of SEQ ID NO: 1, a variant thereof, or a fragment thereof is also referred to herein as “RBD” or “RBD domain”.

In some embodiments, the amino acid sequence comprising a SARS-CoV-2 S protein, an immunogenic variant thereof, or an immunogenic fragment of the SARS-CoV-2 S protein or the immunogenic variant thereof is able to form a multimeric complex, in particular a trimeric complex. To this end, the amino acid sequence comprising a SARS-CoV-2 S protein, an immunogenic variant thereof, or an immunogenic fragment ofthe SARS-CoV-2 S protein or the immunogenic variant thereof may comprise a domain allowing the formation of a multimeric complex, in particular a trimeric complex of the amino acid sequence comprising a SARS-CoV-2 S protein, an immunogenic variant thereof, or an immunogenic fragment of the SARS-CoV-2 S protein or the immunogenic variant thereof. In some embodiments, the domain allowing the formation of a multimeric complex comprises a trimerization domain, for example, a trimerization domain as described herein.

In some embodiments, the trimerization domain is fused, either directly or through a linker, e.g., a glycine/serine linker, to a SARS-CoV-2 S protein, a variant thereof, or a fragment thereof, i.e., the antigenic peptide or protein. Accordingly, in some embodiments, a trimerization domain is fused to the above described amino acid sequences derived from SARS-CoV-2 S protein or immunogenic fragments thereof (antigenic peptides or proteins) comprised by the encoded amino acid sequences described above (which may optionally be fused to a signal peptide as described above).

Such trimerization domains are preferably located at the C-terminus of the antigenic peptide or protein, without being limited thereto. Trimerization domains as defined herein preferably allow the trimerization of the antigenic peptide or protein as encoded by the RNA. Examples of trimerization domains as defined herein include, without being limited thereto, foldon, the natural trimerization domain of T4 fibritin. The C-terminal domain of T4 fibritin (foldon) is obligatory for the formation of the fibritin trimer structure and can be used as an artificial trimerization domain. In some embodiments, the trimerization domain as defined herein includes, without being limited thereto, a sequence comprising the amino acid sequence of SEQ ID NO: 10 or a functional variant thereof.

In some embodiments, the trimerization domain as defined herein includes, without being limited thereto, a sequence comprising the amino acid sequence of amino acids 3 to 29 of SEQ ID NO: 10 or a functional variant thereof. In one embodiment, the trimerization domain as defined herein includes, without being limited thereto, a sequence comprising the amino acid sequence of SEQ ID NO: 10 or a functional variant thereof.

In some embodiments, a trimerization domain comprises the amino acid sequence of amino acids 3 to 29 of SEQ ID NO: 10, an amino acid sequence having at least 99%, 98%, 97%, 96%, 95%, 90%, 85%, or 80% identity to the amino acid sequence of amino acids 3 to 29 of SEQ ID NO: 10, or a functional fragment of the amino acid sequence of amino acids 3 to 29 of SEQ ID NO: 10, or the amino acid sequence having at least 99%, 98%, 97%, 96%, 95%, 90%, 85%, or 80% identity to the amino acid sequence of amino acids 3 to 29 of SEQ ID NO: 10. In some embodiments, a trimerization domain comprises the amino acid sequence of amino acids 3 to 29 of SEQ ID NO: 10.

In some embodiments, RNA encoding a trimerization domain (i) comprises the nucleotide sequence of nucleotides 7 to 87 of SEQ ID NO: 11, a nucleotide sequence having at least 99%, 98%, 97%, 96%, 95%, 90%, 85%, or 80% identity to the nucleotide sequence of nucleotides 7 to 87 of SEQ ID NO: 11, or a fragment of the nucleotide sequence of nucleotides 7 to 87 of SEQ ID NO: 11, or the nucleotide sequence having at least 99%, 98%, 97%, 96%, 95%, 90%, 85%, or 80% identity to the nucleotide sequence of nucleotides 7 to 87 of SEQ ID NO: 11; and/or (ii) encodes an amino acid sequence comprising the amino acid sequence of amino acids 3 to 29 of SEQ ID NO: 10, an amino acid sequence having at least 99%, 98%, 97%, 96%, 95%, 90%, 85%, or 80% identity to the amino acid sequence of amino acids 3 to 29 of SEQ ID NO: 10, or a functional fragment of the amino acid sequence of amino acids 3 to 29 of SEQ ID NO: 10, or the amino acid sequence having at least 99%, 98%, 97%, 96%, 95%, 90%, 85%, or 80% identity to the amino acid sequence of amino acids 3 to 29 of SEQ ID NO: 10. In some embodiments, RNA encoding a trimerization domain (i) comprises the nucleotide sequence of nucleotides 7 to 87 of SEQ ID NO: 11; and/or (ii) encodes an amino acid sequence comprising the amino acid sequence of amino acids 3 to 29 of SEQ ID NO: 10.

In some embodiments, the RBD antigen expressed by an RNA encoding a SARS-CoV-2 S protein, an immunogenic variant thereof, or an immunogenic fragment of the SARS-CoV-2 S protein or the immunogenic variant thereof (e.g., as described herein) can be modified by addition of a T4-fibritin-derived “foldon” trimerization domain, for example, to increase its immunogenicity.

In some embodiments, the amino acid sequence comprising a SARS-CoV-2 S protein, an immunogenic variant thereof, or an immunogenic fragment of the SARS-CoV-2 S protein or the immunogenic variant thereof is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 979 to 1584 of SEQ ID NO: 2, 8 or 9, a nucleotide sequence having at least 99%, 98%, 97%, 96%, 95%, 90%, 85%, or 80% identity to the nucleotide sequence of nucleotides 979 to 1584 of SEQ ID NO: 2, 8 or 9, or a fragment of the nucleotide sequence of nucleotides 979 to 1584 of SEQ ID NO: 2, 8 or 9, or the nucleotide sequence having at least 99%, 98%, 97%, 96%, 95%, 90%, 85%, or 80% identity to the nucleotide sequence of nucleotides 979 to 1584 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 327 to 528 of SEQ ID NO: 1, an amino acid sequence having at least 99%, 98%, 97%, 96%, 95%, 90%, 85%, or 80% identity to the amino acid sequence of amino acids 327 to 528 of SEQ ID NO: 1, or an immunogenic fragment of the amino acid sequence of amino acids 327 to 528 of SEQ ID NO: 1, or the amino acid sequence having at least 99%, 98%, 97%, 96%, 95%, 90%, 85%, or 80% identity to the amino acid sequence of amino acids 327 to 528 of SEQ ID NO: 1.

In some embodiments,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 2055 of SEQ ID NO: 2, 8 or 9, a nucleotide sequence having at least 99%, 98%, 97%, 96%, 95%, 90%, 85%, or 80% identity to the nucleotide sequence of nucleotides 49 to 2055 of SEQ ID NO: 2, 8 or 9, or a fragment of the nucleotide sequence of nucleotides 49 to 2055 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2055 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 685 of SEQ ID NO: 1, an amino acid sequence having at least 99%, 98%, 97%, 96%, 95%, 90%, 85%, or 80% identity to the amino acid sequence of amino acids 17 to 685 of SEQ ID NO: 1, or an immunogenic fragment of the amino acid sequence of amino acids 17 to 685 of SEQ ID NO: 1, or the amino acid sequence having at least 99%, 98%, 97%, 96%, 95%, 90%, 85%, or 80% identity to the amino acid sequence of amino acids 17 to 685 of SEQ ID NO: 1.

In some embodiments,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 3819 of SEQ ID NO: 2, 8 or 9, a nucleotide sequence having at least 99%, 98%, 97%, 96%, 95%, 90%, 85%, or 80% identity to the nucleotide sequence of nucleotides 49 to 3819 of SEQ ID NO: 2, 8 or 9, or a fragment of the nucleotide sequence of nucleotides 49 to 3819 of SEQ ID NO: 2, 8 or 9, or the nucleotide sequence having at least 99%, 98%, 97%, 96%, 95%, 90%, 85%, or 80% identity to the nucleotide sequence of nucleotides 49 to 3819 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 1273 of SEQ ID NO: 1 or 7, an amino acid sequence having at least 99%, 98%, 97%, 96%, 95%, 90%, 85%, or 80% identity to the amino acid sequence of amino acids 17 to 1273 of SEQ ID NO: 1 or 7, or an immunogenic fragment of the amino acid sequence of amino acids 17 to 1273 of SEQ ID NO: 1 or 7, or the amino acid sequence having at least 99%, 98%, 97%, 96%, 95%, 90%, 85%, or 80% identity to the amino acid sequence of amino acids 17 to 1273 of SEQ ID NO: 1 or 7.

In some embodiments,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 2049 of SEQ ID NO: 2, 8 or 9, a nucleotide sequence having at least 99%, 98%, 97%, 96%, 95%, 90%, 85%, or 80% identity to the nucleotide sequence of nucleotides 49 to 2049 of SEQ ID NO: 2, 8 or 9, or a fragment of the nucleotide sequence of nucleotides 49 to 2049 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2049 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 683 of SEQ ID NO: 1, an amino acid sequence having at least 99%, 98%, 97%, 96%, 95%, 90%, 85%, or 80% identity to the amino acid sequence of amino acids 17 to 683 of SEQ ID NO: 1, or an immunogenic fragment of the amino acid sequence of amino acids 17 to 683 of SEQ ID NO: 1, or the amino acid sequence having at least 99%, 98%, 97%, 96%, 95%, 90%, 85%, or 80% identity to the amino acid sequence of amino acids 17 to 683 of SEQ ID NO: 1. In one embodiment, RNA encoding a vaccine antigen (i) comprises the nucleotide sequence of nucleotides 49 to 2049 of SEQ ID NO: 2, 8 or 9; and/or (ii) encodes an amino acid sequence comprising the amino acid sequence of amino acids 17 to 683 of SEQ ID NO: 1.

In some embodiments, the encoded amino acid sequence comprises the amino acid sequence of amino acids 17 to 683 of SEQ ID NO: 1, an amino acid sequence having at least 99%, 98%, 97%, 96%, 95%, 90%, 85%, or 80% identity to the amino acid sequence of amino acids 17 to 683 of SEQ ID NO: 1, or an immunogenic fragment of the amino acid sequence of amino acids 17 to 683 of SEQ ID NO: 1, or the amino acid sequence having at least 99%, 98%, 97%, 96%, 95%, 90%, 85%, or 80% identity to the amino acid sequence of amino acids 17 to 683 of SEQ ID NO: 1. In some embodiments, the encoded amino acid sequence comprises the amino acid sequence of amino acids 17 to 683 of SEQ ID NO: 1.

In some embodiments, the encoded amino acid sequence comprises the amino acid sequence of amino acids 1 to 683 of SEQ ID NO: 1, an amino acid sequence having at least 99%, 98%, 97%, 96%, 95%, 90%, 85%, or 80% identity to the amino acid sequence of amino acids 1 to 683 of SEQ ID NO: 1, or an immunogenic fragment of the amino acid sequence of amino acids 1 to 683 of SEQ ID NO: 1, or the amino acid sequence having at least 99%, 98%, 97%, 96%, 95%, 90%, 85%, or 80% identity to the amino acid sequence of amino acids 1 to 683 of SEQ ID NO: 1. In some embodiments, the encoded amino acid sequence comprises the amino acid sequence of amino acids 1 to 683 of SEQ ID NO: 1.

In some embodiments, the encoded amino acid sequence comprises the amino acid sequence of amino acids 1 to 685 of SEQ ID NO: 1, an amino acid sequence having at least 99%, 98%, 97%, 96%, 95%, 90%, 85%, or 80% identity to the amino acid sequence of amino acids 1 to 685 of SEQ ID NO: 1, or an immunogenic fragment of the amino acid sequence of amino acids 1 to 685 of SEQ ID NO: 1, or the amino acid sequence having at least 99%, 98%, 97%, 96%, 95%, 90%, 85%, or 80% identity to the amino acid sequence of amino acids 1 to 685 of SEQ ID NO: 1. In some embodiments, the encoded amino acid sequence comprises the amino acid sequence of amino acids 1 to 685 of SEQ ID NO: 1.

In some embodiments, the amino acid sequence comprising a SARS-CoV-2 S protein, an immunogenic variant thereof, or an immunogenic fragment of the SARS-CoV-2 S protein or the immunogenic variant thereof comprises a secretory signal peptide.

In some embodiments, the secretory signal peptide is fused, preferably N-terminally, to a SARS-CoV-2 S protein, an immunogenic variant thereof, or an immunogenic fragment of the SARS-CoV-2 S protein or the immunogenic variant thereof.

In some embodiments,
(i) the RNA encoding the secretory signal peptide comprises the nucleotide sequence of nucleotides 1 to 48 of SEQ ID NO: 2, 8 or 9, a nucleotide sequence having at least 99%, 98%, 97%, 96%, 95%, 90%, 85%, or 80% identity to the nucleotide sequence of nucleotides 1 to 48 of SEQ ID NO: 2, 8 or 9, or a fragment of the nucleotide sequence of nucleotides 1 to 48 of SEQ ID NO: 2, 8 or 9, or the nucleotide sequence having at least 99%, 98%, 97%, 96%, 95%, 90%, 85%, or 80% identity to the nucleotide sequence of nucleotides 1 to 48 of SEQ ID NO: 2, 8 or 9; and/or
(ii) the secretory signal peptide comprises the amino acid sequence of amino acids 1 to 16 of SEQ ID NO: 1, an amino acid sequence having at least 99%, 98%, 97%, 96%, 95%, 90%, 85%, or 80% identity to the amino acid sequence of amino acids 1 to 16 of SEQ ID NO: 1, or a functional fragment of the amino acid sequence of amino acids 1 to 16 of SEQ ID NO: 1, or the amino acid sequence having at least 99%, 98%, 97%, 96%, 95%, 90%, 85%, or 80% identity to the amino acid sequence of amino acids 1 to 16 of SEQ ID NO: 1.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6, or the nucleotide sequence having at least 99%, 98%, 97%, 96%, 95%, 90%, 85%, or 80% identity to the nucleotide sequence of SEQ ID NO: 6; and/or
(ii) encodes an amino acid sequence comprising the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 4, a nucleotide sequence having at least 99%, 98%, 97%, 96%, 95%, 90%, 85%, or 80% identity to the nucleotide sequence of SEQ ID NO: 4, or a fragment of the nucleotide sequence of SEQ ID NO: 4, or the nucleotide sequence having at least 99%, 98%, 97%, 96%, 95%, 90%, 85%, or 80% identity to the nucleotide sequence of SEQ ID NO: 4; and/or
(ii) encodes an amino acid sequence comprising the amino acid sequence of SEQ ID NO: 3, an amino acid sequence having at least 99%, 98%, 97%, 96%, 95%, 90%, 85%, or 80% identity to the amino acid sequence of SEQ ID NO: 3, or an immunogenic fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of SEQ ID NO: 4; and/or (ii) encodes an amino acid sequence comprising the amino acid sequence of SEQ ID NO: 3.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 716 of SEQ ID NO: 30, a nucleotide sequence having at least 99%, 98%, 97%, 96%, 95%, 90%, 85%, or 80% identity to the nucleotide sequence of nucleotides 54 to 716 of SEQ ID NO: 30, or a fragment of the nucleotide sequence of nucleotides 54 to 716 of SEQ ID NO: 30, or the nucleotide sequence having at least 99%, 98%, 97%, 96%, 95%, 90%, 85%, or 80% identity to the nucleotide sequence of nucleotides 54 to 716 of SEQ ID NO: 30; and/or
(ii) encodes an amino acid sequence comprising the amino acid sequence of amino acids 1 to 221 of SEQ ID NO: 29, an amino acid sequence having at least 99%, 98%, 97%, 96%, 95%, 90%, 85%, or 80% identity to the amino acid sequence of amino acids 1 to 221 of SEQ ID NO: 29, or an immunogenic fragment of the amino acid sequence of amino acids 1 to 221 of SEQ ID NO: 29, or the amino acid sequence having at least 99%, 98%, 97%, 96%, 95%, 90%, 85%, or 80% identity to the amino acid sequence of amino acids 1 to 221 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 716 of SEQ ID NO: 30; and/or (ii) encodes an amino acid sequence comprising the amino acid sequence of amino acids 1 to 221 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 725 of SEQ ID NO: 32, a nucleotide sequence having at least 99%, 98%, 97%, 96%, 95%, 90%, 85%, or 80% identity to the nucleotide sequence of nucleotides 54 to 725 of SEQ ID NO: 32, or a fragment of the nucleotide sequence of nucleotides 54 to 725 of SEQ ID NO: 32, or the nucleotide sequence having at least 99%, 98%, 97%, 96%, 95%, 90%, 85%, or 80% identity to the nucleotide sequence of nucleotides 54 to 725 of SEQ ID NO: 32; and/or
(ii) encodes an amino acid sequence comprising the amino acid sequence of amino acids 1 to 224 of SEQ ID NO: 31, an amino acid sequence having at least 99%, 98%, 97%, 96%, 95%, 90%, 85%, or 80% identity to the amino acid sequence of amino acids 1 to 224 of SEQ ID NO: 31, or an immunogenic fragment of the amino acid sequence of amino acids 1 to 224 of SEQ ID NO: 31, or the amino acid sequence having at least 99%, 98%, 97%, 96%, 95%, 90%, 85%, or 80% identity to the amino acid sequence of amino acids 1 to 224 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 17, 21, or 26, a nucleotide sequence having at least 99%, 98%, 97%, 96%, 95%, 90%, 85%, or 80% identity to the nucleotide sequence of SEQ ID NO: 17, 21, or 26, or a fragment of the nucleotide sequence of SEQ ID NO: 17, 21, or 26, or the nucleotide sequence having at least 99%, 98%, 97%, 96%, 95%, 90%, 85%, or 80% identity to the nucleotide sequence of SEQ ID NO: 17, 21, or 26; and/or
(ii) encodes an amino acid sequence comprising the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of SEQ ID NO: 17, 21, or 26; and/or (ii) encodes an amino acid sequence comprising the amino acid sequence of SEQ ID NO: 5.

In some embodiments, a vaccine antigen comprises the amino acid sequence of SEQ ID NO: 18, an amino acid sequence having at least 99%, 98%, 97%, 96%, 95%, 90%, 85%, or 80% identity to the amino acid sequence of SEQ ID NO: 18, or an immunogenic fragment of the amino acid sequence of SEQ ID NO: 18, or the amino acid sequence having at least 99%, 98%, 97%, 96%, 95%, 90%, 85%, or 80% identity to the amino acid sequence of SEQ ID NO: 18. In one embodiment, a vaccine antigen comprises the amino acid sequence of SEQ ID NO: 18.

In some embodiments, a vaccine antigen comprises the amino acid sequence of amino acids 1 to 257 of SEQ ID NO: 29, an amino acid sequence having at least 99%, 98%, 97%, 96%, 95%, 90%, 85%, or 80% identity to the amino acid sequence of amino acids 1 to 257 of SEQ ID NO: 29, or an immunogenic fragment of the amino acid sequence of amino acids 1 to 257 of SEQ ID NO: 29, or the amino acid sequence having at least 99%, 98%, 97%, 96%, 95%, 90%, 85%, or 80% identity to the amino acid sequence of amino acids 1 to 257 of SEQ ID NO: 29. In one embodiment, a vaccine antigen comprises the amino acid sequence of amino acids 1 to 257 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 824 of SEQ ID NO: 30, a nucleotide sequence having at least 99%, 98%, 97%, 96%, 95%, 90%, 85%, or 80% identity to the nucleotide sequence of nucleotides 54 to 824 of SEQ ID NO: 30, or a fragment of the nucleotide sequence of nucleotides 54 to 824 of SEQ ID NO: 30, or the nucleotide sequence having at least 99%, 98%, 97%, 96%, 95%, 90%, 85%, or 80% identity to the nucleotide sequence of nucleotides 54 to 824 of SEQ ID NO: 30; and/or
(ii) encodes an amino acid sequence comprising the amino acid sequence of amino acids 1 to 257 of SEQ ID NO: 29, an amino acid sequence having at least 99%, 98%, 97%, 96%, 95%, 90%, 85%, or 80% identity to the amino acid sequence of amino acids 1 to 257 of SEQ ID NO: 29, or an immunogenic fragment of the amino acid sequence of amino acids 1 to 257 of SEQ ID NO: 29, or the amino acid sequence having at least 99%, 98%, 97%, 96%, 95%, 90%, 85%, or 80% identity to the amino acid sequence of amino acids 1 to 257 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 824 of SEQ ID NO: 30; and/or (ii) encodes an amino acid sequence comprising the amino acid sequence of amino acids 1 to 257 of SEQ ID NO: 29.

In some embodiments, a vaccine antigen comprises the amino acid sequence of amino acids 1 to 260 of SEQ ID NO: 31, an amino acid sequence having at least 99%, 98%, 97%, 96%, 95%, 90%, 85%, or 80% identity to the amino acid sequence of amino acids 1 to 260 of SEQ ID NO: 31, or an immunogenic fragment of the amino acid sequence of amino acids 1 to 260 of SEQ ID NO: 31, or the amino acid sequence having at least 99%, 98%, 97%, 96%, 95%, 90%, 85%, or 80% identity to the amino acid sequence of amino acids 1 to 260 of SEQ ID NO: 31. In one embodiment, a vaccine antigen comprises the amino acid sequence of amino acids 1 to 260 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 833 of SEQ ID NO: 32, a nucleotide sequence having at least 99%, 98%, 97%, 96%, 95%, 90%, 85%, or 80% identity to the nucleotide sequence of nucleotides 54 to 833 of SEQ ID NO: 32, or a fragment of the nucleotide sequence of nucleotides 54 to 833 of SEQ ID NO: 32, or the nucleotide sequence having at least 99%, 98%, 97%, 96%, 95%, 90%, 85%, or 80% identity to the nucleotide sequence of nucleotides 54 to 833 of SEQ ID NO: 32; and/or
(ii) encodes an amino acid sequence comprising the amino acid sequence of amino acids 1 to 260 of SEQ ID NO: 31, an amino acid sequence having at least 99%, 98%, 97%, 96%, 95%, 90%, 85%, or 80% identity to the amino acid sequence of amino acids 1 to 260 of SEQ ID NO: 31, or an immunogenic fragment of the amino acid sequence of amino acids 1 to 260 of SEQ ID NO: 31, or the amino acid sequence having at least 99%, 98%, 97%, 96%, 95%, 90%, 85%, or 80% identity to the amino acid sequence of amino acids 1 to 260 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 833 of SEQ ID NO: 32; and/or (ii) encodes an amino acid sequence comprising the amino acid sequence of amino acids 1 to 260 of SEQ ID NO: 31.

In some embodiments, a vaccine antigen comprises the amino acid sequence of amino acids 20 to 257 of SEQ ID NO: 29, an amino acid sequence having at least 99%, 98%, 97%, 96%, 95%, 90%, 85%, or 80% identity to the amino acid sequence of amino acids 20 to 257 of SEQ ID NO: 29, or an immunogenic fragment of the amino acid sequence of amino acids 20 to 257 of SEQ ID NO: 29, or the amino acid sequence having at least 99%, 98%, 97%, 96%, 95%, 90%, 85%, or 80% identity to the amino acid sequence of amino acids 20 to 257 of SEQ ID NO: 29. In one embodiment, a vaccine antigen comprises the amino acid sequence of amino acids 20 to 257 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 111 to 824 of SEQ ID NO: 30, a nucleotide sequence having at least 99%, 98%, 97%, 96%, 95%, 90%, 85%, or 80% identity to the nucleotide sequence of nucleotides 111 to 824 of SEQ ID NO: 30, or a fragment of the nucleotide sequence of nucleotides 111 to 824 of SEQ ID NO: 30, or the nucleotide sequence having at least 99%, 98%, 97%, 96%, 95%, 90%, 85%, or 80% identity to the nucleotide sequence of nucleotides 111 to 824 of SEQ ID NO: 30; and/or
(ii) encodes an amino acid sequence comprising the amino acid sequence of amino acids 20 to 257 of SEQ ID NO: 29, an amino acid sequence having at least 99%, 98%, 97%, 96%, 95%, 90%, 85%, or 80% identity to the amino acid sequence of amino acids 20 to 257 of SEQ ID NO: 29, or an immunogenic fragment of the amino acid sequence of amino acids 20 to 257 of SEQ ID NO: 29, or the amino acid sequence having at least 99%, 98%, 97%, 96%, 95%, 90%, 85%, or 80% identity to the amino acid sequence of amino acids 20 to 257 of SEQ ID NO: 29. In one embodiment, RNA encoding a vaccine antigen (i) comprises the nucleotide sequence of nucleotides 111 to 824 of SEQ ID NO: 30; and/or (ii) encodes an amino acid sequence comprising the amino acid sequence of amino acids 20 to 257 of SEQ ID NO: 29.

In some embodiments, a vaccine antigen comprises the amino acid sequence of amino acids 23 to 260 of SEQ ID NO: 31, an amino acid sequence having at least 99%, 98%, 97%, 96%, 95%, 90%, 85%, or 80% identity to the amino acid sequence of amino acids 23 to 260 of SEQ ID NO: 31, or an immunogenic fragment of the amino acid sequence of amino acids 23 to 260 of SEQ ID NO: 31, or the amino acid sequence having at least 99%, 98%, 97%, 96%, 95%, 90%, 85%, or 80% identity to the amino acid sequence of amino acids 23 to 260 of SEQ ID NO: 31. In one embodiment, a vaccine antigen comprises the amino acid sequence of amino acids 23 to 260 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 120 to 833 of SEQ ID NO: 32, a nucleotide sequence having at least 99%, 98%, 97%, 96%, 95%, 90%, 85%, or 80% identity to the nucleotide sequence of nucleotides 120 to 833 of SEQ ID NO: 32, or a fragment of the nucleotide sequence of nucleotides 120 to 833 of SEQ ID NO: 32, or the nucleotide sequence having at least 99%, 98%, 97%, 96%, 95%, 90%, 85%, or 80% identity to the nucleotide sequence of nucleotides 120 to 833 of SEQ ID NO: 32; and/or
(ii) encodes an amino acid sequence comprising the amino acid sequence of amino acids 23 to 260 of SEQ ID NO: 31, an amino acid sequence having at least 99%, 98%, 97%, 96%, 95%, 90%, 85%, or 80% identity to the amino acid sequence of amino acids 23 to 260 of SEQ ID NO: 31, or an immunogenic fragment of the amino acid sequence of amino acids 23 to 260 of SEQ ID NO: 31, or the amino acid sequence having at least 99%, 98%, 97%, 96%, 95%, 90%, 85%, or 80% identity to the amino acid sequence of amino acids 23 to 260 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 120 to 833 of SEQ ID NO: 32; and/or (ii) encodes an amino acid sequence comprising the amino acid sequence of amino acids 23 to 260 of SEQ ID NO: 31.

According to certain embodiments, a transmembrane domain is fused, either directly or through a linker, e.g., a glycine/serine linker, to a SARS-CoV-2 S protein, a variant thereof, or a fragment thereof, i.e., the antigenic peptide or protein. Accordingly, in some embodiments, a transmembrane domain is fused to the above described amino acid sequences derived from SARS-CoV-2 S protein or immunogenic fragments thereof (antigenic peptides or proteins) comprised by the vaccine antigens described above (which may optionally be fused to a signal peptide and/or trimerization domain as described above). Such transmembrane domains are preferably located at the C-terminus of the antigenic peptide or protein, without being limited thereto. Preferably, such transmembrane domains are located at the C-terminus of the trimerization domain, if present, without being limited thereto. In one embodiment, a trimerization domain is present between the SARS-CoV-2 S protein, a variant thereof, or a fragment thereof, i.e., the antigenic peptide or protein, and the transmembrane domain. Transmembrane domains as defined herein preferably allow the anchoring into a cellular membrane of the antigenic peptide or protein as encoded by the RNA.

In some embodiments, the transmembrane domain sequence as defined herein includes, without being limited thereto, the transmembrane domain sequence of SARS-CoV-2 S protein, in particular a sequence comprising the amino acid sequence of amino acids 1207 to 1254 of SEQ ID NO: 1 or a functional variant thereof.

In some embodiments, a transmembrane domain sequence comprises the amino acid sequence of amino acids 1207 to 1254 of SEQ ID NO: 1, an amino acid sequence having at least 99%, 98%, 97%, 96%, 95%, 90%, 85%, or 80% identity to the amino acid sequence of amino acids 1207 to 1254 of SEQ ID NO: 1, or a functional fragment of the amino acid sequence of amino acids 1207 to 1254 of SEQ ID NO: 1, or the amino acid sequence having at least 99%, 98%, 97%, 96%, 95%, 90%, 85%, or 80% identity to the amino acid sequence of amino acids 1207 to 1254 of SEQ ID NO: 1. In some embodiments, a transmembrane domain sequence comprises the amino acid sequence of amino acids 1207 to 1254 of SEQ ID NO: 1.

In some embodiments, RNA encoding a transmembrane domain sequence (i) comprises the nucleotide sequence of nucleotides 3619 to 3762 of SEQ ID NO: 2, 8 or 9, a nucleotide sequence having at least 99%, 98%, 97%, 96%, 95%, 90%, 85%, or 80% identity to the nucleotide sequence of nucleotides 3619 to 3762 of SEQ ID NO: 2, 8 or 9, or a fragment of the nucleotide sequence of nucleotides 3619 to 3762 of SEQ ID NO: 2, 8 or 9, or the nucleotide sequence having at least 99%, 98%, 97%, 96%, 95%, 90%, 85%, or 80% identity to the nucleotide sequence of nucleotides 3619 to 3762 of SEQ ID NO: 2, 8 or 9; and/or (ii) encodes an amino acid sequence comprising the amino acid sequence of amino acids 1207 to 1254 of SEQ ID NO: 1, an amino acid sequence having at least 99%, 98%, 97%, 96%, 95%, 90%, 85%, or 80% identity to the amino acid sequence of amino acids 1207 to 1254 of SEQ ID NO: 1, or a functional fragment of the amino acid sequence of amino acids 1207 to 1254 of SEQ ID NO: 1, or the amino acid sequence having at least 99%, 98%, 97%, 96%, 95%, 90%, 85%, or 80% identity to the amino acid sequence of amino acids 1207 to 1254 of SEQ ID NO: 1. In some embodiments, RNA encoding a transmembrane domain sequence (i) comprises the nucleotide sequence of nucleotides 3619 to 3762 of SEQ ID NO: 2, 8 or 9; and/or (ii) encodes an amino acid sequence comprising the amino acid sequence of amino acids 1207 to 1254 of SEQ ID NO: 1.

In some embodiments, a vaccine antigen comprises the amino acid sequence of amino acids 1 to 311 of SEQ ID NO: 29, an amino acid sequence having at least 99%, 98%, 97%, 96%, 95%, 90%, 85%, or 80% identity to the amino acid sequence of amino acids 1 to 311 of SEQ ID NO: 29, or an immunogenic fragment of the amino acid sequence of amino acids 1 to 311 of SEQ ID NO: 29, or the amino acid sequence having at least 99%, 98%, 97%, 96%, 95%, 90%, 85%, or 80% identity to the amino acid sequence of amino acids 1 to 311 of SEQ ID NO: 29. In some embodiments, a vaccine antigen comprises the amino acid sequence of amino acids 1 to 311 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 986 of SEQ ID NO: 30, a nucleotide sequence having at least 99%, 98%, 97%, 96%, 95%, 90%, 85%, or 80% identity to the nucleotide sequence of nucleotides 54 to 986 of SEQ ID NO: 30, or a fragment of the nucleotide sequence of nucleotides 54 to 986 of SEQ ID NO: 30, or the nucleotide sequence having at least 99%, 98%, 97%, 96%, 95%, 90%, 85%, or 80% identity to the nucleotide sequence of nucleotides 54 to 986 of SEQ ID NO: 30; and/or
(ii) encodes an amino acid sequence comprising the amino acid sequence of amino acids 1 to 311 of SEQ ID NO: 29, an amino acid sequence having at least 99%, 98%, 97%, 96%, 95%, 90%, 85%, or 80% identity to the amino acid sequence of amino acids 1 to 311 of SEQ ID NO: 29, or an immunogenic fragment of the amino acid sequence of amino acids 1 to 311 of SEQ ID NO: 29, or the amino acid sequence having at least 99%, 98%, 97%, 96%, 95%, 90%, 85%, or 80% identity to the amino acid sequence of amino acids 1 to 311 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 986 of SEQ ID NO: 30; and/or (ii) encodes an amino acid sequence comprising the amino acid sequence of amino acids 1 to 311 of SEQ ID NO: 29.

In some embodiments, a vaccine antigen comprises the amino acid sequence of amino acids 1 to 314 of SEQ ID NO: 31, an amino acid sequence having at least 99%, 98%, 97%, 96%, 95%, 90%, 85%, or 80% identity to the amino acid sequence of amino acids 1 to 314 of SEQ ID NO: 31, or an immunogenic fragment of the amino acid sequence of amino acids 1 to 314 of SEQ ID NO: 31, or the amino acid sequence having at least 99%, 98%, 97%, 96%, 95%, 90%, 85%, or 80% identity to the amino acid sequence of amino acids 1 to 314 of SEQ ID NO: 31. In some embodiments, a vaccine antigen comprises the amino acid sequence of amino acids 1 to 314 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 54 to 995 of SEQ ID NO: 32, a nucleotide sequence having at least 99%, 98%, 97%, 96%, 95%, 90%, 85%, or 80% identity to the nucleotide sequence of nucleotides 54 to 995 of SEQ ID NO: 32, or a fragment of the nucleotide sequence of nucleotides 54 to 995 of SEQ ID NO: 32, or the nucleotide sequence having at least 99%, 98%, 97%, 96%, 95%, 90%, 85%, or 80% identity to the nucleotide sequence of nucleotides 54 to 995 of SEQ ID NO: 32; and/or
(ii) encodes an amino acid sequence comprising the amino acid sequence of amino acids 1 to 314 of SEQ ID NO: 31, an amino acid sequence having at least 99%, 98%, 97%, 96%, 95%, 90%, 85%, or 80% identity to the amino acid sequence of amino acids 1 to 314 of SEQ ID NO: 31, or an immunogenic fragment of the amino acid sequence of amino acids 1 to 314 of SEQ ID NO: 31, or the amino acid sequence having at least 99%, 98%, 97%, 96%, 95%, 90%, 85%, or 80% identity to the amino acid sequence of amino acids 1 to 314 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 995 of SEQ ID NO: 32; and/or (ii) encodes an amino acid sequence comprising the amino acid sequence of amino acids 1 to 314 of SEQ ID NO: 31.

In some embodiments, a vaccine antigen comprises the amino acid sequence of amino acids 20 to 311 of SEQ ID NO: 29, an amino acid sequence having at least 99%, 98%, 97%, 96%, 95%, 90%, 85%, or 80% identity to the amino acid sequence of amino acids 20 to 311 of SEQ ID NO: 29, or an immunogenic fragment of the amino acid sequence of amino acids 20 to 311 of SEQ ID NO: 29, or the amino acid sequence having at least 99%, 98%, 97%, 96%, 95%, 90%, 85%, or 80% identity to the amino acid sequence of amino acids 20 to 311 of SEQ ID NO: 29. In some embodiments, a vaccine antigen comprises the amino acid sequence of amino acids 20 to 311 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 111 to 986 of SEQ ID NO: 30, a nucleotide sequence having at least 99%, 98%, 97%, 96%, 95%, 90%, 85%, or 80% identity to the nucleotide sequence of nucleotides 111 to 986 of SEQ ID NO: 30, or a fragment of the nucleotide sequence of nucleotides 111 to 986 of SEQ ID NO: 30, or the nucleotide sequence having at least 99%, 98%, 97%, 96%, 95%, 90%, 85%, or 80% identity to the nucleotide sequence of nucleotides 111 to 986 of SEQ ID NO: 30; and/or
(ii) encodes an amino acid sequence comprising the amino acid sequence of amino acids 20 to 311 of SEQ ID NO: 29, an amino acid sequence having at least 99%, 98%, 97%, 96%, 95%, 90%, 85%, or 80% identity to the amino acid sequence of amino acids 20 to 311 of SEQ ID NO: 29, or an immunogenic fragment of the amino acid sequence of amino acids 20 to 311 of SEQ ID NO: 29, or the amino acid sequence having at least 99%, 98%, 97%, 96%, 95%, 90%, 85%, or 80% identity to the amino acid sequence of amino acids 20 to 311 of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 111 to 986 of SEQ ID NO: 30; and/or (ii) encodes an amino acid sequence comprising the amino acid sequence of amino acids 20 to 311 of SEQ ID NO: 29.

In some embodiments, a vaccine antigen comprises the amino acid sequence of amino acids 23 to 314 of SEQ ID NO: 31, an amino acid sequence having at least 99%, 98%, 97%, 96%, 95%, 90%, 85%, or 80% identity to the amino acid sequence of amino acids 23 to 314 of SEQ ID NO: 31, or an immunogenic fragment of the amino acid sequence of amino acids 23 to 314 of SEQ ID NO: 31, or the amino acid sequence having at least 99%, 98%, 97%, 96%, 95%, 90%, 85%, or 80% identity to the amino acid sequence of amino acids 23 to 314 of SEQ ID NO: 31. In one embodiment, a vaccine antigen comprises the amino acid sequence of amino acids 23 to 314 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of nucleotides 120 to 995 of SEQ ID NO: 32, a nucleotide sequence having at least 99%, 98%, 97%, 96%, 95%, 90%, 85%, or 80% identity to the nucleotide sequence of nucleotides 120 to 995 of SEQ ID NO: 32, or a fragment of the nucleotide sequence of nucleotides 120 to 995 of SEQ ID NO: 32, or the nucleotide sequence having at least 99%, 98%, 97%, 96%, 95%, 90%, 85%, or 80% identity to the nucleotide sequence of nucleotides 120 to 995 of SEQ ID NO: 32; and/or
(ii) encodes an amino acid sequence comprising the amino acid sequence of amino acids 23 to 314 of SEQ ID NO: 31, an amino acid sequence having at least 99%, 98%, 97%, 96%, 95%, 90%, 85%, or 80% identity to the amino acid sequence of amino acids 23 to 314 of SEQ ID NO: 31, or an immunogenic fragment of the amino acid sequence of amino acids 23 to 314 of SEQ ID NO: 31, or the amino acid sequence having at least 99%, 98%, 97%, 96%, 95%, 90%, 85%, or 80% identity to the amino acid sequence of amino acids 23 to 314 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 120 to 995 of SEQ ID NO: 32; and/or (ii) encodes an amino acid sequence comprising the amino acid sequence of amino acids 23 to 314 of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 30, a nucleotide sequence having at least 99%, 98%, 97%, 96%, 95%, 90%, 85%, or 80% identity to the nucleotide sequence of SEQ ID NO: 30, or a fragment of the nucleotide sequence of SEQ ID NO: 30, or the nucleotide sequence having at least 99%, 98%, 97%, 96%, 95%, 90%, 85%, or 80% identity to the nucleotide sequence of SEQ ID NO: 30; and/or
(ii) encodes an amino acid sequence comprising the amino acid sequence of SEQ ID NO: 29, an amino acid sequence having at least 99%, 98%, 97%, 96%, 95%, 90%, 85%, or 80% identity to the amino acid sequence of SEQ ID NO: 29, or an immunogenic fragment of the amino acid sequence of SEQ ID NO: 29, or the amino acid sequence having at least 99%, 98%, 97%, 96%, 95%, 90%, 85%, or 80% identity to the amino acid sequence of SEQ ID NO: 29.

In some embodiments, RNA encoding a vaccine antigen (i) comprises the nucleotide sequence of SEQ ID NO: 30; and/or (ii) encodes an amino acid sequence comprising the amino acid sequence of SEQ ID NO: 29.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 32, a nucleotide sequence having at least 99%, 98%, 97%, 96%, 95%, 90%, 85%, or 80% identity to the nucleotide sequence of SEQ ID NO: 32, or a fragment of the nucleotide sequence of SEQ ID NO: 32, or the nucleotide sequence having at least 99%, 98%, 97%, 96%, 95%, 90%, 85%, or 80% identity to the nucleotide sequence of SEQ ID NO: 32; and/or
(ii) encodes an amino acid sequence comprising the amino acid sequence of SEQ ID NO: 31, an amino acid sequence having at least 99%, 98%, 97%, 96%, 95%, 90%, 85%, or 80% identity to the amino acid sequence of SEQ ID NO: 31, or an immunogenic fragment of the amino acid sequence of SEQ ID NO: 31, or the amino acid sequence having at least 99%, 98%, 97%, 96%, 95%, 90%, 85%, or 80% identity to the amino acid sequence of SEQ ID NO: 31.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of SEQ ID NO: 32; and/or (ii) encodes an amino acid sequence comprising the amino acid sequence of SEQ ID NO: 31.

In some embodiments, a vaccine antigen comprises the amino acid sequence of SEQ ID NO: 28, an amino acid sequence having at least 99%, 98%, 97%, 96%, 95%, 90%, 85%, or 80% identity to the amino acid sequence of SEQ ID NO: 28, or an immunogenic fragment of the amino acid sequence of SEQ ID NO: 28, or the amino acid sequence having at least 99%, 98%, 97%, 96%, 95%, 90%, 85%, or 80% identity to the amino acid sequence of SEQ ID NO: 28. In some embodiments, a vaccine antigen comprises the amino acid sequence of SEQ ID NO: 28.

In some embodiments, the RNA encoding a SARS-CoV-2 S protein, an immunogenic variant thereof, or an immunogenic fragment of the SARS-CoV-2 S protein or the immunogenic variant thereof
(i) comprises the nucleotide sequence of SEQ ID NO: 27, a nucleotide sequence having at least 99%, 98%, 97%, 96%, 95%, 90%, 85%, or 80% identity to the nucleotide sequence of SEQ ID NO: 27, or a fragment of the nucleotide sequence of SEQ ID NO: 27, or the nucleotide sequence having at least 99%, 98%, 97%, 96%, 95%, 90%, 85%, or 80% identity to the nucleotide sequence of SEQ ID NO: 27; and/or
(ii) encodes an amino acid sequence comprising the amino acid sequence of SEQ ID NO: 28, an amino acid sequence having at least 99%, 98%, 97%, 96%, 95%, 90%, 85%, or 80% identity to the amino acid sequence of SEQ ID NO: 28, or an immunogenic fragment of the amino acid sequence of SEQ ID NO: 28, or the amino acid sequence having at least 99%, 98%, 97%, 96%, 95%, 90%, 85%, or 80% identity to the amino acid sequence of SEQ ID NO: 28.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of SEQ ID NO: 27; and/or (ii) encodes an amino acid sequence comprising the amino acid sequence of SEQ ID NO: 28.

In some embodiments, the vaccine antigens described above comprise a contiguous sequence of SARS-CoV-2 coronavirus spike (S) protein that consists of or essentially consists of the above described amino acid sequences derived from SARS-CoV-2 S protein or immunogenic fragments thereof (antigenic peptides or proteins) comprised by the vaccine antigens described above. In one embodiment, the vaccine antigens described above comprise a contiguous sequence of SARS-CoV-2 coronavirus spike (S) protein of no more than 220 amino acids, 215 amino acids, 210 amino acids, or 205 amino acids.

In some embodiments, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) described herein as BNT162b1 (RBPO20.3), BNT162b2 (RBPO20.1 or RBPO20.2). In one embodiment, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) described herein as RBP020.2.

In some embodiments, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 21, a nucleotide sequence having at least 99%, 98%, 97%, 96%, 95%, 90%, 85%, or 80% identity to the nucleotide sequence of SEQ ID NO: 21, and/or (ii) encodes an amino acid sequence comprising the amino acid sequence of SEQ ID NO: 5, or an amino acid sequence having at least 99%, 98%, 97%, 96%, 95%, 90%, 85%, or 80% identity to the amino acid sequence of SEQ ID NO: 5. In some embodiments, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 21; and/or (ii) encodes an amino acid sequence comprising the amino acid sequence of SEQ ID NO: 5.

In some embodiments, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 19, or 20, a nucleotide sequence having at least 99%, 98%, 97%, 96%, 95%, 90%, 85%, or 80% identity to the nucleotide sequence of SEQ ID NO: 19, or 20, and/or (ii) encodes an amino acid sequence comprising the amino acid sequence of SEQ ID NO: 7, or an amino acid sequence having at least 99%, 98%, 97%, 96%, 95%, 90%, 85%, or 80% identity to the amino acid sequence of SEQ ID NO: 7. In some embodiments, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 19, or 20; and/or (ii) encodes an amino acid sequence comprising the amino acid sequence of SEQ ID NO: 7.

In some embodiments, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 20, a nucleotide sequence having at least 99%, 98%, 97%, 96%, 95%, 90%, 85%, or 80% identity to the nucleotide sequence of SEQ ID NO: 20, and/or (ii) encodes an amino acid sequence comprising the amino acid sequence of SEQ ID NO: 7, or an amino acid sequence having at least 99%, 98%, 97%, 96%, 95%, 90%, 85%, or 80% identity to the amino acid sequence of SEQ ID NO: 7. In some embodiments, the RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and (i) comprises the nucleotide sequence of SEQ ID NO: 20; and/or (ii) encodes an amino acid sequence comprising the amino acid sequence of SEQ ID NO: 7.

In some embodiments, the RNA encoding a SARS-CoV-2 S protein, an immunogenic variant thereof, or an immunogenic fragment of the SARS-CoV-2 S protein or the immunogenic variant thereof (i) comprises the nucleotide sequence of nucleotides 54 to 725 of SEQ ID NO: 32; and/or (ii) encodes an amino acid sequence comprising the amino acid sequence of amino acids 1 to 224 of SEQ ID NO: 31.

In some embodiments, a vaccine antigen comprises the amino acid sequence of SEQ ID NO: 42, an amino acid sequence having at least 99.5%, 99%, 98.5%, 98%, 98.5% or 97% identity to the amino acid sequence of SEQ ID NO: 42, or an immunogenic fragment of the amino acid sequence of SEQ ID NO: 42, or the amino acid sequence having at least 99.5%, 99%, 98.5%, 98%, 98.5% or 97% identity to the amino acid sequence of SEQ ID NO: 42. In some embodiments, a vaccine antigen comprises the amino acid sequence of SEQ ID NO: 42.

In some embodiments, RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and
(i) comprises the nucleotide sequence of SEQ ID NO: 43, a nucleotide sequence having at least 99.5%, 99%, 98.5%, 98%, 98.5% or 97% identity to the nucleotide sequence of SEQ ID NO: 43, and/or
(ii) encodes an amino acid sequence comprising the amino acid sequence of SEQ ID NO: 42, or an amino acid sequence having at least 99.5%, 99%, 98.5%, 98%, 98.5% or 97% identity to the amino acid sequence of SEQ ID NO: 42. In one embodiment, RNA encoding a vaccine antigen is nucleoside modified messenger RNA (modRNA) and (i) comprises the nucleotide sequence of SEQ ID NO: 43; and/or (ii) encodes an amino acid sequence comprising the amino acid sequence of SEQ ID NO: 42.

In some embodiments, RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and
(i) comprises the nucleotide sequence of SEQ ID NO: 44, a nucleotide sequence having at least 99.5%, 99%, 98.5%, 98%, 98.5% or 97% identity to the nucleotide sequence of SEQ ID NO: 44, and/or
(ii) encodes an amino acid sequence comprising the amino acid sequence of SEQ ID NO: 42, or an amino acid sequence having at least 99.5%, 99%, 98.5%, 98%, 98.5% or 97% identity to the amino acid sequence of SEQ ID NO: 42. In one embodiment, RNA encoding a vaccine antigen is nucleoside modified messenger RNA (modRNA) and (i) comprises the nucleotide sequence of SEQ ID NO: 44; and/or (ii) encodes an amino acid sequence comprising the amino acid sequence of SEQ ID NO: 42.

In one embodiment, a vaccine antigen comprises the amino acid sequence of SEQ ID NO: 45, an amino acid sequence having at least 99.5%, 99%, 98.5%, 98%, 98.5% or 97% identity to the amino acid sequence of SEQ ID NO: 45, or an immunogenic fragment of the amino acid sequence of SEQ ID NO: 45, or the amino acid sequence having at least 99.5%, 99%, 98.5%, 98%, 98.5% or 97% identity to the amino acid sequence of SEQ ID NO: 45. In one embodiment, a vaccine antigen comprises the amino acid sequence of SEQ ID NO: 45.

In some embodiments, RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and
(i) comprises the nucleotide sequence of SEQ ID NO: 46, a nucleotide sequence having at least 99.5%, 99%, 98.5%, 98%, 98.5% or 97% identity to the nucleotide sequence of SEQ ID NO: 46, and/or
(ii) encodes an amino acid sequence comprising the amino acid sequence of SEQ ID NO: 45, or an amino acid sequence having at least 99.5%, 99%, 98.5%, 98%, 98.5% or 97% identity to the amino acid sequence of SEQ ID NO: 45. In one embodiment, RNA encoding a vaccine antigen is nucleoside modified messenger RNA (modRNA) and (i) comprises the nucleotide sequence of SEQ ID NO: 46; and/or (ii) encodes an amino acid sequence comprising the amino acid sequence of SEQ ID NO: 45.

In some embodiments, RNA encoding a SARS-CoV-2 S protein, an immunogenic variant thereof, or an immunogenic fragment of the SARS-CoV-2 S protein or the immunogenic variant thereof is nucleoside modified messenger RNA (modRNA) and
(i) comprises the nucleotide sequence of SEQ ID NO: 47, a nucleotide sequence having at least 99.5%, 99%, 98.5%, 98%, 98.5% or 97% identity to the nucleotide sequence of SEQ ID NO: 47, and/or
(ii) encodes an amino acid sequence comprising the amino acid sequence of SEQ ID NO: 45, or an amino acid sequence having at least 99.5%, 99%, 98.5%, 98%, 98.5% or 97% identity to the amino acid sequence of SEQ ID NO: 45. In one embodiment, RNA encoding a vaccine antigen is nucleoside modified messenger RNA (modRNA) and (i) comprises the nucleotide sequence of SEQ ID NO: 47; and/or (ii) encodes an amino acid sequence comprising the amino acid sequence of SEQ ID NO: 45.

In some embodiments of the RNA encoding a SARS-CoV-2 S protein, an immunogenic variant thereof, or an immunogenic fragment of the SARS-CoV-2 S protein or the immunogenic variant thereof, said RNA contains one or more of the above described RNA modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (T) or N(1)-methylpseudouridine (m1Ψ) or 5-methyluridine (m5U) for uridine), and codon optimization. In one embodiment, said RNA contains a combination of the above described modifications, preferably a combination of at least two, at least three, at least four or all five of the above-mentioned modifications, i.e., (i) incorporation of a 5′-cap structure, (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (T) or N(1)-methylpseudouridine (m1Ψ) or 5-methyluridine (m5U) for uridine), and (v) codon optimization.

In some embodiments of the RNA encoding a SARS-CoV-2 S protein, an immunogenic variant thereof, or an immunogenic fragment of the SARS-CoV-2 S protein or the immunogenic variant thereof, said RNA is a modified RNA, in particular a stabilized mRNA. In some embodiments, said RNA comprises a modified nucleoside in place of at least one uridine. In some embodiments, said RNA comprises a modified nucleoside in place of uridine, such as in place of each uridine. In some embodiments, the modified nucleoside is independently selected from pseudouridine (yr), N1-methyl-pseudouridine (m1ψ), and 5-methyl-uridine (m5U). In some embodiments, said RNA comprises a 5′ cap, preferably a cap1 or cap2 structure, more preferably a cap1 structure. In some embodiments, said RNA comprises a 5′-UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In some embodiments, said RNA comprises a 3′-UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13. In some embodiments, said RNA comprises a poly-A sequence. In some embodiments, the poly-A sequence comprises at least 100 nucleotides. In some embodiments, the poly-A sequence comprises or consists of the nucleotide sequence of SEQ ID NO: 14.

In some embodiments of the RNA encoding a SARS-CoV-2 S protein, an immunogenic variant thereof, or an immunogenic fragment of the SARS-CoV-2 S protein or the immunogenic variant thereof, said variants include mutations in RBD (e.g., but not limited to Q321L, V341I, A348T, N354D, S359N, V367F, K378R, R408I, Q409E, A435S, N439K, K458R, 1472V, G476S, S477N, V483A, Y508H, H519P, etc., as compared to SEQ ID NO: 1), and/or mutations in spike protein (e.g., but not limited to D614G, etc., as compared to SEQ ID NO: 1). Those skilled in the art are aware of various spike variants, and/or resources that document them (e.g., the Table of mutating sites in Spike maintained by the COVID-19 Viral Genome Analysis Pipeline and found at https://cov.lanl.gov/components/sequence/COV/int_sites_tbls.comp) (last accessed 24 Aug. 2020), and, reading the present specification, will appreciate that RNA compositions and/or methods described herein can be characterized for their ability to induce sera in vaccinated subject that display neutralizing activity with respect to any or all of such variants and/or combinations thereof.

In some embodiments of the RNA encoding a SARS-CoV-2 S protein, an immunogenic variant thereof, or an immunogenic fragment of the SARS-CoV-2 S protein or the immunogenic variant thereof, said variants include a mutation at position 501 in spike protein as compared to SEQ ID NO: 1 and optionally may include one or more further mutations as compared to SEQ ID NO: 1 (e.g., but not limited to H69/V70 deletion, Y144 deletion, A570D, D614G, P6811H, T716I, S982A, D1118H, D80A, D215G, E484K, A701V, L18F, R246I, K417N, L242/A243/L244 deletion etc., as compared to SEQ ID NO: 1).

In some embodiments of the RNA encoding a SARS-CoV-2 S protein, an immunogenic variant thereof, or an immunogenic fragment of the SARS-CoV-2 S protein or the immunogenic variant thereof, said variants include “Variant of Concern 202012/01” (VOC-202012/01; also known as lineage B.1.1.7). The variant had previously been named the first Variant Under Investigation in December 2020 (VUI-202012/01) by Public Health England, but was reclassified to a Variant of Concern (VOC-202012/01). VOC-202012/01 is a variant of SARS-CoV-2 which was first detected in October 2020 during the COVID-19 pandemic in the United Kingdom from a sample taken the previous month, and it quickly began to spread by mid-December. It is correlated with a significant increase in the rate of COVID-19 infection in United Kingdom; this increase is thought to be at least partly because of change N501Y inside the spike glycoprotein's receptor-binding domain, which is needed for binding to ACE2 in human cells. The VOC-202012/01 variant is defined by 23 mutations: 13 non-synonymous mutations, 4 deletions, and 6 synonymous mutations (i.e., there are 17 mutations that change proteins and six that do not). The spike protein changes in VOC 202012/01 include deletion 69-70, deletion 144, N501Y, A570D, D614G, P681H, T716I, S982A, and D1118H. One of the most important changes in VOC-202012/01 seems to be N501Y, a change from asparagine (N) to tyrosine (Y) at amino-acid site 501. This mutation alone or in combination with the deletion at positions 69/70 in the N terminal domain (NTD) may enhance the transmissibility of the virus.

Thus, in particular embodiments of the RNA encoding a SARS-CoV-2 S protein, an immunogenic variant thereof, or an immunogenic fragment of the SARS-CoV-2 S protein or the immunogenic variant thereof, said variants include a SARs-CoV-2 spike variant including the following mutations: deletion 69-70, deletion 144, N501Y, A570D, D614G, P68111, T716I, S982A, and D1118H as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include variant “501.V2”. This variant was first observed in samples from October 2020, and since then more than 300 cases with the 501.V2 variant have been confirmed by whole genome sequencing (WGS) in South Africa, where in December 2020 it was the dominant form of the virus. Preliminary results indicate that this variant may have an increased transmissibility. The 501.V2 variant is defined by multiple spike protein changes including: D80A, D215G, E484K, N501Y and A701V, and more recently collected viruses have additional changes: L18F, R246I, K417N, and deletion 242-244.

Thus, in particular embodiments of the RNA encoding a SARS-CoV-2 S protein, an immunogenic variant thereof, or an immunogenic fragment of the SARS-CoV-2 S protein or the immunogenic variant thereof, said variants include a SARs-CoV-2 spike variant including the following mutations: D80A, D215G, E484K, N501Y and A701V as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a 1169/V70 deletion in spike protein as compared to SEQ ID NO: 1. Said SARs-CoV-2 spike variant may also include one or more further mutations as compared to SEQ ID NO: 1 (e.g., but not limited to Y144 deletion, N501Y, A570D, D614G, P68111, T716I, S982A, D1118H, D80A, D215G, E484K, A701V, L18F, R246I, K417N, L242/A243/L244 deletion, Y453F, I692V, S1147L, M1229I etc., as compared to SEQ ID NO: 1). In particular embodiments, said SARs-CoV-2 spike variant includes the following mutations: deletion 69-70, deletion 144, N501Y, A570D, D614G, P681H, T716I, S982A, and D1118H as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include variant “Cluster 5”, also referred to as ΔFVI-spike by the Danish State Serum Institute (SSI). It was discovered in North Jutland, Denmark, and is believed to have been spread from minks to humans via mink farms. In cluster 5, several different mutations in the spike protein of the virus have been confirmed. The specific mutations include 69-70deltaHV (a deletion of the histidine and valine residues at the 69th and 70th position in the protein), Y453F (a change from tyrosine to phenylalanine at position 453), 1692V (isoleucine to valine at position 692), M1229I (methionine to isoleucine at position 1229), and optionally S1147L (serine to leucine at position 1147).

Thus, in particular embodiments of the RNA encoding a SARS-CoV-2 S protein, an immunogenic variant thereof, or an immunogenic fragment of the SARS-CoV-2 S protein or the immunogenic variant thereof, said variants include a SARs-CoV-2 spike variant including the following mutations: deletion 69-70, Y453F, I692V, M1229I, and optionally S1147L, as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at position 614 in spike protein as compared to SEQ ID NO: 1, such as a D614G mutation in spike protein as compared to SEQ ID NO: 1. Said SARs-CoV-2 spike variants including a mutation at position 614 in spike protein as compared to SEQ ID NO: 1 may also include one or more further mutations as compared to SEQ ID NO: 1 (e.g., but not limited to H69/V70 deletion, Y144 deletion, N501Y, A570D, P681H, T716I, S982A, D1118H, D80A, D215G, E484K, A701V, L18F, R246I, K417N, L242/A243/L244 deletion, Y453F, I692V, 51147L, M1229I etc., as compared to SEQ ID NO: 1).

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: deletion 69-70, deletion 144, N501Y, A570D, D614G, P681H, T716I, S982A, and D1118H as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V, and D614G as compared to SEQ ID NO: 1, and optionally: L18F, R246I, K417N, and deletion 242-244 as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at positions 501 and 614 in spike protein as compared to SEQ ID NO: 1. In some embodiments, said SARs-CoV-2 spike variants include a N501Y mutation and a D614G mutation in spike protein as compared to SEQ ID NO: 1. In some embodiments, said SARs-CoV-2 spike variants include one or more further mutations as compared to SEQ IDNO: 1 (e.g., but not limited to H69/V70 deletion, Y144 deletion, A570D, P681H, T716I, S982A, D1118H, D80A, D215G, E484K, A701V, L18F, R246I, K417N, L242/A243/L244 deletion, Y453F, I692V, S1147L, M1229I etc., as compared to SEQ ID NO: 1).

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: deletion 69-70, deletion 144, N501Y, A570D, D614G, P681H, T716I, S982A, and D1 18118H as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V, and D614G as compared to SEQ ID NO: 1, and optionally: L18F, R246I, K417N, and deletion 242-244 as compared to SEQ ID NO: 1 In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at position 484 in spike protein as compared to SEQ ID NO: 1, such as a E484K mutation in spike protein as compared to SEQ ID NO: 1. In some embodiments, said SARs-CoV-2 spike variants may include one or more further mutations as compared to SEQ ID NO: 1 (e.g., but not limited to H69/V70 deletion, Y144 deletion, N501Y, A570D, D614G, P681H, T716I, S982A, D1118H, D80A, D215G, A701V, L18F, R246I, K417N, L242/A243/L244 deletion, Y453F, 1692V, S1147L, M1229I, T20N, P26S, D138Y, R190S, K417T, H655Y, T1027I, V1176F etc., as compared to SEQ ID NO: 1).

In some embodiments of the RNA encoding a SARS-CoV-2 S protein, an inunnogenic variant thereof, or an immunogenic fragment of the SARS-CoV-2 S protein or the immunogenic variant thereof, said variants include SARs-CoV-2 spike variants including the following mutations: D80A, D215G, E484K, N501Y, and A701V, as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include variant lineage B.1.1.248, known as the Brazil(ian) variant. This variant of SARS-CoV-2 has been named P.1 lineage and has 17 unique amino acid changes, 10 of which in its spike protein, including N501Y and E484K. B.1.1.248 originated from B.1.1.28. E484K is present in both B.1.1.28 and B.1.1.248. B.1.1.248 has a number of S-protein polymorphisms [L18F, T20N, P26S, D138Y, R190S, K417T, E484K, N501Y, H655Y, T1027I, V1176F] and is similar in certain key RBD positions (K417, E484, N501) to variant described from South Africa.

Thus, in particular embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: L18F, T20N, P26S, D138Y, R190S, K417T, E484K, N501Y, H655Y, T1027I, and V1176F as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at positions 501 and 484 in spike protein as compared to SEQ ID NO: 1, such as a N501Y mutation and a E484K mutation in spike protein as compared to SEQ ID NO: 1. In some embodiments, said SARs-CoV-2 spike variants may include one or more further mutations as compared to SEQ ID NO: 1 (e.g., but not limited to H69/V70 deletion, Y144 deletion, A570D, D614G, P681H, T716I, S982A, D1 118H, D80A, D215G, A701V, L18F, R246I, K417N, L242/A243/L244 deletion, Y453F, I692V, S1147L, M1229I, T20N, P26S, D138Y, R190S, K417T, H655Y, T1027I, V1176F etc., as compared to SEQ ID NO: 1).

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y and A701V as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: L18F, T20N, P26S, D138Y, R190S, K417T, E484K, N501Y, H655Y, T1027I, and V1 176F as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at positions 501, 484 and 614 in spike protein as compared to SEQ ID NO: 1, such as a N501Y mutation, a E484K mutation and a D614G mutation in spike protein as compared to SEQ ID NO: 1. In some embodiments, said SARs-CoV-2 spike variants may include one or more further mutations as compared to SEQ ID NO: 1 (e.g., but not limited to H69/V70 deletion, Y144 deletion, A570D, P681H, T716I, S982A, D118H, D80A, D215G, A701V, L18F, R246I, K417N, L242/A243/L244 deletion, Y453F, I692V, 51147L, M1229I, T20N, P26S, D138Y, R190S, K417T, H655Y, T1027I, V1 176F etc., as compared to SEQ ID NO: 1).

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V, and D614G as compared to SEQ ID NO: 1, and optionally: L18F, R246I, K417N, and deletion 242-244 as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a L242/A243/L244 deletion in spike protein as compared to SEQ ID NO: 1. In some embodiments, said SARs-CoV-2 spike variants may include one or more further mutations as compared to SEQ ID NO: 1 (e.g., but not limited to H69/V70 deletion, Y144 deletion, N501Y, A570D, D614G, P681H, T716I, S982A, D1118H, D80A, D215G, E484K, A701V, L18F, R246I, K417N, Y453F, 1692V, S1147L, M1229I, T20N, P26S, D138Y, R190S, K417T, H655Y, T1027I, V1 176F etc., as compared to SEQ ID NO: 1).

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V and deletion 242-244 as compared to SEQ ID NO: 1, and optionally: L18F, R246I, and K417N, as compared to SEQ ID NO: 1. Said SARs-CoV-2 spike variant may also include a D614G mutation as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at position 417 in spike protein as compared to SEQ ID NO: 1, such as a K417N or K417T mutation in spike protein as compared to SEQ ID NO: 1. In some embodiments, said SARs-CoV-2 spike variants may include one or more further mutations as compared to SEQ ID NO: 1 (e.g., but not limited to H69/V70 deletion, Y144 deletion, N501Y, A570D, D614G, P681H, T716I, S982A, D1118H, D80A, D215G, E484K, A701V, L18F, R246I, L242/A243/L244 deletion, Y453F, 1692V, 51147L, M1229I, T20N, P26S, D138Y, R190S, H655Y, T1027I, V1176F etc., as compared to SEQ ID NO: 1).

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A7011V and K417N, as compared to SEQ ID NO: 1, and optionally: L18F, R246I, and deletion 242-244 as compared to SEQ ID NO: 1. Said SARs-CoV-2 spike variant may also include a D614G mutation as compared to SEQ ID NO: 1.

In some embodiments of the RNA encoding a SARS-CoV-2 S protein, an immunogenic variant thereof, or an immunogenic fragment of the SARS-CoV-2 S protein or the imunuogenic variant thereof, said variants include SARs-CoV-2 spike variants including the following mutations: L18F, T20N, P26S, D138Y, R190S, K417T, E484K, N501Y, H655Y, T1027I, and V1176F as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including a mutation at positions 417 and 484 and/or 501 in spike protein as compared to SEQ ID NO: 1, such as a K417N or K417T mutation and a E484K and/or N501Y mutation in spike protein as compared to SEQ ID NO: 1. In some embodiments, said SARs-CoV-2 spike variants may include one or more further mutations as compared to SEQ ID NO: 1 (e.g., but not limited to H69/V70 deletion, Y144 deletion, A570D, D614G, P681H, T716I, S982A, D1118H, D80A, D215G, A701V, L18F, R246I, L242/A243/L244 deletion, Y453F, 1692V, S1147L, M1229I, T20N, P26S, D138Y, R190S, H655Y, T1027I, V1176F etc., as compared to SEQ ID NO: 1).

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: D80A, D215G, E484K, N501Y, A701V and K417N, as compared to SEQ ID NO: 1, and optionally: L18F, R246I, and deletion 242-244 as compared to SEQ ID NO: 1. Said SARs-CoV-2 spike variant may also include a D614G mutation as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including following mutations: L18F, T20N, P26S, D138Y, R190S, K417T, E484K, N501Y, H655Y, T1027I, and Vi 176F as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include variant lineage B.1.1.529, known as the Omicron variant. This variant has a large number of mutations, including A67V, A69-70, T95I, G142D, A143-145, A211, L212I, ins214EPE (insertion of EPE following amino acid 214), G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

Thus, in some embodiments of the RNA encoding a SARS-CoV-2 S protein, an immunogenic variant thereof, or an immunogenic fragment of the SARS-CoV-2 S protein or the immunogenic variant thereof, said variants include SARs-CoV-2 spike variants including the following mutations: A67V, A69-70, T95I, G142D, A143-145, A211, L212I, ins214EPE (insertion of EPE following amino acid 214), G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37 of the following mutations: T547K, H655Y, D614G, N679K, P6811H, N969K, S373P, S371L, N440K, G339D, G446S, N856K, N764K, K417N, D796Y, Q954H, T95I, A67V, L981F, S477N, G496S, T478K, Q498R, Q493R, E484A, N501Y, S375F, Y505H, V143del, H69del, V70del, N211del, L212I, ins214EPE, G142D, Y144del, Y145del, 1141del, Y144F, Y145D, G142del, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include at least 10, at least 15, at least 20, at least 21, at least 22, at least 23, at least 24, or all of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, as compared to SEQ ID NO: 1. Said SARs-CoV-2 spike variants may include at least 1, at least 2, at least 3, at least 4, at least 5, or all of the following mutations: N501Y, S375F, Y505H, V143del, H69del, V70del, as compared to SEQ ID NO: 1, and/or may include at least 1, at least 2, at least 3, at least 4, at least 5, or all of the following mutations: N211 del, L212I, ins214EPE, G142D, Y144del, Y145del, as compared to SEQ ID NO: 1. In some embodiments, said SARs-CoV-2 spike variants may include at least 1, at least 2, at least 3, or all of the following mutations: L141del, Y144F, Y145D, G142del, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include at least 10, at least 15, at least 20, at least 21, at least 22, at least 23, at least 24, at least 25, at least 26, at least 27, at least 28, at least 29, at least 30, at least 31, at least 32, at least 33 of the following mutations: A67V, Δ69-70, T95I, G142D, A143-145, A211, L212I, ins214EPE, G339D, 5371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include the following mutations: A67V, Δ69-70, T95I, G142D, A143-145, A211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments of the RNA encoding a SARS-CoV-2 S protein, an immunogenic variant thereof, or an immunogenic fragment of the SARS-CoV-2 S protein or the immunogenic variant thereof, said variants include the following mutations: A67V, Δ69-70, T95I, G142D, A143-145, A211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

The SARs-CoV-2 spike variants described herein may or may not include a D614G mutation as compared to SEQ ID NO: 1.

The SARs-CoV-2 spike variants may have one or more of the mutations disclosed under the section “Use of pharmaceutical compositions”.

In some embodiments of the present disclosure, the antigen (such as a tumor antigen or vaccine antigen) is preferably administered as single-stranded, 5′ capped RNA (preferably mRNA) that is translated into the respective protein upon entering cells of a subject being administered the RNA. Preferably, the RNA contains structural elements optimized for maximal efficacy of the RNA with respect to stability and translational efficiency (5′ cap, 5′ UTR, 3′ UTR, poly(A) sequence).

In some embodiments, beta-S-ARCA(D1) is utilized as specific capping structure at the 5′-end of the RNA. In one embodiment, m₂^{7,3′-O}-Gppp(m₁^{2′-O})ApG is utilized as specific capping structure at the 5′-end of the RNA. In some embodiments, the 5′-UTR sequence is derived from the human alpha-globin mRNA and optionally has an optimized ‘Kozak sequence’ to increase translational efficiency. In some embodiments, a combination of two sequence elements (FI element) derived from the “amino terminal enhancer of split” (AES) mRNA (called F) and the mitochondrial encoded 12S ribosomal RNA (called I) are placed between the coding sequence and the poly(A) sequence to assure higher maximum protein levels and prolonged persistence of the mRNA. In some embodiments, two re-iterated 3′-UTRs derived from the human beta-globin mRNA are placed between the coding sequence and the poly(A) sequence to assure higher maximum protein levels and prolonged persistence of the mRNA. In some embodiments, a poly(A) sequence measuring 110 nucleotides in length, consisting of a stretch of 30 adenosine residues, followed by a 10 nucleotide linker sequence and another 70 adenosine residues is used. This poly(A) sequence was designed to enhance RNA stability and translational efficiency.

In the following, embodiments of three different RNA platforms are described each of which encodes a SARS-CoV-2 S protein, an immunogenic variant thereof, or an immunogenic fragment of the SARS-CoV-2 S protein or the immunogenic variant thereof.

In general, vaccine RNA described herein may comprise, from 5′ to 3′, one of the following structures:
Cap-5′-UTR-Vaccine Antigen-Encoding Sequence-3′-UTR-Poly(A)
   or
beta-S-ARCA(D1)-hAg-Kozak-Vaccine Antigen-Encoding Sequence-FI-A30L70.

In general, a vaccine antigen described herein may comprise, from N-terminus to C-terminus, one of the following structures:
Signal Sequence-RBD-Trimerization Domain
   or
Signal Sequence-RBD-Trimerization Domain-Transmembrane Domain.

RBD and Trimerization Domain may be separated by a linker, in particular a GS linker such as a linker having the amino acid sequence GSPGSGSGS (SEQ ID NO: 33). Trimerization Domain and Transmembrane Domain may be separated by a linker, in particular a GS linker such as a linker having the amino acid sequence GSGSGS (SEQ ID NO: 34).

Signal Sequence may be a signal sequence as described herein. RBD may be a RBD domain as described herein. Trimerization Domain may be a trimerization domain as described herein. Transmembrane Domain may be a transmembrane domain as described herein.

In one embodiment,
Signal sequence comprises the amino acid sequence of amino acids 1 to 16 or 1 to 19 of SEQ ID NO: 1 or the amino acid sequence of amino acids 1 to 22 of SEQ ID NO: 31, or an amino acid sequence having at least 99%, 98%, 97%, 96%, 95%, 90%, 85%, or 80% identity to this amino acid sequence, RBD comprises the amino acid sequence of amino acids 327 to 528 of SEQ ID NO: 1, or an amino acid sequence having at least 99%, 98%, 97%, 96%, 95%, 90%, 85%, or 80% identity to this amino acid sequence, Trimerization Domain comprises the amino acid sequence of amino acids 3 to 29 of SEQ ID NO: 10 or the amino acid sequence of SEQ ID NO: 10, or an amino acid sequence having at least 99%, 98%, 97%, 96%, 95%, 90%, 85%, or 80% identity to this amino acid sequence; and Transmembrane Domain comprises the amino acid sequence of amino acids 1207 to 1254 of SEQ ID NO: 1, or an amino acid sequence having at least 99%, 98%, 97%, 96%, 95%, 90%, 85%, or 80% identity to this amino acid sequence.

In some embodiments,
Signal sequence comprises the amino acid sequence of amino acids 1 to 16 or 1 to 19 of SEQ ID NO: 1 or the amino acid sequence of amino acids 1 to 22 of SEQ ID NO: 31, RBD comprises the amino acid sequence of amino acids 327 to 528 of SEQ ID NO: 1, Trimerization Domain comprises the amino acid sequence of amino acids 3 to 29 of SEQ ID NO: 10 or the amino acid sequence of SEQ ID NO: 10; and Transmembrane Domain comprises the amino acid sequence of amino acids 1207 to 1254 of SEQ ID NO: 1.

The above described RNA or RNA encoding the above described vaccine antigen may be non-modified uridine containing mRNA (uRNA), nucleoside modified mRNA (modRNA) or self-amplifying RNA (saRNA). In some embodiments, the above described RNA or RNA encoding the above described vaccine antigen is nucleoside modified mRNA (modRNA).

Non-Modified Uridine Messenger RNA (uRNA)

The active principle of the non-modified messenger RNA (uRNA) is a single-stranded mRNA that is translated upon entering a cell. In addition to the sequence encoding the coronavirus vaccine antigen (i.e. open reading frame), each uRNA preferably contains common structural elements optimized for maximal efficacy of the RNA with respect to stability and translational efficiency (5′-cap, 5′-UTR, 3′-UTR, poly(A)-tail). The preferred 5′ cap structure is beta-S-ARCA(D1) (m₂^{7,2′-O}GppSpG). The preferred 5′-UTR and 3′-UTR comprise the nucleotide sequence of SEQ ID NO: 12 and the nucleotide sequence of SEQ ID NO: 13, respectively. The preferred poly(A)-tail comprises the sequence of SEQ ID NO: 14.

Different embodiments of this platform are as follows:

RBL063.1 (SEQ ID NO: 15; SEQ ID NO: 7)

| | |
|---|---|
| Structure | beta-S-ARCA(D1)-hAg-Kozak-S1S2-PP-FI-A30L70 |
| Encoded | Viral spike protein (S1S2 protein) of the SARS-CoV-2 (S1S2 |
| antigen | full-length protein, sequence variant) |

RBL063.2 (SEQ ID NO: 16; SEQ ID NO: 7)

| | |
|---|---|
| Structure | beta-S-ARCA(D1)-hAg-Kozak-S1S2-PP-FI-A30L70 |
| Encoded | Viral spike protein (S1S2 protein) of the SARS-CoV-2 (S1S2 |
| antigen | full-length protein, sequence variant) |

BNT162a1; RBL063.3 (SEQ ID NO: 17; SEQ ID NO: 5)

| | |
|---|---|
| Structure | beta-S-ARCA(D1)-hAg-Kozak-RBD-GS-Fibritin-FI-A30L70 |
| Encoded | Viral spike protein (S protein) of the SARS-CoV-2 (partial |
| antigen | sequence, Receptor Binding Domain (RBD) of S1S2 protein) |

In this respect, “hAg-Kozak” mean the 5′-UTR sequence of the human alpha-globin mRNA with an optimized ‘Kozak sequence’ to increase translational efficiency; “S1S2 protein”/“SIS2 RBD” means the sequences encoding the respective antigen of SARS-CoV-2; “FI element” means that the 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression; “A30L70” means a poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells; “GS” means a glycine-serine linker, i.e., sequences coding for short linker peptides predominantly consisting of the amino acids glycine (G) and serine (S), as commonly used for fusion proteins.

Nucleoside Modified Messenger RNA (modRNA)

The active principle of the nucleoside modified messenger RNA (modRNA) drug substance is as well a single-stranded mRNA that is translated upon entering a cell. In addition to the sequence encoding the coronavirus vaccine antigen (i.e., open reading frame), each modRNA contains common structural elements optimized for maximal efficacy of the RNA as the uRNA (5′-cap, 5′-UTR, 3′-UTR, poly(A)-tail). Compared to the uRNA, modRNA contains 1-methyl-pseudouridine instead of uridine. The preferred 5′ cap structure is m₂^{7,3′-O}Gppp(m₁^{2′-O})ApG. The preferred 5′-UTR and 3′-UTR comprise the nucleotide sequence of SEQ ID NO: 12 and the nucleotide sequence of SEQ ID NO: 13, respectively. The preferred poly(A)-tail comprises the sequence of SEQ ID NO: 14. An additional purification step is applied for modRNA to reduce dsRNA contaminants generated during the in vitro transcription reaction.

Different embodiments of this platform are as follows:

BNT162b2; RBP020.1 (SEQ ID NO: 19; SEQ ID NO: 7)

| | |
|---|---|
| Structure | m₂^{7,3′-O}Gppp(m₁^{2′-O})ApG)-hAg-Kozak-S1S2-PP-FI-A30L70 |
| Encoded | Viral spike protein (S1S2 protein) of the SARS-CoV-2 (S1S2 |
| antigen | full-length protein, sequence variant) |

BNT162b2; RBP020.2 (SEQ ID NO: 20; SEQ ID NO: 7)

| | |
|---|---|
| Structure | m₂^{7,3′-O}Gppp(m₁^{2′-O})ApG)-hAg-Kozak-S1S2-PP-FI-A30L70 |
| Encoded | Viral spike protein (S1S2 protein) of the SARS-CoV-2 (S1S2 |
| antigen | full-length protein, sequence variant) |

BNT162b1; RBP020.3 (SEQ ID NO: 21; SEQ ID NO: 5)

| | |
|---|---|
| Structure | m₂^{7,3′-O}Gppp(m₁^{2′-O})ApG)-hAg-Kozak-RBD-GS-Fibritin- |
| | FI-A30L70 |
| Encoded | Viral spike protein (S1S2 protein) of the SARS-CoV-2 (partial |
| antigen | sequence, Receptor Binding Domain (RBD) of S1S2 protein |
| | fused to fibritin) |

BNT162b3c (SEQ ID NO: 29; SEQ ID NO: 30)

| | |
|---|---|
| Structure | m₂^{7,3′-O}Gppp(m₁^{2′-O})ApG-hAg-Kozak-RBD-GS-Fibritin-GS- |
| | TM-FI-A30L70 |
| Encoded | Viral spike protein (S1S2 protein) of the SARS-CoV-2 (partial |
| antigen | sequence, Receptor Binding Domain (RBD) of S1S2 protein |
| | fused to Fibritin fused to Transmembrane Domain (TM) of |
| | S1S2 protein); intrinsic S1S2 protein secretory signal peptide |
| | (aa 1-19) at the N-terminus of the antigen sequence |

BNT162b3d (SEQ ID NO: 31; SEQ ID NO: 32)

| | |
|---|---|
| Structure | m₂^{7,3′-O}Gppp(m₁^{2′-O})ApG-hAg-Kozak-RBD-GS-Fibritin-GS- |
| | TM-FI-A30L70 |
| Encoded | Viral spike protein (S1S2 protein) of the SARS-CoV-2 (partial |
| antigen | sequence, Receptor Binding Domain (RBD) of S1S2 protein |
| | fused to Fibritin fused to Transmembrane Domain (TM) of |
| | S1S2 protein); immunoglobulin secretory signal peptide |
| | (aa 1-22) at the N-terminus of the antigen sequence |

Self-Amphfying RNA (saRNA)

The active principle of the self-amplifying mRNA (saRNA) drug substance is a single-stranded RNA, which self-amplifies upon entering a cell, and the coronavirus vaccine antigen is translated thereafter. In contrast to uRNA and modRNA that preferably code for a single protein, the coding region of saRNA contains two open reading frames (ORFs). The 5′-ORF encodes the RNA-dependent RNA polymerase such as Venezuelan equine encephalitis virus (VEEV) RNA-dependent RNA polymerase (replicase). The replicase ORF is followed 3′ by a subgenomic promoter and a second ORF encoding the antigen. Furthermore, saRNA UTRs contain 5′ and 3′ conserved sequence elements (CSEs) required for self-amplification. The saRNA contains common structural elements optimized for maximal efficacy of the RNA as the uRNA (5′-cap, 5′-UTR, 3′-UTR, poly(A)-tail). The saRNA preferably contains uridine. The preferred 5′ cap structure is beta-S-ARCA(D1) (m₂^{7,2′-O}GppSpG).

Cytoplasmic delivery of saRNA initiates an alphavirus-like life cycle. However, the saRNA does not encode for alphaviral structural proteins that are required for genome packaging or cell entry, therefore generation of replication competent viral particles is very unlikely to not possible. Replication does not involve any intermediate steps that generate DNA. The use/uptake of saRNA therefore poses no risk of genomic integration or other permanent genetic modification within the target cell. Furthermore, the saRNA itself prevents its persistent replication by effectively activating innate immune response via recognition of dsRNA intermediates.

Different embodiments of this platform are as follows:

RBS004.1 (SEQ ID NO: 24; SEQ ID NO: 7)

| | |
|---|---|
| Structure | beta-S-ARCA(D1)-replicase-S1S2-PP-FI-A30L70 |
| Encoded | Viral spike protein (S protein) of the SARS-CoV-2 (S1S2 |
| antigen | full-length protein, sequence variant) |

RBS004.2 (SEQ ID NO: 25; SEQ ID NO: 7)

| | |
|---|---|
| Structure | beta-S-ARCA(D1)-replicase-S1S2-PP-FI-A30L70 |
| Encoded | Viral spike protein (S protein) of the SARS-CoV-2 (S1S2 |
| antigen | full-length protein, sequence variant) |

BNT162c1; RBS004.3 (SEQ ID NO: 26; SEQ ID NO: 5)

| | |
|---|---|
| Structure | beta-S-ARCA(D1)-replicase-RBD-GS-Fibritin-FI-A30L70 |
| Encoded | Viral spike protein (S protein) of the SARS-CoV-2 (partial |
| antigen | sequence, Receptor Binding Domain (RBD) of S1S2 protein) |

RBS004.4 (SEQ ID NO: 27; SEQ ID NO: 28)

| | |
|---|---|
| Structure | beta-S-ARCA(D1)-replicase-RBD-GS-Fibritin-TM-FI- |
| | A30L70 |
| Encoded | Viral spike protein (S protein) of the SARS-CoV-2 (partial |
| antigen | sequence, Receptor Binding Domain (RBD) of S1S2 protein) |

Furthermore, a secretory signal peptide (sec) may be fused to the antigen-encoding regions preferably in a way that the sec is translated as N terminal tag. In some embodiments, sec corresponds to the secretory signal peptide of the S protein. Sequences coding for short linker peptides predominantly consisting of the amino acids glycine (G) and serine (S), as commonly used for fusion proteins may be used as GS/Linkers.

In some embodiments, RNA (preferably mRNA) encoding an antigen (such as a tumor antigen or a vaccine antigen) is expressed in cells of the subject treated to provide the antigen. In some embodiments, the RNA is transiently expressed in cells of the subject. In some embodiments, the RNA is in vitro transcribed. In some embodiments, expression of the antigen is at the cell surface. In some embodiments, the antigen is expressed and presented in the context of MHC. In some embodiments, expression of the antigen is into the extracellular space, i.e., the antigen is secreted.

The antigen molecule or a procession product thereof, e.g., a fragment thereof, may bind to an antigen receptor such as a BCR or TCR carried by immune effector cells, or to antibodies.

A peptide and protein antigen which is provided to a subject according to the present disclosure by administering RNA (such as mRNA) encoding a peptide and protein antigen, wherein the antigen is a vaccine antigen, preferably results in the induction of an immune response, e.g., a humoral and/or cellular immune response in the subject being provided the peptide or protein antigen. Said immune response is preferably directed against a target antigen. Thus, a vaccine antigen may comprise the target antigen, a variant thereof, or a fragment thereof. In one embodiment, such fragment or variant is immunologically equivalent to the target antigen. In the context of the present disclosure, the term “fragment of an antigen” or “variant of an antigen” means an agent which results in the induction of an immune response which immune response targets the antigen, i.e. a target antigen. Thus, the vaccine antigen may correspond to or may comprise the target antigen, may correspond to or may comprise a fragment of the target antigen or may correspond to or may comprise an antigen which is homologous to the target antigen or a fragment thereof. Thus, according to the present disclosure, a vaccine antigen may comprise an immunogenic fragment of a target antigen or an amino acid sequence being homologous to an immunogenic fragment of a target antigen. An “immunogenic fragment of an antigen” according to the disclosure preferably relates to a fragment of an antigen which is capable of inducing an immune response against the target antigen. The vaccine antigen may be a recombinant antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject induces an immune reaction having a specificity of reacting with the reference amino acid sequence. Thus, in some embodiments, a molecule which is immunologically equivalent to an antigen exhibits the same or essentially the same properties and/or exerts the same or essentially the same effects regarding the stimulation, priming and/or expansion of T cells as the antigen to which the T cells are targeted.

In one embodiment, the RNA (preferably mRNA) used in the present disclosure is non-immunogenic. RNA encoding an immunostimulant may be administered according to the present disclosure to provide an adjuvant effect. The RNA encoding an immunostimulant may be standard RNA or non-immunogenic RNA.

The term “non-immunogenic RNA” (such as “non-immunogenic mRNA”) as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In certain embodiments, non-immunogenic RNA, which is also termed modified RNA (modRNA) herein, is rendered non-immunogenic by incorporating modified nucleosides suppressing RNA-mediated activation of innate immune receptors into the RNA and/or limiting the amount of double-stranded RNA (dsRNA), e.g., by limiting the formation of double-stranded RNA (dsRNA), e.g., during in vitro transcription, and/or by removing double-stranded RNA (dsRNA), e.g., following in vitro transcription. In certain embodiments, non-immunogenic RNA is rendered non-immunogenic by incorporating modified nucleosides suppressing RNA-mediated activation of innate immune receptors into the RNA and/or by removing double-stranded RNA (dsRNA), e.g., following in vitro transcription.

For rendering the non-immunogenic RNA (especially mRNA) non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprise a replacement of one or more uridines with a nucleoside comprising a modified nucleobase.

In one embodiment, the modified nucleobase is a modified uracil. In some embodiments, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (nCm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (Tm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), I-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ym), 2-thio-2′-O-methyl-uridine (s²Um), 5-methoxycarbonylmethyl-2′-O-methyl-uridine (mem⁵Um), 5-carbamoylmethyl-2′-O-methyl-uridine (nCm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In one particularly preferred embodiment, the nucleoside comprising a modified nucleobase is pseudouridine (ψ), N1-methyl-pseudouridine (m1ψ) or 5-methyl-uridine (m5U), in particular N1-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of RNA (preferably mRNA) by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. Formation of dsRNA can be limited during synthesis of mRNA by in vitro transcription (IVT), for example, by limiting the amount of uridine triphosphate (UTP) during synthesis. Optionally, UTP may be added once or several times during synthesis of mRNA. Also, dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In one embodiment, an RNA preparation is contacted with a cellulose material and the ssRNA is separated from the cellulose material under conditions which allow binding of dsRNA to the cellulose material and do not allow binding of ssRNA to the cellulose material. Suitable methods for providing ssRNA are disclosed, for example, in WO 2017/182524.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the removal of dsRNA (especially dsmRNA) from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In one embodiment, the non-immunogenic RNA (especially mRNA) is free or essentially free of dsRNA. In some embodiments, the non-immunogenic RNA (especially mRNA) composition comprises a purified preparation of single-stranded nucleoside modified RNA. For example, in some embodiments, the purified preparation of single-stranded nucleoside modified RNA (especially mRNA) is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

Various methods can be used to determine the amount of dsRNA. For example, a sample may be contacted with dsRNA-specific antibody and the amount of antibody binding to RNA may be taken as a measure for the amount of dsRNA in the sample. A sample containing a known amount of dsRNA may be used as a reference.

For example, RNA may be spotted onto a membrane, e.g., nylon blotting membrane. The membrane may be blocked, e.g., in TBS-T buffer (20 mM TRIS pH 7.4, 137 mM NaCl, 0.1% (v/v) TWEEN-20) containing 5% (w/v) skim milk powder. For detection of dsRNA, the membrane may be incubated with dsRNA-specific antibody, e.g., dsRNA-specific mouse mAb (English & Scientific Consulting, Szirik, Hungary). After washing, e.g., with TBS-T, the membrane may be incubated with a secondary antibody, e.g., HRP-conjugated donkey anti-mouse IgG (Jackson ImmunoResearch, Cat #715-035-150), and the signal provided by the secondary antibody may be detected.

In some embodiments, the non-immunogenic RNA (especially mRNA) is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor. In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In one embodiment, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10⁻³⁰⁰-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the non-immunogenic RNA (especially mRNA) exhibits significantly less innate immunogenicity than standard RNA with the same sequence. In some embodiments, the non-immunogenic RNA (especially mRNA) exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by a 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor.

The term “exhibits significantly less innate immunogenicity” refers to a detectable decrease in innate immunogenicity. In some embodiments, the term refers to a decrease such that an effective amount of the non-immunogenic RNA (especially mRNA) can be administered without triggering a detectable innate immune response. In some embodiments, the term refers to a decrease such that the non-immunogenic RNA (especially mRNA) can be repeatedly administered without eliciting an innate immune response sufficient to detectably reduce production of the protein encoded by the non-immunogenic RNA. In some embodiments, the decrease is such that the non-immunogenic RNA (especially mRNA) can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked”, “fused”, or “fusion” are used interchangeably. These terms refer to the joining together of two or more elements or components or domains.

### Particles

Nucleic acids such as RNA, in particular mRNA, described herein may be present in particles comprising (i) the nucleic acid, and (ii) at least one cationic or cationically ionizable compound such as a polymer or lipid complexing the nucleic acid.

Different types of RNA containing particles have been described previously to be suitable for delivery of RNA in particulate form (cf., e.g., Kaczmarek, J. C. et al., 2017, Genome Medicine 9, 60). For non-viral RNA delivery vehicles, nanoparticle encapsulation of RNA physically protects RNA from degradation and, depending on the specific chemistry, can aid in cellular uptake and endosomal escape.

Electrostatic interactions between positively charged molecules such as polymers and lipids and negatively charged nucleic acid are involved in particle formation. This results in complexation and spontaneous formation of nucleic acid particles.

During the manufacturing process, introduction of an aqueous solution of RNA to an ethanolic lipid mixture containing a cationically ionizable lipid at pH of, e.g., 5 leads to an electrostatic interaction between the negatively charged RNA drug substance and the positively charged cationically ionizable lipid. This electrostatic interaction leads to particle formation coincident with efficient encapsulation of RNA drug substance. After RNA encapsulation, adjustment of the medium surrounding the resulting RNA-LNP to, e.g., pH 8 results in neutralization of the surface charge on the LNP. When all other variables are held constant, charge-neutral particles display longer in vivo circulation lifetimes and better delivery to hepatocytes compared to charged particles, which are cleared rapidly by the reticuloendothelial system. Upon endosomal uptake, the low pH of the endosome renders the LNP fusogenic and allows for release of the RNA into the cytosol of the target cell.

In the context of the present disclosure, the term “particle” relates to a structured entity formed by molecules or molecule complexes, in particular particle forming compounds. In some embodiments, the particle contains an envelope (e.g., one or more layers or lamellas) made of one or more types of amphiphilic substances (e.g., amphiphilic lipids, amphiphilic polymers, and/or amphiphilic proteins/polypeptides). In this context, the expression “amphiphilic substance” means that the substance possesses both hydrophilic and lipophilic properties. The envelope may also comprise additional substances (e.g., additional lipids and/or additional polymers) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids, amphiphilic polymers, and/or amphiphilic proteins/polypeptides) optionally in combination with additional substances (e.g., additional lipids and/or additional polymers) which do not have to be amphiphilic. In some embodiments, the term “particle” relates to a micro- or nano-sized structure, such as a micro- or nano-sized compact structure. In this respect, the term “micro-sized” means that all three external dimensions of the particle are in the microscale, i.e., between 1 and 5 μm. According to the present disclosure, the term “particle” includes lipoplex particles (LPXs), lipid nanoparticles (LNPs), polyplex particles, lipopolyplex particles, virus-like particles (VLPs), and mixtures thereof (e.g., a mixture of two or more of particle types, such as a mixture of LPXs and VLPs or a mixture of LNPs and VLPs).

A “nucleic acid particle” can be used to deliver nucleic acid (such as RNA, in particular mRNA) to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and/or the cationic polymer combine together with the nucleic acid to form aggregates, and this aggregation results in colloidally stable particles. In some embodiments, particles comprise an amphiphilic lipid, in particular cationic or cationically ionizable amphiphilic lipid, and RNA (especially mRNA) as described herein. In some embodiments, particles comprise or consist of a cationic/cationically ionizable lipid (in particular, a cationically ionizable lipid of formula (X) disclosed herein; a cationically ionizable lipid having one of the structures A to G disclosed herein; or a cationically ionizable lipid of formula (XI) disclosed herein) and helper lipids such as polymer-conjugated lipids (e.g., a polyethylene glycol (PEG) lipid; or a polysarcosine-lipid conjugate or a conjugate of polysarcosine and a lipid-like material); a neutral lipid (such as a phospholipid); a steroid (such as cholesterol); and combinations thereof. In some embodiments, in the RNA particles described herein the RNA (in particular, mRNA) is bound by cationically ionizable lipid (in particular a cationically ionizable lipid of formula (X) disclosed herein; a cationically ionizable lipid having one of the structures A to G disclosed herein; or a cationically ionizable lipid of formula (XI) disclosed herein) that, in the case of LNPs, occupies the central core of the LNPs. In some embodiments, polymer-conjugated lipid (e.g., a PEG lipid; or a polysarcosine-lipid conjugate or a conjugate of polysarcosine and a lipid-like material) forms the surface of the particles (such as LNPs), along with phospholipids. In some embodiments, the surface comprises a bilayer. In some embodiments, cholesterol and cationically ionizable lipid (in particular a cationically ionizable lipid of formula (X) disclosed herein; a cationically ionizable lipid having one of the structures A to G disclosed herein; or a cationically ionizable lipid of formula (XI) disclosed herein) in charged and uncharged forms can be distributed throughout the particles such as LNPs.

In general, a lipoplex (LPX) is obtainable from mixing two aqueous phases, namely a phase comprising RNA and a phase comprising a dispersion of lipids. In some embodiments, the lipid phase comprises liposomes.

In some embodiments, liposomes are self-closed unilamellar or multilamellar vesicular particles wherein the lamellae comprise lipid bilayers and the encapsulated lumen comprises an aqueous phase. A prerequisite for using liposomes for nanoparticle formation is that the lipids in the mixture as required are able to form lamellar (bilayer) phases in the applied aqueous environment.

In some embodiments, liposomes comprise unilamellar or multilamellar phospholipid bilayers enclosing an aqueous core (also referred to herein as an aqueous lumen). They may be prepared from materials possessing polar head (hydrophilic) groups and nonpolar tail (hydrophobic) groups. In some embodiments, cationic lipids employed in formulating liposomes designed for the delivery of nucleic acids are amphiphilic in nature and consist of a positively charged (cationic) amine head group linked to a hydrocarbon chain or cholesterol derivative via glycerol.

In some embodiments, lipoplexes are multilamellar liposome-based formulations that form upon electrostatic interaction of cationic liposomes with RNAs. In some embodiments, formed lipoplexes possess distinct internal arrangements of molecules that arise due to the transformation from liposomal structure into compact RNA-lipoplexes. In some embodiments, these formulations are characterized by their poor encapsulation of the RNA and incomplete entrapment of the RNA.

In some embodiments, an LPX particle comprises an amphiphilic lipid, in particular cationic or cationically ionizable amphiphilic lipid, and RNA (especially mRNA) as described herein. In some embodiments, electrostatic interactions between positively charged liposomes (made from one or more amphiphilic lipids, in particular cationic or cationically ionizable amphiphilic lipids) and negatively charged RNA (especially mRNA) results in complexation and spontaneous formation of RNA lipoplex particles. Positively charged liposomes may be generally synthesized using a cationic or cationically ionizable amphiphilic lipid, such as a cationically ionizable lipid of formula (I), DOTMA and/or DODMA, and additional lipids, such as DOPE or DSPC. In some embodiments, an RNA (especially mRNA) lipoplex particle is a nanoparticle.

In general, a lipid nanoparticle (LNP) is obtainable from direct mixing of RNA in an aqueous phase with lipids in a phase comprising an organic solvent, such as ethanol. In that case, lipids or lipid mixtures can be used for particle formation, which do not form lamellar (bilayer) phases in water.

In some embodiments, particles described herein further comprise at least one lipid or lipid-like material other than a cationically ionizable lipid.

In some embodiments, nucleic acid particles (especially RNA particles such as RNA LNPs (e.g., mRNA particles such as mRNA LNPs)) comprise more than one type of nucleic acid molecules, where the molecular parameters of the nucleic acid molecules may be similar or different from each other, like with respect to molar mass or fundamental structural elements such as molecular architecture, capping, coding regions or other features,

In some embodiments, RNA (e.g., mRNA) described herein may be noncovalently associated with a particle as described herein. In some embodiments, the RNA (especially mRNA) may be adhered to the outer surface of the particle (surface RNA (especially surface mRNA)) and/or may be contained in the particle (encapsulated RNA (especially encapsulated mRNA)).

As used in the present disclosure, “nanoparticle” refers to a particle comprising nucleic acid (especially RNA such as mRNA) as described herein and at least one cationic lipid, wherein all three external dimensions of the particle are in the nanoscale, i.e., at least about 1 nm and below about 1000 nm (preferably, between 10 and 990 nm, such as between 15 and 900 nm, between 20 and 800 nm, between 30 and 700 nm, between 40 and 600 nm, or between 50 and 500 nm). Preferably, the longest and shortest axes do not differ significantly. Preferably, the size of a particle is its diameter.

Nucleic acid particles described herein (especially RNA particles, such as mRNA particles) may exhibit a polydispersity index (PDI) less than about 0.5, less than about 0.4, less than about 0.3, less than about 0.2, less than about 0.1, or less than about 0.05. By way of example, the nucleic acid particles can exhibit a polydispersity index in a range of about 0.01 to about 0.4 or about 0.1 to about 0.3.

In the context of the present disclosure, the term “lipoplex particle” relates to a particle that contains an amphiphilic lipid, in particular cationic amphiphilic lipid, and nucleic acid (especially RNA such as mRNA) as described herein. Electrostatic interactions between positively charged liposomes (made from one or more amphiphilic lipids, in particular cationic amphiphilic lipids) and negatively charged nucleic acid (especially RNA such as mRNA) results in complexation and spontaneous formation of nucleic acid lipoplex particles. Positively charged liposomes may be generally synthesized using a cationic amphiphilic lipid, such as DOTMA, and additional lipids, such as DOPE. In one embodiment, a nucleic acid (especially RNA such as mRNA) lipoplex particle is a nanoparticle.

The term “lipid nanoparticle” relates to a nano-sized lipid containing particle.

In the context of the present disclosure, the term “polyplex particle” relates to a particle that contains an amphiphilic polymer, in particular a cationic amphiphilic polymer, and nucleic acid (especially RNA such as mRNA) as described herein. Electrostatic interactions between positively charged cationic amphiphilic polymers and negatively charged nucleic acid (especially RNA such as mRNA) results in complexation and spontaneous formation of nucleic acid polyplex particles. Positively charged amphiphilic polymers suitable for the preparation of polyplex particle include protamine, polyethyleneimine, poly-L-lysine, poly-L-arginine and histone. In one embodiment, a nucleic acid (especially RNA such as mRNA) polyplex particle is a nanoparticle.

The term “lipopolyplex particle” relates to particle that contains amphiphilic lipid (in particular cationic amphiphilic lipid) as described herein, amphiphilic polymer (in particular cationic amphiphilic polymer) as described herein, and nucleic acid (especially RNA such as mRNA) as described herein. In one embodiment, a nucleic acid (especially RNA such as mRNA) lipopolyplex particle is a nanoparticle.

The term “virus-like particle” (abbreviated herein as VLP) refers to a molecule that closely resembles a virus, but which does not contain any genetic material of said virus and, thus, is non-infectious. Preferably, VLPs contain nucleic acid (preferably RNA) as described herein, said nucleic acid (preferably RNA) being heterologous to the virus(es) from which the VLPs are derived. VLPs can be synthesized through the individual expression of viral structural proteins, which can then self-assemble into the virus-like structure. In one embodiment, combinations of structural capsid proteins from different viruses can be used to create recombinant VLPs. VLPs can be produced from components of a wide variety of virus families including Hepatitis B virus (HBV) (small HBV derived surface antigen (HBsAg)), Parvoviridae (e.g., adeno-associated virus), Papillomaviridae (e.g., HPV), Retroviridae (e.g., HIV), Flaviviridae (e.g., Hepatitis C virus) and bacteriophages (e.g. Qβ, AP205).

The term “nucleic acid containing particle” relates to a particle as described herein to which nucleic acid (especially RNA such as mRNA) is bound. In this respect, the nucleic acid (especially RNA such as mRNA) may be adhered to the outer surface of the particle (surface nucleic acid (especially surface RNA such as surface mRNA)) and/or may be contained in the particle (encapsulated nucleic acid (especially encapsulated RNA such as encapsulated mRNA)).

In one embodiment, the particles utilized in the methods and uses of the present disclosure have a size (preferably a diameter, i.e., double the radius such as double the radius of gyration (R_{g}) value or double the hydrodynamic radius) in the range of about 10 to about 2000 nm, such as at least about 15 nm (preferably at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 inn, at least about 75 nm, at least about 80 nm, at least about 85 nm, at least about 90 nm, at least about 95 nm, or at least about 100 nm) and/or at most 1900 nm (preferably at most about 1900 nm, at most about 1800 nm, at most about 1700 nm, at most about 1600 nm, at most about 1500 nm, at most about 1400 nm, at most about 1300 nm, at most about 1200 nm, at most about 1100 nm, at most about 1000 nm, at most about 950 nm, at most about 900 nm, at most about 850 nm, at most about 800 nm, at most about 750 nm, at most about 700 nm, at most about 650 inn, at most about 600 nm, at most about 550 nm, or at most about 500 nm), preferably in the range of about 20 to about 1500 nm, such as about 30 to about 1200 nm, about 40 to about 1100 nm, about 50 to about 1000 nm, about 60 to about 900 nm, about 70 to 800 nm, about 80 to 700 nm, about 90 to 600 nm, or about 50 to 500 nm or about 100 to 500 nm, such as in the range of 10 to 1000 nm, 15 to 500 nm, 20 to 450 nm, 25 to 400 nm, 30 to 350 nm, 40 to 300 nm, 50 to 250 nm, 60 to 200 urn, or 70 to 150 nm.

In some embodiments, the particles (e.g., LNPs and LPXs) described herein have an average diameter that in some embodiments ranges from about 50 nm to about 1000 nm, from about 50 nm to about 800 nm, from about 50 nm to about 700 nm, from about 50 nm to about 600 run, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 un to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nrm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 run, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, or from about 200 nm to about 250 nm.

With respect to RNA lipid particles (especially RNA LNPs such as mRNA LNPs), the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles (especially RNA particles such as mRNA particles) described herein can be prepared using a wide range of methods that may involve obtaining a colloid from at least one cationic or cationically ionizable lipid and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the nucleic acid (especially RNA such as mRNA) lipoplex particles described herein are obtainable by adding nucleic acid (especially RNA such as mRNA) to a colloidal liposome dispersion. Using the ethanol injection technique, such colloidal liposome dispersion is, in one embodiment, formed as follows: an ethanol solution comprising lipids, such as cationically ionizable lipids (like a cationically ionizable lipid of formula (X) disclosed herein; a cationically ionizable lipid having one of the structures A to G disclosed herein; a cationically ionizable lipid of formula (XI) disclosed herein; DOTMA and/or DODMA) and additional lipids (such as a polymer-conjugated lipid (e.g., a polyethylene glycol (PEG) lipid; or a polysarcosine-lipid conjugate or a conjugate of polysarcosine and a lipid-like material); a neutral lipid (such as a phospholipid); a steroid (such as cholesterol); and combinations), is injected into an aqueous solution under stirring. In some embodiments, the nucleic acid (especially RNA such as mRNA) lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid. Each component is responsible for payload protection, and enables effective intracellular delivery. LNPs may be prepared by mixing lipids dissolved in ethanol rapidly with nucleic acid in an aqueous buffer. While RNA particles described herein may comprise a polymer-conjugated lipid such as a PEG lipid, or a sarcosinalyted lipid, provided herein are also RNA particles which do not comprise (i) PEG lipids, or do not comprise (ii) sarcosinylated lipids, or not comprise any combination of (i) and (ii). In some embodiments, the RNA particles do not comprise any polymer-conjugated lipid (in other words, those RNA particles are substantially free of polymer-conjugated lipids).

Different types of nucleic acid containing particles have been described previously to be suitable for delivery of nucleic acid in particulate form (cf., e.g., Kaczmarek, J. C. el al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

In one preferred embodiment, the LNPs comprising RNA and at least one cationically ionizable lipid described herein further comprise one or more additional lipids.

In some embodiments, the LNPs comprising RNA and at least one cationically ionizable lipid described herein are prepared by (a) preparing an RNA solution containing water and a first buffer system; (b) preparing an ethanolic solution comprising the cationically ionizable lipid and, if present, one or more additional lipids; (c) mixing the RNA solution prepared under (a) with the ethanolic solution prepared under (b), thereby preparing a first intermediate formulation comprising the LNPs dispersed in a first aqueous phase comprising the first buffer system; and (d) filtrating the first intermediate formulation prepared under (c) using a final aqueous buffer solution comprising the final buffer system, thereby preparing the formulation comprising LNPs dispersed in a final aqueous phase comprising the final buffer system. After step (c) one or more steps selected from diluting and filtrating, such as tangential flow filtrating or diafiltrating, can follow. In some embodiments, the first buffer system differs from the final buffer system. In alternative embodiments, the first buffer system and the final buffer system are the same.

In some embodiments, the LNPs comprising RNA and at least one cationically ionizable lipid described herein are prepared by (a′) preparing liposomes or a colloidal preparation of the cationically ionizable lipid and, if present, one or more additional lipids in an aqueous phase; (b′) preparing an RNA solution containing water and a buffering system; and (c′) mixing the liposomes or colloidal preparation prepared under (a′) with the mRNA solution prepared under (b′). After step (c′) one or more steps selected from diluting and filtrating, such as tangential flow filtrating, can follow.

The present disclosure describes compositions which comprise RNA (especially mRNA) and at least one cationically ionizable lipid which associates with the RNA to form nucleic acid particles. The RNA particles may comprise RNA which is complexed in different forms by non-covalent interactions to the particle. The particles described herein are not viral particles, in particular infectious viral particles, i.e., they are not able to virally infect cells.

Suitable cationically ionizable lipids are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

In some embodiments, RNA particles (especially mRNA particles) comprise more than one type of RNA molecules, where the molecular parameters of the RNA molecules may be similar or different from each other, like with respect to molar mass or fundamental structural elements such as molecular architecture, capping, coding regions or other features.

In particulate formulation, it is possible that each RNA species is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the present disclosure are obtainable by forming, separately, individual particulate formulations, followed by a step of mixing of the individual particulate formulations. By the step of mixing, a formulation comprising a mixed population of RNA-containing particles is obtainable. Individual particulate populations may be together in one container, comprising a mixed population of individual particulate formulations. Alternatively, it is possible that all RNA species of the pharmaceutical composition are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The monovalent radical of a hydrocarbon is referred to as hydrocarbyl herein. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes non-cyclic, e.g., linear (straight) or branched, hydrocarbyl groups, such as alkyl, alkenyl, or alkynyl as defined herein. It should be appreciated that one or more of the hydrogen atoms in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds. The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids comprising two long alkyl chains and a polar head group are generally cylindrical. The area occupied by the two alkyl chains is similar to the area occupied by the polar head group. Such lipids have low solubility as monomers and tend to aggregate into planar bilayers that are water insoluble. Traditional surfactant monomers comprising only one linear alkyl chain and a hydrophilic head group are generally cone shaped. The hydrophilic head group tends to occupy more molecular space than the linear alkyl chain. In some embodiments, surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents, Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits).

Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

### Cationically Ionizable Lipids

The RNA compositions described herein and the nucleic acid particles (especially RNA LNPs) described herein comprise at least one cationically ionizable lipid as particle forming agent. Cationically ionizable lipids contemplated for use herein include any cationically ionizable lipids or lipid-like materials which are able to electrostatically bind nucleic acid. In one embodiment, cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., a lipid which is not permanently cationic. Thus, depending on the pH of the composition in which the cationically ionizable lipid is solved, the cationically ionizable lipid is either positively charged or neutral. For purposes of the present disclosure, such “cationically ionizable” lipids are comprised by the term “cationic lipid” unless contradicted by the circumstances.

In one embodiment, the cationically ionizable lipid comprises a head group which includes at least one nitrogen atom (N) which is positive charged or capable of being protonated, preferably under physiological conditions.

Examples of cationic lipids include, but are not limited to N,N-dimethyl-2,3-dioleyloxypropylamine (DODMA), 1,2-di-O-octadecenyl-3-trimethylammonium propane (DOTMA), 3-(N-(N′,N′-dimethylaminoethane)-carbamoyl)cholesterol (DC-Chol), dimethyldioctadecylammonium (DDAB); 1,2-dioleoyl-3-trimethylanmonium propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (t)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (pAE-DMRIE), N-(4-carboxybenzyl)-N,N-dimethyl-2,3-bis(oleoyloxy)propan-1-aminium (DOBAQ), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)-dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), D1((Z)-non-2-en-1-yl)-9-((4-(dimethylaminobutanoyl)-oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethyl-amino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200). Preferred are DODMA, DOTMA, DOTAP, DODAC, and DOSPA. In specific embodiments, the cationic or cationically ionizable lipid is DODMA.

DOTMA is a cationic lipid with a quaternary amine headgroup. The structure of DOTMA may be represented as follows:

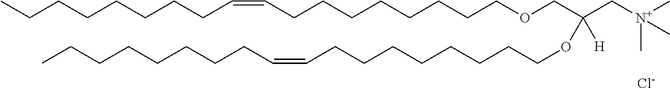 [SMILES: CCCCCCCC/C=C\CCCCCCCCOCC(C[N+](C)(C)C)OCCCCCCCC/C=C\CCCCCCCC ; [Cl-]]

DODMA is an ionizable cationic lipid with a tertiary amine headgroup. The structure of DODMA may be represented as follows:

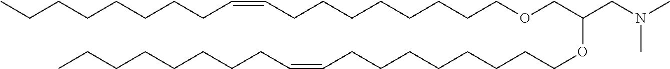 [SMILES: CCCCCCCC/C=C\CCCCCCCCOCC(CN(C)C)OCCCCCCCC/C=C\CCCCCCCC]

In certain embodiments, the composition comprises a cationically ionizable lipid.

In some embodiments, the cationically ionizable lipid comprises a head group which includes at least one nitrogen atom (N) which is positive charged or capable of being protonated, preferably under physiological conditions.

Examples of cationically ionizable lipids are disclosed, for example, in WO 2016/176330 and WO 2018/078053. In some embodiments, the cationically ionizable lipid has the structure of Formula (X):

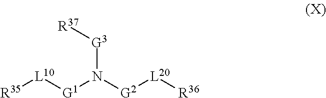

or a pharmaceutically acceptable salt, tautomer, prodrug or stereoisomer thereof, wherein: one of L¹⁰ and L²⁰ is —O(C═O)—, —(C═O)O—, —C(═O)—, —O—, —S(O)ₓ-, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and
L²⁰ is —O(C═O)—, —(C═O)O—, —C(═O)—, —O—, —S(O)ₓ—, —S—S—, —C(═O)S—, SC(═O)—, —NRWC(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆₋₂₄ alkyl or C₆₋₂₄ alkenyl;
R³⁷ is H, OR⁵⁰, CN, —C(═O)OR⁴⁰, —OC(═O)R⁴⁰ or NR⁵⁰⁰C(═O)R⁴⁰;
R⁴⁰ is C₁₋₁₂ alkyl;
R⁵⁰ is H or C₁₋₆ alkyl; and
x is 0, 1 or 2.

In some of the foregoing embodiments of Formula (X), the lipid has one of the following structures (XA) or (XB):

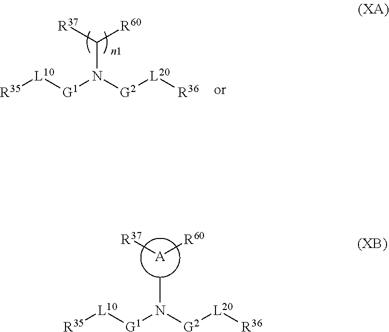

wherein:
A is a 3 to 8-membered cycloalkyl or cycloalkylene group;
R⁶⁰ is, at each occurrence, independently H, OH or C₁-C₂₄ alkyl;
n1 is an integer ranging from 1 to 15.

In some of the foregoing embodiments of Formula (X), the lipid has structure (XA), and in other embodiments, the lipid has structure (XB).

In other embodiments of Formula (X), the lipid has one of the following structures (XC) or (XD):

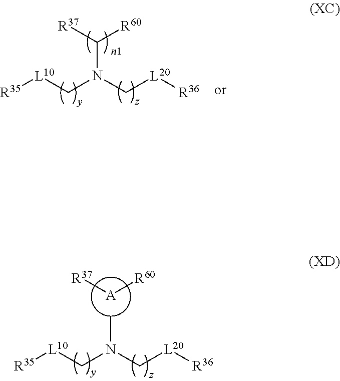

wherein y and z are each independently integers ranging from 1 to 12.

In any of the foregoing embodiments of Formula (X), one of L¹⁰ and L²⁰ is —O(C═O)—. For example, in some embodiments each of L¹⁰ and L²⁰ are —O(C═O)—. In some different embodiments of any of the foregoing, L¹⁰ and L²⁰ are each independently —(C═O)O— or —O(C═O)—. For example, in some embodiments each of L¹⁰ and L²⁰ is —(C═O)O—.

In some different embodiments of Formula (X), the lipid has one of the following structures (XE) or (XF):

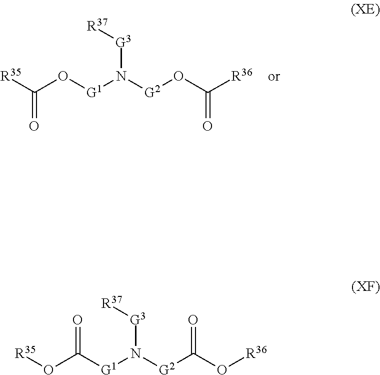

In some of the foregoing embodiments of Formula (X), the lipid has one of the following structures (XG), (XH), (XJ), or (XK):

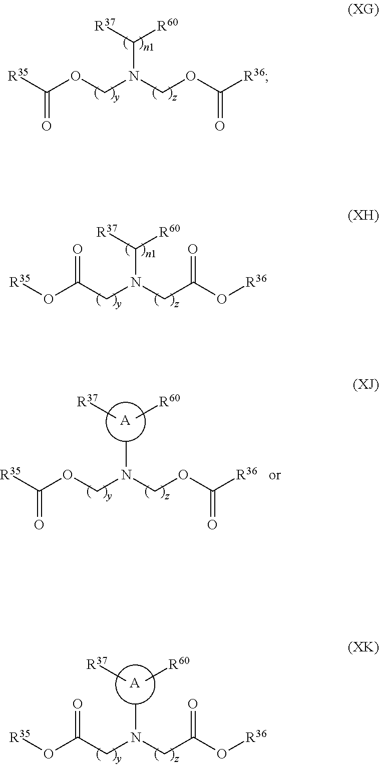

In some of the foregoing embodiments of Formula (X), n1 is an integer ranging from 2 to 12, for example from 2 to 8 or from 2 to 4. For example, in some embodiments, n1 is 3, 4, 5 or 6. In some embodiments, n1 is 3. In some embodiments, n1 is 4. In some embodiments, n1 is 5. In some embodiments, n1 is 6.

In some other of the foregoing embodiments of Formula (X), y and z are each independently an integer ranging from 2 to 10. For example, in some embodiments, y and z are each independently an integer ranging from 4 to 9 or from 4 to 6.

In some of the foregoing embodiments of Formula (X), R⁶⁰ is H. In other of the foregoing embodiments, R⁶⁰ is C₁-C₂₄ alkyl. In other embodiments, R⁶⁰ is OH.

In some embodiments of Formula (X), G³ is unsubstituted. In other embodiments, G³ is substituted. In various different embodiments, G³ is linear C₁-C₂₄ alkylene or linear C₂-C₂₄ alkenylene.

In some other foregoing embodiments of Formula (X), R³⁵ or R³⁶, or both, is C₆-C₂₄ alkenyl. For example, in some embodiments, R³⁵ and R³⁶ each, independently have the following structure:

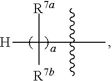

wherein:
R^{7a} and R^{7b} are, at each occurrence, independently H or C₁-C₁₂ alkyl; and
a is an integer from 2 to 12,
wherein R^{7a}, R^{7b} and a are each selected such that R³⁵ and R³⁶ each independently comprise from 6 to 20 carbon atoms. For example, in some embodiments a is an integer ranging from 5 to 9 or from 8 to 12,

In some of the foregoing embodiments of Formula (X), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (X), R³⁵ or R³⁶, or both, has one of the following structures:

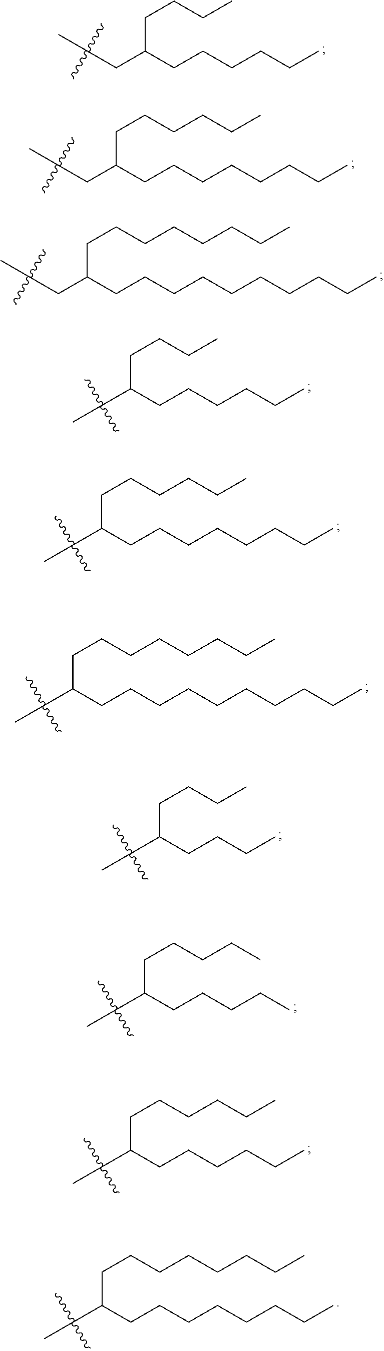

In some of the foregoing embodiments of Formula (X), R³⁷ is OH, CN, —C(═O)OR⁴⁰, —OC(═O)R⁴⁰ or —NHC(═O)R⁴⁰. In some embodiments, R⁴⁰ is methyl or ethyl.

In various different embodiments, the cationic lipid of Formula (X) has one of the structures set forth below.

Representative Compounds of Formula (X).

| | |
|---|---|
| X-1 | 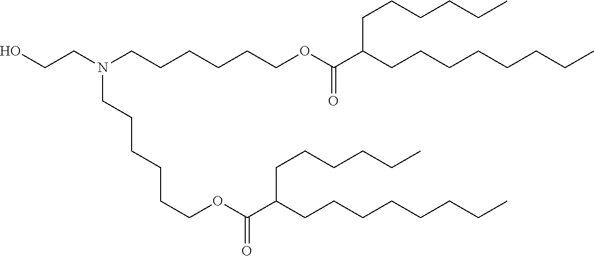 [SMILES: CCCCCCCCC(CCCCCC)C(=O)OCCCCCCN(CCO)CCCCCCOC(=O)C(CCCCCC)CCCCCCCC] |
| X-2 | 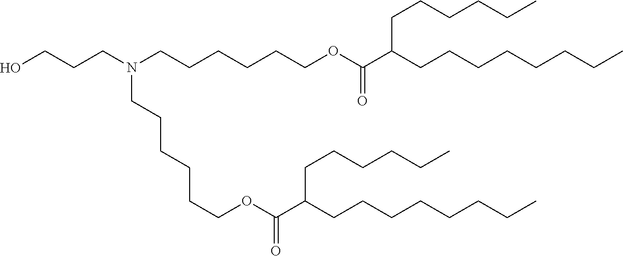 [SMILES: CCCCCCCCC(CCCCCC)C(=O)OCCCCCCN(CCCO)CCCCCCOC(=O)C(CCCCCC)CCCCCCCC] |
| X-3 | 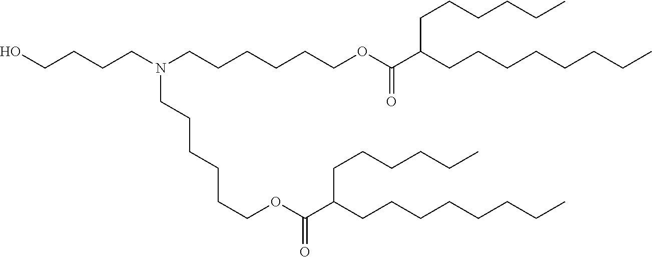 [SMILES: CCCCCCCCC(CCCCCC)C(=O)OCCCCCCN(CCCCO)CCCCCCOC(=O)C(CCCCCC)CCCCCCCC] |
| X-4 | 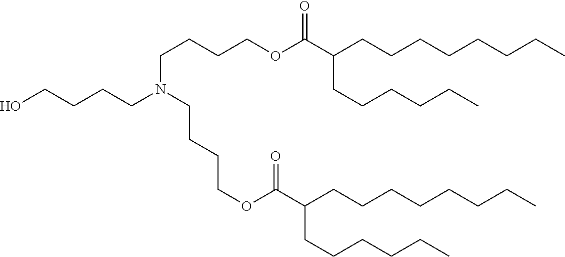 [SMILES: CCCCCCCCC(CCCCCC)C(=O)OCCCCN(CCCCO)CCCCOC(=O)C(CCCCCC)CCCCCCCC] |
| X-5 | 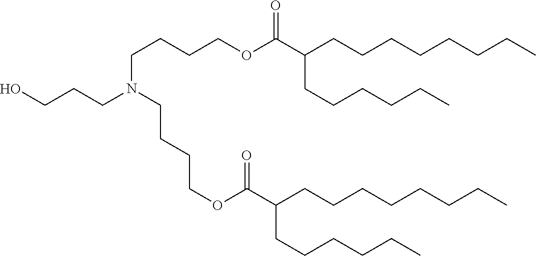 [SMILES: CCCCCCCCC(CCCCCC)C(=O)OCCCCN(CCCO)CCCCOC(=O)C(CCCCCC)CCCCCCCC] |
| X-6 | 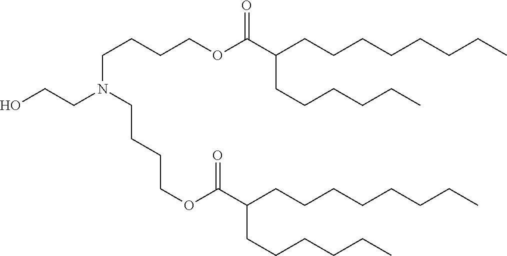 [SMILES: CCCCCCCCC(CCCCCC)C(=O)OCCCCN(CCO)CCCCOC(=O)C(CCCCCC)CCCCCCCC] |
| X-7 | 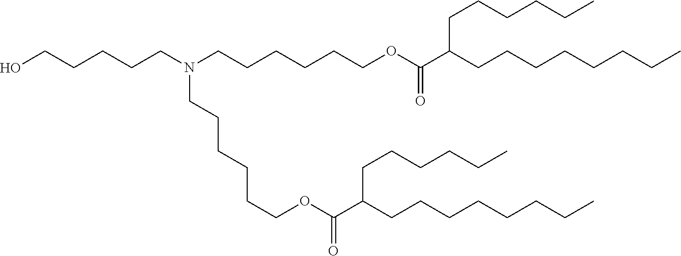 [SMILES: CCCCCCCCC(CCCCCC)C(=O)OCCCCCCN(CCCCCO)CCCCCCOC(=O)C(CCCCCC)CCCCCCCC] |
| X-8 | 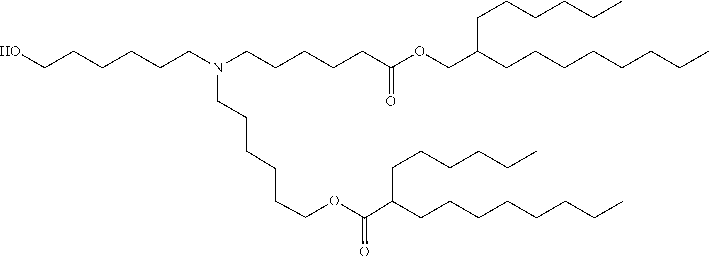 [SMILES: CCCCCCCCC(CCCCCC)COC(=O)CCCCCN(CCCCCCO)CCCCCCOC(=O)C(CCCCCC)CCCCCCCC] |
| X-9 | 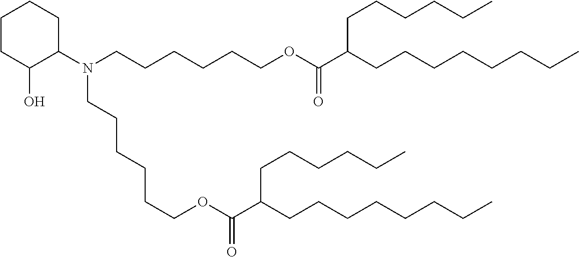 [SMILES: CCCCCCCCC(CCCCCC)C(=O)OCCCCCCN(CCCCCCOC(=O)C(CCCCCC)CCCCCCCC)C1CCCCC1O] |
| X-10 | 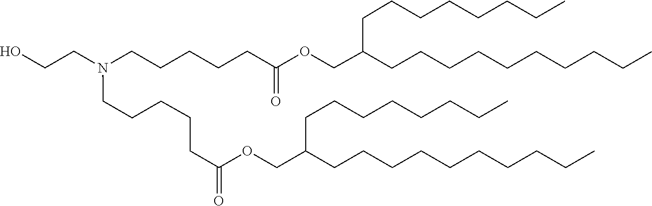 [SMILES: CCCCCCCCCCC(CCCCCCCC)COC(=O)CCCCCN(CCO)CCCCCC(=O)OCC(CCCCCCCC)CCCCCCCCCC] |
| X-11 | 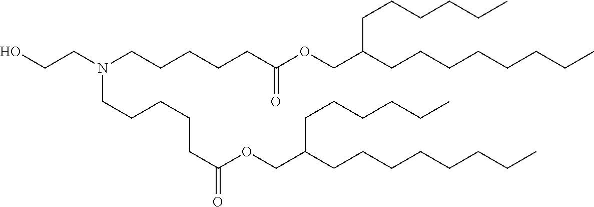 [SMILES: CCCCCCCCC(CCCCCC)COC(=O)CCCCCN(CCO)CCCCCC(=O)OCC(CCCCCC)CCCCCCCC] |
| X-12 | 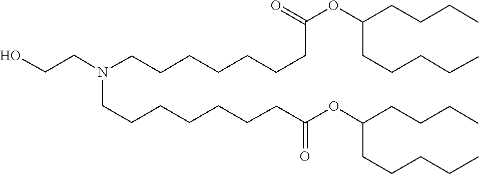 [SMILES: CCCCCC(CCCC)OC(=O)CCCCCCCN(CCO)CCCCCCCC(=O)OC(CCCC)CCCCC] |
| X-13 | 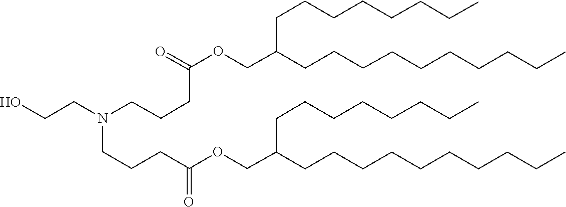 [SMILES: CCCCCCCCCCC(CCCCCCCC)COC(=O)CCCN(CCO)CCCC(=O)OCC(CCCCCCCC)CCCCCCCCCC] |
| X-14 | 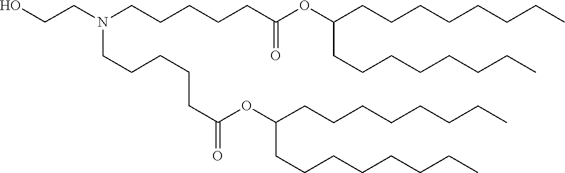 [SMILES: CCCCCCCCC(CCCCCCCC)OC(=O)CCCCCCN(CCO)CCCCCC(=O)OC(CCCCCCCC)CCCCCCCC] |
| X-15 | 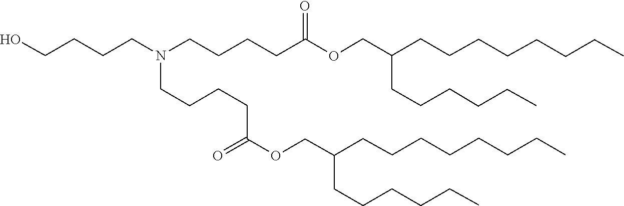 [SMILES: CCCCCCCCC(CCCCCC)COC(=O)CCCCN(CCCCO)CCCCC(=O)OCC(CCCCCC)CCCCCCCC] |
| X-16 | 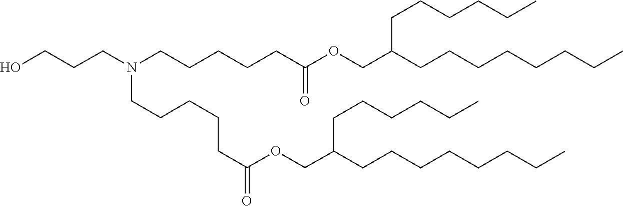 [SMILES: CCCCCCCCC(CCCCCC)COC(=O)CCCCCCN(CCCO)CCCCCC(=O)OCC(CCCCCC)CCCCCCCC] |
| X-17 | 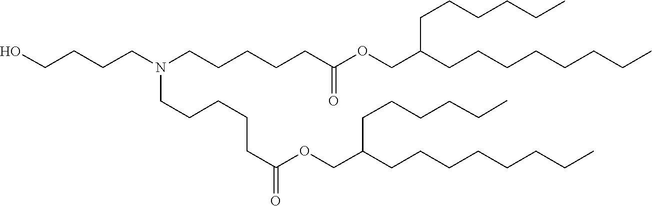 [SMILES: CCCCCCCCC(CCCCCC)COC(=O)CCCCCCN(CCCCO)CCCCCC(=O)OCC(CCCCCC)CCCCCCCC] |
| X-18 | 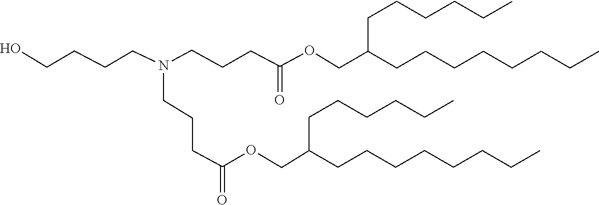 [SMILES: CCCCCCCCC(CCCCCC)COC(=O)CCCCN(CCCCO)CCCC(=O)OCC(CCCCCC)CCCCCCCC] |
| X-19 | 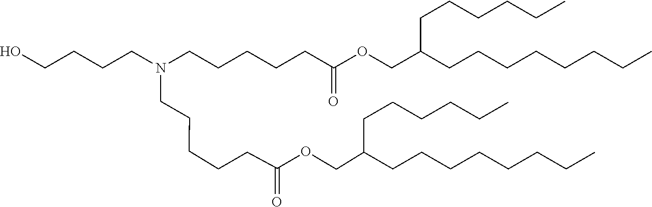 [SMILES: CCCCCCCCC(CCCCCC)COC(=O)CCCCCN(CCCCO)CCCCCC(=O)OCC(CCCCCC)CCCCCCCC] |
| X-20 | 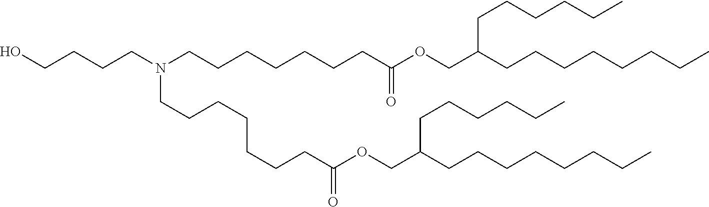 [SMILES: CCCCCCCCC(CCCCCC)COC(=O)CCCCCCCN(CCCCO)CCCCCCCC(=O)OCC(CCCCCC)CCCCCCCC] |
| X-21 | 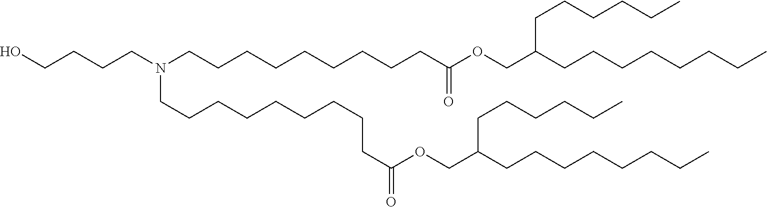 [SMILES: CCCCCCCCC(CCCCCC)COC(=O)CCCCCCCCCN(CCCCO)CCCCCCCCCC(=O)OCC(CCCCCC)CCCCCCCC] |
| X-22 | 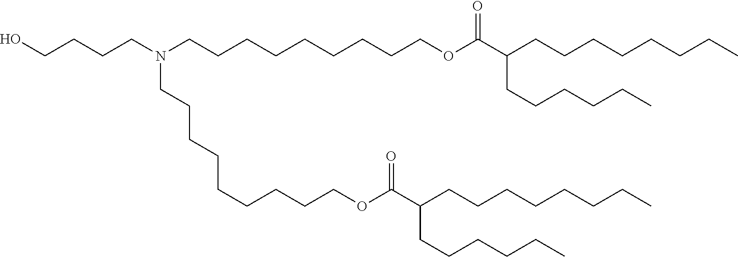 [SMILES: CCCCCCCCC(CCCCCC)C(=O)OCCCCCCCCCN(CCCCO)CCCCCCCCCOC(=O)C(CCCCCC)CCCCCCCC] |
| X-23 | 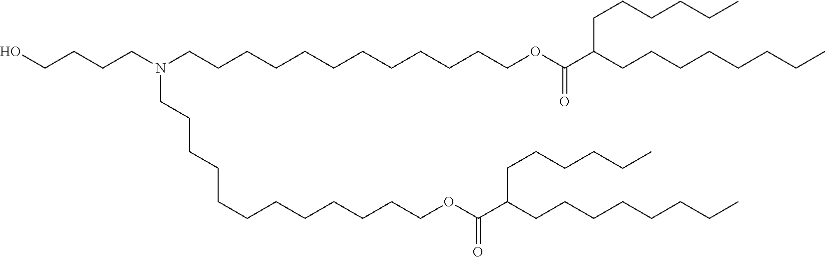 [SMILES: CCCCCCCCC(CCCCCC)C(=O)OCCCCCCCCCCCCN(CCCCO)CCCCCCCCCCCCOC(=O)C(CCCCCC)CCCCCCCC] |
| X-24 | 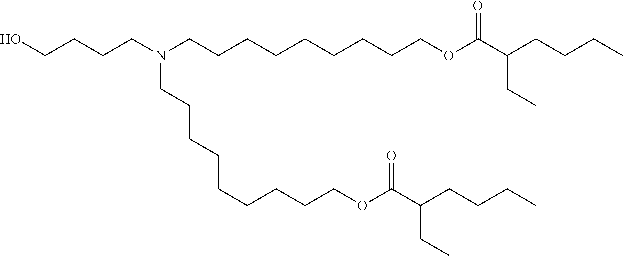 [SMILES: CCCCC(CC)C(=O)OCCCCCCCCCN(CCCCO)CCCCCCCCCOC(=O)C(CC)CCCC] |
| X-25 | 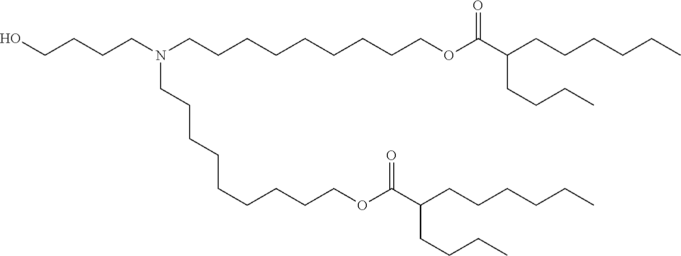 [SMILES: CCCCCCC(CCCC)C(=O)OCCCCCCCCCN(CCCCO)CCCCCCCCCOC(=O)C(CCCC)CCCCCC] |
| X-26 | 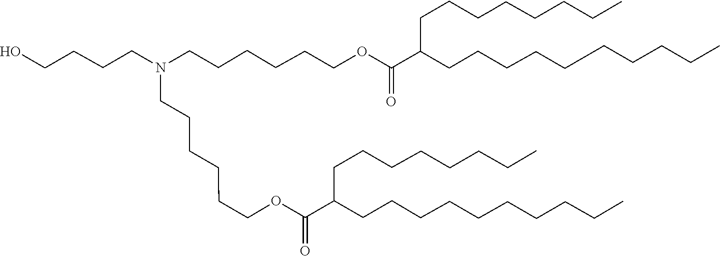 [SMILES: CCCCCCCCCCCC(CCCCCCCCC)C(=O)OCCCCCCN(CCCCO)CCCCCCOC(=O)C(CCCCCCCC)CCCCCCCCCC] |
| X-27 | 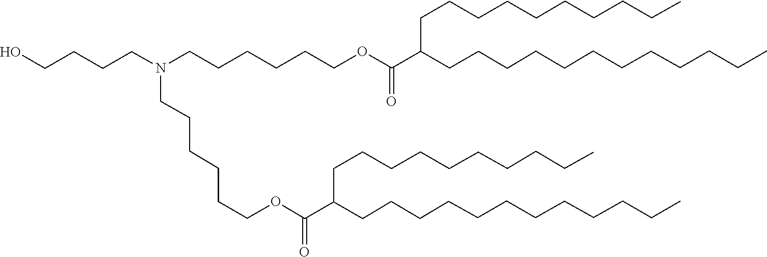 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| X-28 | 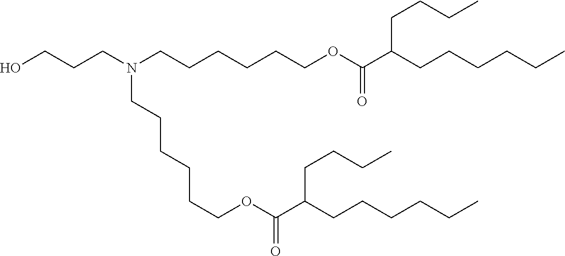 [SMILES: CCCCCCC(CCCC)C(=O)OCCCCCCN(CCCO)CCCCCCOC(=O)C(CCCC)CCCCCC] |
| X-29 | 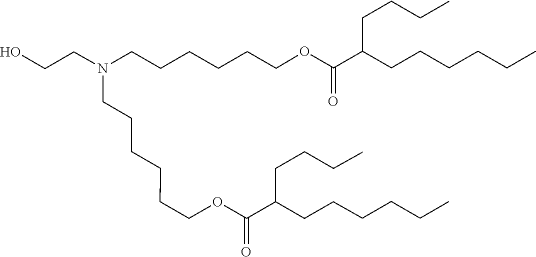 [SMILES: CCCCCCC(CCCC)C(=O)OCCCCCCN(CCO)CCCCCCOC(=O)C(CCCC)CCCCCC] |
| X-30 | 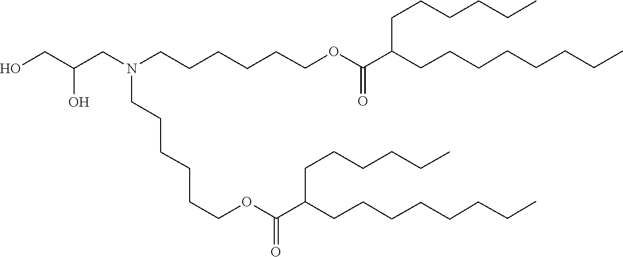 [SMILES: CCCCCCCCC(CCCCCC)C(=O)OCCCCCCN(CCCCCCOC(=O)C(CCCCCC)CCCCCCCC)CC(O)CO] |
| X-31 | 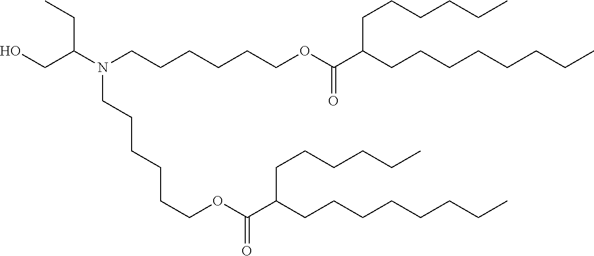 |
| X-32 | 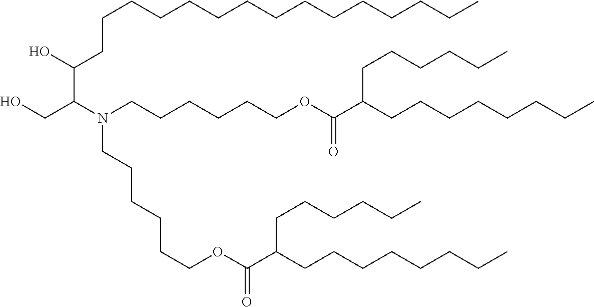 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| X-33 | 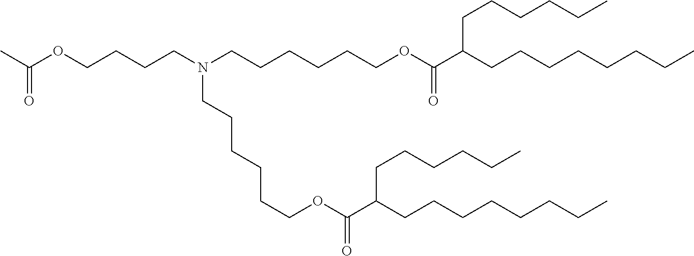 |
| X-34 | 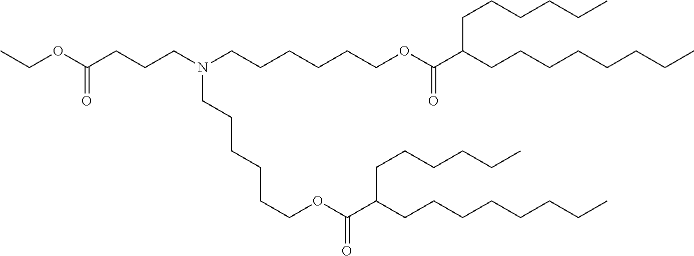 [SMILES: CCCCCCCCC(CCCCCC)C(=O)OCCCCCCN(CCCCCCOC(=O)C(CCCCCC)CCCCCCCC)CCCC(=O)OCC] |
| X-35 | 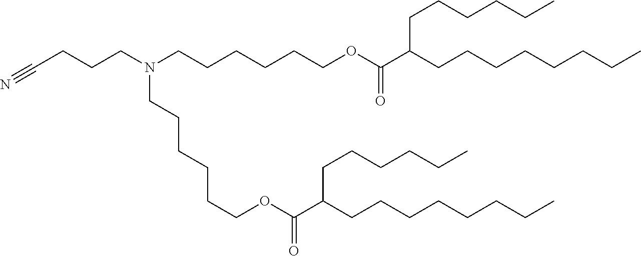 |
| X-36 | 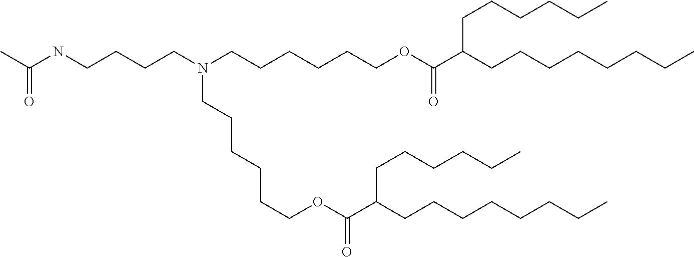 [SMILES: CCCCCCCCC(CCCCCC)C(=O)OCCCCCCN(CCCCCCOC(=O)C(CCCCCC)CCCCCCCC)CCCCNC(C)=O] |

In various different embodiments, the cationically ionizable lipid has one of the structures set forth in the table below.

| No. | Structure |
|---|---|
| A | 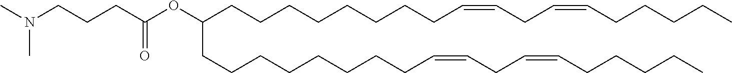 [SMILES: CCCCC/C=C\C/C=C\CCCCCCCCC(CCCCCCCC/C=C\C/C=C\CCCCC)OC(=O)CCCN(C)C] |
| B | 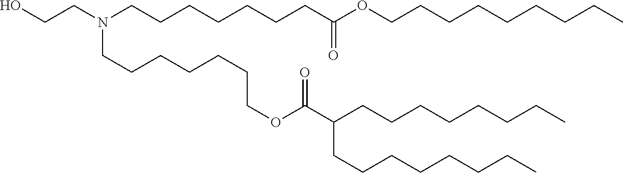 [SMILES: CCCCCCCCCOC(=O)CCCCCCCN(CCO)CCCCCCCOC(=O)C(CCCCCCCC)CCCCCCCC] |
| C | 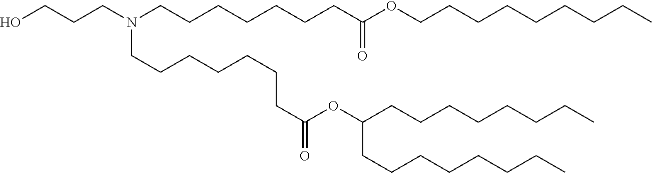 [SMILES: CCCCCCCCCOC(=O)CCCCCCCN(CCCO)CCCCCCCC(=O)OC(CCCCCCCC)CCCCCCCC] |
| D | 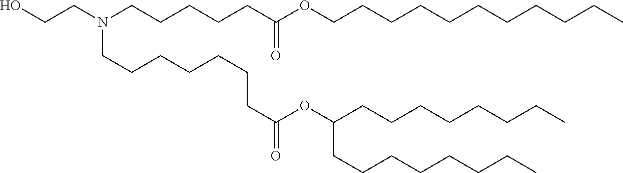 [SMILES: CCCCCCCCCCCOC(=O)CCCCCN(CCO)CCCCCCCC(=O)OC(CCCCCCCC)CCCCCCCC] |
| E | 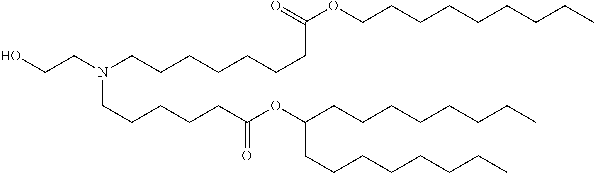 [SMILES: CCCCCCCCCOC(=O)CCCCCCCN(CCO)CCCCCC(=O)OC(CCCCCCCC)CCCCCCCC] |
| F | 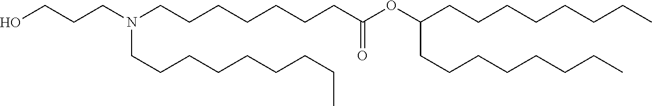 [SMILES: CCCCCCCCCN(CCCO)CCCCCCCC(=O)OC(CCCCCCCC)CCCCCCCC] |
| G | 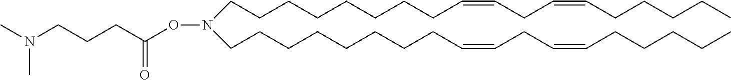 [SMILES: CCCCC/C=C\C/C=C\CCCCCCCCN(CCCCCCCC/C=C\C/C=C\CCCCC)OC(=O)CCCN(C)C] |

In some embodiments, the cationically ionizable lipid has the structure of Formula (XI):

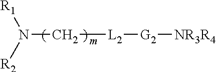

wherein
each of R₁ and R₂ is independently R₅ or -G₁-L₁-R₆, wherein at least one of R₁ and R₂ is -G₁-L₁-R₆; each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—,
—C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}— and —NR^{a}C(═O)O—;
R^{a} is H or C₁₋₁₂ alkyl;
m is 0, 1, 2, 3, or 4; and
x is 0, 1 or 2.

In some of the foregoing embodiments of Formula (XI), G₁ is independently unsubstituted C₁-C₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, each G₁ is independently unsubstituted C₆₋₁₂ alkylene or unsubstituted C₆₋₁₂ alkenylene, e.g., unsubstituted, straight C₆₋₁₂ alkylene or unsubstituted, straight C₆₋₁₂ alkenylene. In some embodiments, each G, is independently unsubstituted C₈₋₁₂ alkylene or unsubstituted C₈₋₁₂ alkenylene, e.g., unsubstituted, straight C₈₋₁₂ alkylene or unsubstituted, straight C₈₋₁₂ alkenylene. In some embodiments, each G, is independently unsubstituted C₆₋₁₀ alkylene or unsubstituted C₆₋₁₀ alkenylene, e.g., unsubstituted, straight C₆₋₁₀ alkylene or unsubstituted, straight C₆₋₁₀ alkenylene. In some embodiments, each G₁ is independently unsubstituted alkylene having 8, 9 or 10 carbon atoms, e.g., unsubstituted, straight alkylene having 8, 9 or 10 carbon atoms. In some embodiments, where R₁ and R₂ are both independently -G₁-L₁-R₆, G₁ for R, may be different from G₁ for R₂. In some of these embodiments, for example, G₁ for R₁ is unsubstituted, straight C₁₋₁₂ alkylene and G₁ for R₂ is unsubstituted, straight C₂₋₁₂ alkenylene; or G₁ for R₁ is an unsubstituted, straight C₁₋₂alkylene group and G₁ for R₂ is a different unsubstituted, straight C₁₋₁₂ alkylene group. In some embodiments, where R₁ and R₂ are both independently -G₁-L₁-R₆, G₁ for R₁ may be identical to G₁ for R₂. In some of these embodiments, for example, each G₁ is the same unsubstituted, straight C₂₋₁₂ alkylene, such as unsubstituted, straight C₈₋₁₀ alkylene, or each G₁ is the same unsubstituted, straight C₆₋₁₂ alkenylene.

In some of the foregoing embodiments of Formula (XI), each L₁ is independently selected from the group consisting of —O(C═O)—, —(C═O)O—, —C(═O)S—, —SC(═O)—, —NR^{a}C(═O)—, and —C(═O)NR^{a}—. In some embodiments, Ra of L₁ is H or C₁₋₁₂ alkyl. In some embodiments, Ra of L₁ is H or C₁₋₆ alkyl, e.g., H or C₁₋₃ alkyl. In some embodiments, Ra of L₁ is H, methyl, or ethyl. In some embodiments, each L₁ is independently selected from the group consisting of —O(C═O)—, —(C═O)O—, —C(═O)S—, and —SC(═O)—. In some embodiments, each L₁ is independently —O(C═O)— or —(C═O)O—. In some embodiments, where R₁ and R₂ are both independently -G₁-L₁-R₆, L, for R₁ may be different from L₁ for R₂. In some of these embodiments, for example, L, for R₁ is one moiety selected from the group consisting of —O(C═O)—, —(C═O)O—, —C(═O)S—, —SC(═O)—, —NR^{a}C(═O)—, and —C(═O)NR^{a}— (e.g., L₁ for R₁ is —O(C═O)—), and L₁ for R₂ is a different moiety selected from the group consisting of —O(C═O)—, —(C═O)O—, —C(═O)S—, —SC(═O)—, —NR^{a}C(═O)—, and —C(═O)NR^{a}— (e.g., L₁ for R₂ is —(C═O)O—). In some embodiments, where R₁ and R₂ are both independently -G₁-L₁-R₆, L₁ for R, may be identical to L₁ for R₂. In some of these embodiments, for example, each L, is the same moiety selected from the group consisting of —O(C═O)—, —(C═O)O—, —C(═O)S—, —SC(═O)—, —NR^{a}C(═O)—, and —C(═O)NR^{a}—, e.g., each L₁ is —O(C═O)— or each L, is —(C═O)O—.

In some of the foregoing embodiments of Formula (XI), each R₆ is independently a non-cyclic hydrocarbyl group having at least 10 carbon atoms, e.g., a straight hydrocarbyl group having at least 10 carbon atoms. In some embodiments, each R₆ has independently at most 30 carbon atoms, such as at most 28, at most 26, at most 24, at most 22, or at most 20 carbon atoms. In some embodiments, each R₆ is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms). In some embodiments, each R₆ is attached to L¹ via an internal carbon atom of R₆. In some embodiments, each R₆ has independently at most 30 carbon atoms (such as at most 28, at most 26, at most 24, at most 22, or at most 20 carbon atoms), and each R₆ is attached to L₁ via an internal carbon atom of R₆. In some embodiments, each R₆ is independently a non-cyclic hydrocarbyl group having at least 10 carbon atoms, e.g., a straight hydrocarbyl group having at least 10 carbon atoms, and each R₆ is attached to L₁ via an internal carbon atom of R₆. In some embodiments, each R₆ is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms), and each R₆ is attached to L₁ via an internal carbon atom of R₆. In some embodiments, the hydrocarbyl group of R₆ is an alkyl or alkenyl group, e.g., a C₁₀₋₃₀ alkyl or alkenyl group. Thus, in some embodiments, each R₆ is independently a non-cyclic alkyl group having at least 10 carbon atoms or a non-cyclic alkenyl group having at least 10 carbon atoms, e.g., a straight alkyl group having at least 10 carbon atoms or a straight alkenyl group having at least 10 carbon atoms. In some embodiments, each R₆ is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms). In some embodiments, each R₆ is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms). In some embodiments, each R₆ is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms), and each R₆ is attached to L₁ via an internal carbon atom of R₆. In some embodiments, each R₆ is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms), and each R₆ is attached to L₁ via an internal carbon atom of R₆. The expression “internal carbon atom” means that the carbon atom of R₆ by which R₆ is attached to L₁ is directly bonded to at least 2 other carbon atoms of R₆. For example, for the following C₁₁ alkyl group, each carbon atom at any one of positions 2, 3, 4, 5, and 7 qualifies as “internal carbon atom” according to the present disclosure, whereas the carbon atoms at positions 1, 6, 8, 9, 10, and 11 do not.

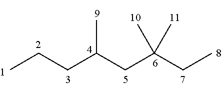

Consequently, R₆ being a C₁₁ alkyl group attached to L₁ via an internal carbon of R₆ includes the following groups:

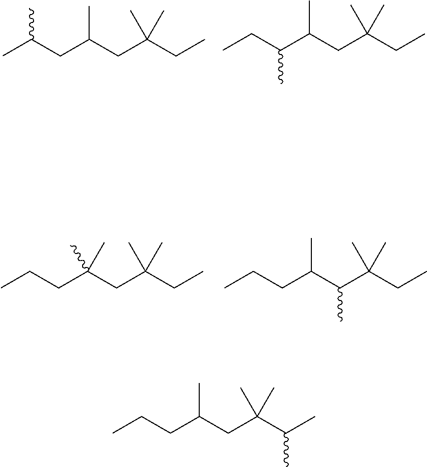

wherein [symbol] represents the bond by which R₆ is bound to L₁. Furthermore, for a straight alkyl group, e.g., a straight C₁₁ alkyl group, each carbon atom except for the first and last carbon atoms of the straight alkyl group (i.e., except the carbon atoms at positions 1 and 11 of the straight C₁₁ alkyl group) qualifies as “internal carbon atom”. Thus, in some embodiments, R₆ being a straight alkyl group having p carbon atoms and being attached to L₁ via an internal carbon atom of R₆ means that R₆ is attached to L₁ via a carbon atom of R₆ at any one of positions 2 to (p-1) (thereby excluding the terminal C atoms at positions 1 and p). In some embodiments, where R₆ is a straight alkyl group having p′ carbon atoms (wherein p′ is an even number) and being attached to L₁ via an internal carbon atom of R₆, R₆ is attached to L₁ via a carbon at any one of positions (p′/2−1), (p′/2), and (p′/2+1) of R₆ (e.g., if p′ is 10, R₆ is attached to L₁ via a carbon atom at any one of positions 4, 5, and 6 of R₆). In some embodiments, where R₆ is a straight alkyl group having p″ carbon atoms (wherein p″ is an uneven number) and being attached to L₁ via an internal carbon atom of R₆, R₆ is attached to L₁ via a carbon atom at any one of positions (p″−1)/2 and (p″+1)/2 of R₆ (e.g., if p″ is 11, R₆ is attached to L₁ via a carbon at any one of positions 5 and 6 of R₆). Generally, it is to be understood that if both R, and R₂ are -G₁-L₁-R₆ and each R₆ is attached to L, via an internal carbon atom of R₆, R₆ of R₁ is attached to L₁ of R₁(and not to L₁ of R₂) via an internal carbon atom of R₆ of R, and R₆ of R₂ is attached to L₁ of R₂ (and not to L₁ of R₁) via an internal carbon atom of R₆ of R₂. In some embodiments, each R₆ is independently selected from the group consisting of:

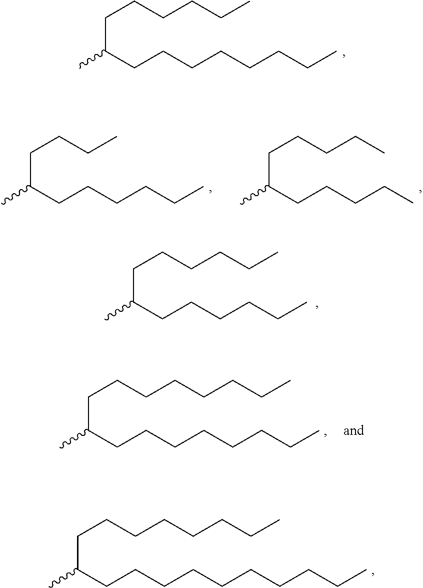

wherein [symbol] represents the bond by which R₆ is bound to L₁. In some embodiments, where R₁ and R₂ are both independently -G₁-L₁-R₆, R₆ for R₁ is different from R₆ for R₂. In some of these embodiments, for example, R₆ for R₁ may be a non-cyclic, preferably straight, hydrocarbyl group having at least 10 carbon atoms (e.g., R₆ for R₁ is

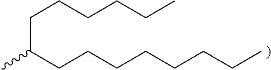 [SMILES: CCCCCCCCC(C)CCCCCC]

and R₆ for R₂ may be a different non-cyclic, preferably straight, hydrocarbyl group having at least 10 carbon atoms (e.g., R₆ for R₂ is

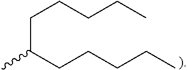

In some embodiments, where R₁ and R₂ are both independently -G₁-L₁-R₆, R₆ for R₁ is identical to R₆ for R₂. In some of these embodiments, for example, each R₆ is the same non-cyclic, preferably straight, hydrocarbyl group having at least 10 carbon atoms (e.g., each R₆ is

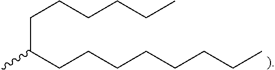 [SMILES: CCCCCCCCC(C)CCCCCC]

In some of the foregoing embodiments of Formula (XI), R₅ is a non-cyclic hydrocarbyl group having at least 10 carbon atoms, e.g., a straight hydrocarbyl group having at least 10 carbon atoms. In some embodiments, R₅ is a non-cyclic hydrocarbyl group having at least 12 carbon atoms, such as at least 14, at least 16, or at least 18 carbon atoms, e.g., a straight hydrocarbyl group having at least 12, at least 14, at least 16, or at least 18 carbon atoms. In some embodiments, R₅ has at most 30 carbon atoms, such as at most 28, at most 26, at most 24, at most 22, or at most 20 carbon atoms. In some embodiments, R₅ is a non-cyclic hydrocarbyl group, e.g., a straight hydrocarbyl group, wherein each hydrocarbyl group has 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the hydrocarbyl group of R₅ is an alkyl or alkenyl group, e.g., a C₁₀₋₃₀ alkyl or alkenyl group. Thus, in some embodiments, R₅ is a non-cyclic alkyl group having at least 10 carbon atoms (such as at least 12, at least 14, at least 16, or at least 18 carbon atoms) or a non-cyclic alkenyl group having at least 10 carbon atoms (such as at least 12, at least 14, at least 16, or at least 18 carbon atoms), e.g., a straight alkyl group having at least 10 carbon atoms (such as at least 12, at least 14, at least 16, or at least 18 carbon atoms) or a straight alkenyl group having at least 10 carbon atoms (such as at least 12, at least 14, at least 16, or at least 18 carbon atoms). In some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments, R₅ has the following structure:

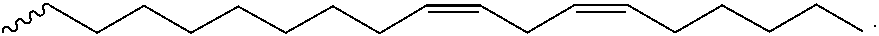

wherein [symbol] represents the bond by which R⁵ is bound to the remainder of the compound.

In some of the foregoing embodiments of Formula (XI), L₂ is selected from the group consisting of —O(C═O)—, —(C═O)O—, —C(═O)—, —S—S—, —C(═O)S—, —SC(═O)—, —NR^{a}C(═O)—, —C(═O)NR^{a}—, —NR^{a}C(═O)NR^{a}—, —OC(═O)NR^{a}— and —NR^{a}C(═O)O—. In some embodiments, L₂ is selected from the group consisting of —O(C═O)—, —(C═O)O—, —C(═O)—, —C(═O)S—, —SC(═O)—, —NR^{a}C(═O)—, and —C(═O)NR^{a}—. In some embodiments, R^{a} of L₂ is H or C₁₋₂ alkyl. In some embodiments, R^{a} of L₂ is H or C₁₋₆ alkyl, e.g., H or C₁₋₃ alkyl. In some embodiments, R^{a} of L₂ is H, methyl, or ethyl. In some embodiments, L₂ is selected from the group consisting of —O(C═O)—, —(C═O)O—, —C(═O)S—, and —SC(═O)—. In some embodiments, L₂ is —O(C═O)— or —(C═O)O—.

In some of the foregoing embodiments of Formula (XI), G₂ is unsubstituted C₁₋₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, G₂ is unsubstituted C₂₋₁₀ alkylene or unsubstituted C₂₋₁₀ alkenylene, e.g., unsubstituted, straight C₂₋₁₀ alkylene or unsubstituted, straight C₂₋₁₀ alkenylene. In some embodiments, G₂ is unsubstituted C₂₋₆ alkylene or unsubstituted C₂₋₆ alkenylene, e.g., unsubstituted, straight C₂₋₆ alkylene or unsubstituted, straight C₂₋₆ alkenylene. In some embodiments, G₂ is unsubstituted C₂₋₄ alkylene or unsubstituted C₂₋₄ alkenylene, e.g., unsubstituted, straight C₂₋₄ alkylene or unsubstituted, straight C₂₋₄ alkenylene. In some embodiments, G₂ is ethylene or trimethylene.

In some of the foregoing embodiments of Formula (XI), each of R₃ and R₄ is independently C₁₋₆ alkyl or C₂₋₆ alkenyl. In some embodiments, each of R₃ and R₄ is independently C₁₋₄ alkyl or C₂₋₄ alkenyl. In some embodiments, each of R₃ and R₄ is independently C₁₋₃ alkyl. In some embodiments, each of R₃ and R₄ is independently methyl or ethyl. In some embodiments, each of R₃ and R₄ is methyl.

In some of the foregoing embodiments of Formula (XI), m is 0, 1, 2 or 3. In some embodiments, m is 0 or 2. In some embodiments, m is 0. In some embodiments, m is 2.

In some of the foregoing embodiments of Formula (XI), the cationically ionizable lipid has the structure of Formula (XIIa) or (XIIb):

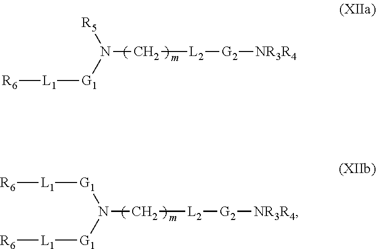

wherein
each of R₃ and R₄ is independently C₁-C₆ alkyl or C₂₋₆ alkenyl;
R₅ is a straight hydrocarbyl group having at least 14 carbon atoms (such as at least 16 carbon atoms), wherein the hydrocarbyl group preferably has at least 2 carbon-carbon double bonds;
each R₆ is independently a straight hydrocarbyl group (e.g., a straight alkyl group) having at least 10 carbon atoms and/or each R₆ is attached to L₁ via an internal carbon atom of R₆, preferably each R₆ is independently a straight hydrocarbyl group (e.g., a straight alkyl group) having at least 10 carbon atoms and each R₆ is attached to L₁ via an internal carbon atom of R₆;
each G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene, such as unsubstituted, straight C₈₋₁₂ alkylene or unsubstituted, straight C₈₋₁₂ alkenylene;
G₂ is unsubstituted C₂-C₁₀ alkylene or C₂₋₁₀ alkenylene, preferably unsubstituted C₂-C₆ alkylene or C₂₋₆ alkenylene;
each of L₁ and L₂ is independently —O(C═O)— or —(C═O)O—; and
m is 0, 1, 2 or 3, preferably 0 or 2.

In some of the foregoing embodiments of Formula (XIIa), R₅ has at most 30 carbon atoms, such as at most 28, at most 26, at most 24, at most 22, or at most 20 carbon atoms. In some embodiments of formulas (XIIa), R₅ is a straight hydrocarbyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), R₅ is a straight alkyl or alkenyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIla), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIa), R⁵ has the following structure:

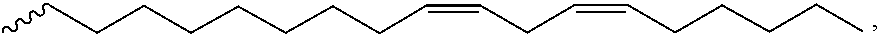

wherein [symbol] represents the bond by which R₅ is bound to the remainder of the compound. In some embodiments of formula (XIIa), R₆ has at most 30 carbon atoms, such as at most 28, at most 26, at most 24, at most 22, or at most 20 carbon atoms. In some embodiments of formula (XIIa), R₆ is a non-cyclic hydrocarbyl group (e.g., a non-cyclic alkyl group) having 10 to 30 carbon atoms (such as 10 to 28, 10 to 26, 10 to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms). In some embodiments of formula (XIIa), R₆ is a straight hydrocarbyl group (e.g., a straight alkyl group) having at least 10 carbon atoms and R⁶ is attached to L₁ via an internal carbon atom of R₆. In some embodiments of formula (XIIa), R₆ is a non-cyclic hydrocarbyl group (e.g., a non-cyclic alkyl group) having 10 to 30 carbon atoms (such as 10 to 28, 10 to 26, 10 to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms), and R₆ is attached to L₁ via an internal carbon atom of R₆. In some embodiments of formula (XIIa), G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene. In some embodiments of formula (XIIa), R₅ is a straight hydrocarbyl group, e.g., a straight alkenyl group, having at least 14 carbon atoms (such as 14 to 30 carbon atoms) and 2 or 3 carbon-carbon double bonds; R₆ is a straight hydrocarbyl group (e.g., a straight alkyl group) having at least 10 carbon atoms (e.g., having 10 to 30 carbon atoms) and R₆ is attached to L₁ via an internal carbon atom of R₆; and G₁ is independently unsubstituted, straight C₄₋₁₂ alkylene or C₄₋₁₂ alkenylene, e.g., unsubstituted, straight C₆₋₂ alkylene or C₆₋₁₂ alkenylene.

In some of the foregoing embodiments of Formula (XIIb), each R₆ has independently at most 30 carbon atoms, such as at most 28, at most 26, at most 24, at most 22, or at most 20 carbon atoms. In some embodiments of formula (XIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms). In some embodiments of formula (XIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R₆ is attached to L₁ via an internal carbon atom of R₆. In some embodiments of formula (XIIb), each R₆ is independently selected from the group consisting of:

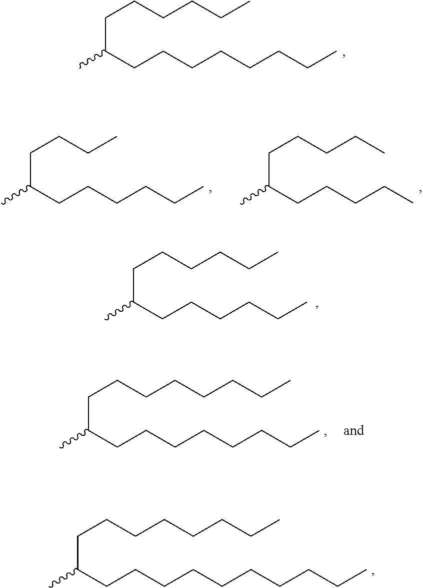

wherein [symbol] represents the bond by which R₆ is bound to L₁. In some embodiments of formula (XIIb), each G₁ is independently unsubstituted, straight C₆₋₁₂ alkylene or C₆₋₁₂ alkenylene. In some embodiments of formula (XIIb), each G, is independently unsubstituted, straight C₈₋₁₂ alkylene or C₈₋₁₂ alkenylene. In some embodiments of formula (XIIb), each R₆ is independently a straight hydrocarbyl group (e.g., a straight alkyl group) having at least 10 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L₁ via an internal carbon atom of R₆; and each G, is independently unsubstituted, straight C₈₋₁₂ alkylene or C₈₋₁₂ alkenylene.

In some of the foregoing embodiments of Formula (XI), the cationically ionizable lipid has the structure of Formula (XIIIa) or (XIIIb):

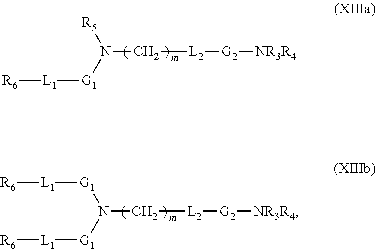

wherein
each of R₃ and R₄ is independently C₁₋₄ alkyl or C₂₋₄ alkenyl, more preferably C₁₋₃ alkyl, such as methyl or ethyl;
R₅ is a straight alkyl or alkenyl group having at least 16 carbon atoms, wherein the alkenyl group preferably has at least 2 carbon-carbon double bonds;
each R₆ is independently a straight hydrocarbyl group having at least 10 carbon atoms, wherein R₆ is attached to L₁ via an internal carbon atom of R₆;
each G₁ is independently unsubstituted, straight C₆₋₁₂ alkylene or unsubstituted, straight C₆₋₁₂ alkenylene, e.g., unsubstituted, straight C₈₋₁₂ alkylene or unsubstituted, straight C₈₋₁₂ alkenylene, such as unsubstituted, straight C₈₋₁₀ alkylene or unsubstituted, straight C₈₋₁₀ alkenylene, such as unsubstituted, straight C₈ alkylene;
G₂ is unsubstituted C₂₋₆ alkylene or C₂₋₆ alkenylene, preferably unsubstituted C₂₋₄ alkylene or C₂₋₄ alkenylene, such as ethylene or trimethylene;
each of L₁ and L₂ is independently —O(C═O)— or —(C═O)O—; and
m is 0, 1, 2 or 3, preferably 0 or 2.

In some of the foregoing embodiments of Formula (XIIIa), R₅ has at most 30 carbon atoms, such as at most 28, at most 26, at most 24, at most 22, or at most 20 carbon atoms. In some embodiments of formulas (XIIIa), R₅ is a straight alkyl or alkenyl group having 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIIa), the alkenyl group has at least 2 carbon-carbon double bonds, e.g., 2 or 3 carbon-carbon double bonds, such as 2 carbon-carbon double bonds.

In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIa), R₅ has the following structure:

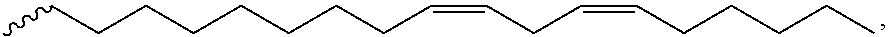

wherein [symbol] represents the bond by which R₅ is bound to the remainder of the compound. In some embodiments of formula (XIIIa), R₆ has at most 30 carbon atoms, such as at most 28, at most 26, at most 24, at most 22, or at most 20 carbon atoms. In some embodiments of formula (XIIIa), R₆ is a straight hydrocarbyl group (e.g., a straight alkyl group) having 10 to 30 carbon atoms (such as 10 to 28, 10 to 26, 10 to 24, 10 to 22, or 10 to 20 carbon atoms) and R₆ is attached to L₁ via an internal carbon atom of R₆. In some embodiments of formula (XIIIa), R₆ is a straight alkyl group having 10 to 30 carbon atoms (such as 10 to 28, 10 to 26, 10 to 24, 10 to 22, or 10 to 20 carbon atoms) and R₆ is attached to L₁ via an internal carbon atom of R₆. In some embodiments of formula (XIIIa), G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene.

In some embodiments of formula (XIIIa), R₅ is a straight hydrocarbyl group, e.g., a straight alkenyl group, having at least 16 carbon atoms (such as 16 to 30 carbon atoms) and 2 or 3 carbon-carbon double bonds; R₆ is a straight hydrocarbyl group (e.g., a straight alkyl group) having at least 10 carbon atoms (e.g., having 10 to 30 carbon atoms) and R₆ is attached to L, via an internal carbon atom of R₆; and G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene.

In some of the foregoing embodiments of Formula (XIIIb), each R₆ has independently at most 30 carbon atoms, such as at most 28, at most 26, at most 24, at most 22, or at most 20 carbon atoms. In some embodiments of formula (XIIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R₆ is attached to L₁ via an internal carbon atom of R₆. In some embodiments of formula (XIIIb), each R₆ is attached to L₁ via an internal carbon atom of R₆ and is independently selected from the group consisting of:

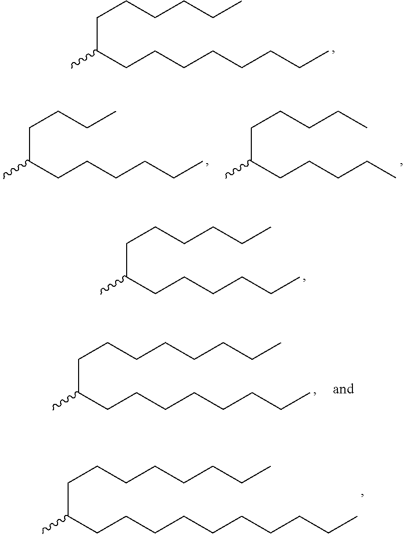

wherein [symbol] represents the bond by which R₆ is bound to L₁₋₆ In some embodiments of formula (XIIIb), each G₁ is independently unsubstituted, straight C₈₋₁₂ alkylene or C₈₋₁₂ alkenylene, e.g., unsubstituted, straight C₈₋₁₀ alkylene or C₈₋₁₀ alkenylene. In some embodiments of formula (XIIIb), each R₆ is independently a straight hydrocarbyl group (e.g., a straight alkyl group) having at least 10 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L₁ via an internal carbon atom of R₆; and each G, is independently unsubstituted, straight C₈₋₁₂ alkylene or C₈₋₁₂ alkenylene, e.g., unsubstituted, straight C₈₋₁₀ alkylene or C₈₋₁₀ alkenylene.

In some of the foregoing embodiments of Formula (XI), the cationically ionizable lipid has one of the following formulas (XIV-1), (XIV-2), and (XIV-3):

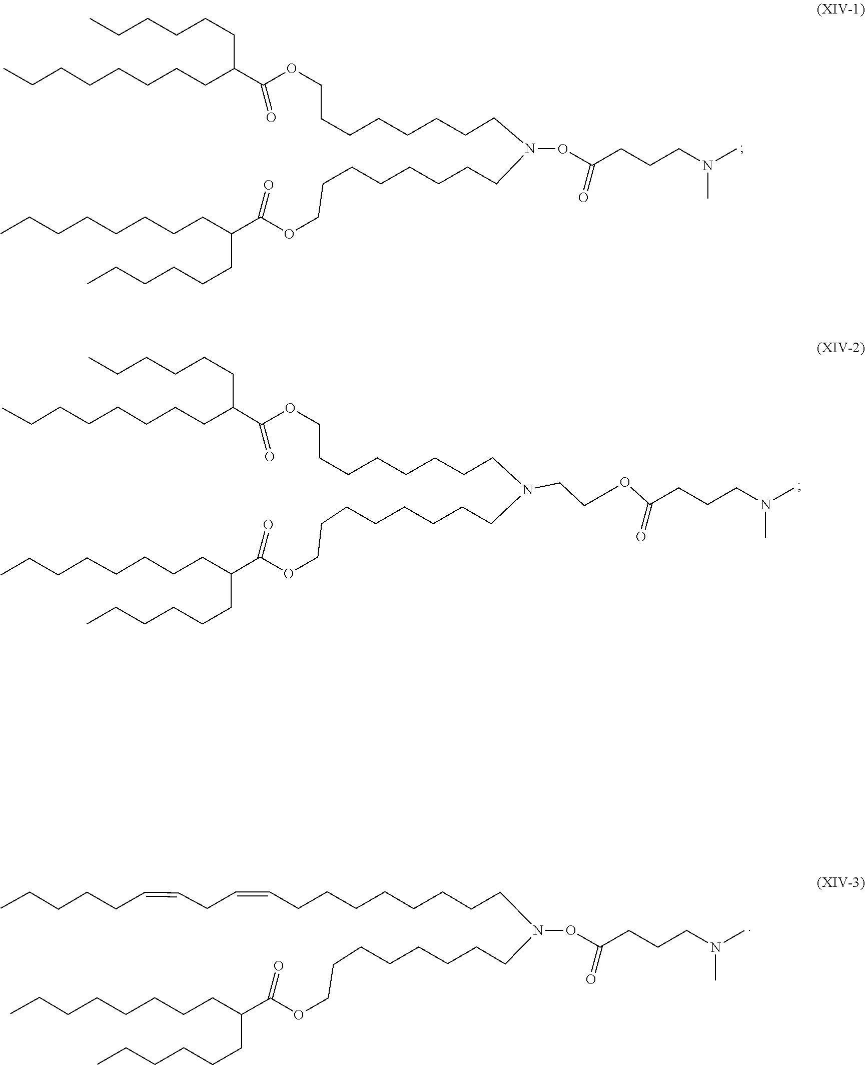

In some embodiments, the cationically ionizable lipid is (6Z,16Z)-12-((Z)-dec-4-en-1-yl)docosa-6,16-dien-1l-yl 5-(dimethylamino)pentanoate (3D-P-DMA). The structure of 3D-P-DMA may be represented as follows:

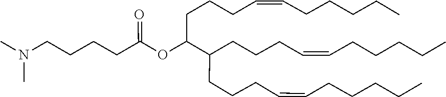 [SMILES: CCCCC/C=C\CCCC(CCC/C=C\CCCCC)C(CCC/C=C\CCCCC)OC(=O)CCCCN(C)C]

In various different embodiments, the cationically ionizable lipid is selected from the group consisting of N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14).

Further examples of cationically ionizable lipids include, but are not limited to, 3-(N-(N′,N′-dimethylaminoethane)-carbamoyl)cholesterol (DC-Chol), 1,2-dioleoyl-3-dimethylammonium-propane (DODAP); 1,2-diacyloxy-3-dimethylammonium propanes; 1,2-dialkyloxy-3-dimethylammonium propanes, 1,2-distearyloxy-N,N-dimethyl-3-aminopropane (DSDMA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-diinethylammonium-propane (DPDAP), N1-[2-((1 S)-1-[(3-aminopropyl)amino]-4-[di(3-amino-propyl)amino]butylcarboxamido)ethyl]-3,4-di[oleyloxy]-benzamide (MVL5), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), di((Z)-non-2-en-1-yl)-9-((4-(dimethylaminobutanoyl)oxy)heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In certain embodiments, the cationically ionizable lipid has the structure X-3.

In some embodiments, the cationic lipid for use herein is or comprises DPL14. As used herein, “DPL14” is a lipid comprising the following general formula:

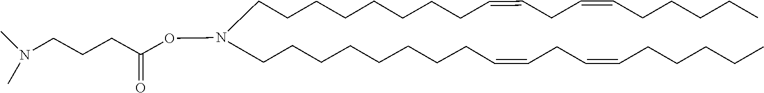

It is to be understood that any reference to a cationaically ionizable lipid disclosed herein also includes the salts (in particular pharmaceutically acceptable salts), tautomers, stereoisomers, solvates (e.g., hydrates), and isotopically labeled forms thereof.

In some embodiments, the cationically ionizable lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the composition/particle.

In some embodiments, wherein the compositions/particles (in particular the RNA compositions/particles) described herein comprise a cationically ionizable lipid and one or more additional lipids, the cationically ionizable lipid comprises from about 10 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipid present in the composition/particles. In some embodiments, the cationically ionizable lipid comprises from about 10 mol % to about 80 mol %, from about 20 mol % to about 80 mol %, from about 25 mol % to about 65 mol %, or from about 30 mol % to about 50 mol %, such as from about 40 mol % to about 50 mol % of the total lipid present in the composition/particles.

In one embodiment, the particles (in particular the RNA LNPs) described herein comprise from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationically ionizable lipid. In one embodiment, the particles (in particular the RNA LNPs) comprise about 47.0, 47.1, 47.2, 47.3, 47.4, 47.5, 47.6, 47.7, 47.8, 47.9 or 48.0 mol percent of the cationically ionizable lipid.

In some embodiments, where at least a portion of the cationically ionizable lipid described herein is associated with at least a portion of the RNA to form particles (e.g., LNPs), the cationically ionizable lipid may comprise from about 10 mol % to about 95 mol %, from about 20 mol % to about 95 mol %, from about 20 mol % to about 90 mol %, from about 30 mol % to about 90 mol %, from about 40 mol % to about 90 mol %, or from about 40 mol % to about 80 mol % of the total lipid present in the composition, such as from about 20 mol % to about 80 mol %, from about 25 mol % to about 70 mol %, from about 30 mol % to about 60 mol %, or from about 30 mol % to about 50 mol %, such as from about 40 mol % to about 60 mol % of the total lipid present in the particles.

In some embodiments, the N/P value is at least about 4. In some embodiments, the N/P value ranges from 4 to 20, 4 to 12, 4 to 10, 4 to 8, or 5 to 7. In some embodiments, the N/P value is about 6.

### Additional Lipids

The RNA compositions described herein may also comprise lipids or lipid-like materials other than cationically ionizable lipids, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to a cationically ionizable lipid may enhance composition and/or particle stability and efficacy of nucleic acid (such as RNA) delivery.

One or more additional lipids may be incorporated which may or may not affect the overall charge of the nucleic acid particles. In certain embodiments, the or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In certain embodiments, the RNA compositions (especially the mRNA compositions) described herein comprise a cationically ionizable lipid and one or more additional lipids.

Without wishing to be bound by theory, the amount of the cationically ionizable lipid compared to the amount of the one or more additional lipids may affect important nucleic acid particle characteristics, such as charge, particle size, stability, tissue selectivity, and bioactivity of the nucleic acid. Accordingly, in some embodiments, the molar ratio of the cationically ionizable lipid to the one or more additional lipids is from about 10:0 to about 1:9, about 4:1 to about 1:2, or about 3:1 to about 1:1.

In some embodiments, the one or more additional lipids comprised in the RNA compositions (especially in the mRNA compositions) described herein comprise one or more of the following: neutral lipids, steroids, polymer conjugated lipids, and combinations thereof.

### Neutral Lipids

In some embodiments, the one or more additional lipids comprise a neutral lipid which is preferably a phospholipid. In some embodiments, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), I-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In some embodiments, the neutral lipid is selected from the group consisting of DSPC, DOPC, DMPC, DPPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

Thus, in some embodiments, the RNA compositions (especially the mRNA compositions) described herein comprise a cationically ionizable lipid and DSPC.

In some embodiments, the neutral lipid is present in the RNA compositions (in particular the mRNA compositions) described herein in a concentration ranging from 5 to 15 mol percent, from 7 to 13 mol percent, or from 9 to 11 mol percent. In some embodiments, the neutral lipid is present in a concentration of about 9.5, 10 or 10.5 mol percent of the total lipids present in the RNA composition (especially the mRNA composition) described herein.

### Steroid

In one embodiment, the steroid is cholesterol. Thus, in some embodiments, the RNA compositions (especially the mRNA compositions) comprise a cationically ionizable lipid and cholesterol.

In one embodiment, the steroid is present in the RNA compositions (in particular the mRNA compositions) in a concentration ranging from 30 to 50 mol percent, from 35 to 45 mol percent or from 38 to 43 mol percent. In some embodiments, the steroid is present in a concentration of about 40, 41, 42, 43, 44, 45 or 46 mol percent of the total lipids present in the compositions (especially the mRNA compositions) described herein.

In certain preferred embodiments, the RNA compositions (especially the mRNA compositions) described herein comprise DSPC and cholesterol, preferably in the concentrations given above.

In some embodiments, the combined concentration of the neutral lipid (in particular, one or more phospholipids) and steroid (in particular, cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, such as from about 20 mol % to about 80 mol %, from about 25 mol % to about 75 mol %, from about 30 mol % to about 70 mol %, from about 35 mol % to about 65 mol %, or from about 40 mol % to about 60 mol %, of the total lipids present in the RNA compositions (especially the mRNA compositions) described herein.

In some embodiments, where at least a portion of (i) the RNA, (ii), the cationically ionizable lipid of formula (I), and (iii) the additional lipid form particles (e.g., LNPs), the additional lipid (e.g., one or more phospholipids and/or cholesterol) may comprise from about 0 mol % to about 60 mol %, from about 2 mol % to about 55 mol %, from about 5 mol % to about 50 mol %, from about 5 mol % to about 45 mol %, from about 10 mol % to about 45 mol %, from about 15 mol % to about 40 mol %, or from about 20 mol % to about 40 mol % of the total lipid present in the particles.

In some embodiments, the phospholipid may comprise from about 5 mol % to about 40 mol %, preferably from about 5 mol % to about 20 mol %, more preferably from about 5 mol % to about 15 mol % of the total lipid present in the particles.

In some embodiments, the steroid (in particular, cholesterol) comprises from about 10 mol % to about 65 mol %, preferably from about 20 mol % to about 60 mol %, more preferably from about 30 mol % to about 50 mol % of the total lipid present in the particles.

### Polymer-Conjuated Lipids

In some embodiments, the RNA compositions described herein may comprise at least one polymer-conjugated lipid. A polymer-conjugated lipid is typically a molecule comprising a lipid portion and a polymer portion conjugated thereto.

In some embodiments, a polymer-conjugated lipid is a PEG-conjugated lipid, also referred to herein as pegylated lipid or PEG-lipid. The term “pegylated lipid” refers to a molecule comprising both a lipid portion and a polyethylene glycol portion. Pegylated lipids are known in the art.

In some embodiments, a polymer-conjugated lipid is a polysarcosine-conjugated lipid, also referred to herein as sarcosinylated lipid or pSar-lipid. The term “sarcosinylated lipid” refers to a molecule comprising both a lipid portion and a polysarcosine portion.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties. If more than one type of repeat unit is present within the polymer, then the polymer is said to be a “copolymer.” The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In some embodiments, a polymer-conjugated lipid is designed to sterically stabilize a lipid particle by forming a protective hydrophilic layer that shields the hydrophobic lipid layer. In some embodiments, a polymer-conjugated lipid can reduce its association with serum proteins and/or the resulting uptake by the reticuloendothelial system when such lipid particles are administered in vivo.

In some embodiments, the RNA compositions described herein do not include a pegylated lipid. In some embodiments, the RNA compositions described herein do not include a sarcosinylated lipid. In some embodiments, the RNA compositions described herein do not include a pegylated lipid or a sarcosinylated lipid. In some embodiments, the RNA compositions described herein do not include any polymer-conjugated lipid.

### Polyethyleneglycol (PEG)-Coniugated Lipids

In some embodiments, the polymer conjugated lipid is a pegylated lipid. The term “pegylated lipid” refers to a molecule comprising both a lipid portion and a polyethylene glycol portion. Pegylated lipids are known in the art. In one embodiment, the pegylated lipid has the following structure (XV):

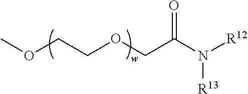

or a pharmaceutically acceptable salt, tautomer or stereoisomer thereof, wherein R¹² and R¹³ are each independently a straight or branched, alkyl or alkenyl chain containing from 10 to 30 carbon atoms, wherein the alkyl or alkenyl chain is optionally interrupted by one or more ester bonds; and w has a mean value ranging from 30 to 60.

In some embodiments of formula (XV), each of R¹² and R¹³ is independently a straight alkyl chain containing from 10 to 18 carbon atoms, preferably from 12 to 16 carbon atoms.

In some embodiments of formula (XV), R¹² and R¹³ are identical. In some embodiments, each of Rn and R¹³ is a straight alkyl chain containing 12 carbon atoms. In some embodiments, each of R¹² and R¹³ is a straight alkyl chain containing 14 carbon atoms. In some embodiments, each of R¹ and R¹³ is a straight alkyl chain containing 16 carbon atoms.

In some embodiments of formula (XV), R¹² and R³ are different. In some embodiments, one of R¹ and R¹³ is a straight alkyl chain containing 12 carbon atoms and the other of R¹² and R¹³ is a straight alkyl chain containing 14 carbon atoms.

In some embodiments of formula (XV), z has a mean value ranging from 40 to 50, such as a mean value of 45.

In some embodiments of formula (XV), z is within a range such that the PEG portion of the pegylated lipid of formula (XV) has an average molecular weight of from about 400 to about 6000 g/mol, such as from about 1000 to about 5000 g/mol, from about 1500 to about 4000 g/mol, or from about 2000 to about 3000 g/mol.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-((ω-methoxy(polyethoxy)ethyl)butanedioate (PEG-S-DMG), a pegylated ceramide (PEG-cer), or a PEG dialkoxypropylcarbamate such as o-methoxy(polyethoxy)ethyl-N-(2,3-di(tetradecanoxy)propyl)carbamate or 2,3-di(tetradecanoxy)propyl-N-((o methoxy(polyethoxy)ethyl)carbamate, and the like.

Further examples of PEG-conjugated lipids include of DSPE-PEG (1,2-distearoyl-sn-glycero-3-phosphoethanolamine-N-[carboxy(polyethylene glycol)methoxy]), such as DSPE-PEG(2000) sodium salt, DOPE-PEG (1,2-dioleoyl-sn-glycero-3-phosphoethanolamine-N-[(polyethylene glycol)methoxy]), such as DOPE-PEG(2000) ammonium salt, DPPE-PEG (1,2-dipalmitoyl-sn-glycero-3-phosphoethanolamine-N-[(polyethylene glycol)methoxy), such as DPPE-PEG(2000) ammonium salt, and DMPE-PEG (1,2-dimyristoyl-sn-glycero-3-phosphoethanolamine-N-[(polyethylene glycol)methoxy]), such as DMPE-PEG(2000) sodium salt.

In some embodiments, the pegylated lipid is 2-[(polyethylene glycol)-2000]-N,N-ditetradecylacetamide/2-[2-(ω-methoxy (polyethyleneglycol2000) ethoxy]-N,N-ditetradecylacetamide, e.g., having the following structure:

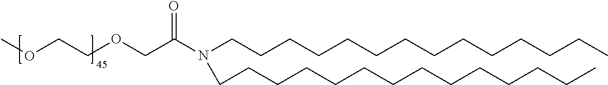

In some embodiments, the pegylated lipid is DMG-PEG 2000, e.g., having the following structure:

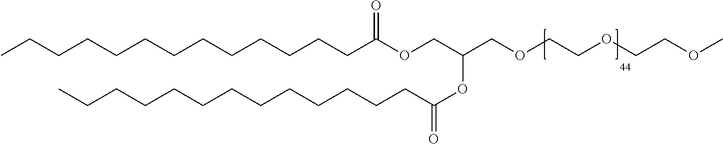

In some embodiments, the pegylated lipid has the following structure:

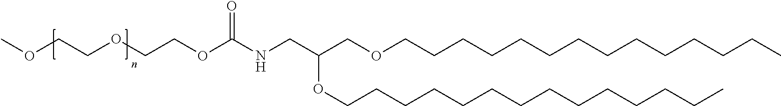

wherein n has a mean value ranging from 30 to 60, such as about 50.

In some embodiments, the pegylated lipid is PEG₂₀₀₀-C-DMA which preferably refers to 3-N-[(ω-methoxy poly(ethylene glycol)2000)carbamoyl]-1,2-dimyristyloxy-propylamine (MPEG-(2 kDa)-C-DMA) or methoxy-polyethylene glycol-2,3-bis(tetradecyloxy)propylcarbamate (2000). PEG₂₀₀₀-C-DMA may be selected in order to provide optimum delivery of RNA to the liver. It has been found that by modulating the alkyl chain length of the PEG lipid anchor, the pharmacology of encapsulated nucleic acid can be controlled in a predictable manner. In a vial, i.e., prior to administration, the particles retain a full complement of PEG₂₀₀₀-C-DMA. In the blood compartment, i.e., after administration, PEG₂₀₀₀-C-DMA dissociates from the RNA particle over time, revealing a more fusogenic particle that is more readily taken up by cells, ultimately leading to release of the RNA payload.

In some embodiments, RNA compositions described herein may comprise one or more PEG-conjugated lipids or pegylated lipids as described in WO 2017/075531 and WO 2018/081480, the entire contents of each of which are incorporated herein by reference for the purposes described herein.

In some embodiments, the pegylated lipid comprises from about 1 mol % to about 10 mol %, preferably from about 1 mol % to about 5 mol %, more preferably from about 1 mol % to about 2.5 mol % of the total lipid present in the RNA compositions described herein.

In some embodiments, where at least a portion of (i) the RNA, (ii), the cationically ionizable lipid of formula (I), and (iii) the pegylated lipid form particles (e.g., LNPs), the pegylated lipid may comprise from about 1 mol % to about 10 mol %, preferably from about 1 mol % to about 5 mol %, more preferably from about 1 mol % to about 2.5 mol % of the total lipid present in the particles.

In some embodiments, RNA compositions described herein comprise a cationically ionizable lipid as disclosed herein, a pegylated lipid, a phospholipid, and cholesterol, wherein the cationically ionizable lipid comprises from 40 to 50 mol percent of the total lipid present in the composition, the pegylated lipid comprises from 1 to 5 mol percent of the total lipid present in the composition, the phospholipid comprises from 5 to 15 mol percent of the total lipid present in the composition, and the cholesterol comprises from 30 to 50 mol percent of the total lipid present in the composition. In some embodiments, the phospholipid is DOPE. In some embodiments, the phospholipid is DSPC. In some embodiments, the pegylated lipid is DMG-PEG 2000. In some embodiments, the buffer substance contained in the RNA compositions described herein comprises or is a tertiary amine as defined herein (i.e., N(R¹)(R²)(R³), wherein none of R¹, R², and R³ is H) or a protonated form thereof. In some embodiments, the buffer substance contained in the RNA compositions described herein comprises or is a cyclic amine as defined herein (i.e., N(R¹)(R²)(R³), wherein two of R¹, R², and R³ join together with the nitrogen atom to form a 5- or 6-membered N-heterocyclic ring which is optionally substituted with one or two R⁵) or a protonated form thereof. In some embodiments, the buffer substance comprises or is TEA or a protonated form thereof.

Polysarcosin (pSar)-Conjugated Lipids

In some embodiments, the polymer conjugated lipid is a polysarcosine-lipid conjugate or a conjugate of polysarcosine and a lipid-like material, i.e., a lipid or lipid-like material which comprises polysarcosine (poly(N-methylglycine)). Thus, in some embodiments, RNA compositions described herein comprise a cationically ionizable lipid as disclosed herein and a pSar-conjugated lipid. In some embodiments, RNA compositions described herein may further comprise a neutral lipid, e.g., a phospholipid, cholesterol or a derivative thereof, or a combination of a neutral lipid, e.g., a phospholipid, and cholesterol or a derivative thereof. In some embodiments, RNA compositions described herein comprise a cationically ionizable lipid as described herein, a pSar-conjugated lipid, a neutral lipid, e.g., a phospholipid, and cholesterol or a derivative thereof. In some embodiments, the phospholipid is DSPC. In some embodiments, the phospholipid is DOPE. In some embodiments, RNA compositions described herein comprise a cationically ionizable lipid as described herein, a pSar-conjugated lipid, DSPC, and cholesterol or a derivative thereof.

The polysarcosine may comprise acetylated (neutral end group) or other functionalized end groups. In the case of RNA-lipid particles, the polysarcosine in one embodiment is conjugated to, preferably covalently bound to a non-cationic lipid or lipid-like material comprised in the particles.

In certain embodiments, the end groups of the polysarcosine may be functionalized with one or more molecular moieties conferring certain properties, such as positive or negative charge, or a targeting agent that will direct the particle to a particular cell type, collection of cells, or tissue.

A variety of suitable targeting agents are known in the art. Non-limiting examples of targeting agents include a peptide, a protein, an enzyme, a nucleic acid, a fatty acid, a hormone, an antibody, a carbohydrate, mono-, oligo- or polysaccharides, a peptidoglycan, a glycopeptide, or the like. For example, any of a number of different materials that bind to antigens on the surfaces of target cells can be employed. Antibodies to target cell surface antigens will generally exhibit the necessary specificity for the target. In addition to antibodies, suitable immunoreactive fragments can also be employed, such as the Fab, Fab′, F(ab′)2 or scFv fragments or single-domain antibodies (e.g. camelids VHH fragments). Many antibody fragments suitable for use in forming the targeting mechanism are already available in the art. Similarly, ligands for any receptors on the surface of the target cells can suitably be employed as targeting agent. These include any small molecule or biomolecule, natural or synthetic, which binds specifically to a cell surface receptor, protein or glycoprotein found at the surface of the desired target cell.

In certain embodiments, the polysarcosine comprises between 2 and 200, between 2 and 190, between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70 sarcosine units.

In certain embodiments, the polysarcosine comprises the following general formula (XVI):

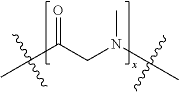

wherein x refers to the number of sarcosine units. The polysarcosine through one of the bonds may be linked to a particle-forming component or a hydrophobic component. The polysarcosine through the other bond may be linked to H, a hydrophilic group, an ionizable group, or to a linker to a functional moiety such as a targeting moiety.

The polysarcosine may be conjugated, in particular covalently bound to or linked to, any particle forming component such as a lipid or lipid-like material. The polysarcosine-lipid conjugate is a molecule wherein polysarcosine is conjugated to a lipid as described herein such as a cationic lipid or cationically ionizable lipid or an additional lipid. Alternatively, polysarcosine is conjugated to a lipid or lipid-like material which is different from the cationically ionizable lipid or the one or more additional lipids.

In certain embodiments, the polysarcosine-lipid conjugate or a conjugate of polysarcosine and a lipid-like material comprises the following general formula (XVIa):

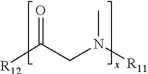

wherein one of R₁₁ and R₁₂ comprises a hydrophobic group and the other is H, a hydrophilic group, an ionizable group or a functional group optionally comprising a targeting moiety. In one embodiment, the hydrophobic group comprises a linear or branched alkyl group or aryl group, preferably comprising from 10 to 50, 10 to 40, or 12 to 20 carbon atoms. In one embodiment, Ru₁ or R₁₂ which comprises a hydrophobic group comprises a moiety such as a heteroatom, in particular N, linked to one or more linear or branched alkyl groups.

In certain embodiments, a polysarcosine-lipid conjugate or a conjugate of polysarcosine and a lipid-like material comprises the following general formula (XVIb):

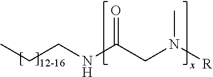

wherein R is H, a hydrophilic group, an ionizable group or a functional group optionally comprising a targeting moiety.

The symbol “x” in the general formulas (XVIa) and (XVIb) refers to the number of sarcosine units and may be a number as defined herein.

In certain embodiments, the polysarcosine-lipid conjugate or a conjugate of polysarcosine and a lipid-like material is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

Typically, the polysarcosine moiety has between 2 and 200, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, or between 10 and 80 sarcosine units.

In some embodiments, the pSar-conjugated lipid has the structure of the following formula (VII-1):

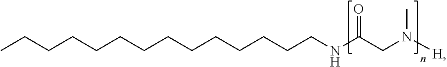

wherein n is 23. The pSar-conjugated lipid of formula (VII-1) is also referred to herein as “C14pSar23”. Thus, in some embodiments, the RNA compositions (especially the mRNA compositions) described herein comprise a cationically ionizable lipid and a polysarcosine-lipid conjugate or a conjugate of polysarcosine and a lipid-like material, e.g., a polysarcosine-lipid conjugate or a conjugate of polysarcosine and a lipid-like material as defined above.

In certain instances, the polysarcosine-lipid conjugate may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipids present in the composition (especially the RNA composition) described herein.

In some embodiments, where at least a portion of (i) the RNA, (ii), the cationically ionizable lipid of formula (I), and (iii) the pSar-conjugated lipid form particles (e.g., LNPs), the pSar-conjugated lipid may comprise from about 0.5 mol % to about 5 mol %, preferably from about 1 mol % to about 5 mol %, more preferably from about 1 mol % to about 4.5 mol % of the total lipid present in the particles.

In some embodiments, RNA compositions described herein comprise a cationically ionizable lipid as described herein, a pSar-conjugated lipid, a phospholipid, and cholesterol, wherein the cationically ionizable lipid comprises from 40 to 50 mol percent of the total lipid present in the composition, the pSar-conjugated lipid comprises from 1 to 4.5 mol percent of the total lipid present in the composition, the phospholipid comprises from 5 to 15 mol percent of the total lipid present in the composition, and the cholesterol comprises from 30 to 50 mol percent of the total lipid present in the composition. In some embodiments, the phospholipid is DOPE. In some embodiments, the phospholipid is DSPC. In some embodiments, the pSar-conjugated lipid is C14pSar23. In some embodiments, the buffer substance contained in the RNA compositions described herein comprises or is a tertiary amine as defined herein (i.e., N(R¹)(R²)(R³), wherein none of R¹, R², and R³ is H) or a protonated form thereof. In some embodiments, the buffer substance contained in the RNA compositions described herein comprises or is a cyclic amine as defined herein (i.e., N(R¹)(R²)(R³), wherein two of R¹, R², and R³ join together with the nitrogen atom to form a 5- or 6-membered N-heterocyclic ring which is optionally substituted with one or two R⁵) or a protonated form thereof. In some embodiments, the buffer substance comprises or is TEA or a protonated form thereof.

### Embodiments of RNA Compositions

In some embodiments, the one or more additional lipids comprise one of the following components: (1) a neutral lipid; (2) a steroid; (3) a polymer conjugated lipid; (4) a mixture of a neutral lipid and a steroid; (5) a mixture of a neutral lipid and a polymer conjugated lipid; (6) a mixture of a steroid and a polymer conjugated lipid; or (7) a mixture of a neutral lipid, a steroid, and a polymer conjugated lipid, preferably each in the concentration given above. In some embodiments, the one or more additional lipids comprise one of the following components: (1) a phospholipid; (2) cholesterol; (3) a pegylated lipid; (4) a mixture of a phospholipid and cholesterol; (5) a mixture of a phospholipid and a pegylated lipid; (6) a mixture of cholesterol and a pegylated lipid; or (7) a mixture of a phospholipid, cholesterol, and a pegylated lipid, preferably each in the concentration given above.

Thus, in preferred embodiments, the RNA compositions (especially the mRNA compositions) described herein comprise a cationically ionizable lipid and one of the following lipids or lipid mixtures: (1) a neutral lipid; (2) a steroid; (3) a polymer conjugated lipid; (4) a mixture of a neutral lipid and a steroid; (5) a mixture of a neutral lipid and a polymer conjugated lipid; (6) a mixture of a steroid and a polymer conjugated lipid; or (7) a mixture of a neutral lipid, a steroid, and a polymer conjugated lipid, preferably each in the concentration given above. In specific embodiments, the cationically ionizable lipid is present in a concentration of from 40 to 50 mol percent; the neutral lipid is present in a concentration of from 5 to 15 mol percent; the steroid is present in a concentration of from 35 to 45 mol; and the polymer conjugated lipid is present in a concentration of from 1 to 10 mol percent, wherein the RNA is encapsulated within or associated with the LNPs. In some embodiments, the buffer substance contained in the RNA compositions (especially the mRNA compositions) described herein comprises or is a tertiary amine as defined herein (i.e., N(R¹)(R²)(R³), wherein none of R¹, R², and R³ is H) or a protonated form thereof. In some embodiments, the buffer substance contained in the RNA compositions (especially the mRNA compositions) described herein comprises or is a cyclic amine as defined herein (i.e., N(R¹)(R²)(R³), wherein two of R¹, R², and R³ join together with the nitrogen atom to form a 5- or 6-membered N-heterocyclic ring which is optionally substituted with one or two R⁵) or a protonated form thereof. In some embodiments, the buffer substance comprises or is TEA or a protonated form thereof.

In some embodiments, the RNA compositions (especially the mRNA compositions) described herein comprise a cationically ionizable lipid and one of the following lipids or lipid mixtures: (1) a phospholipid; (2) cholesterol; (3) a pegylated lipid; (4) a mixture of a phospholipid and cholesterol; (5) a mixture of a phospholipid and a pegylated lipid; (6) a mixture of cholesterol and a pegylated lipid; or (7) a mixture of a phospholipid, cholesterol, and a pegylated lipid, preferably each in the concentration given above. In some specific embodiments, the cationically ionizable lipid is present in a concentration of from 40 to 50 mol percent; the phospholipid is present in a concentration of from 5 to 15 mol percent; the cholesterol is present in a concentration of from 35 to 45 mol; and the pegylated lipid is present in a concentration of from 1 to 10 mol percent, wherein the RNA may be encapsulated within or associated with the LNPs. In some embodiments, the buffer substance contained in the RNA compositions (especially the mRNA compositions) described herein comprises or is a tertiary amine as defined herein (i.e., N(R¹)(R²)(R³), wherein none of R¹, R², and R³ is H) or a protonated form thereof. In some embodiments, the buffer substance contained in the RNA compositions (especially the mRNA compositions) described herein comprises or is a cyclic amine as defined herein (i.e., N(R¹)(R²)(R³), wherein two of R¹, R², and R³ join together with the nitrogen atom to form a 5- or 6-membered N-heterocyclic ring which is optionally substituted with one or two R⁵) or a protonated form thereof. In some embodiments, the buffer substance comprises or is TEA or a protonated form thereof.

The N/P value is preferably at least about 4. In some embodiments, the N/P value ranges from 4 to 20, 4 to 12, 4 to 10, 4 to 8, or 5 to 7. In one embodiment, the N/P value is about 6.

LNPs described herein may have an average diameter that in one embodiment ranges from about 30 nm to about 200 nm, or from about 60 nm to about 120 nm.

Generally, the LNPs comprising RNA (or “RNA LNPs”) described herein are “RNA-lipid particles” that can be used to deliver RNA to a target site of interest (e.g., cell, tissue, organ, and the like). An RNA-lipid particle is typically formed from a cationically ionizable lipid (such as the lipid having the structure I-3) and one or more additional lipids, such as a phospholipid (e.g., DSPC), a steroid (e.g., cholesterol or analogues thereof), and a polymer conjugated lipid (e.g., a pegylated lipid or a polysarcosine-lipid conjugate or a conjugate of polysarcosine and a lipid-like material).

Without intending to be bound by any theory, it is believed that the cationically ionizable lipid and the one or more additional lipids combine together with the RNA to form colloidally stable particles, wherein the nucleic acid is bound to the lipid matrix.

In some embodiments, RNA-lipid particles comprise more than one type of RNA molecules, where the molecular parameters of the RNA molecules may be similar or different from each other, like with respect to molar mass or fundamental structural elements such as molecular architecture, capping, coding regions or other features.

In some embodiments, the RNA-lipid LNPs (such as mRNA-lipid LNPs) in addition to RNA comprise (i) a cationically ionizable lipid which may comprise from about 10 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipids present in the particle, (ii) a neutral lipid and/or a steroid, (e.g., one or more phospholipids and/or cholesterol) which may comprise from about 0 mol % to about 90 mol %, from about 20 mol % to about 80 mol %, from about 25 mol % to about 75 mol %, from about 30 mol % to about 70 mol %, from about 35 mol % to about 65 mol %, or from about 40 mol % to about 60 mol %, of the total lipids present in the particle, and (iii) a polymer conjugated lipid (e.g., a pegylated lipid which may comprise from 1 mol % to 10 mol %, from 1 mol % to 5 mol %, or from 1 mol % to 2.5 mol % of the total lipids present in the particle; or a polysarcosine-lipid conjugate which may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipids present in the particle).

In certain preferred embodiments, the neutral lipid comprises a phospholipid of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipids present in the particle.

In certain preferred embodiments, the steroid comprises cholesterol or a derivative thereof of from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipids present in the particle.

In certain preferred embodiments, the neutral lipid and the steroid comprises a mixture of: (i) a phospholipid such as DSPC of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipids present in the particle; and (ii) cholesterol or a derivative thereof such as cholesterol of from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipids present in the particle. As a non-limiting example, an mRNA LNP comprising a mixture of a phospholipid and cholesterol may comprise DSPC of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipids present in the particle and cholesterol of from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipids present in the particle.

In some embodiments, the RNA-lipid particles in addition to RNA comprise (i) a cationically ionizable lipid (such as the lipid having the structure I-3) which may comprise from about 10 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipids present in the particle, (ii) DSPC which may comprise from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipids present in the particle, (iii) cholesterol which may comprise from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipids present in the particle and (iv) a pegylated lipid which may comprise from 1 mol % to 10 mol %, from 1 mol % to 5 mol %, or from 1 mol % to 2.5 mol % of the total lipids present in the particle; or (iv′) a polysarcosine-lipid conjugate which may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipids present in the particle.

RNA LNPs described herein have an average diameter that in one embodiment ranges from about 30 nm to about 1000 inn, from about 30 nm to about 800 nm, from about 30 nm to about 700 nm, from about 30 nm to about 600 nm, from about 30 nm to about 500 nm, from about 30 nm to about 450 nm, from about 30 nm to about 400 nm, from about 30 nm to about 350 nm, from about 30 nm to about 300 nm, from about 30 nm to about 250 nm, from about 30 nm to about 200 nm, from about 30 nm to about 190 nm, from about 30 nm to about 180 nm, from about 30 nm to about 170 am, from about 30 nm to about 160 nm, from about 30 nm to about 150 nm, from about 50 nm to about 500 nm, from about 50 nm to about 450 nm, from about 50 nm to about 400 am, from about 50 nm to about 350 nm, from about 50 nm to about 300 nm, from about 50 nm to about 250 nm, from about 50 nm to about 200 nm, from about 50 nm to about 190 am, from about 50 nm to about 180 nm, from about 50 nm to about 170 nm, from about 50 nm to about 160 nm, or from about 50 nm to about 150 nm.

In certain embodiments, RNA LNPs described herein have an average diameter that ranges from about 40 nm to about 800 nm, from about 50 nm to about 700 nm, from about 60 nm to about 600 nm, from about 70 nm to about 500 nm, from about 80 nm to about 400 nm, from about 150 nm to about 800 nm, from about 150 nm to about 700 nm, from about 150 nm to about 600 am, from about 200 nm to about 600 rmn, from about 200 nm to about 500 nm, or from about 200 nm to about 400 nm.

RNA LNPs described herein, e.g. prepared by the methods described herein, exhibit a polydispersity index less than about 0.5, less than about 0.4, less than about 0.3, less than about 0.2, less than about 0.1 or about 0.05 or less. By way of example, the RNA LNPs can exhibit a polydispersity index in a range of about 0.05 to about 0.2, such as about 0.05 to about 0.1.

In certain embodiments of the present disclosure, the RNA in the RNA LNPs described herein is at a concentration from about 2 mg/l to about 5 g/l, from about 2 mg/l to about 2 g/1, from about 5 mg/l to about 2 g/l, from about 10 mg/l to about 1 g/l, from about 50 mg/l to about 0.5 g/l or from about 100 mg/l to about 0.5 g/l. In specific embodiments, the RNA is at a concentration from about 5 mg/l to about 150 mg/l, from about 0.005 mg/mL to about 0.09 mg/mL, from about 0.005 mg/mL to about 0.08 mg/mL, from about 0.005 mg/mL to about 0.07 mg/mL, from about 0.005 mg/mL to about 0.06 mg/mL, or from about 0.005 mg/mL to about 0.05 mg/mL.

### Compositions Comprising RNA Particles

The compositions described herein may comprise RNA LNPs, preferably a plurality of RNA LNPs. The term “plurality of RNA LNPs” or “plurality of RNA-lipid particles” refers to a population of a certain number of particles. In certain embodiments, the term refers to a population of more than 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², or 10²³ or more particles.

In some embodiments, the compositions described herein comprise particles with a size of at least 10 m in an amount of less than 4000/ml, preferably at most 3500/ml, such as at most 3400/ml, at most 3300/ml, at most 3200/ml, at most 3100/ml, or at most 3000/ml.

It will be apparent to those of skill in the art that the plurality of particles can include any fraction of the foregoing ranges or any range therein.

In some embodiments, the composition described herein is a liquid or a solid, with a solid referring to a frozen form.

The present inventors have surprisingly found that using a particular buffer system based on the above specified buffer substances, in particular TEA and its protonated form, instead of PBS in a composition comprising LNPs inhibits the formation of a very stable folded form of RNA (called “light migrating species (LMS)” herein).

Furthermore, the present application demonstrates that, surprisingly, by using this buffer system, it is possible to obtain an RNA composition having improved RNA integrity after storage in liquid form for about 3 months. Thus, the composition prepared by the claimed method provides improved stability, can be stored in a temperature range compliant to regular technologies in pharmaceutical practice, and provides a ready-to-use preparation.

The expression “equal to” or “essentially equal to”, as used herein with respect to the size (Z_{average}) of particles (such as LNPs), means that the Z_{average} value of the particles contained in a composition after a processing step (e.g., after a freeze/thaw cycle) corresponds to the Z_{average} value of the particles before the processing step (e.g., before the freeze/thaw cycle)±30% (preferably, ±25%, more preferably ±24%, such as ±20%, ±15%, ±10%, ±5%, or ±1%). For example, if the size (Z_{average}) value of particles (such as LNPs) contained in a composition not yet subjected to a freeze/thaw cycle is 90 nm, and the size (Z_{average}) value of particles (such as LNPs) contained in the composition subjected to a freeze/thaw cycle is 115 nm, then the size (Z_{average}) of particles after the freeze/thaw cycle, i.e., after thawing the frozen composition, is considered being (essentially) equal to the size (Z_{average}) of particles before the freeze/thaw cycle, i.e., before freezing the composition. The expression “equal to” or “essentially equal to”, as used herein with respect to the size distribution or PDI of particles (such as LNPs), is to be interpreted accordingly. For example, if the PDI value of particles (such as LNPs) contained in a composition not yet subjected to a freeze/thaw cycle is 0.30, and the PDI value of particles (such as LNPs) contained in the composition subjected to a freeze/thaw cycle is 0.38, then the PDI of particles after the freeze/thaw cycle, i.e., after thawing the frozen composition, is considered being (essentially) equal to the PDI of particles before the freeze/thaw cycle, i.e., before freezing the composition.

Compositions described herein may also comprise a cryoprotectant (in particular, if the composition is in frozen form or is to be subjected to at least one freezing step or at least one freeze/thaw cycle) and/or a surfactant as stabilizer to avoid substantial loss of the product quality and, in particular, substantial loss of RNA activity during storage and/or freezing, for example to reduce or prevent aggregation, particle collapse, RNA degradation and/or other types of damage.

In some embodiments, the cryoprotectant is a carbohydrate. The term “carbohydrate”, as used herein, refers to and encompasses monosaccharides, disaccharides, trisaccharides, oligosaccharides and polysaccharides.

In some embodiments, the cryoprotectant is a monosaccharide. The term “monosaccharide”, as used herein refers to a single carbohydrate unit (e.g., a simple sugar) that cannot be hydrolyzed to simpler carbohydrate units. Exemplary monosaccharide cryoprotectants include glucose, fructose, galactose, xylose, ribose and the like.

In some embodiments, the cryoprotectant is a disaccharide. The term “disaccharide”, as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide cryoprotectants include sucrose, trehalose, lactose, maltose and the like.

The term “trisaccharide” means three sugars linked together to form one molecule. Examples of a trisaccharides include raffinose and melezitose.

In some embodiments, the cryoprotectant is an oligosaccharide. The term “oligosaccharide”, as used herein refers to a compound or a chemical moiety formed by 3 to about 15, preferably 3 to about 10 monosaccharide units that are bonded together through glycosidic linkages, for example through 1-4 linkages or 1-6 linkages, to form a linear, branched or cyclic structure. Exemplary oligosaccharide cryoprotectants include cyclodextrins, raffinose, melezitose, maltotriose, stachyose, acarbose, and the like. An oligosaccharide can be oxidized or reduced.

In some embodiments, the cryoprotectant is a cyclic oligosaccharide. The term “cyclic oligosaccharide”, as used herein refers to a compound or a chemical moiety formed by 3 to about 15, preferably 6, 7, 8, 9, or 10 monosaccharide units that are bonded together through glycosidic linkages, for example through 1-4 linkages or 1-6 linkages, to form a cyclic structure. Exemplary cyclic oligosaccharide cryoprotectants include cyclic oligosaccharides that are discrete compounds, such as α cyclodextrin, β cyclodextrin, or γ cyclodextrin.

Other exemplary cyclic oligosaccharide cryoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety”, as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate cryoprotectants, e.g., cyclic oligosaccharide cryoprotectants, can be derivatized carbohydrates. For example, in an embodiment, the cryoprotectant is a derivatized cyclic oligosaccharide, e.g., a derivatized cyclodextrin, e.g., 2-hydroxypropyl-β-cyclodextrin, e.g., partially etherified cyclodextrins (e.g., partially etherified β cyclodextrins).

An exemplary cryoprotectant is a polysaceharide. The term “polysaccharide”, as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide cryoprotectants include glycogen, amylase, cellulose, dextran, maltodextrin and the like.

In some embodiments, the cryoprotectant is a sugar alcohol. The term “sugar alcohol”, as used herein, refers to organic compounds containing at least two carbon atoms and one hydroxyl group attached to each carbon atom. Typically, sugar alcohols are derived from sugars (e.g., by hydrogenation of sugars) and are water-soluble solids. The term “sugar”, as used herein, refers sweet-tasting, soluble carbohydrates. Examples of sugar alcohols include ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, and polyglycitol. In one embodiment, the sugar alcohol has the formula HOCH₂(CHOH)ₙCH₂OH, wherein n is 0 to 22 (e.g., 0, 1, 2, 3, or 4), or a cyclic variant thereof (which can formally be derived by dehydration of the sugar alcohol to give cyclic ethers; e.g. isosorbide is the cyclic dehydrated variant of sorbitol).

In some embodiments, the cryoprotectant is a lower alcohol, such as an alcohol (in particular aliphatic alcohol) having up to 6 carbon atoms (preferably at least 2 and up to 5, 4, or 3 carbon atoms). In preferred embodiments, the lower alcohol is completely miscible with water. In some embodiments, the cryoprotectant is selected from the group consisting of ethanol, propanol, 1,2-propanediol, and 1,3-propanediol.

In some embodiments, the cryoprotectant is glycerol and/or sorbitol.

In some embodiments, the RNA compositions (such as RNA LNP compositions) described herein may include sucrose as cryoprotectant. Without wishing to be bound by theory, sucrose functions to promote cryoprotection of the compositions, thereby preventing nucleic acid (especially RNA) particle aggregation and maintaining chemical and physical stability of the composition. Certain embodiments contemplate alternative cryoprotectants to sucrose in the present disclosure. Alternative stabilizers include, without limitation, glucose, glycerol, and sorbitol, preferably glucose and glycerol.

A preferred cryoprotectant is selected from the group consisting of sucrose, glucose, glycerol, sorbitol, and a combination thereof. In some embodiments, the cryoprotectant is selected from the group consisting of sucrose, glycerol, 1,2-propanediol, 1,3-propanediol, and glucose, such as from the group consisting of sucrose, glycerol, and glucose. In a preferred embodiment, the cryoprotectant comprises sucrose and/or glycerol. In a more preferred embodiment, the cryoprotectant is sucrose. In some other preferred embodiments, the cryoprotectant is glycerol.

In some embodiments, the RNA compositions (such as RNA LNP compositions) described herein comprise the cryoprotectant in a concentration of at least 1% w/v, such as at least 2% w/v, at least 3% w/v, at least 4% w/v, at least 5% w/v, at least 6% w/v, at least 7% w/v, at least 8% w/v or at least 9% w/v. In some embodiments, the concentration of the cryoprotectant in the compositions is up to 25% w/v, such as up to 20% w/v, up to 19% w/v, up to 18% w/v, up to 17% w/v, up to 16% w/v, up to 15% w/v, up to 14% w/v, up to 13% w/v, up to 12% w/v, or up to 11% w/v. In some embodiments, the concentration of the cryoprotectant in the compositions is 1% w/v to 20% w/v, such as 2% w/v to 19% w/v, 3% w/v to 18% w/v, 4% w/v to 17% w/v, 5% w/v to 16% w/v, 5% w/v to 15% w/v, 6% w/v to 14% w/v, 7% w/v to 13% w/v, 8% w/v to 12% w/v, 9% w/v to 11% w/v, or about 10% w/v. In some embodiments, the RNA compositions (such as RNA LNP compositions) described herein comprise a cryoprotectant (such as sucrose, glycerol, 1,2-propanediol, 1,3-propanediol, glucose, or a combination thereof, in particular, sucrose and/or glycerol) in a (total) concentration of from 5% w/v to 15% w/v, such as from 6% w/v to 14% w/v, from 7% w/v to 13% w/v, from 8% w/v to 12% w/v, or from 9% w/v to 11% w/v, or in a concentration of about 10% w/v.

In some embodiments, in particular those where the composition is in frozen form, it is preferred that the cryoprotectant is present in a concentration of between about 100 mM and about 600 mM, preferably between about 200 mM and about 600 mM and more preferably between about 300 mM and about 500 mM.

Preferably, the RNA compositions (such as RNA LNP compositions) described herein comprise the cryoprotectant in a concentration resulting in an osmolality of the composition in the range of from about 50×10⁻³ osmol/kg to about 1 osmol/kg (such as from about 100×10⁻³ osmol/kg to about 900×10⁻³ osmol/kg, from about 120×10⁻³ osmol/kg to about 800×10⁻³ osmol/kg, from about 140×10⁻³ osmol/kg to about 700×10⁻³ osmol/kg, from about 160×10⁻³ osmol/kg to about 600×10⁻³ osmol/kg, from about 180×10⁻³ osmol/kg to about 500×10⁻³ osmol/kg, or from about 200×10⁻³ osmol/kg to about 400×10⁻³ osmol/kg), for example, from about 50×10⁻³ osmol/kg to about 400×10⁻³ osmol/kg (such as from about 50×10⁻³ osmol/kg to about 390×10⁻³ osmol/kg, from about 60×10⁻³ osmol/kg to about 380×10⁻³ osmol/kg, from about 70×10⁻³ osmol/kg to about 370×10⁻³ osmol/kg, from about 80×10⁻³ osmol/kg to about 360×10⁻³ osmol/kg, from about 90×10⁻³ osmol/kg to about 350×10⁻³ osmol/kg, from about 100×10⁻³ osmol/kg to about 340×10⁻³ osmol/kg, from about 120×10⁻³ osmol/kg to about 330×10⁻³ osmol/kg, from about 140×10⁻³ osmol/kg to about 320×10⁻³ osmol/kg, from about 160×10⁻³ osmol/kg to about 310×10⁻³ osmol/kg, from about 180×10⁻³ osmol/kg to about 300×10⁻³ osmol/kg, or from about 200×10⁻³ osmol/kg to about 300×10⁻³ osmol/kg), based on the total weight of the composition.

In some embodiments, in particular those where the composition is in frozen form, it is preferred that the composition has a pH between 4.0 and 8.0, preferably between 5.0 and 7.0, more preferably between 5.5 and 6.5 and most preferably about 5.5.

In some embodiments, in particular those where the composition is in frozen form, it is preferred that the composition (a) comprises a cryoprotectant; (b) has a pH between 4.0 and 8.0, preferably between 5.0 and 7.0, more preferably between 5.5 and 6.5 and most preferably about 5.5; or (c) comprises a cryoprotectant and has a pH between 4.0 and 8.0, preferably between 5.0 and 7.0, more preferably between 5.5 and 6.5 and most preferably about 5.5. In some embodiments, the cryoprotectant is (i) selected from the cryoprotectants disclosed herein; and/or (ii) is present in a concentration as disclosed herein. For example, the cryoprotectant may be selected from the group consisting of sucrose, glycerol, 1,2-propanediol, 1,3-propanediol, glucose, and a combination thereof, such as from the group consisting of sucrose, glycerol and glucose; and/or may be present in a concentration of between about 100 mM and about 600 mM, preferably between about 200 mM and about 600 mM and more preferably between about 300 mM and about 500 mM. In some embodiments, the cryoprotectant is glycerol, which is optionally present in a concentration of between about 100 mM and about 600 mM, preferably between about 200 mM and about 600 mM and more preferably between about 300 mM and about 500 mM.

In some embodiments, the RNA compositions (such as RNA LNP compositions) described herein comprise sucrose as cryoprotectant (e.g., in a concentration of between about 100 mM and about 600 mM, preferably between about 200 mM and about 600 mM and more preferably between about 300 mM and about 500 mM) and a tertiary or cyclic amine (of the formula N(R¹)(R²)(R³) as defined herein) as buffer substance, preferably in the amounts/concentrations specified herein. In some embodiments, the tertiary amine comprises or is TEA or a protonated form thereof.

In some embodiments, the RNA compositions (such as RNA LNP compositions) described herein comprise glycerol as cryoprotectant (e.g., in a concentration of between about 100 mM and about 600 mM, preferably between about 200 mM and about 600 mM and more preferably between about 300 mM and about 500 mM) and a tertiary or cyclic amine (of the formula N(R¹)(R²)(R³) as defined herein) as buffer substance, preferably in the amounts/concentrations specified herein. In some embodiments, the tertiary amine comprises or is TEA or a protonated form thereof.

In some embodiments, the RNA compositions (such as RNA LNP compositions) described herein comprise 1,2-propanediol as cryoprotectant (e.g., in a concentration of between about 100 mM and about 600 mM, preferably between about 200 mM and about 600 mM and more preferably between about 300 mM and about 500 mM) and a tertiary or cyclic amine (of the formula N(R¹)(R²)(R³) as defined herein) as buffer substance, preferably in the amounts/concentrations specified herein. In some embodiments, the tertiary amine comprises or is TEA or a protonated form thereof.

In some embodiments, the RNA compositions (such as RNA LNP compositions) described herein comprise 1,3-propanediol as cryoprotectant (e.g., in a concentration of between about 100 mM and about 600 mM, preferably between about 200 mM and about 600 mM and more preferably between about 300 mM and about 500 mM) and a tertiary or cyclic amine (of the formula N(R¹)(R²)(R³) as defined herein) as buffer substance, preferably in the amounts/concentrations specified herein. In some embodiments, the tertiary amine comprises or is TEA or a protonated form thereof.

In some embodiments, the RNA compositions (such as RNA LNP compositions) described herein comprise glucose as cryoprotectant (e.g., in a concentration of between about 100 mM and about 600 mM, preferably between about 200 mM and about 600 mM and more preferably between about 300 mM and about 500 mM) and a tertiary or cyclic amine (of the fonnula N(R¹)(R²)(R³) as defined herein) as buffer substance, preferably in the amounts/concentrations specified herein. In some embodiments, the tertiary amine comprises or is TEA or a protonated form thereof.

In some alternative embodiments, the RNA compositions (such as RNA LNP compositions) described herein are substantially free of a cryoprotectant, for example they do not contain any cryoprotectant, and comprise a tertiary or cyclic amine (of the formula N(R¹)(R²)(R³) as defined herein) as buffer substance, preferably in the amounts/concentrations specified herein. In some embodiments, the tertiary amine comprises or is TEA or a protonated form thereof.

Certain embodiments of the present disclosure contemplate the use of a chelating agent in an RNA composition (such as an RNA LNP composition) described herein. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), and bis(aminoethyl)glycolether-N,N,N′,N′-tetraacetic acid. In certain embodiments, the chelating agent is EDTA or a salt of EDTA. In an exemplary embodiment, the chelating agent is EDTA disodium dihydrate. In some embodiments, the EDTA is at a concentration from about 0.05 mM to about 5 mM, from about 0.1 mM to about 2.5 mM or from about 0.25 mM to about 1 mM.

In some embodiments, the aqueous phase of the RNA compositions (such as RNA LNP compositions) described herein do not comprise a chelating agent. For example, it is preferred that if the RNA compositions (such as RNA LNP compositions) described herein comprise a chelating agent, said chelating agent is only present in the LNPs, if present.

### Pharmaceutical Compositions

The RNA compositions described herein are useful as or for preparing pharmaceutical compositions or medicaments for therapeutic or prophylactic treatments.

The RNA compositions described herein may be administered in the form of any suitable pharmaceutical composition.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. In the context of the present disclosure, the pharmaceutical composition comprises RNA as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cyctokines, such as monokines, lymphokines, interleukins, chemokines. The chemokines maybe IL-1, IL-2, IL-3, IL-4, IL-5, IL-6, IL-7, IL-8, IL-9, IL-10, IL-12, INFa, INF-γ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in in a frozen form or in a “ready-to-use form” (i.e., in a form, in particular a liquid form, which can be immediately administered to a subject, e.g., without any processing such as thawing, reconstituting or diluting). Thus, prior to administration of a storable form of a pharmaceutical composition, this storable forn has to be processed or transferred into a ready-to-use or administrable form. E.g., a frozen pharmaceutical composition has to be thawed. Ready to use injectables can be presented in containers such as vials, ampoules or syringes wherein the container may contain one or more doses.

In some embodiments, the pharmaceutical composition is in frozen form and can be stored at a temperature of about −90° C. or higher, such as about −90° C. to about −10° C. For example, the frozen pharmaceutical compositions described herein (such as the frozen compositions prepared by the methods of the third, fourth, fifth or eighth aspect, or the frozen compositions of the first, second, seventh, seventh, tenth, eleventh, or twelfth aspect) can be stored at a temperature ranging from about −90° C. to about −10° C., such as from about −905° C. to about −40° C. or from about −40° C. to about −25° C., or from about −25° C. to about −10° C., or a temperature of about −20° C.

In some embodiments of the pharmaceutical compositions in frozen form, the pharmaceutical composition can be stored for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. For example, the frozen pharmaceutical composition can be stored for at least 4 weeks, preferably at least 1 month, more preferably at least 2 months, more preferably at least 3 months, more preferably at least 6 months at −20° C.

In some embodiments of the pharmaceutical compositions in frozen form, the RNA integrity after thawing the frozen pharmaceutical composition is at least 50%, such as at least 60%, at least 70%, at least 80%, at least 85%, at least 90%, at least 95%, at least 97%, at least 98%, or substantially 100%, e.g., after thawing the frozen composition which has been stored (e.g., for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks) at −20° C. In some embodiments of the pharmaceutical compositions in frozen form, the RNA integrity after thawing the frozen pharmaceutical composition is at least 90%, at least 95%, at least 97%, at least 98%, or substantially 100%, e.g., after thawing the frozen composition which has been stored (e.g., for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks) at −20° C.

In some embodiments, the initial RNA integrity of the pharmaceutical composition (i.e., after its preparation but before freezing) is at least 50% and the RNA integrity of the composition after thawing the frozen composition is at least 90%, preferably at least 95%, more preferably at least 97%, more preferably at least 98%, more preferably substantially 100%, of the initial RNA integrity.

In some embodiments of the pharmaceutical compositions in frozen form, the size (Z_{average}) and/or size distribution and/or PDI of the LNPs after thawing the frozen pharmaceutical composition is essentially equal to the size (Z_{average}) and/or size distribution and/or PDI of the LNPs before freezing. For example, if a ready-to-use pharmaceutical composition is prepared from a frozen pharmaceutical composition as described herein, it is preferred that the size (Z_{average}) and/or size distribution and/or PDI of the LNPs contained in the ready-to-use pharmaceutical composition is essentially equal to the size (Z_{average}) and/or size distribution and/or PDI of the LNPs contained in the frozen pharmaceutical composition before freezing (such as contained in the formulation prepared in step (I) of the method of the second aspect).

In some embodiments, the size of the RNA particles and the RNA integrity of the pharmaceutical composition after one freeze/thaw cycle, preferably after two freeze/thaw cycles, more preferably after three freeze/thaw cycles, more preferably after four freeze/thaw cycles, more preferably after five freeze/thaw cycles or more, are substantially the same as (i.e., are essentially equal to) the size of the RNA particles and the RNA integrity of the initial pharmaceutical composition (i.e., before the pharmaceutical composition has been frozen for the first time).

In some embodiments, the pharmaceutical compositions is in liquid form and can be stored at a temperature ranging from about 0° C. to about 20° C. For example, the liquid pharmaceutical compositions described herein (such as the liquid compositions prepared by the methods of the second, fourth or seventh aspect, or the liquid compositions of the fifth, eighth, ninth, or tenth aspect) can be stored at a temperature ranging from about 1° C. to about 15° C., such as from about 2° C. to about 10° C., or from about 2° C. to about 8° C., or at a temperature of about 5° C.

In some embodiments of the pharmaceutical compositions in liquid form, the pharmaceutical composition can be stored for at least 1 week, such as at least 2 weeks, at least 3 weeks, at least 4 weeks, at least 1 month, at least 2 months, at least 3 months, at least 6 months, at least 12 months, or at least 24 months, preferably at least 4 weeks. For example, the liquid pharmaceutical composition can be stored for at least 4 weeks, preferably at least 1 month, more preferably at least 2 months, more preferably at least 3 months, more preferably at least 6 months at 5° C.

In some embodiments of the pharmaceutical composition in liquid form, the RNA integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), is sufficient to produce the desired effect, e.g., to induce an immune response. For example, the RNA integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), may be at least 50%, such as at least 60%, at least 70%, at least 80%, at least 85%, at least 90%, at least 95%, at least 97% or at least 98%, compared to the RNA integrity of the initial composition, i.e., the RNA integrity before the composition has been stored. In some embodiments, the RNA integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), is at least 90%, compared to the RNA integrity of the initial composition, i.e., the RNA integrity before the composition has been stored. In some embodiments, the RNA integrity of the composition after storage for at least four weeks (e.g., for at least three months), preferably at a temperature of 0° C. or higher, such as about 2° C. to about 8° C., is at least 50%, such as at least 60%, at least 70%, at least 80%, at least 85%, at least 90%, at least 95%, at least 97% or at least 98%, compared to the RNA integrity before storage. In some embodiments, the RNA integrity of the composition after storage for at least four weeks (e.g., for at least three months), preferably at a temperature of 0° C. or higher, such as about 2° C. to about 8° C., is at least 90%, compared to the RNA integrity before storage.

In some embodiments, the initial RNA integrity of the pharmaceutical composition (i.e., after its preparation but before storage) is at least 50% and the RNA integrity of the pharmaceutical composition after storage for at least one week (such as for at least 2 weeks, at least three weeks, at least four weeks, at least one month, at least two months, or at least 3 months), preferably at a temperature of 0° C. or higher, such as about 2° C. to about 8° C., is at least 90%, preferably at least 95%, more preferably at least 97%, more preferably at least 98%, of the initial RNA integrity. In some embodiments, the initial RNA integrity of the pharmaceutical composition (i.e., after its preparation but before storage) is at least 50% and the RNA integrity of the pharmaceutical composition after storage for at least one week (such as for at least 2 weeks, at least three weeks, at least four weeks, at least one month, at least two months, or at least 3 months), preferably at a temperature of 0° C. or higher, such as about 2° C. to about 8° C., is at least 90% of the initial RNA integrity.

In some embodiments of the pharmaceutical composition in liquid form, the size (Z_{average}) (and/or size distribution and/or polydispersity index (PDI)) of the LNPs of the pharmaceutical composition, when stored, e.g., at 0° C. or higher for at least one week (such as for at least 2 weeks, at least three weeks, at least four weeks, at least one month, at least two months, at least three months, at least 4 months, or at least 6 months), is sufficient to produce the desired effect, e.g., to induce an immune response. For example, the size (Z_{average}) (and/or size distribution and/or polydispersity index (PDI)) of the LNPs of the pharmaceutical composition, when stored, e.g., at 0° C. or higher for at least one week (such as for at least 2 weeks, at least three weeks, at least four weeks, at least one month, at least two months, at least three months, at least 4 months, or at least 6 months), is essentially equal to the size (Z_{average}) (and/or size distribution and/or PDI) of the LNPs of the initial pharmaceutical composition, i.e., before storage. In some embodiments, the size (Z_{average}) of the LNPs after storage of the pharmaceutical composition e.g., at 0° C. or higher for at least one week (such as for at least four weeks or at least three months) is between about 50 nm and about 500 nm, preferably between about 40 nm and about 200 nm, more preferably between about 40 nm and about 120 nm. In some embodiments, the PDI of the LNPs after storage of the pharmaceutical composition e.g., at 0° C. or higher for at least one week (such as for at least four weeks or at least three months) is less than 0.3, preferably less than 0.2, more preferably less than 0.1. In some embodiments, the size (Z_{average}) of the LNPs after storage of the pharmaceutical composition e.g., at 0° C. or higher for at least one week (such as for at least four weeks or at least three months) is between about 50 nm and about 500 nm, preferably between about 40 nm and about 200 nm, more preferably between about 40 nm and about 120 nm, and the size (Z_{average}) (and/or size distribution and/or PDI) of the LNPs after storage of the pharmaceutical composition e.g., at 0° C. or higher for at least one week (such as for at least four weeks or at least three months) is essentially equal to the size (Z_{average}) (and/or size distribution and/or PDI) of the LNPs before storage. In some embodiments, the size (Z_{average}) of the LNPs after storage of the pharmaceutical composition e.g., at 0° C. or higher for at least one week (such as for at least four weeks or at least three months) is between about 50 nm and about 500 nm, preferably between about 40 nm and about 200 nm, more preferably between about 40 nm and about 120 nm, and the PDI of the LNPs after storage of the pharmaceutical composition e.g., at 0° C. or higher for at least one week (such as for at least four weeks or at least three months) is less than 0.3 (preferably less than 0.2, more preferably less than 0.1).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the particles or pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or pharmaceutical compositions described herein may depend on various of such parameters. In the case that a reaction in a patient is insufficient with an initial dose, higher doses (or effectively higher doses achieved by a different, more localized route of administration) may be used.

In particular embodiments, a pharmaceutical composition of the present disclosure (e.g., an immunogenic composition, i.e., a pharmaceutical composition which can be used for inducing an immune response) is formulated as a single-dose in a container, e.g., a vial. In some embodiments, the immunogenic composition is formulated as a multi-dose formulation in a vial. In some embodiments, the multi-dose formulation includes at least 2 doses per vial. In some embodiments, the multi-dose formulation includes a total of 2-20 doses per vial, such as, for example, 2, 3, 4, 5, 6, 7, 8, 9, 10, 11, or 12 doses per vial. In some embodiments, each dose in the vial is equal in volume. In some embodiments, a first dose is a different volume than a subsequent dose.

A “stable” multi-dose formulation preferably exhibits no unacceptable levels of microbial growth, and substantially no or no breakdown or degradation of the active biological molecule component(s). As used herein, a “stable” immunogenic composition includes a formulation that remains capable of eliciting a desired immunologic response when administered to a subject.

The pharmaceutical compositions of the present disclosure may contain buffers (in particular, derived from the RNA compositions with which the pharmaceutical compositions have been prepared), preservatives, and optionally other therapeutic agents. In one embodiment, the pharmaceutical compositions of the present disclosure, in particular the ready-to-use pharmaceutical compositions, comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants “Pharmaceutically acceptable salts” comprise, for example, acid addition salts which may, for example, be formed by using a pharmaceutically acceptable acid such as hydrochloric acid, acetic acid, lactic acid, 2-(N-morpholino)ethanesulfonic acid (MES), 3-(N-morpholino)propanesulfonic acid (MOPS), 2-[4-(2-hydroxyethyl)piperazin-1-yl]ethanesulfonic acid (HEPES) or benzoic acid. Furthermore, suitable pharmaceutically acceptable salts may include alkali metal salts (e.g., sodium or potassium salts); alkaline earth metal salts (e.g., calcium or magnesium salts); ammonium (NH); and salts formed with suitable organic ligands (e.g., quaternary ammonium and amine cations). Illustrative examples of pharmaceutically acceptable salts can be found in the prior art; see, for example, S. M. Berge et al., “Pharmaceutical Salts”, J. Pharm. Sci., 66, pp. 1-19 (1977)). Salts which are not pharmaceutically acceptable may be used for preparing pharmaceutically acceptable salts and are included in the present disclosure.

The term “diluent” relates a diluting and/or thinning agent. Moreover, the term “diluent” includes any one or more of fluid, liquid or solid suspension and/or mixing media. Examples of suitable diluents include ethanol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Routes of Administration of Pharmaceutical Compositions

In one embodiment, the compositions described herein, such as the pharmaceutical compositions or ready-to-use pharmaceutical compositions described herein, may be administered intravenously, intraarterially, subcutaneously, intradermally, dermally, intranodally, intramuscularly or intratumorally. In certain embodiments, the (pharmaceutical) composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the (pharmaceutical) compositions, in particular the ready-to-use pharmaceutical compositions, are formulated for systemic administration. In another preferred embodiment, the systemic administration is by intravenous administration. In another preferred embodiment, the (pharmaceutical) compositions, in particular the ready-to-use pharmaceutical compositions, are formulated for intramuscular administration.

### Use of Pharmaceutical Compositions

RNA compositions described herein may be used in the therapeutic or prophylactic treatment of various diseases, in particular diseases in which provision of a peptide or protein to a subject results in a therapeutic or prophylactic effect. For example, provision of an antigen or epitope which is derived from a virus may be useful in the treatment or prevention of a viral disease caused by said virus. Provision of a tumor antigen or epitope may be useful in the treatment of a cancer disease wherein cancer cells express said tumor antigen. Provision of a functional protein or enzyme may be useful in the treatment of genetic disorder characterized by a dysfunctional protein, for example in lysosomal storage diseases (e.g. Mucopolysaccharidoses) or factor deficiencies. Provision of a cytokine or a cytokine-fusion may be useful to modulate tumor microenvironment.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “infectious disease” refers to any disease which can be transmitted from individual to individual or from organism to organism, and is caused by a microbial agent. Infectious diseases are known in the art and include, for example, a viral disease, a bacterial disease, or a parasitic disease, which diseases are caused by a virus, a bacterium, and a parasite, respectively. In this regard, the infectious disease can be, for example, sexually transmitted diseases (e.g., chlamydia, gonorrhea, or syphilis), SARS, acquired immune deficiency syndrome (AIDS), measles, chicken pox, cytomegalovirus infections, genital herpes, hepatitis (such as hepatitis B or C), influenza (flu, such as human flu, swine flu, dog flu, horse flu, and avian flu), HPV infection, shingles, rabies, common cold, gastroenteritis, rubella, mumps, anthrax, cholera, diphtheria, foodborne illnesses, leprosy, meningitis, peptic ulcer disease, pneumonia, sepsis, septic shock, tetanus, tuberculosis, typhoid fever, urinary tract infection, Lyme disease, Rocky Mountain spotted fever, chlamydia, pertussis, tetanus, meningitis, scarlet fever, malaria, trypanosomiasis, Chagas disease, leishmaniasis, trichomoniasis, dientamoebiasis, giardiasis, amebic dysentery, coccidiosis, toxoplasmosis, sarcocystosis, rhinosporidiosis, and balantidiasis.

In some embodiments, RNA compositions described herein may be used in the therapeutic or prophylactic treatment of an infectious disease.

In the present context, the term “treatment”, “treating” or “therapeutic intervention” relates to the management and care of a subject for the purpose of combating a condition such as a disease or disorder.

The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In some embodiments of the disclosure, the aim is to provide protection against an infectious disease by vaccination.

In some embodiments of the disclosure, the aim is to provide secreted therapeutic proteins, such as antibodies, bispecific antibodies, cytokines, cytokine fusion proteins, enzymes, to a subject, in particular a subject in need thereof.

In some embodiments of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In some embodiments of the disclosure, the aim is to modulate/reprogram immune cells in the blood.

In some embodiments of the disclosure (in particular those relating to inhibitory RNA), the aim is to reduce or inhibit the expression of a peptide or polypeptide (such as the transcription and/or translation of a target mRNA). In some embodiments, the target mRNA comprises an ORF encoding a pharmaceutically active peptide or polypeptide, in particular a pharmaceutically active peptide or polypeptide whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

In some embodiments, the RNA compositions described herein which contain RNA encoding a SARS-CoV-2 S protein, an immunogenic variant thereof, or an immunogenic fragment of the SARS-CoV-2 S protein or the immunogenic variant thereof (in the following simply “SARS-CoV-2 S RNA compositions”) following administration to a subject induce an antibody response, in particular a neutralizing antibody response, in the subject that targets a panel of different S protein variants such as SARS-CoV-2 S protein variants, in particular naturally occurring S protein variants. In some embodiments, the panel of different S protein variants comprises at least 5, at least 10, at least 15, or even more S protein variants. In some embodiments, such S protein variants comprise variants having amino acid modifications in the RBD domain and/or variants having amino acid modifications outside the RBD domain. In one embodiment, such S protein variant comprises SARS-CoV-2 S protein or a naturally occurring variant thereof wherein the amino acid corresponding to position 321 (Q) in SEQ ID NO: 1 is S. In one embodiment, such S protein variant comprises SARS-CoV-2 S protein or a naturally occurring variant thereof wherein the amino acid corresponding to position 321 (Q) in SEQ ID NO: 1 is L. In one embodiment, such S protein variant comprises SARS-CoV-2 S protein or a naturally occurring variant thereof wherein the amino acid corresponding to position 341 (V) in SEQ ID NO: 1 is I. In one embodiment, such S protein variant comprises SARS-CoV-2 S protein or a naturally occurring variant thereof wherein the amino acid corresponding to position 348 (A) in SEQ ID NO: 1 is T. In one embodiment, such S protein variant comprises SARS-CoV-2 S protein or a naturally occurring variant thereof wherein the amino acid corresponding to position 354 (N) in SEQ ID NO: 1 is D. In one embodiment, such S protein variant comprises SARS-CoV-2 S protein or a naturally occurring variant thereof wherein the amino acid corresponding to position 359 (S) in SEQ ID NO: 1 is N. In one embodiment, such S protein variant comprises SARS-CoV-2 S protein or a naturally occurring variant thereof wherein the amino acid corresponding to position 367 (V) in SEQ ID NO: 1 is F. In one embodiment, such S protein variant comprises SARS-CoV-2 S protein or a naturally occurring variant thereof wherein the amino acid corresponding to position 378 (K) in SEQ ID NO: 1 is S. In one embodiment, such S protein variant comprises SARS-CoV-2 S protein or a naturally occurring variant thereof wherein the amino acid corresponding to position 378 (K) in SEQ ID NO: 1 is R. In one embodiment, such S protein variant comprises SARS-CoV-2 S protein or a naturally occurring variant thereof wherein the amino acid corresponding to position 408 (R) in SEQ ID NO: 1 is L. In one embodiment, such S protein variant comprises SARS-CoV-2 S protein or a naturally occurring variant thereof wherein the amino acid corresponding to position 409 (Q) in SEQ ID NO: 1 is E. In one embodiment, such S protein variant comprises SARS-CoV-2 S protein or a naturally occurring variant thereof wherein the amino acid corresponding to position 435 (A) in SEQ ID NO: 1 is S. In one embodiment, such S protein variant comprises SARS-CoV-2 S protein or a naturally occurring variant thereof wherein the amino acid corresponding to position 439 (N) in SEQ ID NO: 1 is K. In one embodiment, such S protein variant comprises SARS-CoV-2 S protein or a naturally occurring variant thereof wherein the amino acid corresponding to position 458 (K) in SEQ ID NO: 1 is R. In one embodiment, such S protein variant comprises SARS-CoV-2 S protein or a naturally occurring variant thereof wherein the amino acid corresponding to position 472 (I) in SEQ ID NO: 1 is V. In one embodiment, such S protein variant comprises SARS-CoV-2 S protein or a naturally occurring variant thereof wherein the amino acid corresponding to position 476 (G) in SEQ ID NO: 1 is S. In one embodiment, such S protein variant comprises SARS-CoV-2 S protein or a naturally occurring variant thereof wherein the amino acid corresponding to position 477 (S) in SEQ ID NO: I is N. In one embodiment, such S protein variant comprises SARS-CoV-2 S protein or a naturally occurring variant thereof wherein the amino acid corresponding to position 483 (V) in SEQ ID NO: 1 is A. In one embodiment, such S protein variant comprises SARS-CoV-2 S protein or a naturally occurring variant thereof wherein the amino acid corresponding to position 508 (Y) in SEQ ID NO: 1 is H. In one embodiment, such S protein variant comprises SARS-CoV-2 S protein or a naturally occurring variant thereof wherein the amino acid corresponding to position 519 (H) in SEQ ID NO: 1 is P. In one embodiment, such S protein variant comprises SARS-CoV-2 S protein or a naturally occurring variant thereof wherein the amino acid corresponding to position 614 (D) in SEQ ID NO: I is G.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a mutation at a position corresponding to position 501 (N) in SEQ ID NO: 1. In one embodiment, the amino acid corresponding to position 501 (N) in SEQ ID NO: 1 is Y.

Said S protein variant comprising a mutation at a position corresponding to position 501 (N) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 614 (D), 681 (P), 716 (T), 982 (S), 1118 (D), 80 (D), 215 (D), 484 (E), 701 (A), 18 (L), 246 (R), 417 (K), 242 (L), 243 (A), and 244 (L). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: I is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: I is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted.

In some embodiments, the SARS-CoV-2 S RNA compositions described herein following administration to a subject induce an antibody response, in particular a neutralizing antibody response, in the subject that targets VOC-202012/01.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: deletion 69-70, deletion 144, N501Y, A570D, D614G, P681H, T716I, S982A, and D1118H.

In some embodiments, the SARS-CoV-2 S RNA compositions described herein following administration to a subject induce an antibody response, in particular a neutralizing antibody response, in the subject that targets 501.V2.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N50LY and A701V, and optionally: L18F, R246I, K417N, and deletion 242-244. Said S protein variant may also comprise a D->G mutation at a position corresponding to position 614 in SEQ ID NO: 1.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a deletion at a position corresponding to positions 69 (H) and 70 (V) in SEQ ID NO: 1.

In some embodiments, a S protein variant comprising a deletion at a position corresponding to positions 69 (H) and 70 (V) in SEQ ID NO: I may comprise one or more further mutations. Such one or more further mutations may be selected from mutations at positions corresponding to the following positions in SEQ ID NO: 1: 144 (Y), 501 (N), 570 (A), 614 (D), 681 (P), 716 (T), 982 (S), 1118 (D), 80 (D), 215 (D), 484 (E), 701 (A), 18 (L), 246 (R), 417 (K), 242 (L), 243 (A), 244 (L), 453 (Y), 692 (I), 1147 (S), and 1229 (M). In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: I is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: I is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is 1. In one embodiment, the amino acid corresponding to position 417 (K) in SEQ ID NO: I is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I.

In some embodiments, the SARS-CoV-2 S RNA compositions described herein following administration to a subject induce an antibody response, in particular a neutralizing antibody response, in the subject that targets VOC-202012/01.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: deletion 69-70, deletion 144, N501Y, A570D, D614G, P681H, T716I, S982A, and D118H.

In some embodiments, the SARS-CoV-2 S RNA compositions described herein following administration to a subject induce an antibody response, in particular a neutralizing antibody response, in the subject that targets “Cluster 5”.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: deletion 69-70, Y453F, I692V, M1229I, and optionally S1147L.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a mutation at a position corresponding to position 614 (D) in SEQ ID NO: 1. In one embodiment, the amino acid corresponding to position 614 (D) in SEQ ID NO: 1 is G.

In some embodiments, a S protein variant comprising a mutation at a position corresponding to position 614 (D) in SEQ ID NO: 1 may comprise one or more further mutations. Such one or more further mutations may be selected from mutations at positions corresponding to the following positions in SEQ ID NO: 1: 69 (H), 70 (V), 144 (Y), 501 (N), 570 (A), 681 (P), 716 (T), 982 (S), 1118 (D), 80 (D), 215 (D), 484 (E), 701 (A), 18 (L), 246 (R), 417 (K), 242 (L), 243 (A), 244 (L), 453 (Y), 692 (1), 1147 (S), and 1229 (M). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: I is A. In one embodiment, the amino acid corresponding to position 215 (D) in SEQ ID NO: 1 is G. In one embodiment, the amino acid corresponding to position 484 (E) in SEQ ID NO: I is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is L. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: I is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I.

In some embodiments, the SARS-CoV-2 S RNA compositions described herein following administration to a subject induce an antibody response, in particular a neutralizing antibody response, in the subject that targets VOC-202012/01.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: deletion 69-70, deletion 144, N501Y, A570D, D614G, P681H, T716I, S982A, and D1 118H.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, D614G and A701V, and optionally: L18F, R246I, K417N, and deletion 242-244.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a mutation at positions corresponding to positions 501 (N) and 614 (D) in SEQ ID NO: 1. In one embodiment, the amino acid corresponding to position 501 (N) in SEQ ID NO: 1 is Y and the amino acid corresponding to position 614 (D) in SEQ ID NO: 1 is G.

In some embodiments, a S protein variant comprising a mutation at positions corresponding to positions 501 (N) and 614 (D) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 681 (P), 716 (T), 982 (S), 1118 (D), 80 (D), 215 (D), 484 (E), 701 (A), 18 (L), 246 (R), 417 (K), 242 (L), 243 (A), 244 (L), 453 (Y), 692 (I), 1147 (S), and 1229 (M). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I.

In some embodiments, the SARS-CoV-2 S RNA compositions described herein following administration to a subject induce an antibody response, in particular a neutralizing antibody response, in the subject that targets VOC-202012/01.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: deletion 69-70, deletion 144, N501Y, A570D, D614G, P681H, T716I, S982A, and D1118H.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, D614G and A701V, and optionally: L18F, R246I, K417N, and deletion 242-244.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a mutation at a position corresponding to position 484 (E) in SEQ ID NO: 1. In one embodiment, the amino acid corresponding to position 484 (E) in SEQ ID NO: 1 is K.

In some embodiments, a S protein variant comprising a mutation at a position corresponding to position 484 (E) in SEQ ID NO: 1 may comprise one or more further mutations. Such one or more further mutations may be selected from mutations at positions corresponding to the following positions in SEQ ID NO: 1: 69 (H), 70 (V), 144 (Y), 501 (N), 570 (A), 614 (D), 681 (P), 716 (T), 982 (S), 1118 (D), 80 (D), 215 (D), 701 (A), 18 (L), 246 (R), 417 (K), 242 (L), 243 (A), 244 (L), 453 (Y), 692 (1), 1147 (S), 1229 (M), 20 (T), 26 (P), 138 (D), 190 (R), 417 (K), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: I is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: I is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: I is 1. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 417 (K) in SEQ ID NO: I is T. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the SARS-CoV-2 S RNA compositions described herein following administration to a subject induce an antibody response, in particular a neutralizing antibody response, in the subject that targets 501.V2.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y and A701V, and optionally: L18F, R246I, K417N, and deletion 242-244. Said S protein variant may also comprise a D->G mutation at a position corresponding to position 614 in SEQ ID NO: 1.

In some embodiments, the SARS-CoV-2 S RNA compositions described herein following administration to a subject induce an antibody response, in particular a neutralizing antibody response, in the subject that targets “B.1.1.28”.

In some embodiments, the SARS-CoV-2 S RNA compositions described herein following administration to a subject induce an antibody response, in particular a neutralizing antibody response, in the subject that targets “B.1.1.248”.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: L18F, T20N, P26S, D138Y, R190S, K417T, E484K, N501Y, H655Y, T1027I, and V1176F.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a mutation at positions corresponding to positions 501 (N) and 484 (E) in SEQ ID NO: 1. In one embodiment, the amino acid corresponding to position 501 (N) in SEQ ID NO: 1 is Y and the amino acid corresponding to position 484 (E) in SEQ ID NO: 1 is K.

In some embodiments, a S protein variant comprising a mutation at positions corresponding to positions 501 (N) and 484 (E) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 614 (D), 681 (P), 716 (T), 982 (S), 1118 (D), 80 (D), 215 (D), 701 (A), 18 (L), 246 (R), 417 (K), 242 (L), 243 (A), 244 (L), 453 (Y), 692 (1), 1147 (S), 1229 (M), 20 (T), 26 (P), 138 (D), 190 (R), 417 (K), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: I is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: I is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: I is G. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is 1. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: I is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is L. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 417 (K) in SEQ ID NO: 1 is T. In one embodiment, the amino acid corresponding to position 655 (H) in SEQ ID NO: 1 is Y. In one embodiment, the amino acid corresponding to position 1027 (T) in SEQ ID NO: 1 is L. In one embodiment, the amino acid corresponding to position 1176 (V) in SEQ ID NO: 1 is F.

In some embodiments, the SARS-CoV-2 S RNA compositions described herein following administration to a subject induce an antibody response, in particular a neutralizing antibody response, in the subject that targets 501.V2.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y and A701V, and optionally: L18F, R246I, K417N, and deletion 242-244. Said S protein variant may also comprise a D->G mutation at a position corresponding to position 614 in SEQ ID NO: 1.

In some embodiments, the SARS-CoV-2 S RNA compositions described herein following administration to a subject induce an antibody response, in particular a neutralizing antibody response, in the subject that targets “B.1.1.248”.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: L18F, T20N, P26S, D138Y, R190S, K417T, E484K, N501Y, H655Y, T1027I, and V1176F.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a mutation at positions corresponding to positions 501 (N), 484 (E) and 614 (D) in SEQ ID NO: 1. In one embodiment, the amino acid corresponding to position 501 (N) in SEQ ID NO: 1 is Y, the amino acid corresponding to position 484 (E) in SEQ ID NO: 1 is K and the amino acid corresponding to position 614 (D) in SEQ ID NO: 1 is G.

In some embodiments, a S protein variant comprising a mutation at positions corresponding to positions 501 (N), 484 (E) and 614 (D) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 681 (P), 716 (T), 982 (S), 1118 (D), 80 (D), 215 (D), 701 (A), 18 (L), 246 (R), 417 (K), 242 (L), 243 (A), 244 (L), 453 (Y), 692 (1), 1147 (S), 1229 (M), 20 (T), 26 (P), 138 (D), 190 (R), 417 (K), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: I is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (1) in SEQ ID NO: I is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 417 (K) in SEQ ID NO: 1 is T. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, A701V, and D614G, and optionally: L18F, R246I, K417N, and deletion 242-244.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a deletion at a position corresponding to positions 242 (L), 243 (A) and 244 (L) in SEQ ID NO: 1.

In some embodiments, a S protein variant comprising a deletion at a position corresponding to positions 242 (L), 243 (A) and 244 (L) in SEQ ID NO: 1 may comprise one or more further mutations. Such one or more further mutations may be selected from mutations at positions corresponding to the following positions in SEQ ID NO: 1: 69 (H), 70 (V), 144 (Y), 501 (N), 570 (A), 614 (D), 681 (P), 716 (T), 982 (S), 1118 (D), 80 (D), 215 (D), 484 (E), 701 (A), 18 (L), 246 (R), 417 (K), 453 (Y), 692 (I), 1147 (S), 1229 (M), 20 (T), 26 (P), 138 (D), 190 (R), 417 (K), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is 1. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: I is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 417 (K) in SEQ ID NO: 1 is T. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the SARS-CoV-2 S RNA compositions described herein following administration to a subject induce an antibody response, in particular a neutralizing antibody response, in the subject that targets 501.V2.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, A701V and deletion 242-244, and optionally: L18F, R246I, and K417N. Said S protein variant may also comprise a D->G mutation at a position corresponding to position 614 in SEQ ID NO: 1.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a mutation at a position corresponding to position 417 (K) in SEQ ID NO: 1. In one embodiment, the amino acid corresponding to position 417 (K) in SEQ ID NO: 1 is N. In one embodiment, the amino acid corresponding to position 417 (K) in SEQ ID NO: 1 is T.

In some embodiments, a S protein variant comprising a mutation at a position corresponding to position 417 (K) in SEQ ID NO: 1 may comprise one or more further mutations. Such one or more further mutations may be selected from mutations at positions corresponding to the following positions in SEQ ID NO: 1: 69 (H), 70 (V), 144 (Y), 501 (N), 570 (A), 614 (D), 681 (P), 716 (T), 982 (S), 1118 (D), 80 (D), 215 (D), 484 (E), 701 (A), 18 (L), 246 (R), 242 (L), 243 (A), 244 (L), 453 (Y), 692 (I), 1147 (S), 1229 (M), 20 (T), 26 (P), 138 (D), 190 (R), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: I is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the SARS-CoV-2 S RNA compositions described herein following administration to a subject induce an antibody response, in particular a neutralizing antibody response, in the subject that targets 501.V2.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, A701V, and K417N, and optionally: L18F, R246I, and deletion 242-244. Said S protein variant may also comprise a D->G mutation at a position corresponding to position 614 in SEQ ID NO: 1.

In some embodiments, the SARS-CoV-2 S RNA compositions described herein following administration to a subject induce an antibody response, in particular a neutralizing antibody response, in the subject that targets “B.1.1.248”.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: L18F, T20N, P26S, D138Y, R190S, K417T, E484K, N501Y, H655Y, T1027I, and V1176F.

In some embodiments, the SARS-CoV-2 S RNA compositions described herein following administration to a subject induce an antibody response, in particular a neutralizing antibody response, in the subject that targets a S protein variant such as SARS-CoV-2 S protein variant, in particular naturally occurring S protein variant comprising a mutation at positions corresponding to positions 417 (K) and 484 (E) and/or 501 (N) in SEQ ID NO: 1. In one embodiment, the amino acid corresponding to position 417 (K) in SEQ ID NO: 1 is N, and the amino acid corresponding to position 484 (E) in SEQ ID NO: 1 is K and/or the amino acid corresponding to position 501 (N) in SEQ ID NO: I is Y. In one embodiment, the amino acid corresponding to position 417 (K) in SEQ ID NO: I is T, and the amino acid corresponding to position 484 (E) in SEQ ID NO: 1 is K and/or the amino acid corresponding to position 501 (N) in SEQ ID NO: 1 is Y.

In some embodiments, a S protein variant comprising a mutation at positions corresponding to positions 417 (K) and 484 (E) and/or 501 (N) in SEQ ID NO: 1 may comprise one or more further mutations.

Such one or more further mutations may be selected from mutations at positions corresponding to the following positions in SEQ ID NO: 1: 69 (H), 70 (V), 144 (Y), 570 (A), 614 (D), 681 (P), 716 (T), 982 (S), 1118 (D), 80 (D), 215 (D), 701 (A), 18 (L), 246 (R), 242 (L), 243 (A), 244 (L), 453 (Y), 692 (I), 1147 (S), 1229 (M), 20 (T), 26 (P), 138 (D), 190 (R), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is 1. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: I is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the SARS-CoV-2 S RNA compositions described herein following administration to a subject induce an antibody response, in particular a neutralizing antibody response, in the subject that targets 501.V2.

In some embodiments, the SARS-CoV-2 S RNA compositions described herein following administration to a subject induce an antibody response, in particular a neutralizing antibody response, 30 in the subject that targets a S protein variant comprising the following mutations at positions corresponding to the following positions in SEQ ID NO: 1: D80A, D215G, E484K, N501Y, A701V, and K417N and optionally: L18F, R246I, and deletion 242-244. Said S protein variant may also comprise a D->G mutation at a position corresponding to position 614 in SEQ ID NO: 1.

In some embodiments, the SARS-CoV-2 S RNA compositions described herein following administration to a subject induce an antibody response, in particular a neutralizing antibody response, in the subject that targets “B.1.1.248”.

In some embodiments, the SARS-CoV-2 S RNA compositions described herein following administration to a subject induce an antibody response, in particular a neutralizing antibody response, in the subject that targets a S protein variant comprising the following mutations at positions corresponding to the following positions in SEQ ID NO: 1: L18F, T20N, P26S, D138Y, R190S, K417T, E484K, N501Y, H655Y, T1027I, and V1176F.

In some embodiments, the SARS-CoV-2 S RNA compositions described herein following administration to a subject induce an antibody response, in particular a neutralizing antibody response, in the subject that targets the Omicron (B.1.1.529) variant.

In some embodiments, the SARS-CoV-2 S RNA compositions described herein following administration to a subject induce an antibody response, in particular a neutralizing antibody response, in the subject that targets a S protein variant comprising at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37 of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, N501Y, S375F, Y505H, V143del, H69del, V70del, N211del, L212I, ins214EPE, G142D, Y144del, Y145del, L141del, Y144F, Y145D, G142del, as compared to SEQ ID NO: 1.

In some embodiments, the SARS-CoV-2 S RNA compositions described herein following administration to a subject induce an antibody response, in particular a neutralizing antibody response, in the subject that targets a S protein variant comprising at least 10, at least 15, at least 20, at least 21, at least 22, at least 23, at least 24, or all of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, as compared to SEQ ID NO: 1. Said S protein variant may include at least 1, at least 2, at least 3, at least 4, at least 5, or all of the following mutations: N501Y, S375F, Y5051H, V143del, H69del, V70del, as compared to SEQ ID NO: 1 and/or may include at least 1, at least 2, at least 3, at least 4, at least 5, or all of the following mutations: N211del, L212I, ins214EPE, G142D, Y144del, Y145del, as compared to SEQ ID NO: 1. In some embodiments, said S protein variant may include at least 1, at least 2, at least 3, or all of the following mutations: L141del, Y144F, Y145D, G142del, as compared to SEQ ID NO: 1.

In some embodiments, the SARS-CoV-2 S RNA compositions described herein following administration to a subject induce an antibody response, in particular a neutralizing antibody response, in the subject that targets a S protein variant comprising at least 10, at least 15, at least 20, at least 21, at least 22, at least 23, at least 24, at least 25, at least 26, at least 27, at least 28, at least 29, at least 30, at least 31, at least 32, or at least 33 of the following mutations: A67V, Δ69-70, T95I, G142D, A143-145, A211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the SARS-CoV-2 S RNA compositions described herein following administration to a subject induce an antibody response, in particular a neutralizing antibody response, in the subject that targets a S protein variant comprising the following mutations: A67V, Δ69-70, T95I, G142D, A143-145, A211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the SARS-CoV-2 S RNA compositions described herein following administration to a subject induce an antibody response, in particular a neutralizing antibody response, in the subject that targets a S protein variant comprising the following mutations: A67V, Δ69-70, T95I, G142D, A143-145, A211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the SARS-CoV-2 S RNA compositions described herein following administration to a subject induce an immune response (cellular and/or antibody response, in particular neutralizing antibody response) in the subject that targets the Omicron (B.1.1.529) variant.

In some embodiments, the SARS-CoV-2 S RNA compositions described herein following administration to a subject induce an immune response (cellular and/or antibody response, in particular neutralizing antibody response) in the subject that targets a S protein variant comprising at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37 of the following mutations: T547K, H655Y, D614G, N679K, P681H, N969K, S373P, S371 L, N440K, G339D, G446S, N856K, N764K, K417N, D796Y, Q954H, T95I, A67V, L981F, S477N, G496S, T478K, Q498R, Q493R, E484A, N501Y, S375F, Y505H, V143del, H69del, V70del, N21Idel, L212I, ins214EPE, G142D, Y144del, Y145del, L141del, Y144F, Y145D, G142del, as compared to SEQ ID NO: 1.

In some embodiments, the SARS-CoV-2 S RNA compositions described herein following administration to a subject induce an immune response (cellular and/or antibody response, in particular neutralizing antibody response) in the subject that targets a S protein variant comprising at least 10, at least 15, at least 20, at least 21, at least 22, at least 23, at least 24, or all of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, as compared to SEQ ID NO: 1. Said S protein variant may include at least 1, at least 2, at least 3, at least 4, at least 5, or all of the following mutations: N501Y, S375F, Y505H, V143del, 1169del, V70del, as compared to SEQ ID NO: I and/or may include at least 1, at least 2, at least 3, at least 4, at least 5, or all of the following mutations: N21Idel, L212I, ins214EPE, G142D, Y144del, Y145del, as compared to SEQ ID NO: 1. In some embodiments, said S protein variant may include at least 1, at least 2, at least 3, or all of the following mutations: L141del, Y144F, Y145D, G142del, as compared to SEQ ID NO: 1.

In some embodiments, the SARS-CoV-2 S RNA compositions described herein following administration to a subject induce an immune response (cellular and/or antibody response, in particular neutralizing antibody response) in the subject that targets a S protein variant comprising at least 10, at least 15, at least 20, at least 21, at least 22, at least 23, at least 24, at least 25, at least 26, at least 27, at least 28, at least 29, at least 30, at least 31, at least 32, or at least 33 of the following mutations: A67V, Δ69-70, T95I, G142D, A143-145, A211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the SARS-CoV-2 S RNA compositions described herein following administration to a subject induce an immune response (cellular and/or antibody response, in particular neutralizing antibody response) in the subject that targets a S protein variant comprising the following mutations: A67V, Δ69-70, T95I, G142D, A143-145, A211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the SARS-CoV-2 S RNA compositions described herein following administration to a subject induce an immune response (cellular and/or antibody response, in particular neutralizing antibody response) in the subject that targets a S protein variant comprising the following mutations: A67V, Δ69-70, T95I, G142D, A143-145, A211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

The term “amino acid corresponding to position . . . ” as used herein refers to an amino acid position number corresponding to an amino acid position number in SARS-CoV-2 S protein, in particular the amino acid sequence shown in SEQ ID NO: 1. The phrase “as compared to SEQ ID NO: 1” is equivalent to “at positions corresponding to the following positions in SEQ ID NO: 1”. Corresponding amino acid positions in other coronavirus S protein variants such as SARS-CoV-2 S protein variants may be found by alignment with SARS-CoV-2 S protein, in particular the amino acid sequence shown in SEQ ID NO: 1. It is considered well-known in the art how to align a sequence or segment in a sequence and thereby determine the corresponding position in a sequence to an amino acid position according to the present invention. Standard sequence alignment programs such as ALIGN, ClustalW or similar, typically at default settings may be used.

In some embodiments, the panel of different S protein variants to which an antibody response is targeted comprises at least 5, at least 10, at least 15, or even more S protein variants selected from the group consisting of the Q321S, V341I, A348T, N354D, S359N, V367F, K378S, R408I, Q409E, A435S, K458R, 1472V, G476S, V483A, Y508H, H519P and D614G variants described above. In some embodiments, the panel of different S protein variants to which an antibody response is targeted comprises all S protein variants from the group consisting of the Q321S, V341I, A348T, N354D, S359N, V367F, K378S, R408I, Q409E, A435S, K458R, 1472V, G476S, V483A, Y508H, H519P and D614G variants described above.

In some embodiments, the panel of different S protein variants to which an antibody response is targeted comprises at least 5, at least 10, at least 15, or even more S protein variants selected from the group consisting of the Q321L, V341I, A348T, N354D, S359N, V367F, K378R, R408I, Q409E, A435S, N439K, K458R, 1472V, G476S, S477N, V483A, Y508H, H519P and D614G variants described above.

In some embodiments, the panel of different S protein variants to which an antibody response is targeted comprises all S protein variants from the group consisting of the Q321L, V341I, A348T, N354D, S359N, V367F, K378R, R408I, Q409E, A435S, N439K, K458R, 1472V, G476S, S477N, V483A, Y508H, H519P and D614G variants described above.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises one or more of the mutations described herein for S protein variants such as SARS-CoV-2 S protein variants, in particular naturally occurring S protein variants. In one embodiment, a SARS-CoV-2 S protein, an immunogenic variant thereof, or an immunogenic fragment of the SARS-CoV-2 S protein or the immunogenic variant thereof, e.g., as encoded by the RNA described herein, comprises a mutation at a position corresponding to position 501 (N) in SEQ ID NO: 1. In one embodiment, the amino acid corresponding to position 501 (N) in SEQ ID NO: 1 is Y. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises one or more mutations, such as all mutations, of a SARS-CoV-2 S protein of a SARS-CoV-2 variant selected from the group consisting of VOC-202012/01, 501.V2, Cluster 5 and B.1.1.248. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises an amino acid sequence with alanine substitution at position 80, glycine substitution at position 215, lysine substitution at position 484, tyrosine substitution at position 501, valine substitution at position 701, phenylalanine substitution at position 18, isoleucine substitution at position 246, asparagine substitution at position 417, glycine substitution at position 614, deletions at positions 242 to 244, and proline substitutions at positions 986 and 987 of SEQ ID NO:1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37 of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, N501Y, S375F, Y50511, V143del, H69del, V70del, N21 Idel, L212I, ins214EPE, G142D, Y144del, Y145del, 1141del, Y144F, Y145D, G142del, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises at least 10, at least 15, at least 20, at least 21, at least 22, at least 23, at least 24, or all of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, as compared to SEQ ID NO: 1. Said SARs-CoV-2 S protein, variant, or fragment may include at least 1, at least 2, at least 3, at least 4, at least 5, or all of the following mutations: N501Y, S375F, Y505H, V143del, H69del, V70del, as compared to SEQ ID NO: 1 and/or may include at least 1, at least 2, at least 3, at least 4, at least 5, or all of the following mutations: N211del, L212I, ins214EPE, G142D, Y144del, Y145del, as compared to SEQ ID NO: 1. In some embodiments, said SARs-CoV-2 S protein, variant, or fragment may include at least 1, at least 2, at least 3, or all of the following mutations: L141del, Y144F, Y145D, G142del, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises at least 10, at least 15, at least 20, at least 21, at least 22, at least 23, at least 24, at least 25, at least 26, at least 27, at least 28, at least 29, at least 30, at least 31, at least 32, or at least 33 of the following mutations: A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises the following mutations: A67V, Δ69-70, T95I, G142D, A143-145, A211, L212I, ins214EPE, G339D, S371L, S373P, S375F, K417N, N440K, G446S, S477N, T478K, E484A, Q493R, G496S, Q498R, N501Y, Y50511, T547K, D614G, H655Y, N679K, P681H, N764K, D796Y, N856K, Q954H, N969K, and L981F, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises the following mutations:
A67V, Δ69-70, T95I, G142D, Δ143-145, A211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises the following mutations:
A67V, Δ69-70, T95I, G142D, Δ143-145, A211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprises the following mutations: A67V, Δ69-70, T95I, G142D, Δ143-145, A211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises the amino acid sequence of SEQ ID NO: 42, an amino acid sequence having at least 99.5%, 99%, 98.5%, 98%, 98.5%, 97%, 96%, 95%, 90%, 85%, or 80% identity to the amino acid sequence of SEQ ID NO: 42, or an immunogenic fragment of the amino acid sequence of SEQ ID NO: 42, or the amino acid sequence having at least 99.5%, 99%, 98.5%, 98%, 98.5%, 97%, 96%, 95%, 90%, 85%, or 80% identity to the amino acid sequence of SEQ ID NO: 42. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises the amino acid sequence of SEQ ID NO: 42.

In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises the amino acid sequence of SEQ ID NO: 45, an amino acid sequence having at least 99.5%, 99%, 98.5%, 98%, 98.5%, 97%, 96%, 95%, 90%, 85%, or 80% identity to the amino acid sequence of SEQ ID NO: 45, or an immunogenic fragment of the amino acid sequence of SEQ ID NO: 45, or the amino acid sequence having at least 99.5%, 99%, 98.5%, 98%, 98.5%, 97%, 96%, 95%, 90%, 85%, or 80% identity to the amino acid sequence of SEQ ID NO: 45. In some embodiments, a SARS-CoV-2 S protein, an immunogenic variant thereof, or an immunogenic fragment of the SARS-CoV-2 S protein or the immunogenic variant thereof, e.g., as encoded by the RNA described herein, comprising said mutations comprises the amino acid sequence of SEQ ID NO: 45.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

The terms “immunization” or “vaccination” describe the process of administering an antigen to an individual with the purpose of inducing an immune response, for example, for therapeutic or prophylactic reasons.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Methods

### Manufacturing of the RNA LNPs

Manufacturing protocols are described here with taking lipid X-3 as an example for the cationically ionizable lipid. The same protocols apply as well for other cationically ionizable lipids. Accordingly, also other formulations with ratios between cationically ionizable lipid and RNA (N/P ratio), e.g., higher or lower N/P ratios, including those with negative charge excess, can be manufactured and stabilized as described. In addition, other lipid ratios (phospholipid, cholesterol, polymer conjugated lipid), as well as other types of polymer conjugated lipids (e.g., polysarcosine lipids) can be used. Protocols also apply for products without any polymer conjugated lipid.

RNA LNPs were prepared by an aqueous-ethanol mixing protocol. Briefly, RNA (such as BNT162b2 encoding an amino acid sequence comprising a SARS-CoV-2 S protein) in aqueous buffer conditions (e.g., 50 mM citrate, pH 4.0) is mixed with ethanolic lipid mix comprising of lipid X-3, DSPC, cholesterol and 2-[(polyethylene glycol)-2000]-N,N-ditetradecylacetamide in molar ratio of 47.5:10:40.7:1.8, respectively in the volume ratio of 3 parts of RNA and 1 part of lipid mix. The mixing is achieved using standard pump based set-up using a T mixing element. The lipid nanoparticle raw colloid is further diluted with 2 parts of buffer (e.g., with citrate buffer 50 mM, pH 4.0). The total flow is between 400 and 2000 mL/min, e.g. 720 ml/min. TFF (tangential flow filtration) against citrate buffer (50 mM pH 4.0) was used to reduce ethanol content and concentrate the LNP product. Buffer exchange was performed using dialysis cassettes. Dialysis was for 4 hours against 20 mM target buffer pH 7.4.

After dialysis RNA concentrations of the LNP suspensions were determined. By addition of 10 mM target buffer pH7.4 the stock suspensions were adjusted to equal concentration. Then 4 aliquots of each individual suspension were formulated in 10 mM, 20 mM, 50 mM or 100 mM of that buffer and 10% sucrose by addition of appropriate volumes of 1 M buffer p117.4 and 40% sucrose. Samples of 1 mL each were sealed and stored at 20-25° C.

### LNP Size and Polydispersity

Mean particle size and size distribution of LNPs in an RNA LNP formulation/composition (or a sample thereof) is evaluated by dynamic light scattering (DLS). The method employs a particle sizer that uses back-scatter at 173° to determine particle size. The results are reported as the Z_{averge} size or hydrodynamic diameter of the particles and the polydispersity index. The polydispersity values are used to describe the width of fitted log-normal distribution around the measured Z_{average} size and are generated using proprietary mathematical calculations within the particle sizing software. Results for size and polydispersity are reported as nm and polydispersity index value, respectively. Samples are diluted to an appropriate concentration in buffer or water. For the experiments described here, a Wyatt DynaPro plate reader was used with clear flat bottom 96 well plates, containing 20 μL of sample diluted in 100 μL of water. Hydrodynamic diameter in nm and polydispersity index were calculated using DYNAMICS 7.

### RNA Integrity

RNA integrity is determined by capillary electrophoresis. RNA-LNPs treated with Triton™ X-100/ethanol are applied to a gel matrix contained in a capillary. The RNA and its derivates, degradants and impurities are separated according to their sizes. The gel matrix contains a fluorescence dye which binds specifically to the RNA components which allows detection by blue LED-induced fluorescence, detected by a CCD detector. The excitation wavelength is 470 nm. The integrity of the RNA is determined by comparing the peak area of the main RNA peak to the total detected peak area and is reported as percentage. Late migrating species (LMS) are represented by all signals detected later than the main peak and are also expressed as percentage of total peak area.

### Example 1

RNA LNPs were prepared as described above and dialysed to one of the buffers listed in Table 2.

TABLE 2
Buffer substances tested for prolonged RNA and colloidal stability in LNPs
surface

| | | | | | | | polar surface |
|---|---|---|---|---|---|---|---|
| | | | | | | | area |
| | | | Type of | Nr. of | Nr. of | LogP | (Å\hat{ }2, |
| Nr. | Buffer | Abbr. | Amine | Amines | Anions | (ACD) | ACD) |
| 1 | Tris-(hydroxymethyl) | Tris | prim | 1 | 0 | −1.38 | 87 |
| | aminomethane | | | | | | |
| 2 | BisTrismethane | BTM | tert | 1 | 0 | −0.67 | 104 |
| 3 | Triethanolamine | TEA | tert | 1 | 0 | −1.11 | 64 |
| 4 | Ethylenediamine | EDA | prim | 2 | 0 | −2.04 | 52 |
| 5 | Glycinamide | GlAm | prim | 1 | 0 | −1.65 | 70 |
| 6 | 2-(Hydroxyethyl) | HEPES | tert | 2 | 1 | −2.67 | 89 |
| | piperazine | | | | | | |
| | ethanesulfonic acid | | | | | | |
| 7 | Phosphate | Phos | — | — | 2 | −2.15 | 96 |
| 8 | N-(tri(hydroxymethyl) | Tricine | sec | 1 | 1 | −0.28 | 110 |
| | methyl)glycine | | | | | | |
| 9 | N-Ethyldiethanolamine | EDEA | tert | 1 | 0 | −0.19 | 44 |
| 10 | 2-(Diethylamino)ethanol | DEAE | tert | 1 | 0 | 0.74 | 23 |
| 11 | Triethylamine | 3-ethyl-N | tert | 1 | 0 | 1.66 | 3 |
| 12 | 2-(2-(Diethylamino) | Ethoxyamine | tert | 1 | 0 | −0.41 | 33 |
| | ethoxy)ethanol | | | | | | |
| 13 | Diethanolamine | DEA | sec | 1 | 0 | −1.5 | 52 |
| 14 | N,N′-Bis(2-hydroxyethyl) | BHEED | sec | 2 | 0 | −1.81 | 65 |
| | ethylenediamine | | | | | | |
| 15 | N,N,N′,N′-Tetrakis(2- | THEED | tert | 2 | 0 | −1.26 | 87 |
| | hydroxyethyl) | | | | | | |
| | ethylenediamine | | | | | | |
| 16 | 1,4-Bis(2- | BHEP | tert | 2 | 0 | −1.14 | 47 |
| | hydroxyethyl)piperazine | | | | | | |
| 17 | L-Histidine | His | sec | 2 | 1 | −1.26 | 92 |
| 18 | Trimethylamine N-oxide | TMAO | quart | 1 | 0 | −2.57 | 17 |
| 19 | Morpholine | Morph | sec | 1 | 0 | −1.08 | 21 |

The buffers had the concentrations listed in Table 2 and all samples did further comprise 300 mM sucrose. RNA integrity was analysed over time and is reported as % intact RNA, % RNA appearing as late migrating species (LMS) and % of fragmented RNA (from left to right). Starting integrity of RNA in all samples was 75%.

TABLE 3
RNA integrity, LMS and degradation after storage at room temperature in 19 buffers.

| | | | RNA integrity (%) | LMS (%) | degradation (%) |
|---|---|---|---|---|---|
| | | | Wks 20°-25° C. | Wks 20°-25° C. | Wks 20°-25° C. |
| | Buffer | mM | 12 | 12 | 12 |
| 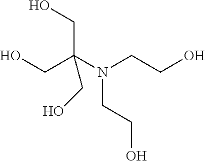 [SMILES: OCCN(CCO)C(CO)(CO)CO] | BTM:Cl BTM:Cl BTM:Cl BTM:Cl | 10 20 50 100 | 31.2 30.3 32.6 37.2 | 16.5 15.5 13.9 4.5 | 52.3 54.2 53.5 58.3 |
| 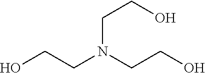 | TEA:Cl TEA:Cl TEA:Cl TEA:Cl | 10 20 50 100 | 40.6 50.2 43.9 37.8 | 6.6 2.6 3.9 5.3 | 52.8 47.2 52.2 56.9 |
| 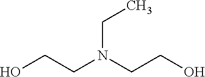 | EDEA:Cl EDEA:Cl EDEA:Cl EDEA:Cl | 10 20 50 100 | 33.1 34.6 43.0 40.5 | 10.2 11.9 6.9 9.1 | 56.7 53.5 50.1 50.4 |
| 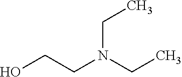 | DEAE:Cit DEAE:Cit DEAE:Cit DEAE:Clt | 10 20 50 100 | 35.0 33.1 42.0 39.7 | 10.5 11.9 10.2 6.8 | 54.5 55.0 47.8 53.5 |
| 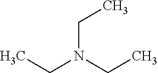 | 3-ethyl-N:Cit 3-ethyl-N:Cit 3-ethyl-N:Cit 3-ethyl-N:Cit | 10 20 50 100 | 36.4 34.8 39.5 51.2 | 6.9 5.7 5.4 3.2 | 56.7 59.5 55.1 45.6 |
| 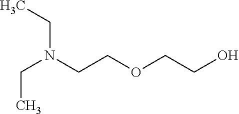 | -ethoxy-:Cit -ethoxy-:Cit -ethoxy-:Cit -ethoxy-:Cit | 10 20 50 100 | 35.9 30.4 41.4 48.1 | 8.1 8.3 8.7 7.2 | 56.0 61.3 49.9 44.7 |
| 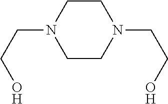 [SMILES: OCCN1CCN(CCO)CC1] | BHEP:Cl BHEP:Cl BHEP:Cl BHEP:Cl | 10 20 50 100 | 7.9 36.1 36.4 31.7 | 4.3 5.0 3.1 3.9 | 87.8 58.9 60.5 64.4 |
| 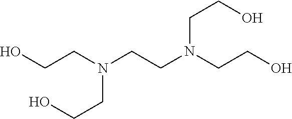 [SMILES: OCCN(CCO)CCN(CCO)CCO] | THEED:Cl THEED:Cl THEED:Cl THEED:Cl | 10 20 50 100 | 32.3 31.5 25.9 19.7 | 3.7 2.6 3.2 3.2 | 64.0 65.9 70.9 77.1 |
| 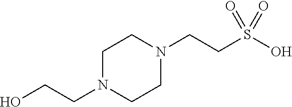 [SMILES: O=S(=O)(O)CCN1CCN(CCO)CC1] | HEPES:Na HEPES:Na HEPES:Na HEPES:Na | 10 20 50 100 | 32.8 29.7 28.5 28.6 | 14.7 13.4 15.7 18.1 | 52.5 56.9 55.8 53.3 |
| 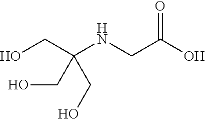 [SMILES: O=C(O)CNC(CO)(CO)CO] | Tricine:Na Tricine:Na Tricine:Na Tricine:Na | 10 20 50 100 | 28.6 28.9 28.0 27.3 | 16.7 14.6 13.8 14.6 | 54.7 56.5 58.2 58.1 |
| 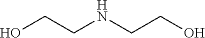 | DEA:Cl DEA:Cl DEA:Cl | 10 20 50 | 36.1 34.1 15.4 | 7.3 5.8 1.0 | 56.6 60.1 83.6 |
| | DEA:Cl | 100 | 3.5 | 1.1 | 95.4 |
| 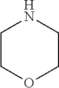 | Morph:Cl Morph:Cl Morph:Cl Morph:Cl | 10 20 50 100 | 22.6 12.8 3.2 1.3 | 10.3 7.9 3.3 2.5 | 67.1 79.3 93.5 96.2 |
| 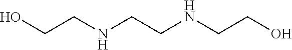 | BHEED:Cl BHEED:Cl BHEED:Cl | 10 20 50 | 0.0 0.1 0.2 | 0.0 0.3 0.3 | 100.0 99.6 99.5 |
| | BHEED:Cl | 100 | 0.4 | 1.2 | 98.4 |
| 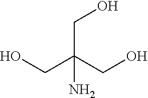 | Tris:Cl Tris:Cl Tris:Cl Tris:Cl | 10 20 50 100 | 33.7 25.4 8.5 1.8 | 12.7 4.5 3.1 1.1 | 53.6 70.1 88.4 97.1 |
| 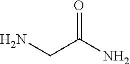 | GlAm:Na GlAm:Na GlAm:Na GlAm:Na | 10 20 50 100 | 21.2 3.4 0.1 0.2 | 8.6 1.1 0.2 0.3 | 70.2 95.5 99.7 99.5 |
| 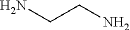 | EDA:Cl EDA:Cl | 10 20 | 0.1 0.0 | 0.3 0.0 | 99.6 100.0 |
| | EDA:Cl | 50 | 0.0 | 0.0 | 100.0 |
| | EDA:Cl | 100 | 0.2 | 1.3 | 98.5 |
| 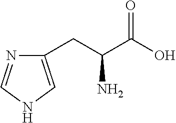 [SMILES: N[C@@H](Cc1c[nH]cn1)C(=O)O] | His:Cl His:Cl His:Cl His:Cl | 10 20 50 100 | 34.6 29.1 27.6 24.2 | 6.8 5.9 5.6 6.1 | 58.6 65.0 66.8 69.7 |
| 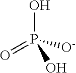 | Phosph:Na Phosph:Na Phosph:Na Phosph:Na | 10 20 50 100 | 32.5 28.9 27.1 27.0 | 15.1 18.0 19.1 21.9 | 52.4 53.1 53.8 51.1 |
| 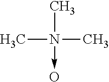 | TMAO:Cl TMAO:Cl TMAO:Cl TMAO:Cl | 10 20 50 100 | 37.2 36.1 34.2 33.3 | 8.1 6.8 7.7 6.0 | 54.7 57.1 58.1 60.7 |

TABLE 4
LNP size after storage at room temperature in 19 buffers.

| | | | hydr. diameter | |
|---|---|---|---|---|
| | | | (nm) | |
| | | | Wks 20°-25° C. | |
| | Buffer | mM | 0.1 | 12 |
| 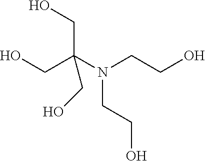 [SMILES: OCCN(CCO)C(CO)(CO)CO] | BTM:Cl BTM:Cl BTM:Cl BTM:Cl | 10 20 50 100 | 89 90 89 88 | 107 85 90 92 |
| 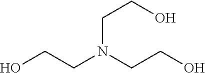 | TEA:Cl TEA:Cl TEA:Cl TEA:Cl | 10 20 50 100 | 86 88 90 94 | 87 91 90 90 |
| 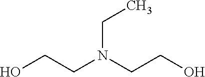 | EDEA:Cl EDEA:Cl EDEA:Cl EDEA:Cl | 10 20 50 100 | 90 92 89 94 | 93 94 95 96 |
| 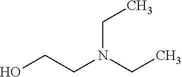 | DEAE:Cit DEAE:Cit DEAE:Cit DEAE:Cit | 10 20 50 100 | 93 90 90 95 | 127 93 98 109 |
| 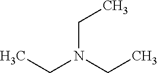 | 3-ethyl-N:Cit 3-ethyl-N:Cit 3-ethyl-N:Cit 3-ethyl-N:Cit | 10 20 50 100 | 92 89 92 91 | 127 97 95 103 |
| 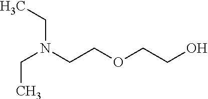 | -ethoxy-:Cit -ethoxy-:Cit -ethoxy-:Cit -ethoxy-:Cit | 10 20 50 100 | 93 92 92 94 | 133 90 100 105 |
| 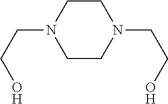 [SMILES: OCCN1CCN(CCO)CC1] | BHEP:Cl BHEP:Cl BHEP:Cl BHEP:Cl | 10 20 50 100 | 92 90 91 91 | 83 84 86 87 |
| 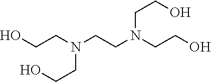 [SMILES: OCCN(CCO)CCN(CCO)CCO] | THEED:Cl THEED:Cl THEED:Cl THEED:Cl | 10 20 50 100 | 86 87 86 90 | 86 86 88 91 |
| 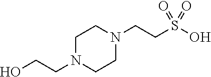 [SMILES: O=S(=O)(O)CCN1CCN(CCO)CC1] | HEPES:Na HEPES:Na HEPES:Na HEPES:Na | 10 20 50 100 | 98 87 85 85 | 99 90 89 91 |
| 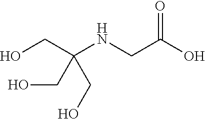 [SMILES: O=C(O)CNC(CO)(CO)CO] | Tricine:Na Tricine:Na Tricine:Na Tricine:Na | 10 20 50 100 | 87 93 88 85 | 110 115 86 88 |
| 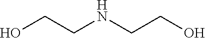 | DEA:Cl DEA:Cl DEA:Cl | 10 20 50 | 93 93 89 | 92 98 98 |
| | DEA:Cl | 100 | 90 | 94 |
| 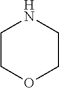 | Morph:Cl Morph:Cl Morph:Cl Morph:Cl | 10 20 50 100 | 88 88 85 85 | 84 86 83 86 |
| 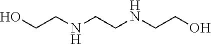 | BHEED:Cl BHEED:Cl BHEED:Cl | 10 20 50 | 86 87 86 | 85 86 88 |
| | BHEED:Cl | 100 | 88 | 88 |
| 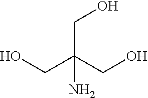 | Tris:Cl Tris:Cl Tris:Cl Tris:Cl | 10 20 50 100 | 86 82 85 84 | 85 84 88 87 |
| 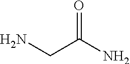 | GlAm:Na GlAm:Na GlAm:Na GlAm:Na | 10 20 50 100 | 90 87 87 87 | 83 88 91 88 |
| 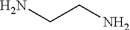 | EDA:Cl EDA:Cl | 10 20 | 84 86 | 87 87 |
| | EDA:Cl | 50 | 85 | 85 |
| | EDA:Cl | 100 | 86 | 88 |
| 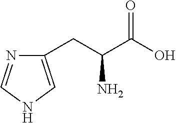 [SMILES: N[C@@H](Cc1c[nH]cn1)C(=O)O] | His:Cl His:Cl His:Cl His:Cl | 10 20 50 100 | 88 92 101 98 | 112 147 102 85 |
| 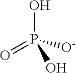 | Phosph:Na Phosph:Na Phosph:Na Phosph:Na | 10 20 50 100 | 81 81 81 81 | 87 87 87 89 |
| 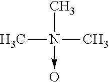 | TMAO:Cl TMAO:Cl TMAO:Cl TMAO:Cl | 10 20 50 100 | 87 88 90 88 | 121 117 121 123 |

The data presented in Tables 3 and 4 when viewed in light of the structural elements of the buffer ions shown in Table 1 allow the following conclusions:
1. Fragmentation of RNA relates to the type of amine and reduced degradation is found in the order of tertiary or quaternary amines<secondary amines<primary amines.
2. Fragmentation of RNA is lower for monoamines compared to diamines.
3. The generation of a late migrating species relates to the presence of anionic moieties in the buffer compound.
4. Tertiary monoamines protect RNA from both fragmentation and LMS formation.

The conclusions are further supported by the FIGS. 1 to 3.

The correlation between type of amine and RNA degradation is presented more clearly in FIG. 1, showing a clear enrichment of tertiary monoamines among buffers inflicting low degradation of RNA.

Primary and some secondary amines, on the other hand, result in substantial or complete degradation of the RNA (Correlation 0.86).

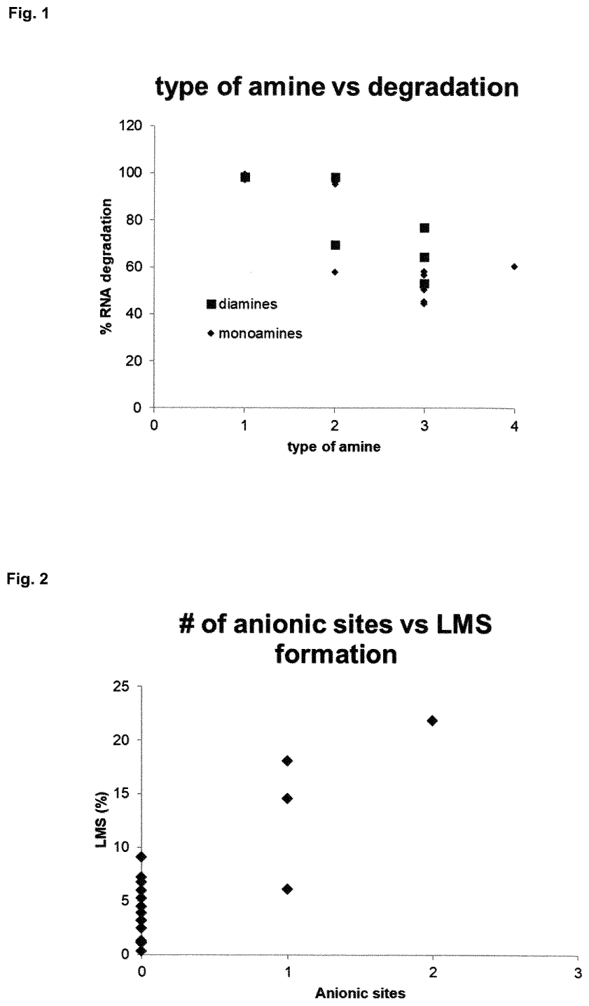

Similarly, formation of LMS shows a clear correlation with the number of anionic sites in the buffer substances, as shown in FIG. 2. Anionic moieties such as phosphate, sulfonic acid or carboxyl promote the formation of LMS (Correlation 0.78).

FIG. 3 shows degradation of RNA for each buffer at its optimal concentration. Best performing buffers are consistently tertiary monoamines at concentrations between 20 and 100 mM.

### Example 2

Lipid nanoparticles were prepared by mixing an aqueous mRNA solution and an ethanolic lipid solution. Thereafter, a purification step (using a 0.2 m filter) and a compounding step followed. In details, the mRNA was provided in 40 mM citrate buffer pH 4.0 to a final concentration of 0.4 mg/mL. The lipids were dissolved in absolute EtOH at 35° C. to a final concentration of 30.1 mg/mL and filtered through a 0.2 μm PES filter. To form lipid nanoparticles, one volume of the lipid solution was combined with three volumes of the mRNA solution at RT using a mixer. Shortly after this mixing step, an online dilution step with two volumes of citrate buffer pH 4.0 was performed, to reduce the EtOH concentration. The intermediate product was then diafiltered using tangential flow filtration, against citrate buffer pH 4.0 (2 volume exchanges) to reduce further the EtOH concentration. Following diafiltration, the product was split into 6 equal aliquots and each aliquot was dialyzed against a different buffer as listed in Table 5.

TABLE 5
Buffers used for dialysis of each aliquot

| Aliquot (#) | Buffer | Concentration (mM) | pH |
|---|---|---|---|
| 1 | TEA | 50 | 5.5 |
| 2 | | | 6.9 |
| 3 | | | 7.5 |
| 4 | Tris | 10 | 5.5 |
| 5 | | | 6.9 |
| 6 | | | 7.5 |

After dialysis, each aliquot was collected, 0.2 m filtered using syringe filter, and analyzed for mRNA content. After mRNA quantification each sample was split in 32 equal aliquots and each of them was topped with one of the eight different cryoprotectants to be tested at varying concentrations, as listed in Table 6.

TABLE 6
Cryoprotectants and their concentrations used for topping
of each aliquot in the freeze thaw experiments.

| | |
|---|---|
| Cryoprotectant | Concentration (mM) |

| | | | | |
|---|---|---|---|---|
| 1,2-Propanediol | 120 | 240 | 360 | 480 |
| 1,3-Propanediol | | | | |
| Glycerol | | | | |
| Glucose | | | | |
| Sorbitol | | | | |
| Mannitol | | | | |
| Sucrose | | | | |
| Trehalose | | | | |

The target mRNA concentration was 0.1 mg/mL. All aliquots were then filled in deep 96-well plates that were used as starting material for the freeze thaw experiments.

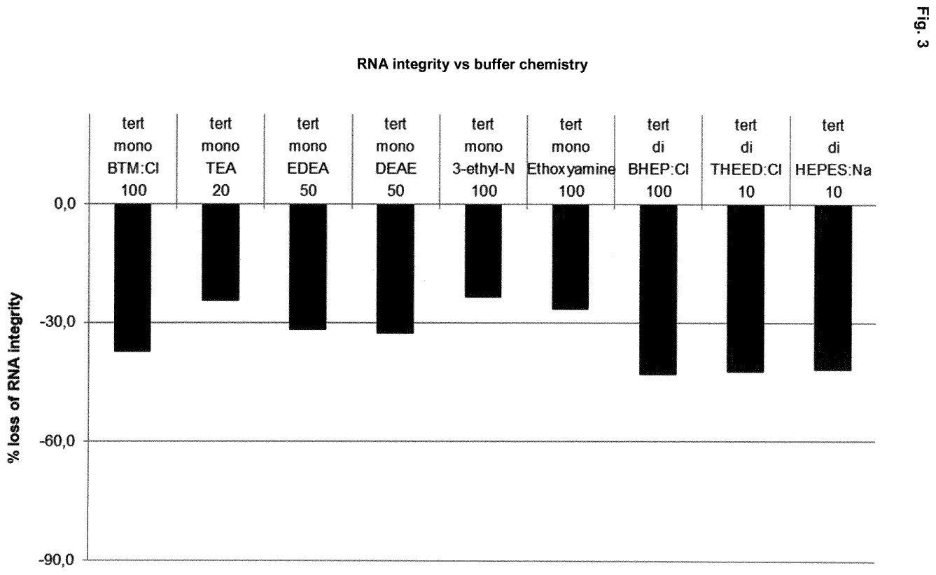

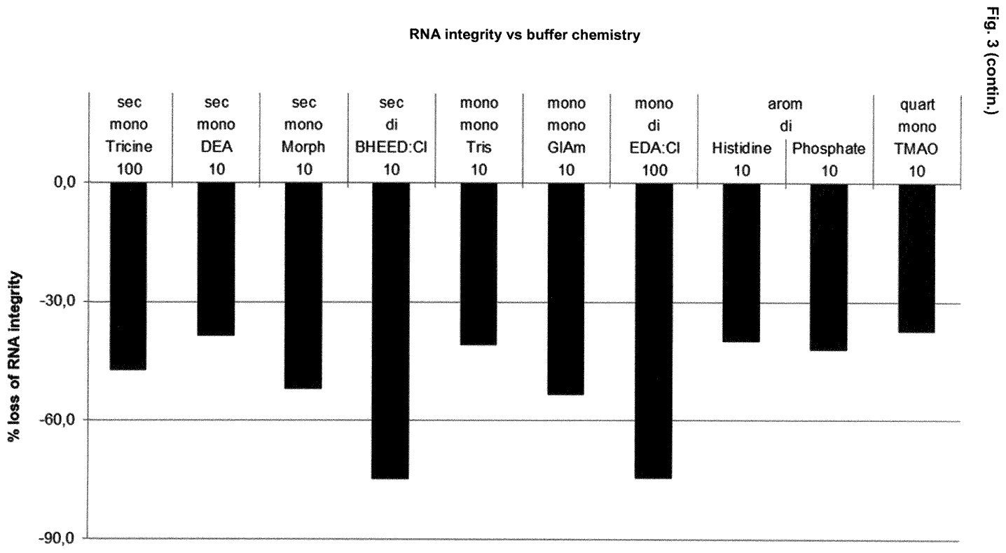

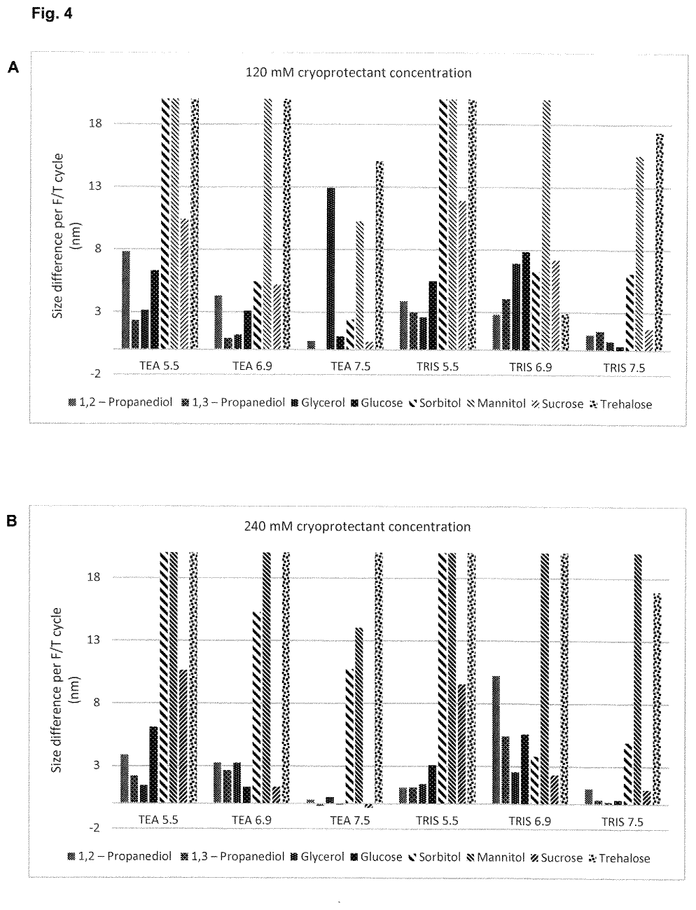

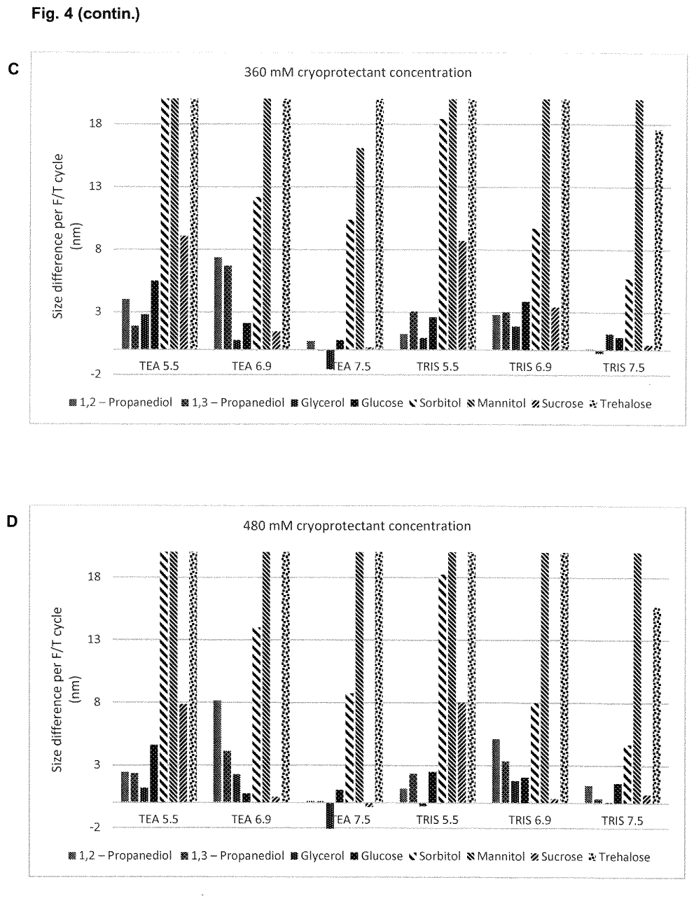

The results of these experiments are shown in FIGS. 4A-D.

As can be seen from FIGS. 4A-D, the compositions comprising sucrose or glycerol as cryoprotectant perform best, i.e., they exhibit good colloidal stability even after 5 freeze/thaw cycles.

1. A composition comprising (i) RNA; (ii) a cationically ionizable lipid; and (iii) an aqueous phase, wherein the aqueous phase comprises a buffer system comprising a buffer substance having the formula N(R¹)(R²)(R³), its N-oxide, or a protonated form thereof, wherein:
each of R¹, R², and R³ is independently selected from H, C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is H, CH(C₁₋₅alkylene-R⁴)₂, or C(C₁₋₅ alkylene-R⁴)₃; or two of R¹, R², and R³ join together with the nitrogen atom to form a 5- or 6-membered N-heterocyclic ring which is optionally substituted with one or two R⁵;
each R⁴ is independently selected from —OH, —O-(C₁₋₆ alkylene-OH), and —N(R⁶)_{z}(C₁₋₆ alkylene-OH)_{2-z}, wherein each z is independently selected from 0 and 1; and each R⁶ is independently selected from H and C₁₋₃ alkyl; and
each R⁵ is independently selected from C₁₋₆ alkyl, C₁₋₆ alkylene-R⁴, CH(C₁₋₅ alkylene-R⁴)₂, and C(C₁₋₅ alkylene-R⁴)₃.

2. The composition of claim 1, wherein each of R¹, R², and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is CH(C₁₋₅ alkylene-R⁴)₂ or C(C₁₋₅ alkylene-R⁴)₃, preferably each of R¹, R², and
R³ is independently selected from C₁₋₄ alkyl, C₁₋₄ alkylene-R⁴, CH(C₁₋₃ alkylene-R⁴)₂, and C(C₁₋₃ alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₃ alkyl, C₁₋₃alkylene-R⁴, CH(C₁₋₃ alkylene-R⁴)₂, and C(C₁₋₃ alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₂ alkyl, C₁₋₂ alkylene-R⁴, CH(C₁₋₂ alkylene-R⁴)₂, and C(C₁₋₂alkylene-R⁴)₃, wherein at most one of R¹, R², and R³ is CH(C₁₋₂ alkylene-R⁴)₂ or C(C₁₋₂ alkylene-R⁴)₃.

3. The composition of claim 1 or 2, wherein each of R¹, R², and R³ is independently selected from C₁₋₆ alkyl, C₁₋₆ alkylene-R⁴, and C(C₁₋₅ alkylene-R⁴)₃, wherein at most one of R¹, R², and R³ is C(C₁₋₅ alkylene-R⁴)₃, preferably each of R¹, R², and R³ is independently selected from C₁₋₄ alkyl, C₁₋₄ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₃ alkyl, C₁₋₃ alkylene-R⁴, and C(C₁₋₃ alkylene-R⁴)₃, wherein at most one of R¹, R², and R³ is C(C₁₋₃alkylene-R⁴)₃, more preferably each of R¹, R², and R³ is independently selected from C₁₋₂ alkyl, C₁₋₂ alkylene-R⁴, and C(C₁₋₂ alkylene-R⁴)₃, wherein at most one of R¹, R², and R³ is C(C₁₋₂alkylene-R⁴)₃.

4. The composition of any one of claims 1 to 3, wherein each of R¹, R², and R³ is independently selected from C₁₋₆ alkyl and C₁₋₆ alkylene-R⁴, preferably each of R¹, R², and R³ is independently selected from C₁₋₄ alkyl and C₁₋₄ alkylene-R⁴, more preferably each of R¹, R², and R³ is independently selected from C₁₋₃ alkyl and C₁₋₃ alkylene-R⁴, more preferably each of R¹, R², and R³ is independently selected from C₁₋₂ alkyl and C₁₋₂ alkylene-R⁴.

5. The composition of any one of claims 1 to 4, wherein each R⁴ is independently selected from —OH, —O-(C₁₋₄ alkylene-OH), and —N(R⁶)_{z}-(C₁₋₄ alkylene-OH)_{2-z}, wherein each z is independently selected from 0 and 1; and each R⁶ is independently selected from H and C₁₋₃ alkyl, preferably each R⁴ is independently selected from —OH, —O-(C₁₋₃ alkylene-OH), and —N(R⁶)_{z}-(C₁₋₃ alkylene-OH)_{2-z}, wherein each z is independently selected from 0 and 1; and each R⁶ is independently selected from H and C₁₋₃ alkyl, more preferably each R⁴ is independently selected from —OH, —O-(C₁₋₂ alkylene-OH), and —N(R⁶)_{z}-(C₁₋₂ alkylene-OH)_{2-z}, wherein each z is independently selected from 0 and 1; and each R⁶ is independently selected from H and C₁₋₂ alkyl.

6. The composition of any one of claims 1 to 5, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

7. The composition of any one of claims 1 to 6, wherein each R⁴ is independently selected from —OH, 2-hydroxyethoxy, and bis(2-hydroxyethyl)amino.

8. The composition of any one of claims 1 to 7, wherein each of R¹, R², and R³ is independently selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

9. The composition of any one of claims 1 to 8, wherein all of R¹, R², and R³ are the same.

10. The composition of claim 9, wherein all of R¹, R², and R³ are methyl, ethyl, or 2-hydroxyethyl.

11. The composition of any one of claims 1 to 8, wherein R¹ and R² are the same and R³ differs from R¹ and R².

12. The composition of claim 11, wherein each of R¹ and R² is 2-hydroxyethyl, ethyl, or methyl.

13. The composition of claim 11 or 12, wherein R³ is selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

14. The composition of claim 1, wherein R¹ and R² join together with the nitrogen atom to form a 5- or 6-membered N-heterocyclic ring which is optionally substituted with one or two R⁵.

15. The composition of claim 14, wherein R³ is selected from C₁₋₆ alkyl, C₁₋₆ alkylene-R⁴, and C(C₁₋₅ alkylene-R⁴)₃, preferably R³ is selected from C₁₋₄ alkyl, C₁₋₄ alkylene-R⁴, and C(C₁₋₃ alkylene-R⁴)₃, more preferably R³ is selected from C₁₋₃ alkyl, C₁₋₂ alkylene-R⁴, and C(C₁₋₃ alkylene-R⁴)₃, more preferably R³ is selected from C₁₋₂ alkyl, C₁₋₂ alkylene-R⁴, and C(C₁₋₂ alkylene-R⁴)₃, more preferably R³ is selected from selected from methyl, ethyl, 2-hydroxyethyl, 2-(2-hydroxyethoxy)ethyl, and 2-[bis(2-hydroxyethyl)amino]ethyl.

16. The composition of claim 14 or 15, wherein the N-heterocyclic ring is a monocyclic ring containing at least one nitrogen ring atom and optionally one further ring heteroatom selected from O and S.

17. The composition of any one of claims 14 to 16, wherein the N-heterocyclic ring is a monocyclic ring containing (i) one nitrogen ring atom; (ii) two nitrogen ring atoms; (iii) one nitrogen ring atom and one oxygen ring atom; (iv) one nitrogen ring atom and one sulfur ring atom; or (v) three nitrogen ring atoms.

18. The composition of any one of claim 14 to 17, wherein the N-heterocyclic ring is a monocyclic 5- or 6-membered N-heterocyclic ring, such as is a monocyclic 6-membered N-heterocyclic ring.

19. The composition of any one of claims 14 to 18, wherein the N-heterocyclic ring is selected from pyrrolidinyl, imidazolidinyl, pyrazolidinyl, oxazolidinyl, thiazolidinyl, piperidinyl, piperazinyl, 1,2-diazinanyl, 1,3-diazinanyl, 1,3,5-triazinanyl, morpholinyl, and thiomorpholinyl, preferably selected from piperidinyl, piperazinyl, 1,2-diazinanyl, 1,3-diazinanyl, morpholinyl, and thiomorpholinyl.

20. The composition of any one of claims 14 to 19, wherein, if the N-heterocyclic ring contains only one nitrogen ring atom, this nitrogen ring atom is substituted with R³, R³ being other than H, or, if the N-heterocyclic ring contains more than one nitrogen ring atom, one nitrogen ring atom is substituted with R³, R³ being other than H, and at least one of the other nitrogen ring atoms, preferably each of the other nitrogen ring atoms, is substituted with R⁵.

21. The composition of any one of claims 14 to 20, wherein each R⁵ is independently selected from C₁₋₆ alkyl, C₁₋₆ alkylene-R⁴, and C(C₁₋₅ alkylene-R⁴)₃, preferably R is selected from C₁₋₄ alkyl, C₁₋₄ alkylene-R⁴, and C(C₁₋₃ alkylene-R⁴)₃, more preferably R⁵ is selected from C₁₋₃ alkyl, C₁₋₃ alkylene-R⁴, and C(C₁₋₃ alkylene-R⁴)₃, more preferably R⁵ is selected from C₁₋₂ alkyl, C₁₋₂alkylene-R⁴, and C(C₁₋₂ alkylene-R⁴)₃, more preferably R is selected from selected from methyl, ethyl, 2-hydroxyethyl, 2-(2-hydroxyethoxy)ethyl, and 2-[bis(2-hydroxyethyl)amino]ethyl.

22. The composition of any one of claims 14 to 21, wherein the N-heterocyclic ring is piperidinyl and the ring N atom is substituted with R³, R³ being other than H.

23. The composition of any one of claims 14 to 21, wherein the N-heterocyclic ring is piperazinyl, one the two ring N atoms is substituted with R³, R³ being other than H, and the other ring N atom is optionally substituted with R⁵, preferably the other ring N atom is substituted with R⁵.

24. The composition of claim 23, wherein both ring N atoms are substituted and R⁵ is selected from C₁₋₆ alkyl, C₁₋₆ alkylene-R⁴, and C(C₁₋₅ alkylene-R⁴)₃, preferably R⁵ is selected from C₁₋₄ alkyl, C₁₋₄ alkylene-R⁴, and C(C₁₋₃ alkylene-R⁴)₃, more preferably R⁵ is selected from C₁₋₃ alkyl, C₁₋₃alkylene-R⁴, and C(C₁₋₃ alkylene-R⁴)₃, more preferably R⁵ is selected from C₁₋₂ alkyl, C₁₋₂ alkylene-R⁴, and C(C₁₋₂ alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl.

25. The composition of any one of claims 14 to 24, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

26. The composition of any one of claims 14 to 25, wherein each R⁴ is independently selected from —OH, 2-hydroxyethoxy, and bis(2-hydroxyethyl)amino.

27. The composition of any one of claims 14 to 26, wherein R³ and R⁵ are the same.

28. The composition of claim 27, wherein both of R³ and R₁ are methyl, ethyl, 2-hydroxyethyl, or 2-(2-hydroxyethoxy)ethyl, preferably, both of R³ and R⁵ are 2-hydroxyethyl.

29. The composition of any one of claims 14 to 26, wherein R³ and R⁵ differ from each other.

30. The composition of claim 1, wherein R¹ is H.

31. The composition of claim 30, wherein each of R² and R³ is independently selected from C₁₋₆alkyl, C₁₋₆ alkylene-R⁴, CH(C₁₋₅ alkylene-R⁴)₂, and C(C₁₋₅ alkylene-R⁴)₃, wherein at most one of R² and R³ is CH(C₁₋₅ alkylene-R⁴)₂ or C(C₁₋₅ alkylene-R⁴)₃, preferably each of R² and R³ is independently selected from C₁₋₄ alkyl, C₁₋₄ alkylene-R⁴, CH(C₁₋₃ alkylene-R⁴)₂, and C(C₁₋₃ alkylene-R⁴)₃, wherein at most one of R² and R³ is CH(C₁₋₃ alkylene-R⁴)₂ or C(C₁₋₃ alkylene-R⁴)₃, more preferably each of R² and R³ is independently selected from C₁₋₃ alkyl, C₁₋₃ alkylene-R⁴, CH(C₁₋₃ alkylene-R⁴)₂, and C(C₁₋₃ alkylene-R⁴)₃, wherein at most one of R² and R³ is CH(C₁₋₃ alkylene-R⁴)₂ or C(C₁₋₃ alkylene-R⁴)₃, more preferably each of R² and R³ is independently selected from C₁₋₂ alkyl, C₁₋₂ alkylene-R⁴, CH(C₁₋₂ alkylene-R⁴)₂, and C(C₁₋₂ alkylene-R⁴)₃, wherein at most one of R² and R³ is CH(C₁₋₂ alkylene-R⁴)₂ or C(C₁₋₂ alkylene-R⁴)₃.

32. The composition of claim 30 or 31, wherein each of R² and R³ is independently selected from C₁₋₆ alkyl, C₁₋₆alkylene-R⁴, and C(C₁₋₅ alkylene-R⁴)₃, wherein at most one of R² and R³ is C(C₁₋₅ alkylene-R⁴)₃, preferably each of R² and R³ is independently selected from C₁₋₄ alkyl, C₁₋₄alkylene-R⁴, and C(C₁₋₃ alkylene-R⁴)₃, wherein at most one of R² and R³ is C(C₁₋₃ alkylene-R⁴)₃, more preferably each of R² and R³ is independently selected from C₁₋₃ alkyl, C₁₋₃ alkylene-R⁴, and C(C₁₋₃ alkylene-R⁴)₃, wherein at most one of R² and R³ is C(C₁₋₃ alkylene-R⁴)₃, more preferably each of R² and R³ is independently selected from C₁₋₂ alkyl, C₁₋₂ alkylene-R⁴, and C(C₁₋₂ alkylene-R⁴)₃, wherein at most one of R² and R³ is C(C₁₋₂ alkylene-R⁴)₃.

33. The composition of any one of claims 30 to 32, wherein each of R² and R³ is independently selected from C₁₋₆ alkyl and C₁₋₆ alkylene-R⁴, preferably each of R² and R³ is independently selected from C₁₋₄ alkyl and C₁₋₄ alkylene-R⁴, more preferably each of R² and R³ is independently selected from C₁₋₃ alkyl and C₁₋₃ alkylene-R⁴, more preferably each of R² and R³ is independently selected from C₁₋₂ alkyl and C₁₋₂ alkylene-R⁴.

34. The composition of any one of claims 30 to 33, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

35. The composition of any one of claims 30 to 34, wherein each R⁴ is independently selected from —OH, 2-hydroxyethoxy, and bis(2-hydroxyethyl)amino.

36. The composition of any one of claims 30 to 35, wherein each of R² and R³ is independently selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl, preferably, both of R² and R³ are 2-hydroxyethyl or 2-(2-hydroxyethoxy)ethyl.

37. The composition of any one of claims 1 to 36, wherein the buffer substance is selected from bis(2-hydroxyethyl)amino-tris(hydroxymethyl)methane (Bis-Tris-methane or BTM) and its protonated form, triethanolamine (TEA) and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form.

38. The composition of any one of claims 1 to 37, wherein the buffer substance comprises at least one C₁₋₆ alkylene-R⁴ (such as 2-hydroxyethyl) moiety.

39. A composition comprising (i) RNA; and (ii) an aqueous phase, wherein the aqueous phase comprises a buffer system comprising a buffer substance having the formula N(R¹)(R²)(R³), its N-oxide, or a protonated form thereof, wherein:
each of R¹, R², and R³ is independently selected from H, C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is H, CH(C₁₋₅ alkylene-R⁴)₂, or C(C₁₋₅ alkylene-R⁴)₃; or two of R¹, R², and R³ join together with the nitrogen atom to form a 5- or 6-membered N-heterocyclic ring which is optionally substituted with one or two R⁵;
each R⁴ is independently selected from —OH, —O-(C₁₋₆ alkylene-OH), and —N(R⁶)_{z}-(C₁₋₆ alkylene-OH)_{2-z}, wherein each z is independently selected from 0 and 1; and each R⁶ is independently selected from H and C₁₋₃ alkyl; and
each R⁵ is independently selected from C₁₋₆ alkyl, C₁₋₆ alkylene-R⁴, CH(C₁₋₅ alkylene-R⁴)₂, and C(C₁₋₅ alkylene-R⁴)₃.

40. The composition of claim 39, wherein each of R¹, R², and R³ is independently selected from C₁₋₆ alkyl, C₁₋₆ alkylene-R⁴, CH(C₁₋₅ alkylene-R⁴)₂, and C(C₁₋₅ alkylene-R⁴)₃, wherein at most one of R¹, R², and R³ is CH(C₁₋₅ alkylene-R⁴)₂ or C(C₁₋₅ alkylene-R⁴)₃, preferably each of R¹, R², and R³ is independently selected from C₁₋₄ alkyl, C₁₋₄ alkylene-R⁴, CH(C₁₋₃ alkylene-R⁴)₂, and C(C₁₋₃ alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₃ alkyl, C₁₋₃alkylene-R⁴, CH(C₁₋₃ alkylene-R⁴)₂, and C(C₁₋₃ alkylene-R⁴)₃, wherein at most one of R¹, R², and R³ is CH(C₁₋₃ alkylene-R⁴)₂ or C(C₁₋₃ alkylene-R⁴)₃, more preferably each of R¹, R², and R³ is independently selected from C₁₋₂ alkyl, C₁₋₂ alkylene-R⁴, CH(C₁₋₂ alkylene-R⁴)₂, and C(C₁₋₂alkylene-R⁴)₃, wherein at most one of R¹, R², and R³ is CH(C₁₋₂ alkylene-R⁴)₂ or C(C₁₋₂ alkylene-R⁴)₃.

41. The composition of claim 39 or 40, wherein each of R¹, R², and R³ is independently selected from C₁₋₆ alkyl, C₁₋₆ alkylene-R⁴, and C(C₁₋₅ alkylene-R⁴)₃, wherein at most one of R¹, R², and R³ is C(C₁₋₅ alkylene-R⁴)₃, preferably each of R¹, R², and R³ is independently selected from C₁₋₄ alkyl, C₁₋₄ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₃ alkyl, C₁₋₃ alkylene-R⁴, and C(C₁₋₃ alkylene-R⁴)₃, wherein at most one of R¹, R², and R³ is C(C₁₋₃ alkylene-R⁴)₃, more preferably each of R¹, R², and R³ is independently selected from C₁₋₂ alkyl, C₁₋₂ alkylene-R⁴, and C(C₁₋₂ alkylene-R⁴)₃, wherein at most one of R¹, R², and R³ is C(C₁₋₂alkylene-R⁴)₃.

42. The composition of any one of claims 39 to 41, wherein each of R¹, R², and R³ is independently selected from C₁₋₆ alkyl and C₁₋₆ alkylene-R⁴, preferably each of R¹, R², and R³ is independently selected from C₁₋₄ alkyl and C₁₋₄ alkylene-R⁴, more preferably each of R¹, R², and R³ is independently selected from C₁₋₃ alkyl and C₁₋₃ alkylene-R⁴, more preferably each of R¹, R², and R³ is independently selected from C₁₋₂ alkyl and C₁₋₂ alkylene-R⁴.

43. The composition of any one of claims 39 to 42, wherein each R⁴ is independently selected from —OH, —O-(C₁₋₄ alkylene-OH), and —N(R⁶)_{z}-(C₁₋₄ alkylene-OH)_{2-z}, wherein each z is independently selected from 0 and 1; and each R⁶ is independently selected from H and C₁₋₃ alkyl, preferably each R⁴ is independently selected from —OH, —O-(C₁₋₃ alkylene-OH), and —N(R⁶)_{z}-(C₁₋₃ alkylene-OH)_{2-z}, wherein each z is independently selected from 0 and 1; and each R⁶ is independently selected from H and C₁₋₃ alkyl, more preferably each R⁴ is independently selected from —OH, —O-(C₁₋₂ alkylene-OH), and —N(R⁶)_{z}-(C₁₋₂ alkylene-OH)_{2-z}, wherein each z is independently selected from 0 and 1; and each R⁶ is independently selected from H and C₁₋₂ alkyl.

44. The composition of any one of claims 39 to 43, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

45. The composition of any one of claims 39 to 44, wherein each R⁴ is independently selected from —OH, 2-hydroxyethoxy, and bis(2-hydroxyethyl)amino.

46. The composition of any one of claims 39 to 45, wherein each of R¹, R², and R³ is independently selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

47. The composition of any one of claims 39 to 46, wherein all of R¹, R², and R³ are the same.

48. The composition of claim 47, wherein all of R¹, R², and R³ are methyl, ethyl, or 2-hydroxyethyl.

49. The composition of any one of claims 39 to 46, wherein R¹ and R² are the same and R³ differs from R¹ and R².

50. The composition of claim 49, wherein each of R¹ and R² is 2-hydroxyethyl, ethyl, or methyl.

51. The composition of claim 49 or 50, wherein R³ is selected from methyl, ethyl, 2-hydroxyethyl, 2-(2-hydroxyethoxy)ethyl, 2-[bis(2-hydroxyethyl)amino]ethyl, and 1,5-dihydroxy-3-(2-hydroxyethyl)pentan-3-yl.

52. The composition of claim 39, wherein R¹ and R² join together with the nitrogen atom to form a 5- or 6-membered N-heterocyclic ring which is optionally substituted with one or two R⁵.

53. The composition of claim 52, wherein R³ is selected from C₁₋₆ alkyl, C₁b alkylene-R⁴, and C(C₁₋₅alkylene-R⁴)₃, preferably R³ is selected from C₁₋₄ alkyl, C₁₋₄ alkylene-R⁴, and C(C₁₋₃ alkylene-R⁴)₃, more preferably R³ is selected from C₁₋₃ alkyl, C₁a alkylene-R⁴, and C(C₁₋₃ alkylene-R⁴)₃, more preferably R³ is selected from C₁₋₂ alkyl, C₁₋₂ alkylene-R⁴, and C(C₁₋₂ alkylene-R⁴)₃, more preferably R³ is selected from selected from methyl, ethyl, 2-hydroxyethyl, 2-(2-hydroxyethoxy)ethyl, and 2-[bis(2-hydroxyethyl)amino]ethyl.

54. The composition of any one of claims 52 to 53, wherein, if the N-heterocyclic ring contains only one nitrogen ring atom, this nitrogen ring atom is substituted with R³, R³ being other than H, or, if the N-heterocyclic ring contains more than one nitrogen ring atom, one nitrogen ring atom is substituted with R³, R³ being other than H, and at least one of the other nitrogen ring atoms, preferably each of the other nitrogen ring atoms, is substituted with R⁵.

55. The composition of any one of claims 52 to 54, wherein each R⁵ is independently selected from C₁₋₆ alkyl, C₁₋₆ alkylene-R⁴, and C(C₁₋₅ alkylene-R⁴)₃, preferably R⁵ is selected from C₁₋₄ alkyl, C₁₋₄ alkylene-R⁴, and C(C₁₋₃ alkylene-R⁴)₃, more preferably R⁵ is selected from C₁₋₃ alkyl, C₁₋₃alkylene-R⁴, and C(C₁₋₅ alkylene-R⁴)₃, more preferably R⁵ is selected from C₁₋₂ alkyl, C₁₋₂ alkylene-R⁴, and C(C₁₋₂ alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl.

56. The composition of any one of claims 52 to 55, wherein the N-heterocyclic ring is piperidinyl and the ring N atom is substituted with R³, wherein R³ is other than H.

57. The composition of any one of claims 52 to 55, wherein the N-heterocyclic ring is piperazinyl, one the two ring N atoms is substituted with R³, R³ being other than H, and the other ring N atom is optionally substituted with R⁵, preferably the other ring N atom is substituted with R⁵.

58. The composition of claim 57, wherein both ring N atoms are substituted and R⁵ is selected from C₁₋₆ alkyl, C₁(alkylene-R⁴, and C(C₁₋₅ alkylene-R⁴)₃, preferably R⁵ is selected from C₁₋₄ alkyl, C₁₋₄ alkylene-R⁴, and C(C₁₋₃ alkylene-R⁴)₃, more preferably R⁵ is selected from C₁₋₃ alkyl, C₁₋₃alkylene-R⁴, and C(C₁₋₃ alkylene-R⁴)₃, more preferably R⁵ is selected from C₁₋₂ alkyl, C₁₋₂ alkylene-R⁴, and C(C₁₋₂ alkylene-R⁴)₃, more preferably R⁵ is selected from selected from methyl, ethyl, 2-hydroxyethyl, 2-(2-hydroxyethoxy)ethyl, and 2-[bis(2-hydroxyethyl)amino]ethyl.

59. The composition of any one of claims 52 to 58, wherein each R⁴ is independently selected from —OH, —O-(C₁₋₄ alkylene-OH), and —N(C₁₋₄ alkylene-OH)₂, preferably each R⁴ is independently selected from —OH, —O-(C₁₋₃ alkylene-OH), and —N(C₁₋₃ alkylene-OH)₂, more preferably each R⁴ is independently selected from —OH, —O-(C₁₋₂ alkylene-OH), and —N(C₁₋₂ alkylene-OH)₂.

60. The composition of any one of claims 52 to 59, wherein each R⁴ is independently selected from —OH, 2-hydroxyethoxy, and bis(2-hydroxyethyl)amino.

61. The composition of any one of claims 52 to 60, wherein R³ and R⁵ are the same.

62. The composition of claim 61, wherein both of R³ and R⁵ are methyl, ethyl, 2-hydroxyethyl, or 2-(2-hydroxyethoxy)ethyl, preferably, both of R³ and R⁵ are 2-hydroxyethyl.

63. The composition of any one of claims 52 to 60, wherein R³ and R⁵ differ from each other.

64. The composition of any one of claims 39 to 63, wherein the buffer substance is selected from bis(2-hydroxyethyl)amino-tris(hydroxymethyl)methane (Bis-Tris-methane or BTM) and its protonated form, triethanolamine (TEA) and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form.

65. The composition of any one of claims 39 to 64, wherein the buffer substance comprises at least one C₁₋₆ alkylene-R⁴ (such as 2-hydroxyethyl) moiety.

66. The composition of any one of claims 1 to 65, wherein the buffer system further comprises an anion selected from the group consisting of chloride, acetate, glycolate, lactate, and the anion of a di- or tricarboxylic acid, such as the anion of citric acid, succinic acid, malonic acid, glutaric acid, or adipic acid.

67. The composition of any one of claims 1 to 66, wherein the concentration of the buffer substance in the composition is between about 10 mM and about 200 mM, preferably between about 15 mM and about 100 mM, more preferably between about 20 mM and about 80 mM, more preferably between about 40 mM and about 60 mM, such as about 50 mM.

68. The composition of any one of claims 1 to 67, wherein the pH of the composition is between about 4.0 and about 8.0, preferably between about 4.5 and about 8.0, such as between about 5.0 and about 8.0, between about 5.5 and about 8.0, between about 6.0 and about 8.0, between about 6.5 and about 8.0, between about 6.8 and about 7.9, or between about 7.0 and about 7.8.

69. The composition of any one of claims 1 to 68, wherein water is the main component in the composition and/or the total amount of solvent(s) other than water contained in the composition is less than about 0.5% (v/v).

70. The composition of any one of claims 1 to 69, wherein the osmolality of the composition is at most about 1000×10⁻³ osmol/kg, preferably between about 100×10⁻³ osmol/kg and about 750×10⁻³ osmol/kg, such as between about 100×10⁻³ osmol/kg and about 500×10⁻³ osmol/kg, more preferably about 300×10⁻³ osmol/kg.

71. The composition of any one of claims 1 to 70, wherein the concentration of the RNA in the composition is about 5 mg/l to about 500 mg/l, such as about 10 mg/l to about 400 mg/l, about 10 mg/l to about 300 mg/l, about 10 mg/l to about 200 mg/l, about 10 mg/l to about 150 mg/1, or about 10 mg/l to about 100 mg/l, preferably about 10 mg/l to about 140 mg/l, more preferably about 20 mg/l to about 130 mg/l, more preferably about 30 mg/l to about 120 mg/l.

72. The composition of any one of claims 1 to 71, wherein the composition comprises a cryoprotectant.

73. The composition of any one of claims 1 to 38 and 66 to 72, wherein the cationically ionizable lipid comprises a head group which includes at least one nitrogen atom which is capable of being protonated under physiological conditions.

74. The composition of any one of claims 1 to 38 and 66 to 73, wherein the cationically ionizable lipid has the structure of Formula (X) 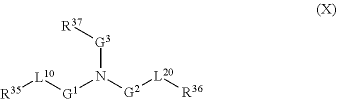
or a pharmaceutically acceptable salt, tautomer, prodrug or stereoisomer thereof, wherein:
one of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₁₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆₋₂₄ alkyl or C₆₋₂₄ alkenyl;
R³⁷ is H, OR⁵⁰, CN, —C(═O)OR⁴⁰, —OC(═O)R⁴⁰ or —NR⁵⁰C(═O)R⁴⁰;
R⁴⁰ is C₁₋₁₂ alkyl;
R⁵⁰ is H or C₁₋₆ alkyl; and
x is 0, 1 or 2.

75. The composition of any one of claims 1 to 38 and 66 to 73, wherein the cationic or cationically ionizable lipid has the structure of Formula (XI): 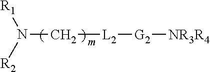
wherein
each of R₁ and R₂ is independently R₅ or -G₁-L₁-R₆, wherein at least one of R₁ and R₂ is -G₁-L₁-R₆;
each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}— and —NR^{a}C(═O)O—;
Ra is H or C₁₋₁₂ alkyl;
m is 0, 1, 2, 3, or 4; and
x is 0, 1 or 2.

76. The composition of any one of claims 1 to 38 and 66 to 75, wherein the cationically ionizable lipid comprises from about 20 mol % to about 80 mol %, preferably from about 25 mol % to about 65 mol %, more preferably from about 30 mol % to about 50 mol %, such as from about 40 mol % to about 50 mol %, of the total lipid present in the composition.

77. The composition of any one of claims 1 to 76, which further comprises one or more additional lipids, preferably selected from the group consisting of polymer conjugated lipids, neutral lipids, steroids, and combinations thereof, more preferably the composition comprises the cationically ionizable lipid, a polymer conjugated lipid, a neutral lipid (e.g., a phospholipid), and a steroid.

78. The composition of claim 77, wherein the polymer conjugated lipid comprises a pegylated lipid, wherein the pegylated lipid preferably (i) is selected from the group consisting of DSPE-PEG, DOPE-PEG, DPPE-PEG, and DMPE-PEG; or (ii) has the following structure: 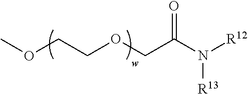
or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

79. The composition of claim 77, wherein the polymer conjugated lipid comprises a polysarcosine-lipid conjugate or a conjugate of polysarcosine and a lipid-like material, wherein the polysarcosine-lipid conjugate or conjugate of polysarcosine and a lipid-like material preferably is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

80. The composition of any one of claims 77 to 79, wherein the polymer conjugated lipid comprises from about 0.5 mol % to about 5 mol %, preferably from about 1 mol % to about 5 mol %, more preferably from about 1 mol % to about 4.5 mol % of the total lipid present in the composition.

81. The composition of any one of claims 77 to 80, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

82. The composition of any one of claims 77 to 81, wherein the neutral lipid comprises from about 5 mol % to about 40 mol %, preferably from about 5 mol % to about 20 mol %, more preferably from about 5 mol % to about 15 mol % of the total lipid present in the composition.

83. The composition of any one of claims 77 to 82, wherein the steroid comprises a sterol such as cholesterol.

84. The composition of any one of claims 77 to 83, wherein the steroid comprises from about 10 mol % to about 65 mol %, preferably from about 20 mol % to about 60 mol %, more preferably from about 30 mol % to about 50 mol % of the total lipid present in the composition.

85. The composition of any one of claims 77 to 84, which comprises a cationically ionizable lipid, a polymer conjugated lipid, a neutral lipid (e.g., a phospholipid), and a steroid, wherein the cationically ionizable lipid comprises from about 30 mol % to about 50 mol %, such as from about 40 mol % to about 50 mol %, of the total lipid present in the composition; the polymer conjugated lipid comprises from about 1 mol % to about 4.5 mol % of the total lipid present in the composition; the neutral lipid (e.g., phospholipid) comprises from about 5 mol % to about 15 mol % of the total lipid present in the composition; and the steroid comprises from about 30 mol % to about 50 mol % of the total lipid present in the composition.

86. The composition of any one of claims 1 to 85, wherein at least a portion of the RNA and, if present, of one or more lipids, is present in particles, such as lipid nanoparticles (LNPs), liposomes, and/or lipoplexes (LPXs).

87. The composition of claim 86, wherein the particles have a size of from about 30 nm to about 500 nm.

88. The composition of any one of claims 1 to 87, wherein the RNA is mRNA or inhibitory RNA.

89. The composition of any one of claims 1 to 88, wherein the RNA (i) comprises a modified nucleoside in place of uridine, wherein the modified nucleoside is preferably selected from pseudouridine (ψ), N1-methyl-pseudouridine (m1ψ), and 5-methyl-uridine (m5U); (ii) has a coding sequence which is codon-optimized; and/or (iii) has a coding sequence whose G/C content is increased compared to the wild-type coding sequence.

90. The composition of any one of claims 1 to 89, wherein the RNA comprises at least one of the following, preferably all of the following: a 5′ cap; a 5′ UTR; a 3′ UTR; and a poly-A sequence.

91. The composition of claim 90, wherein the poly-A sequence comprises at least 100 A nucleotides, wherein the poly-A sequence preferably is an interrupted sequence of A nucleotides.

92. The composition of claim 90 or 91, wherein the 5′ cap is a cap1 or cap2 structure.

93. The composition of any one of claims 1 to 92, wherein the RNA encodes one or more polypeptides, wherein preferably the one or more polypeptides are pharmaceutically active polypeptides and/or comprise an epitope for inducing an immune response against an antigen in a subject.

94. The composition of claim 93, wherein the pharmaceutically active polypeptide and/or the antigen or epitope is derived from or is a protein of a pathogen, an immunogenic variant of the protein, or an immunogenic fragment of the protein or the immunogenic variant thereof.

95. The composition of claim 93 or 94, wherein the pharmaceutically active polypeptide and/or the antigen or epitope is derived from or is a SARS-CoV-2 spike (S) protein, an immunogenic variant thereof, or an immunogenic fragment of the SARS-CoV-2 S protein or the immunogenic variant thereof.

96. The composition of any one of claims 1 to 95, wherein the composition is in liquid form, preferably at a temperature of about 2° C. to about 10° C.

97. The composition of any one of claims 1 to 96, wherein the RNA integrity of the composition after storage for at least one week, preferably at a temperature of about 2° C. to about 8° C., is at least 50% compared to the RNA integrity before storage.

98. The composition of any one of claims 87 to 97, wherein the size (Z_{average}) and/or size distribution and/or polydispersity index (PDI) of RNA particles (in particular LNPs) after storage of the composition is essentially equal to the size (Z_{average}) and/or size distribution and/or PDI of the RNA particles before storage.

99. The composition of any one of claims 1 to 95, wherein the composition is in frozen form.

100. The composition of claim 99, wherein the RNA integrity after thawing the frozen composition is at least 50% compared to the RNA integrity before the composition has been frozen.

101. The composition of claim 99 or 100, wherein the size (Z_{average}) and/or size distribution and/or polydispersity index (PDI) of RNA particles (in particular LNPs) after thawing the frozen composition is essentially equal to the size (Z_{average}) and/or size distribution and/or PDI of the RNA particles before the composition has been frozen.

102. A method of preparing a composition comprising LNPs dispersed in a final aqueous phase, wherein the LNPs comprise a cationically ionizable lipid and RNA; the final aqueous phase comprises a final buffer system comprising a final buffer substance, the final buffer substance having the formula N(R¹)(R²)(R³), its N-oxide, or a protonated form thereof, wherein R¹, R², and R³ are as defined in any one of claims 1 to 38;
wherein the method comprises:
(I) preparing a formulation comprising LNPs dispersed in the final aqueous phase, wherein the LNPs comprise the cationically ionizable lipid and RNA; and
(II) optionally freezing the formulation to about −10° C. or below,
thereby obtaining the composition,
wherein step (I) comprises:
(a) preparing an RNA solution containing water and a first buffer system;
(b) preparing an ethanolic solution comprising the cationically ionizable lipid and, if present, one or more additional lipids;
(c) mixing the RNA solution prepared under (a) with the ethanolic solution prepared under (b), thereby preparing a first intermediate formulation comprising the LNPs dispersed in a first aqueous phase comprising the first buffer system; and
(d) filtrating the first intermediate formulation prepared under (c) using a final aqueous buffer solution comprising the final buffer system,
thereby preparing the formulation comprising the LNPs dispersed in the final aqueous phase.

103. The method of claim 102, wherein step (I) further comprises one or more steps selected from diluting and filtrating.

104. The method of claim 102 or 103, wherein step (I) comprises:
(a′) providing an aqueous RNA solution;
(b′) providing a first aqueous buffer solution comprising a first buffer system;
(c′) mixing the aqueous RNA solution provided under (a′) with the first aqueous buffer solution provided under (b′) thereby preparing an RNA solution containing water and the first buffer system;
(d′) preparing an ethanolic solution comprising the cationically ionizable lipid and, if present, one or more additional lipids;
(e′) mixing the RNA solution prepared under (c′) with the ethanolic solution prepared under (d′), thereby preparing a first intermediate formulation comprising LNPs dispersed in a first aqueous phase comprising the first buffer system;
(f′) optionally filtrating the first intermediate formulation prepared under (e′) using a further aqueous buffer solution comprising a further buffer system, thereby preparing a further intermediate formulation comprising the LNPs dispersed in a further aqueous phase comprising the further buffer system, wherein the further aqueous buffer solution may be identical to or different from the first aqueous buffer solution;
(g′) optionally repeating step (f′) once or two or more times, wherein the further intermediate formulation comprising the LNPs dispersed in the further aqueous phase comprising the further buffer system obtained after step (f′) of one cycle is used as the first intermediate formulation of the next cycle, wherein in each cycle the further aqueous buffer solution may be identical to or different from the first aqueous buffer solution;
(h′) filtrating the first intermediate formulation obtained in step (e′), if step (f′) is absent, or the further intermediate formulation obtained in step (f′), if step (f′) is present and step (g′) is not present, or the further intermediate formulation obtained after step (g′), if steps (f′) and (g′) are present, using a final aqueous buffer solution comprising the final buffer system; and
(i′) optionally diluting the formulation obtained in step (h′) with a dilution solution;
thereby preparing the formulation comprising the LNPs dispersed in the final aqueous phase.

105. The method of any one of claims 102 to 104, wherein filtrating is tangential flow filtrating or diafiltrating, preferably tangential flow filtrating.

106. The method of any one of claims 102 to 105, which comprises (II) freezing the formulation to about −10° C. or below.

107. The method of claim 106, wherein the formulation obtained in step (I) and the composition comprise a cryoprotectant.

108. The method of any one of claims 102 to 107, wherein the final buffer substance is selected from BTM and its protonated form, TEA and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form.

109. The method of any one of claims 102 to 108, wherein the concentration of the final buffer substance in the composition is between about 10 mM to about 200 mM, preferably between about 15 mM to about 100 mM, more preferably between about 20 mM to about 80 mM, more preferably between about 40 mM to about 60 mM, such as about 50 mM.

110. The method of any one of claims 102 to 109, wherein (i) the RNA solution obtained in step (a) has a pH of below 6.0, preferably at most about 5.0, more preferably at most about 4.5; or (ii) the first aqueous buffer solution has a pH of below 6.0, preferably at most about 5.0, more preferably at most about 4.5.

111. The method of any one of claims 102 to 110, wherein (i) the first buffer system used in step (a) comprises the final buffer substance used in step (d), preferably the buffer system and pH of the first buffer system used in step (a) are identical to the buffer system and pH of the final aqueous buffer solution used in step (d); or (ii) each of the first buffer system and every further buffer system used in steps (b′), (f′) and (g′) comprises the final buffer substance used in step (h′), preferably the buffer system and pH of each of the first aqueous buffer solution and of every further aqueous buffer solution used in steps (b′), (f′) and (g′) are identical to the buffer system and pH of the final aqueous buffer solution.

112. The method of any one of claims 102 to 111, wherein the pH of the composition is between about 4.0 and about 8.0, preferably between about 4.5 and about 8.0, such as between about 5.0 and about 8.0, between about 5.5 and about 8.0, between about 6.0 and about 8.0, between about 6.5 and about 8.0, between about 6.8 and about 7.9, or between about 7.0 and about 7.8.

113. The method of any one of claims 102 to 112, wherein water is the main component in the formulation and/or composition and/or the total amount of solvent(s) other than water contained in the composition is less than about 0.5% (v/v).

114. The method of any one of claims 102 to 113, wherein the osmolality of the composition is at most about 1000×10⁻³ osmol/kg, preferably between about 100×10⁻³ osmol/kg and about 750×10⁻³ osmol/kg, such as between about 100×10⁻³ osmol/kg and about 500×10⁻³ osmol/kg, more preferably about 300×10⁻³ osmol/kg.

115. The method of any one of claims 102 to 114, wherein the concentration of the RNA in the composition is about 5 mg/l to about 500 mg/l, such as about 10 mg/l to about 400 mg/l, about 10 mg/l to about 300 mg/l, about 10 mg/l to about 200 mg/l, about 10 mg/l to about 150 mg/l, or about 10 mg/l to about 100 mg/l, preferably about 10 mg/l to about 140 mg/l, more preferably about 20 mg/l to about 130 mg/l, more preferably about 30 mg/l to about 120 mg/l.

116. The method of any one of claims 102 to 115, wherein the cationically ionizable lipid comprises a head group which includes at least one nitrogen atom which is capable of being protonated under physiological conditions.

117. The method of any one of claims 102 to 116, wherein the ethanolic solution prepared in step (b) or (d′) further comprises one or more additional lipids and the LNPs further comprise the one or more additional lipids, wherein the one or more additional lipids are preferably selected from the group consisting of polymer conjugated lipids, neutral lipids, steroids, and combinations thereof, more preferably the one or more additional lipids comprise a polymer conjugated lipid, a neutral lipid (e.g., a phospholipid), and a steroid.

118. The method of any one of claims 102 to 117, wherein the cationically ionizable lipid, the polymer conjugated lipid, the neutral lipid, and the steroid are present in the ethanolic solution in a molar ratio of 20% to 60% of the cationically ionizable lipid, 0.5% to 15% of the polymer conjugated lipid, 5% to 25% of the neutral lipid (e.g., phospholipid), and 25% to 55% of the steroid, preferably in a molar ratio of 45% to 55% of the cationically ionizable lipid, 1.0% to 5% of the polymer conjugated lipid, 8% to 12% of the neutral lipid, and 35% to 45% of the steroid.

119. The method of any one of claims 102 to 118, wherein the RNA is as defined in any one of claims 88 and 89 to 95.

120. The method of any one of claims 102 to 105 and 107 to 119, which does not comprise step (II).

121. A method of preparing an aqueous RNA composition, wherein the method comprises:
(I) preparing a formulation comprising RNA and an aqueous phase, wherein the aqueous phase comprises a buffer substance, the buffer substance having the formula N(R¹)(R²)(R³), its N-oxide, or a protonated form thereof, wherein R¹, R², and R³ are as defined in any one of claims 1 to 38; and
(II) optionally freezing the formulation to about −10° C. or below,
thereby obtaining the composition.

122. The method of claim 121, which comprises (II) freezing the formulation to about −10° C. or below.

123. The method of claim 122, wherein the composition comprises a cryoprotectant.

124. The method of claim 121, which does not comprise step (II).

125. The method of any one of claims 121 to 124, wherein the buffer substance is selected from BTM and its protonated form, TEA and its protonated form, ethyldiethanolamine and its protonated form, 2-(diethylamino)ethan-1-ol and its protonated form, triethylamine and its protonated form, 2-[2-(diethylamino)ethoxy]ethan-1-ol and its protonated form, diethanolamine and its protonated form, N,N′-bis(2-hydroxyethyl)piperazine and its protonated form, N,N,N′,N′-tetrakis(2-hydroxyethyl)ethylenediamine and its protonated form, and trimethylamine N-oxide and its protonated form.

126. A method of storing a composition, comprising preparing a composition according to the method of any one of claims 102 to 119, 121, 122, and 125 and storing the composition at a temperature ranging from about −90° C. to about −10° C., such as from about −90° C. to about −40° C. or from about −25° C. to about −10° C.

127. The method of 126, wherein the composition comprises a cryoprotectant.

128. The method of claim 127, wherein storing the composition is for at least 1 month, such as at least 2 months, at least 3 months, at least 6 months, at least 12 months, at least 24 months, or at least 36 months.

129. A method of storing a composition, comprising preparing a composition according to the method of any one of claims 102 to 128 and storing the composition at a temperature ranging from about 0° C. to about 20° C., such as from about 1° C. to about 15° C., from about 2° C. to about 10° C., or from about 2° C. to about 8° C., or at a temperature of about 5° C.

130. The method of claim 129, wherein storing the composition is for at least 1 week, such as at least 2 weeks, at least 3 weeks, at least 4 weeks, at least 1 month, at least 2 months, at least 3 months, at least 6 months, at least 12 months, or at least 24 months.

131. A composition preparable by the method of any one of claims 102 to 130.

132. The composition of claim 131, which is in frozen form.

133. The composition of claim 132, further comprising a cryoprotectant.

134. The composition of claim 132 or 133, wherein the RNA integrity after thawing the frozen composition is at least 50% compared to the RNA integrity of the composition before the composition has been frozen.

135. The composition of any one of claims 132 to 134, wherein the size (Z_{average}) and/or size distribution and/or polydispersity index (PDI) of RNA particles after thawing the frozen composition is essentially equal to the size (Z_{average}) and/or size distribution and/or PDI of the RNA particles before the composition has been frozen.

136. The composition of claim 131, which is in liquid form.

137. The composition of claim 136, wherein the RNA integrity after storage of the composition for at least 1 week is at least 50% compared to the RNA integrity before storage.

138. The composition of claim 136 or 137, wherein the size (Z_{average}) and/or size distribution and/or polydispersity index (PDI) of RNA particles after storage of the composition for at least one week is essentially equal to the size (Z_{average}) and/or size distribution and/or PDI of the RNA particles before storage.

139. A method for preparing a ready-to-use pharmaceutical composition, the method comprising the steps of providing a frozen composition prepared by the method of any one of claims 102 to 119, 121, 122, 123, and 125 to 128, and thawing the frozen composition thereby obtaining the ready-to-use pharmaceutical composition.

140. A method for preparing a ready-to-use pharmaceutical composition, the method comprising the step of providing a liquid composition prepared by the method of any one of claims 102 to 105, 107 to 121, 124, 125, 129, and 130, thereby obtaining the ready-to-use pharmaceutical composition.

141. A ready-to-use pharmaceutical composition preparable by the method of claim 139 or 140.

142. A composition of any one of claims 1 to 101, 131 to 138, and 141 for use in therapy.

143. A composition of any one of claims 1 to 101, 131 to 138, and 141 for use in inducing an immune response in a subject.
